SEQUENCE LISTING (259 sequences)

SEQ ID NO: 1 - DNA, 3162 bp, Artificial Sequence
  Synthetic
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 2 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 3 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 4 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 5 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 6 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 7 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 8 - protein, 1093 aa, Artificial Sequence
  Synthetic
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 9 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 10 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 11 - protein, 1098 aa, Artificial Sequence
  Synthetic
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 12 - RNA, 36 nt, Artificial Sequence
  Synthetic
guuggaaugacuaauuuuugugcccaccguuggcac

SEQ ID NO: 13 - RNA, 24 nt, Artificial Sequence
  Synthetic
aauuuuugugcccaucguuggcac

SEQ ID NO: 14 - RNA, 23 nt, Artificial Sequence
  Synthetic
auuuuugugcccaucguuggcac

SEQ ID NO: 15 - RNA, 36 nt, Artificial Sequence
  Synthetic
guugcaaaacccaagaaauccgucuuucauugacgg

SEQ ID NO: 16 - RNA, 34 nt, Artificial Sequence
  Synthetic
gcaacaccuaagaaauccgucuuucauugacggg

SEQ ID NO: 17 - RNA, 23 nt, Artificial Sequence
  Synthetic
agaaauccgucuuucauugacgg

SEQ ID NO: 18 - RNA, 36 nt, Artificial Sequence
  Synthetic
cuagcaaugaccuaauaguguguccuuaguugacau

SEQ ID NO: 19 - RNA, 36 nt, Artificial Sequence
  Synthetic
ccuacaauaccuaagaaauccguccuaaguugacgg

SEQ ID NO: 20 - RNA, 22 nt, Artificial Sequence
  Synthetic
auaguguguccuuaguugacau

SEQ ID NO: 21 - RNA, 36 nt, Artificial Sequence; identical to SEQ ID NO: 12
  Synthetic

SEQ ID NO: 22 - RNA, 36 nt, Artificial Sequence
  Synthetic
cccacaauaccugagaaauccguccuacguugacgg

SEQ ID NO: 23 - RNA, 36 nt, Artificial Sequence
  Synthetic
ucucaacgauagucagacauguguccucagugacac

SEQ ID NO: 24 - RNA, 22 nt, Artificial Sequence
  Synthetic
agacauguguccucagugacac

SEQ ID NO: 25 - protein, 1798 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 26 - protein, 1798 aa, Artificial Sequence
  Synthetic
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSKRPAATKKAGQAKKKK

SEQ ID NO: 27 - RNA, 43 nt, Artificial Sequence
  Synthetic
agaaauccgucuuucauugacgguuaaacucuccauggaccag

SEQ ID NO: 28 - RNA, 43 nt, Artificial Sequence; identical to SEQ ID NO: 27
  Synthetic
  misc_feature (1)..(4): modified with phosphorothioate
  misc_feature (40)..(43): modified with phosphorothioate

SEQ ID NO: 29 - protein, 678 aa, Artificial Sequence
  Synthetic
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 30 - DNA, 20 bp, Artificial Sequence
  Synthetic
gtagcctctcccgctctggt

SEQ ID NO: 31 - RNA, 43 nt, Artificial Sequence; identical to SEQ ID NO: 27
  Synthetic
  misc_feature (1)..(3): modified with 2′-O-methylation
  misc_feature (40)..(42): modified with 2′-O-methylation

SEQ ID NO: 32 - DNA, 20 bp, Artificial Sequence
  Synthetic
gggaagtggttggtcagcat

SEQ ID NO: 33 - RNA, 43 nt, Artificial Sequence; identical to SEQ ID NO: 27
  Synthetic
  misc_feature (1)..(4): modified with phosphorothioate
  misc_feature (1)..(3): modified with 2′-O-methylation
  misc_feature (40)..(42): modified with 2′-O-methylation
  misc_feature (40)..(43): modified with phosphorothioate

SEQ ID NO: 34 - DNA, 20 bp, Artificial Sequence
  Synthetic
gagccagtgttgctagtcaa

SEQ ID NO: 35 - RNA, 90 nt, Artificial Sequence
  Synthetic
agaaauccgucuuucauugacgguuaaacucuccauggaccagaaaagacuuuuuaaacucuccaaccuccaggcucauccagcuuccca

SEQ ID NO: 36 - DNA, 20 bp, Artificial Sequence
  Synthetic
aatcctccaccagtcatggt

SEQ ID NO: 37 - RNA, 90 nt, Artificial Sequence; identical to SEQ ID NO: 35
  Synthetic
  misc_feature (1)..(4): modified with phosphorothioate
  misc_feature (87)..(90): modified with phosphorothioate

SEQ ID NO: 38 - DNA, 20 bp, Artificial Sequence
  Synthetic
ttaaactctccatggaccag

SEQ ID NO: 39 - RNA, 90 nt, Artificial Sequence; identical to SEQ ID NO: 35
  Synthetic
  misc_feature (1)..(3): modified with 2′-O-methylation
  misc_feature (87)..(89): modified with 2′-O-methylation

SEQ ID NO: 40 - RNA, 137 nt, Artificial Sequence
  Synthetic
agaaauccgucuuucauugacggguagccucucccgcucuggugacauucuuuguagccucuccccgagugguucagggcccagcuagggauccagaucugggugauuuaggcucccucugucuggaucaguccucc

SEQ ID NO: 41 - RNA, 107 nt, Artificial Sequence
  Synthetic
agaaauccgucuuucauugacggguagccucucccgcucuggugacauucuuuguagccucuccccgagugguucagggcccagcuagggauccagaucugggugau

SEQ ID NO: 42 - RNA, 107 nt, Artificial Sequence
  Synthetic
agaaauccgucuuucauugacggguagccucucccgcucuggugcacccccauaaggggggacauucuuuguagccucuccccgagugguucagggcccagcuaggg

SEQ ID NO: 43 - RNA, 90 nt, Artificial Sequence
  Synthetic
agaaauccgucuuucauugacggguagccucucccgcucuggugacauucuuuguagccucuccccgagugguucagggcccagcuaggg

SEQ ID NO: 44 - RNA, 94 nt, Artificial Sequence
  Synthetic
agaaauccgucuuucauugacggguagccucucccgcucuggugacauucuuuguagccucucccagcggcucugguucagggcccagcuaggg

SEQ ID NO: 45 - RNA, 86 nt, Artificial Sequence
  Synthetic
agaaauccgucuuucauugacggguagccucucccgcucuggugacauucuuuguagccucucccugguucagggcccagcuaggg

SEQ ID NO: 46 - RNA, 90 nt, Artificial Sequence; identical to SEQ ID NO: 35
  Synthetic
  misc_feature (1)..(4): modified with phosphorothioate
  misc_feature (1)..(3): modified with 2′-O-methylation
  misc_feature (87)..(89): modified with 2′-O-methylation
  misc_feature (87)..(90): modified with phosphorothioate

SEQ ID NO: 47 - RNA, 107 nt, Artificial Sequence
  Synthetic
agaaauccgucuuucauugacgguuaaacucuccauggaccagaaaagacuuuuuaaacucuccaaccuccaggcucauccagcuucccaaacaaagcccccaagaa

SEQ ID NO: 48 - RNA, 107 nt, Artificial Sequence; identical to SEQ ID NO: 47
  Synthetic
  misc_feature (1)..(4): modified with phosphorothioate
  misc_feature (104)..(107): modified with phosphorothioate

SEQ ID NO: 49 - RNA, 107 nt, Artificial Sequence; identical to SEQ ID NO: 47
  Synthetic
  misc_feature (1)..(3): modified with 2′-O-methylation
  misc_feature (104)..(106): modified with 2′-O-methylation

SEQ ID NO: 50 - RNA, 107 nt, Artificial Sequence; identical to SEQ ID NO: 47
  Synthetic
  misc_feature (1)..(4): modified with phosphorothioate
  misc_feature (1)..(3): modified with 2′-O-methylation
  misc_feature (104)..(106): modified with 2′-O-methylation
  misc_feature (104)..(107): modified with phosphorothioate

SEQ ID NO: 51 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKAHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 52 - protein, 1832 aa, Artificial Sequence
  Synthetic
MKIEEGKGHHHHHHMASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKAHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQKKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 53 - protein, 1818 aa, Artificial Sequence
  Synthetic
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSMASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKAHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQKKRPAATKKAGQAKKKK

SEQ ID NO: 54 - RNA, 137 nt, Artificial Sequence
  Synthetic
agaaauccgucuuucauugacgggggaagugguuggucagcaucagcuacuuugggaagugguugcgaggcauggauuauagccgaaggccccagcuuugccuuguucuagcaguuccacuccugggcagcccgaga

SEQ ID NO: 55 - RNA, 107 nt, Artificial Sequence
  Synthetic
agaaauccgucuuucauugacgggggaagugguuggucagcaucagcuacuuugggaagugguugcgaggcauggauuauagccgaaggccccagcuuugccuuguu

SEQ ID NO: 56 - RNA, 107 nt, Artificial Sequence
  Synthetic
agaaauccgucuuucauugacgggggaagugguuggucagcaugcacccccauaagggggcagcuacuuugggaagugguugcgaggcauggauuauagccgaaggc

SEQ ID NO: 57 - RNA, 90 nt, Artificial Sequence
  Synthetic
agaaauccgucuuucauugacgggggaagugguuggucagcaucagcuacuuugggaagugguugcgaggcauggauuauagccgaaggc

SEQ ID NO: 58 - RNA, 94 nt, Artificial Sequence
  Synthetic
agaaauccgucuuucauugacgggggaagugguuggucagcaucagcuacuuugggaagugguugagcggucagcauggauuauagccgaaggc

SEQ ID NO: 59 - RNA, 86 nt, Artificial Sequence
  Synthetic
agaaauccgucuuucauugacgggggaagugguuggucagcaucagcuacuuugggaagugguuggcauggauuauagccgaaggc

SEQ ID NO: 60 - RNA, 42 nt, Artificial Sequence
  Synthetic
agacauguguccucagugacacgggaagugguuggucagcau

SEQ ID NO: 61 - RNA, 95 nt, Artificial Sequence
  Synthetic
agacauguguccucagugacacgggaagugguuggucagcauugauuccagcuacucugggaagugguugcagugcauggauuauagccgaaggc

SEQ ID NO: 62 - RNA, 149 nt, Artificial Sequence
  Synthetic
agaaauccgucuuucauugacgggcacacgacgauguaaucgccagcuacuaugggaagugguugcagugcauggauuauagccgaaggccccagcuuugccuuguuagaaauccgucuuucauugacgggacgcuacuauagcugcac

SEQ ID NO: 63 - RNA, 149 nt, Artificial Sequence
  Synthetic
agaaauccgucuuucauugacgggcacacgacgauguaaucgcuuccuucuaugagccaguguuggaucucaagggcagcaugcugggcccgucccacuacaggccaagaaauccgucuuucauugacgggacgcuacuauagcugcac

SEQ ID NO: 64 - RNA, 149 nt, Artificial Sequence
  Synthetic
agaaauccgucuuucauugacgggcacacgacgauguaaucgcagauaccuauaauccuccaccacaguuggugacaaccccaagcagcccacacauucucaagugcagaaauccgucuuucauugacgggacgcuacuauagcugcac

SEQ ID NO: 65 - DNA, 57 bp, Artificial Sequence
  Synthetic
gattccagctactttgggaagtggttggtcagcatggattatagccgaaggccccag

SEQ ID NO: 66 - DNA, 57 bp, Artificial Sequence
  Synthetic
ctaaggtcgatgaaacccttcaccaaccagtcgtacctaatatcggcttccggggtc

SEQ ID NO: 67 - RNA, 90 nt, Artificial Sequence
  Synthetic
agaaauccgucuuucauugacgggggaagugguuggucagcaucagcuacuuugggaagugguuggucagcauggauuauagccgaaggc

SEQ ID NO: 68 - RNA, 137 nt, Artificial Sequence
  Synthetic
agaaauccgucuuucauugacgggagccaguguugcuagucaauuccuucuuugagccaguguugcgagucaagggcagcaugcugggcccgucccacuacaggccaaugugaccgucagucuccuuccugaaggac

SEQ ID NO: 69 - RNA, 107 nt, Artificial Sequence
  Synthetic
agaaauccgucuuucauugacgggagccaguguugcuagucaauuccuucuuugagccaguguugcgagucaagggcagcaugcugggcccgucccacuacaggcca

SEQ ID NO: 70 - RNA, 107 nt, Artificial Sequence
  Synthetic
agaaauccgucuuucauugacgggagccaguguugcuagucaagcacccccauaaggggguuccuucuuugagccaguguugcgagucaagggcagcaugcugggcc

SEQ ID NO: 71 - RNA, 90 nt, Artificial Sequence
  Synthetic
agaaauccgucuuucauugacgggagccaguguugcuagucaauuccuucuuugagccaguguugcgagucaagggcagcaugcugggcc

SEQ ID NO: 72 - RNA, 94 nt, Artificial Sequence
  Synthetic
agaaauccgucuuucauugacgggagccaguguugcuagucaauuccuucuuugagccaguguugagcgcuagucaagggcagcaugcugggcc

SEQ ID NO: 73 - RNA, 86 nt, Artificial Sequence
  Synthetic
agaaauccgucuuucauugacgggagccaguguugcuagucaauuccuucuuugagccaguguugucaagggcagcaugcugggcc

SEQ ID NO: 74 - DNA, 20 bp, Artificial Sequence
  Synthetic
gggaagtggttggtcagcat

SEQ ID NO: 75 - RNA, 90 nt, Artificial Sequence; identical to SEQ ID NO: 67
  Synthetic

SEQ ID NO: 76 - RNA, 90 nt, Artificial Sequence; identical to SEQ ID NO: 57
  Synthetic

SEQ ID NO: 77 - RNA, 94 nt, Artificial Sequence; identical to SEQ ID NO: 58
  Synthetic

SEQ ID NO: 78 - RNA, 86 nt, Artificial Sequence; identical to SEQ ID NO: 59
  Synthetic

SEQ ID NO: 79 - RNA, 107 nt, Artificial Sequence; identical to SEQ ID NO: 56
  Synthetic

SEQ ID NO: 80 - RNA, 90 nt, Artificial Sequence; identical to SEQ ID NO: 57
  Synthetic

SEQ ID NO: 81 - RNA, 137 nt, Artificial Sequence
  Synthetic
agaaauccgucuuucauugacggaauccuccaccagucaugguagauaccuuuaauccuccaccacgaguggugacaaccccaagcagcccacacauuuucaagugcccccaggaugcguggagggaggggucugug

SEQ ID NO: 82 - RNA, 107 nt, Artificial Sequence
  Synthetic
agaaauccgucuuucauugacggaauccuccaccagucaugguagauaccuuuaauccuccaccacgaguggugacaaccccaagcagcccacacauuuucaagugc

SEQ ID NO: 83 - RNA, 107 nt, Artificial Sequence
  Synthetic
agaaauccgucuuucauugacggaauccuccaccagucauggugcacccccauaagggggagauaccuuuaauccuccaccacgaguggugacaaccccaagcagcc

SEQ ID NO: 84 - RNA, 90 nt, Artificial Sequence
  Synthetic
agaaauccgucuuucauugacggaauccuccaccagucaugguagauaccuuuaauccuccaccacgaguggugacaaccccaagcagcc

SEQ ID NO: 85 - RNA, 94 nt, Artificial Sequence
  Synthetic
agaaauccgucuuucauugacggaauccuccaccagucaugguagauaccuuuaauccuccaccaagcggucauggugacaaccccaagcagcc

SEQ ID NO: 86 - RNA, 86 nt, Artificial Sequence
  Synthetic
agaaauccgucuuucauugacggaauccuccaccagucaugguagauaccuuuaauccuccaccauggugacaaccccaagcagcc

SEQ ID NO: 87 - RNA, 90 nt, Artificial Sequence; identical to SEQ ID NO: 57
  Synthetic

SEQ ID NO: 88 - DNA, 96 bp, Artificial Sequence
  Synthetic
ugggaagugguuggucagcagttttagagctagaaatagcaagttaaaataaggctagtccgttatcaacttgaaaaagtggcaccgagtcggtgc

SEQ ID NO: 89 - RNA, 29 nt, Artificial Sequence
  Synthetic
ggcuauaauccaugcaagugaccaaccac

SEQ ID NO: 90 - DNA, 20 bp, Artificial Sequence
  Synthetic
gggaagtggttgcagtgcat

SEQ ID NO: 91 - DNA, 20 bp, Artificial Sequence
  Synthetic
gagccagtgttggatctcaa

SEQ ID NO: 92 - DNA, 20 bp, Artificial Sequence
  Synthetic
ttaaactctccaacctccag

SEQ ID NO: 93 - RNA, 20 nt, Artificial Sequence
  Synthetic
uaauuucuacuguuguagau

SEQ ID NO: 94 - RNA, 21 nt, Artificial Sequence
  Synthetic
agaaaugcaugguucucaugc

SEQ ID NO: 95 - RNA, 137 nt, Artificial Sequence
  Synthetic
agaaauccgucuuucauugacgguuaaacucuccauggaccagaaaagacuuuuuaaacucuccacgagccaggcucauccagcuucccaaacaaagcccccaagaagggggggcacucaggacucucuccaagaga

SEQ ID NO: 96 - RNA, 107 nt, Artificial Sequence
  Synthetic
agaaauccgucuuucauugacgguuaaacucuccauggaccagaaaagacuuuuuaaacucuccacgagccaggcucauccagcuucccaaacaaagcccccaagaa

SEQ ID NO: 97 - RNA, 107 nt, Artificial Sequence
  Synthetic
agaaauccgucuuucauugacgguuaaacucuccauggaccaggcacccccauaagggggaaaagacuuuuuaaacucuccacgagccaggcucauccagcuuccca

SEQ ID NO: 98 - RNA, 90 nt, Artificial Sequence
  Synthetic
agaaauccgucuuucauugacgguuaaacucuccauggaccagaaaagacuguuuaaacucuccacgagccaggcucauccagcuuccca

SEQ ID NO: 99 - RNA, 94 nt, Artificial Sequence
  Synthetic
agaaauccgucuuucauugacgguuaaacucuccauggaccagaaaagacuguuuaaacucuccaagcguggaccaggcucauccagcuuccca

SEQ ID NO: 100 - RNA, 86 nt, Artificial Sequence
  Synthetic
agaaauccgucuuucauugacgguuaaacucuccauggaccagaaaagacuguuuaaacucuccaccaggcucauccagcuuccca

SEQ ID NO: 101 - RNA, 21 nt, Artificial Sequence
  Synthetic
aaaauuaccuaguaauuaggu

SEQ ID NO: 102 - RNA, 20 nt, Artificial Sequence
  Synthetic
ggauuucuacuuuuguagau

SEQ ID NO: 103 - RNA, 20 nt, Artificial Sequence
  Synthetic
aaauuucuacuuuuguagau

SEQ ID NO: 104 - RNA, 21 nt, Artificial Sequence
  Synthetic
cgcgcccacgcggggcgcgac

SEQ ID NO: 105 - RNA, 20 nt, Artificial Sequence
  Synthetic
uaauuucuacucuuguagau

SEQ ID NO: 106 - RNA, 20 nt, Artificial Sequence
  Synthetic
gaauuucuacuauuguagau

SEQ ID NO: 107 - RNA, 21 nt, Artificial Sequence
  Synthetic
gaaucucuacucuuuguagau

SEQ ID NO: 108 - RNA, 19 nt, Artificial Sequence
  Synthetic
uaauuucuacuuuguagau

SEQ ID NO: 109 - RNA, 21 nt, Artificial Sequence
  Synthetic
aaauuucuacuguuuguagau

SEQ ID NO: 110 - RNA, 43 nt, Artificial Sequence
  Synthetic
agaaauccgucuuucauugacggguagccucucccgcucuggu

SEQ ID NO: 111 - RNA, 20 nt, Artificial Sequence
  Synthetic
gaauuucuacuuuuguagau

SEQ ID NO: 112 - RNA, 43 nt, Artificial Sequence
  Synthetic
agaaauccgucuuucauugacgggggaagugguuggucagcau

SEQ ID NO: 113 - RNA, 21 nt, Artificial Sequence
  Synthetic
uaauuucuacuaaguguagau

SEQ ID NO: 114 - RNA, 43 nt, Artificial Sequence
  Synthetic
agaaauccgucuuucauugacgggagccaguguugcuagucaa

SEQ ID NO: 115 - RNA, 20 nt, Artificial Sequence
  Synthetic
uaauuucuacuauuguagau

SEQ ID NO: 116 - RNA, 43 nt, Artificial Sequence
  Synthetic
agaaauccgucuuucauugacggaauccuccaccagucauggu

SEQ ID NO: 117 - RNA, 20 nt, Artificial Sequence
  Synthetic
uaauuucuacuucgguagau

SEQ ID NO: 118 - RNA, 43 nt, Artificial Sequence; identical to SEQ ID NO: 27
  Synthetic

SEQ ID NO: 119 - RNA, 20 nt, Artificial Sequence
  Synthetic
uaauuucuacuauuguagau

SEQ ID NO: 120 - protein, 1368 aa, Artificial Sequence [sequence omitted]

SEQ ID NO: 121 - protein, 1368 aa, Artificial Sequence
  Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 122 - protein, 1053 aa, Artificial Sequence
  Synthetic
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 123 - RNA, 90 nt, Artificial Sequence
  Synthetic
agaaauccgucuuucauugacgggggaagugguuggucagcaucagcuacuaugggaagugguugcagugcauggauuauagccgaaggc

SEQ ID NO: 124 - RNA, 107 nt, Artificial Sequence
  Synthetic
agaaauccgucuuucauugacgggggaagugguuggucagcaucagcuacuaugggaagugguugcagugcauggauuauagccgaaggccccagcuuugccuuguu

SEQ ID NO: 125 - RNA, 137 nt, Artificial Sequence
  Synthetic
agaaauccgucuuucauugacgggggaagugguuggucagcaucagcuacuaugggaagugguugcagugcauggauuauagccgaaggccccagcuuugccuuguucuagcaguuccacuccugggcagcccgaga

SEQ ID NO: 126 - RNA, 133 nt, Artificial Sequence
  Synthetic
agaaauccgucuuucauugacgggggaagugguuggucagcaucagcuacuaugggaagugguugcagugcauggauuauagccgaaggcagaaauccgucuuucauugacgggcacacgacgauguaaucgc

SEQ ID NO: 127 - RNA, 150 nt, Artificial Sequence
  Synthetic
agaaauccgucuuucauugacgggggaagugguuggucagcaucagcuacuaugggaagugguugcagugcauggauuauagccgaaggccccagcuuugccuuguuagaaauccgucuuucauugacgggcacacgacgauguaaucgc

SEQ ID NO: 128 - RNA, 180 nt, Artificial Sequence
  Synthetic
agaaauccgucuuucauugacgggggaagugguuggucagcaucagcuacuaugggaagugguugcagugcauggauuauagccgaaggccccagcuuugccuuguucuagcaguuccacuccugggcagcccgagaagaaauccgucuuucauugacgggcacacgacgauguaaucgc

SEQ ID NO: 129 - RNA, 90 nt, Artificial Sequence
  Synthetic
cagcuacuaugggaagugguugcagugcauggauuauagccgaaggcagaaauccgucuuucauugacgggggaagugguuggucagcau

SEQ ID NO: 130 - RNA, 107 nt, Artificial Sequence
  Synthetic
cagcuacuaugggaagugguugcagugcauggauuauagccgaaggccccagcuuugccuuguuagaaauccgucuuucauugacgggggaagugguuggucagcau

SEQ ID NO: 131 - RNA, 137 nt, Artificial Sequence
  Synthetic
cagcuacuaugggaagugguugcagugcauggauuauagccgaaggccccagcuuugccuuguucuagcaguuccacuccugggcagcccgagaagaaauccgucuuucauugacgggggaagugguuggucagcau

SEQ ID NO: 132 - RNA, 133 nt, Artificial Sequence
  Synthetic
agaaauccgucuuucauugacgggcacacgacgauguaaucgccagcuacuaugggaagugguugcagugcauggauuauagccgaaggcagaaauccgucuuucauugacgggggaagugguuggucagcau

SEQ ID NO: 133 - RNA, 150 nt, Artificial Sequence
  Synthetic
agaaauccgucuuucauugacgggcacacgacgauguaaucgccagcuacuaugggaagugguugcagugcauggauuauagccgaaggccccagcuuugccuuguuagaaauccgucuuucauugacgggggaagugguuggucagcau

SEQ ID NO: 134 - RNA, 180 nt, Artificial Sequence
  Synthetic
agaaauccgucuuucauugacgggcacacgacgauguaaucgccagcuacuaugggaagugguugcagugcauggauuauagccgaaggccccagcuuugccuuguucuagcaguuccacuccugggcagcccgagaagaaauccgucuuucauugacgggggaagugguuggucagcau

SEQ ID NO: 135 - RNA, 139 nt, Artificial Sequence
  Synthetic
agaaauccgucuuucauugacgggcacacgacgauguaaucgcugauuccagcuacuaugggaagugguugcagugcauggauuauagccgaaggcagaaauccgucuuucauugacgggggaagugguuggucagcau

SEQ ID NO: 136 - RNA, 156 nt, Artificial Sequence
  Synthetic
agaaauccgucuuucauugacgggcacacgacgauguaaucgcugauuccagcuacuaugggaagugguugcagugcauggauuauagccgaaggccccagcuuugccuuguuagaaauccgucuuucauugacgggggaagugguuggucagcau

SEQ ID NO: 137 - RNA, 186 nt, Artificial Sequence
  Synthetic
agaaauccgucuuucauugacgggcacacgacgauguaaucgcugauuccagcuacuaugggaagugguugcagugcauggauuauagccgaaggccccagcuuugccuuguucuagcaguuccacuccugggcagcccgagaagaaauccgucuuucauugacgggggaagugguuggucagcau

SEQ ID NO: 138 - RNA, 90 nt, Artificial Sequence
  Synthetic
agaaauccgucuuucauugacgggagccaguguugcuagucaauuccuucuaugagccaguguuggaucucaagggcagcaugcugggcc

SEQ ID NO: 139 - RNA, 107 nt, Artificial Sequence
  Synthetic
agaaauccgucuuucauugacgggagccaguguugcuagucaauuccuucuaugagccaguguuggaucucaagggcagcaugcugggcccgucccacuacaggcca

SEQ ID NO: 140 - RNA, 137 nt, Artificial Sequence
  Synthetic
agaaauccgucuuucauugacgggagccaguguugcuagucaauuccuucuaugagccaguguuggaucucaagggcagcaugcugggcccgucccacuacaggccaaugugaccgucagucuccuuccugaaggac

SEQ ID NO: 141 - RNA, 133 nt, Artificial Sequence
  Synthetic
agaaauccgucuuucauugacgggagccaguguugcuagucaauuccuucuaugagccaguguuggaucucaagggcagcaugcugggccagaaauccgucuuucauugacgggcacacgacgauguaaucgc

SEQ ID NO: 142 - RNA, 150 nt, Artificial Sequence
  Synthetic
agaaauccgucuuucauugacgggagccaguguugcuagucaauuccuucuaugagccaguguuggaucucaagggcagcaugcugggcccgucccacuacaggccaagaaauccgucuuucauugacgggcacacgacgauguaaucgc

SEQ ID NO: 143 - RNA, 180 nt, Artificial Sequence
  Synthetic
agaaauccgucuuucauugacgggagccaguguugcuagucaauuccuucuaugagccaguguuggaucucaagggcagcaugcugggcccgucccacuacaggccaaugugaccgucagucuccuuccugaaggacagaaauccgucuuucauugacgggcacacgacgauguaaucgc

SEQ ID NO: 144 - RNA, 90 nt, Artificial Sequence
  Synthetic
uuccuucuaugagccaguguuggaucucaagggcagcaugcugggccagaaauccgucuuucauugacgggagccaguguugcuagucaa

SEQ ID NO: 145 - RNA, 107 nt, Artificial Sequence
  Synthetic
uuccuucuaugagccaguguuggaucucaagggcagcaugcugggcccgucccacuacaggccaagaaauccgucuuucauugacgggagccaguguugcuagucaa

SEQ ID NO: 146 - RNA, 137 nt, Artificial Sequence
  Synthetic
uuccuucuaugagccaguguuggaucucaagggcagcaugcugggcccgucccacuacaggccaaugugaccgucagucuccuuccugaaggacagaaauccgucuuucauugacgggagccaguguugcuagucaa

SEQ ID NO: 147 - RNA, 133 nt, Artificial Sequence
  Synthetic
agaaauccgucuuucauugacgggcacacgacgauguaaucgcuuccuucuaugagccaguguuggaucucaagggcagcaugcugggccagaaauccgucuuucauugacgggagccaguguugcuagucaa

SEQ ID NO: 148 - RNA, 150 nt, Artificial Sequence
  Synthetic
agaaauccgucuuucauugacgggcacacgacgauguaaucgcuuccuucuaugagccaguguuggaucucaagggcagcaugcugggcccgucccacuacaggccaagaaauccgucuuucauugacgggagccaguguugcuagucaa

SEQ ID NO: 149 - RNA, 180 nt, Artificial Sequence
  Synthetic
agaaauccgucuuucauugacgggcacacgacgauguaaucgcuuccuucuaugagccaguguuggaucucaagggcagcaugcugggcccgucccacuacaggccaaugugaccgucagucuccuuccugaaggacagaaauccgucuuucauugacgggagccaguguugcuagucaa

SEQ ID NO: 150 - RNA, 139 nt, Artificial Sequence
  Synthetic
agaaauccgucuuucauugacgggcacacgacgauguaaucgcaaagcuuuccuucuaugagccaguguuggaucucaagggcagcaugcugggccagaaauccgucuuucauugacgggagccaguguugcuagucaa

SEQ ID NO: 151 - RNA, 156 nt, Artificial Sequence
  Synthetic
agaaauccgucuuucauugacgggcacacgacgauguaaucgcaaagcuuuccuucuaugagccaguguuggaucucaagggcagcaugcugggcccgucccacuacaggccaagaaauccgucuuucauugacgggagccaguguugcuagucaa

SEQ ID NO: 152 - RNA, 186 nt, Artificial Sequence
  Synthetic
agaaauccgucuuucauugacgggcacacgacgauguaaucgcaaagcuuuccuucuaugagccaguguuggaucucaagggcagcaugcugggcccgucccacuacaggccaaugugaccgucagucuccuuccugaaggacagaaauccgucuuucauugacgggagccaguguugcuagucaa

SEQ ID NO: 153 - RNA, 90 nt, Artificial Sequence
  Synthetic
agaaauccgucuuucauugacggaauccuccaccagucaugguagauaccuauaauccuccaccacaguuggugacaaccccaagcagcc

SEQ ID NO: 154 - RNA, 107 nt, Artificial Sequence
  Synthetic
agaaauccgucuuucauugacggaauccuccaccagucaugguagauaccuauaauccuccaccacaguuggugacaaccccaagcagcccacacauucucaagugc

SEQ ID NO: 155 - RNA, 137 nt, Artificial Sequence
  Synthetic
agaaauccgucuuucauugacggaauccuccaccagucaugguagauaccuauaauccuccaccacaguuggugacaaccccaagcagcccacacauucucaagugcccccaggaugcguggagggaggggucugug

SEQ ID NO: 156 - RNA, 133 nt, Artificial Sequence
  Synthetic
agaaauccgucuuucauugacggaauccuccaccagucaugguagauaccuauaauccuccaccacaguuggugacaaccccaagcagccagaaauccgucuuucauugacgggcacacgacgauguaaucgc

SEQ ID NO: 157 - RNA, 150 nt, Artificial Sequence
  Synthetic
agaaauccgucuuucauugacggaauccuccaccagucaugguagauaccuauaauccuccaccacaguuggugacaaccccaagcagcccacacauucucaagugcagaaauccgucuuucauugacgggcacacgacgauguaaucgc

SEQ ID NO: 158 - RNA, 180 nt, Artificial Sequence
  Synthetic
agaaauccgucuuucauugacggaauccuccaccagucaugguagauaccuauaauccuccaccacaguuggugacaaccccaagcagcccacacauucucaagugcccccaggaugcguggagggaggggucugugagaaauccgucuuucauugacgggcacacgacgauguaaucgc

SEQ ID NO: 159 - RNA, 90 nt, Artificial Sequence
  Synthetic
agauaccuauaauccuccaccacaguuggugacaaccccaagcagccagaaauccgucuuucauugacggaauccuccaccagucauggu

SEQ ID NO: 160 - RNA, 107 nt, Artificial Sequence
  Synthetic
agauaccuauaauccuccaccacaguuggugacaaccccaagcagcccacacauucucaagugcagaaauccgucuuucauugacggaauccuccaccagucauggu

SEQ ID NO: 161 - RNA, 137 nt, Artificial Sequence
  Synthetic
agauaccuauaauccuccaccacaguuggugacaaccccaagcagcccacacauucucaagugcccccaggaugcguggagggaggggucugugagaaauccgucuuucauugacggaauccuccaccagucauggu

SEQ ID NO: 162 - RNA, 133 nt, Artificial Sequence
  Synthetic
agaaauccgucuuucauugacgggcacacgacgauguaaucgcagauaccuauaauccuccaccacaguuggugacaaccccaagcagccagaaauccgucuuucauugacggaauccuccaccagucauggu

SEQ ID NO: 163 - RNA, 150 nt, Artificial Sequence
  Synthetic
agaaauccgucuuucauugacgggcacacgacgauguaaucgcagauaccuauaauccuccaccacaguuggugacaaccccaagcagcccacacauucucaagugcagaaauccgucuuucauugacggaauccuccaccagucauggu

SEQ ID NO: 164 - RNA, 180 nt, Artificial Sequence
  Synthetic
agaaauccgucuuucauugacgggcacacgacgauguaaucgcagauaccuauaauccuccaccacaguuggugacaaccccaagcagcccacacauucucaagugcccccaggaugcguggagggaggggucugugagaaauccgucuuucauugacggaauccuccaccagucauggu

SEQ ID NO: 165 - RNA, 139 nt, Artificial Sequence
  Synthetic
agaaauccgucuuucauugacgggcacacgacgauguaaucgcaauacuagauaccuauaauccuccaccacaguuggugacaaccccaagcagccagaaauccgucuuucauugacggaauccuccaccagucauggu

SEQ ID NO: 166 - RNA, 156 nt, Artificial Sequence
  Synthetic
agaaauccgucuuucauugacgggcacacgacgauguaaucgcaauacuagauaccuauaauccuccaccacaguuggugacaaccccaagcagcccacacauucucaagugcagaaauccgucuuucauugacggaauccuccaccagucauggu

SEQ ID NO: 167 - RNA, 186 nt, Artificial Sequence
  Synthetic
agaaauccgucuuucauugacgggcacacgacgauguaaucgcaauacuagauaccuauaauccuccaccacaguuggugacaaccccaagcagcccacacauucucaagugcccccaggaugcguggagggaggggucugugagaaauccgucuuucauugacggaauccuccaccagucauggu

SEQ ID NO: 168 - RNA, 90 nt, Artificial Sequence
  Synthetic
agaaauccgucuuucauugacgguuaaacucuccauggaccagaaaagacuacuuaaacucuccaaccuccaggcucauccagcuuccca

SEQ ID NO: 169 - RNA, 107 nt, Artificial Sequence
  Synthetic
agaaauccgucuuucauugacgguuaaacucuccauggaccagaaaagacuacuuaaacucuccaaccuccaggcucauccagcuucccaaacaaagcccccaagaa

SEQ ID NO: 170 - RNA, 137 nt, Artificial Sequence
  Synthetic
agaaauccgucuuucauugacgguuaaacucuccauggaccagaaaagacuacuuaaacucuccaaccuccaggcucauccagcuucccaaacaaagcccccaagaagggggggcacucaggacucucuccaagaga

SEQ ID NO: 171 - RNA, 133 nt, Artificial Sequence
  Synthetic
agaaauccgucuuucauugacgguuaaacucuccauggaccagaaaagacuacuuaaacucuccaaccuccaggcucauccagcuucccaagaaauccgucuuucauugacgggcacacgacgauguaaucgc

SEQ ID NO: 172 - RNA, 150 nt, Artificial Sequence
  Synthetic
agaaauccgucuuucauugacgguuaaacucuccauggaccagaaaagacuacuuaaacucuccaaccuccaggcucauccagcuucccaaacaaagcccccaagaaagaaauccgucuuucauugacgggcacacgacgauguaaucgc

SEQ ID NO: 173 - RNA, 180 nt, Artificial Sequence
  Synthetic
agaaauccgucuuucauugacgguuaaacucuccauggaccagaaaagacuacuuaaacucuccaaccuccaggcucauccagcuucccaaacaaagcccccaagaagggggggcacucaggacucucuccaagagaagaaauccgucuuucauugacgggcacacgacgauguaaucgc

SEQ ID NO: 174 - RNA, 90 nt, Artificial Sequence
  Synthetic
aaaagacuacuuaaacucuccaaccuccaggcucauccagcuucccaagaaauccgucuuucauugacgguuaaacucuccauggaccag

SEQ ID NO: 175 - RNA, 107 nt, Artificial Sequence
  Synthetic
aaaagacuacuuaaacucuccaaccuccaggcucauccagcuucccaaacaaagcccccaagaaagaaauccgucuuucauugacgguuaaacucuccauggaccag

SEQ ID NO: 176 - RNA, 137 nt, Artificial Sequence
  Synthetic
aaaagacuacuuaaacucuccaaccuccaggcucauccagcuucccaaacaaagcccccaagaagggggggcacucaggacucucuccaagagaagaaauccgucuuucauugacgguuaaacucuccauggaccag

SEQ ID NO: 177 - RNA, 133 nt, Artificial Sequence
  Synthetic
agaaauccgucuuucauugacgggcacacgacgauguaaucgcaaaagacuacuuaaacucuccaaccuccaggcucauccagcuucccaagaaauccgucuuucauugacgguuaaacucuccauggaccag

SEQ ID NO: 178 - RNA, 150 nt, Artificial Sequence
  Synthetic
agaaauccgucuuucauugacgggcacacgacgauguaaucgcaaaagacuacuuaaacucuccaaccuccaggcucauccagcuucccaaacaaagcccccaagaaagaaauccgucuuucauugacgguuaaacucuccauggaccag

SEQ ID NO: 179 - RNA, 180 nt, Artificial Sequence
  Synthetic
agaaauccgucuuucauugacgggcacacgacgauguaaucgcaaaagacuacuuaaacucuccaaccuccaggcucauccagcuucccaaacaaagcccccaagaagggggggcacucaggacucucuccaagagaagaaauccgucuuucauugacgguuaaacucuccauggaccag

SEQ ID NO: 180 - RNA, 139 nt, Artificial Sequence
  Synthetic
agaaauccgucuuucauugacgggcacacgacgauguaaucgcaacaccaaaagacuacuuaaacucuccaaccuccaggcucauccagcuucccaagaaauccgucuuucauugacgguuaaacucuccauggaccag

SEQ ID NO: 181 - RNA, 156 nt, Artificial Sequence
  Synthetic
agaaauccgucuuucauugacgggcacacgacgauguaaucgcaacaccaaaagacuacuuaaacucuccaaccuccaggcucauccagcuucccaaacaaagcccccaagaaagaaauccgucuuucauugacgguuaaacucuccauggaccag

SEQ ID NO: 182 - RNA, 186 nt, Artificial Sequence
  Synthetic
agaaauccgucuuucauugacgggcacacgacgauguaaucgcaacaccaaaagacuacuuaaacucuccaaccuccaggcucauccagcuucccaaacaaagcccccaagaagggggggcacucaggacucucuccaagagaagaaauccgucuuucauugacgguuaaacucuccauggaccag

SEQ ID NO: 183 - RNA, 80 nt, Artificial Sequence
  Synthetic
agaaauccgucuuucauugacgggggaagugguuggucagcaucagcuacuuugggaagugguugcagugcauggauuau

SEQ ID NO: 184 - RNA, 85 nt, Artificial Sequence
  Synthetic
agaaauccgucuuucauugacgggggaagugguuggucagcaucagcuacuuugggaagugguugcagugcauggauuauagccg

SEQ ID NO: 185 - RNA, 90 nt, Artificial Sequence
  Synthetic
agaaauccgucuuucauugacgggggaagugguuggucagcaucagcuacuuugggaagugguugcagugcauggauuauagccgaaggc

SEQ ID NO: 186 - RNA, 107 nt, Artificial Sequence
  Synthetic
agaaauccgucuuucauugacgggggaagugguuggucagcaucagcuacuuugggaagugguugcagugcauggauuauagccgaaggccccagcuuugccuuguu

SEQ ID NO: 187 - RNA, 137 nt, Artificial Sequence
  Synthetic
agaaauccgucuuucauugacgggggaagugguuggucagcaucagcuacuuugggaagugguugcagugcauggauuauagccgaaggccccagcuuugccuuguucuagcaguuccacuccugggcagcccgaga

SEQ ID NO: 188 - RNA, 70 nt, Artificial Sequence
  Synthetic
agaaauccgucuuucauugacgggggaagugguuggucagcauugcagugcauggauuauagccgaaggc

SEQ ID NO: 189 - RNA, 80 nt, Artificial Sequence
  Synthetic
agaaauccgucuuucauugacgggggaagugguuggucagcaugggaagugguugcagugcauggauuauagccgaaggc

SEQ ID NO: 190 - RNA, 90 nt, Artificial Sequence; identical to SEQ ID NO: 185
  Synthetic

SEQ ID NO: 191 - RNA, 100 nt, Artificial Sequence
  Synthetic
agaaauccgucuuucauugacgggggaagugguuggucagcaugugaugauuccagcuacuuugggaagugguugcagugcauggauuauagccgaaggc

SEQ ID NO: 192 - RNA, 110 nt, Artificial Sequence
  Synthetic
agaaauccgucuuucauugacgggggaagugguuggucagcauggugggaaaggugaugauuccagcuacuuugggaagugguugcagugcauggauuauagccgaaggc

SEQ ID NO: 193 - RNA, 80 nt, Artificial Sequence
  Synthetic
agaaauccgucuuucauugacgggagccaguguugcuagucaauuccuucuuugagccaguguuggaucucaagggcagc

SEQ ID NO: 194 - RNA, 85 nt, Artificial Sequence
  Synthetic
agaaauccgucuuucauugacgggagccaguguugcuagucaauuccuucuuugagccaguguuggaucucaagggcagcaugcu

SEQ ID NO: 195 - RNA, 90 nt, Artificial Sequence
  Synthetic
agaaauccgucuuucauugacgggagccaguguugcuagucaauuccuucuuugagccaguguuggaucucaagggcagcaugcugggcc

SEQ ID NO: 196 - RNA, 107 nt, Artificial Sequence
  Synthetic
agaaauccgucuuucauugacgggagccaguguugcuagucaauuccuucuuugagccaguguuggaucucaagggcagcaugcugggcccgucccacuacaggcca

SEQ ID NO: 197 - RNA, 137 nt, Artificial Sequence
  Synthetic
agaaauccgucuuucauugacgggagccaguguugcuagucaauuccuucuuugagccaguguuggaucucaagggcagcaugcugggcccgucccacuacaggccaaugugaccgucagucuccuuccugaaggac

SEQ ID NO: 198 - RNA, 70 nt, Artificial Sequence
  Synthetic
agaaauccgucuuucauugacgggagccaguguugcuagucaauggaucucaagggcagcaugcugggcc

SEQ ID NO: 199 - RNA, 80 nt, Artificial Sequence
  Synthetic
agaaauccgucuuucauugacgggagccaguguugcuagucaagagccaguguuggaucucaagggcagcaugcugggcc

SEQ ID NO: 200 - RNA, 90 nt, Artificial Sequence; identical to SEQ ID NO: 195
  Synthetic

SEQ ID NO: 201 - RNA, 100 nt, Artificial Sequence
  Synthetic
agaaauccgucuuucauugacgggagccaguguugcuagucaaauccaaagcuuuccuucuuugagccaguguuggaucucaagggcagcaugcugggcc

SEQ ID NO: 202 - RNA, 110 nt, Artificial Sequence
  Synthetic
agaaauccgucuuucauugacgggagccaguguugcuagucaaugaagucgccauccaaagcuuuccuucuuugagccaguguuggaucucaagggcagcaugcugggcc

SEQ ID NO: 203 - RNA, 80 nt, Artificial Sequence
  Synthetic
agaaauccgucuuucauugacgguuaaacucuccauggaccagaaaagacuucuuaaacucuccaaccuccaggcucauc

SEQ ID NO: 204 - RNA, 85 nt, Artificial Sequence
  Synthetic
agaaauccgucuuucauugacgguuaaacucuccauggaccagaaaagacuucuuaaacucuccaaccuccaggcucauccagcu

SEQ ID NO: 205 - RNA, 90 nt, Artificial Sequence
  Synthetic
agaaauccgucuuucauugacgguuaaacucuccauggaccagaaaagacuucuuaaacucuccaaccuccaggcucauccagcuuccca

SEQ ID NO: 206 - RNA, 107 nt, Artificial Sequence
  Synthetic
agaaauccgucuuucauugacgguuaaacucuccauggaccagaaaagacuucuuaaacucuccaaccuccaggcucauccagcuucccaaacaaagcccccaagaa

SEQ ID NO: 207 - RNA, 137 nt, Artificial Sequence
  Synthetic
agaaauccgucuuucauugacgguuaaacucuccauggaccagaaaagacuucuuaaacucuccaaccuccaggcucauccagcuucccaaacaaagcccccaagaagggggggcacucaggacucucuccaagaga

SEQ ID NO: 208 - RNA, 70 nt, Artificial Sequence
  Synthetic
agaaauccgucuuucauugacgguuaaacucuccauggaccagcaaccuccaggcucauccagcuuccca

SEQ ID NO: 209 - RNA, 80 nt, Artificial Sequence
  Synthetic
agaaauccgucuuucauugacgguuaaacucuccauggaccaguuaaacucuccaaccuccaggcucauccagcuuccca

SEQ ID NO: 210 - RNA, 90 nt, Artificial Sequence; identical to SEQ ID NO: 205
  Synthetic

SEQ ID NO: 211 - RNA, 100 nt, Artificial Sequence
  Synthetic
agaaauccgucuuucauugacgguuaaacucuccauggaccagagguaacaccaaaagacuucuuaaacucuccaaccuccaggcucauccagcuuccca

SEQ ID NO: 212 - RNA, 110 nt, Artificial Sequence
  Synthetic
agaaauccgucuuucauugacgguuaaacucuccauggaccagccccauuaccagguaacaccaaaagacuucuuaaacucuccaaccuccaggcucauccagcuuccca

SEQ ID NO: 213 - DNA, 141 bp, Artificial Sequence
  Synthetic
tccatgtctcgttatacgctgtggttcgccaacaacactagtactactgataaattaccccccaagtccctcacctctccaaagctgcccatactactacactagtttgacagctagctcagtcctaggtataatgctagc

SEQ ID NO: 214 - DNA, 141 bp, Artificial Sequence
  Synthetic
tccatgtctcgttatacgctgtggttcgccaacatatagttcactactagcatgctgcccttgactagcaacactggctcaaagaaggaaagactacttatagttcttgacagctagctcagtcctaggtataatgctagc

SEQ ID NO: 215 - DNA, 143 bp, Artificial Sequence
  Synthetic
tccatgtctcgttatacgctgtggttcgccaacgcaggaacgacactactagctggatgagcctggtccatggagagtttaaaaagtcttttggactactggaacgacttgacagctagctcagtcctaggtataatgctagc

SEQ ID NO: 216 - DNA, 43 bp, Artificial Sequence
  Synthetic
agaaatccgtctttcattgacggggagaggtgagggacttggg

SEQ ID NO: 217 - DNA, 43 bp, Artificial Sequence
  Synthetic
agaaatccgtctttcattgacgggagccagtgttgctagtcaa

SEQ ID NO: 218 - DNA, 43 bp, Artificial Sequence
  Synthetic
agaaatccgtctttcattgacggttaaactctccatggaccag

SEQ ID NO: 219 - protein, 1812 aa, Artificial Sequence
  Synthetic
MKIEEGKGHHHHHHMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 220 - protein, 1620 aa, Artificial Sequence
  Synthetic
MKIEEGKGHHHHHHMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTSKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPMKRTADGSEFESPKKKRKV

SEQ ID NO: 221 - protein, 1562 aa, Artificial Sequence
  Synthetic
MKIEEGKGHHHHHHMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTSKRPAATKKAGQAKKKKSGGSSGGSSGSETPGTSESATPESSGGSSGGSMDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCMKRTADGSEFESPKKKRKV

SEQ ID NO: 222 - protein, 1548 aa, Artificial Sequence
  Synthetic
MKRTADGSEFESPKKKRKVMDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSSGGSSGSETPGTSESATPESSGGSSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSKRPAATKKAGQAKKKK

SEQ ID NO: 223 - protein, 1548 aa, Artificial Sequence
  Synthetic
MKRTADGSEFESPKKKRKVMDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSSGGSSGSETPGTSESATPESSGGSSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTSKRPAATKKAGQAKKKK

SEQ ID NO: 224 - protein, 486 aa, Artificial Sequence
  Synthetic
STLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLP

SEQ ID NO: 225-229 - skipped (5 entries)

SEQ ID NO: 230 - protein, 487 aa, Artificial Sequence
  Synthetic
MTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEILQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDL

SEQ ID NO: 231 - protein, 231 aa, Artificial Sequence
  Synthetic
MLQLGHLEESNSPWNTPVFVIKKKSGKWRLLQDLRAVNATMHDMGALQPGLPSPVAVPKGWEIIIIDLQDCFFNIKLHPEDCKRFAFSVPSPNFKRPYQRFQWKVLPQGMKNSPTLCQKFVDKAILTVRDKYQDSYIVHYMDDILLAHPSRSIVDEILTSMIQALNKHGLVVSTEKIQKYDNLKYLGTHIQGDSVSYQKLQIRTDKLRTLNDFQKLLGNINWIRPFLKLTT

SEQ ID NO: 232 - protein, 427 aa, Artificial Sequence
  Synthetic
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 233 - protein, 774 aa, Artificial Sequence
  Synthetic
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 234 - protein, 774 aa, Artificial Sequence
  Synthetic
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYDGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 235 - protein, 774 aa, Artificial Sequence
  Synthetic
MILDTDYITEDGKPVIRIFKKENGEFKIEYDRTFEPYLYALLKDDSAIEEVKKITAERHSTVVTVKRVEKVQKKFLGRSVEVWKLYFTHPQDVPAIMDKIREHPAVIDIYEYDIPFAIRYLIDKGLVPMEGDEELKLLALDIGTPCHEGEVFAEGPILMISYADEEGTRVITWRNVDLPYVDVLSTEREMIQRFLRVVKEKDPDVLITYNGDNFDFAYLKKRCEKLGINFTLGREGSEPKIQRMGDRFAVEVKGRIHFDLYPVIRRTVNLPIYTLEAVYEAVFGQPKEKVYAEEITTAWETGENLERVARYSMEDAKVTYELGKEFMPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYITMTIKEIEEKYGFKLIYSDTDGFFATIPGAEAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIENQVLPAVERILRAYGYRKEDLWYQKTRQVGLSARLKPKGT

SEQ ID NO: 236 - protein, 683 aa, Artificial Sequence
  Synthetic
MDHLLLKTQTQTEQVMNVTNPNSIYIKGRLYFKGYKKIELHCFVDTGASLCIASKFVIPEEHWVNAERPIMVKIADGSSITISKVCKDIDLIIAGEIFRIPTVYQQESGIDFIIGNNFCQLYEPFIQFTDRVIFTKNKSYPVHIAKLTRAVRVGTEGFLESMKKRSKTQQPEPVNISTNKIENPLEEIAILSEGRRLSEEKLFITQQRMQKIEELLEKVCSENPLDPNKTKQWMKASIKLSDPSKAIKVKPMKYSPMDREEFDKQIKELLDLKVIKPSKSPHMAPAFLVNNEAEKRRGKKRMVVNYKAMNKATVGDAYNLPNKDELLTLIRGKKIFSSFDCKSGFWQVLLDQESRPLTAFTCPQGHYEWNVVPFGLKQAPSIFQRHMDEAFRVFRKFCCVYVDDILVFSNNEEDHLLHVAMILQKCNQHGIILSKKKAQLFKKKINFLGLEIDEGTHKPQGHILEHINKFPDTLEDKKQLQRFLGILTYASDYIPKLAQIRKPLQAKLKENVPWRWTKEDTLYMQKVKKNLQGFPPLHHPLPEEKLIIETDASDDYWGGMLKAIKINEGTNTELICRYASGSFKAAEKNYHSNDKETLAVINTIKKFSIYLTPVHFLIRTDNTHFKSFVNLNYKGDSKLGRNIRWQAWLSHYSFDVEHIKGTDNHFADFLSREFNKVNSSGGS

SEQ ID NO: 237 - protein, 329 aa, Artificial Sequence
  Synthetic
MGIHGVPAAMKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 238 - protein, 235 aa, Artificial Sequence
  Synthetic
MEKEGQLEEAPPTNPYNTPTFAIKKKDKNKWRMLIDFRELNKVTQDFTEIQLGIPHPAGLAKKRRITVLDVGDAYFSIPLHEDFRPYTAFTLPSVNNAEPGKRYIYKVLPQGWKGSPAIFQHTMRQVLEPFRKANKDVIIIQYMDDILIASDRTDLEHDRVVLQLKELLNGLGFSTPDEKFQKDPPIHWMGYELWPTKWKLQKIQLPQKEIWTVNDIQKLVGVLNWAAQLYPGIK

SEQ ID NO: 239 - protein, 235 aa, Artificial Sequence
  Synthetic
MEEEGKISKIGPENPYNTPVFAIKKKDSTKWRKLVDFRELNKRTQDFWEVQLGIPHPAGLKKKKSVTVLDVGDAYFSVPLDESFRKYTAFTIPSINNETPGIRYQYNVLPQGWKGSPAIFQSSMTKILEPFRIKNPEIVIYQYMDDLYVGSDLEIGQHRTKIEELRAHLLSWGFTTPDKKHQKEPPFLWMGYELHPDRWTVQPIDLPEKDSWTVNDIQKLVGKLNWASQIYAGIK

SEQ ID NO: 240 - protein, 441 aa, Artificial Sequence
  Synthetic
MPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 241 - RNA, 36 nt, Artificial Sequence
  Synthetic
aauagcggcccuaagaaauccgucuuucauugacgg

SEQ ID NO: 242 - RNA, 36 nt, Artificial Sequence
  Synthetic
auuggaacuggcgagaaauccgucuuucauugacgg

SEQ ID NO: 243 - RNA, 36 nt, Artificial Sequence
  Synthetic
ccagcaacaccuaagaaauccgucuuucauugacgg

SEQ ID NO: 244 - RNA, 36 nt, Artificial Sequence
  Synthetic
cggcgcucgaauaggaaauccgucuuucauugacgg

SEQ ID NO: 245 - RNA, 36 nt, Artificial Sequence
  Synthetic
guggcaacaccuaagaaauccgucuuucauugacgg

SEQ ID NO: 246 - RNA, 36 nt, Artificial Sequence
  Synthetic
guugcaacaccuaagaaauccgucuuucauugacgg

SEQ ID NO: 247 - RNA, 36 nt, Artificial Sequence
  Synthetic
guugcaaugccuaagaaauccgucuuucauugacgg

SEQ ID NO: 248 - DNA, 20 bp, Artificial Sequence
  Synthetic
gggaagtggttggtcagcat

SEQ ID NO: 249 - DNA, 20 bp, Artificial Sequence
  Synthetic
gggaagtggttgcgaggcat

SEQ ID NO: 250 - DNA, 20 bp, Artificial Sequence
  Synthetic
gggaagtggttgagcggtca

SEQ ID NO: 251 - DNA, 16 bp, Artificial Sequence
  Synthetic
gggaagtggttggcat

SEQ ID NO: 252 - DNA, 20 bp, Artificial Sequence
  Synthetic
gagccagtgttgctagtcaa

SEQ ID NO: 253 - DNA, 20 bp, Artificial Sequence
  Synthetic
gagccagtgttgcgagtcaa

SEQ ID NO: 254 - DNA, 20 bp, Artificial Sequence
  Synthetic
gagccagtgttgagcgctag

SEQ ID NO: 255 - DNA, 16 bp, Artificial Sequence
  Synthetic
gagccagtgttgtcaa

SEQ ID NO: 256 - DNA, 20 bp, Artificial Sequence
  Synthetic
ttaaactctccatggaccag

SEQ ID NO: 257 - DNA, 20 bp, Artificial Sequence
  Synthetic
ttaaactctccacgagccag

SEQ ID NO: 258 - DNA, 20 bp, Artificial Sequence
  Synthetic
ttaaactctccaagcgtgga

SEQ ID NO: 259 - DNA, 16 bp, Artificial Sequence
  Synthetic
ttaaactctccaccag

(19) United States
(12) Patent Application Publication
(10) Pub. No.: US 2023/0023791 A1
(43) Pub. Date: Jan. 26, 2023

(54) GENE EDITING SYSTEMS COMPRISING A CRISPR NUCLEASE AND USES THEREOF

(71) Applicant: Arbor Biotechnologies, Inc., Cambridge, MA (US)

(72) Inventors: David A. SCOTT, San Francisco, CA (US); Noah Michael JAKIMO, San Francisco, CA (US); Pratyusha HUNNEWELL, San Francisco, CA (US); Fu-Kai HSIEH, Cambridge, MA (US)

(21) Appl. No.: 17/830,212

(22) Filed: Jun. 1, 2022

Related U.S. Application Data
(60) Provisional application No. 63/195,621, filed on Jun. 1, 2021.
(60) Provisional application No. 63/236,047, filed on Aug. 23, 2021.
(60) Provisional application No. 63/272,937, filed on Oct. 28, 2021.
(60) Provisional application No. 63/299,695, filed on Jan. 14, 2022.

(51) Int. Cl. C12N 15/11; C12N 9/22; C12N 15/86; C12N 15/90

(52) U.S. Cl. CPC C12N 15/11; C12N 9/22; C12N 15/86; C12N 15/907; C12N 2310/20; C12N 2800/80; C12N 2310/531; C12N 2750/14143; C07K 2319/00

(57) ABSTRACT

A gene editing system comprising: (a) a Type V CRISPR nuclease polypeptide or a first nucleic acid encoding the Type V CRISPR nuclease polypeptide; (b) a reverse transcriptase (RT) polypeptide or a second nucleic acid encoding the RT polypeptide; (c) a guide RNA (gRNA) or a third nucleic acid encoding the gRNA, wherein the gRNA comprises one or more binding sites recognizable by the Type V CRISPR nuclease (CRISPR nuclease binding sites) and a spacer sequence specific to a target sequence within a genomic site of interest, the target sequence being adjacent to a protospacer adjacent motif (PAM); and (d) a reverse transcription donor RNA (RT donor RNA) or a fourth nucleic acid encoding the RT donor RNA, wherein the RT donor RNA comprises a primer binding site (PBS) and a template sequence.

### CROSS REFERENCE TO RELATED APPLICATIONS

This application claims the benefit under 35 U.S.C. § 119(e) of U.S. Provisional Application No. 63/195,621, filed Jun. 1, 2021, U.S. Provisional Application No. 63/236,047, filed Aug. 23, 2021, U.S. Provisional Application No. 63/272,937, filed Oct. 28, 2021, and U.S. Provisional Application No. 63/299,695, filed Jan. 14, 2022, the contents of each of which are incorporated by reference herein in their entirety.

### SEQUENCE LISTING

The instant application contains a Sequence Listing which has been filed electronically in ASCII format and is hereby incorporated by reference in its entirety. Said ASCII copy, created on Jun. 1, 2022, is named 116928-0043-0001US00_SEQ.txt and is 388,315 bytes in size.

### BACKGROUND

Clustered Regularly Interspaced Short Palindromic Repeats (CRISPR) and CRISPRassociated (Cas) genes, collectively known as CRISPR-Cas or CRISPR/Cas systems, are adaptive immune systems in archaea and bacteria that defend particular species against foreign genetic elements.

### SUMMARY OF THE INVENTION

The present disclosure is based, at least in part, on the development of a gene editing system involving a Type V CRISPR nuclease polypeptide (e.g., a Cas12i2 polypeptide) and a reverse transcriptase, as well as a guide RNA (gRNA) mediating cleavage at a genetic site of interest by the CRISPR nuclease polypeptide and a reverse transcription donor RNA mediating synthesis of desired sequences to be incorporated into the genomic site of interest. As reported herein, the gene editing system disclosed herein has achieved successful gene editing at various genomic sites with high editing efficiency and accuracy. Without being bound by theory, the gene editing system disclosed herein show at least one of the following advantageous features:

1. Many of the editing template RNAs described herein, such as those specific to a Cas12i polypeptide, do not require a trans-activating CRISPR RNA (tracrRNA) component and are thus smaller than prime editing guide RNAs (pegRNAs). Additionally, many of the CRISPR nuclease-reverse transcriptase fusions described herein, such as Cas12i polypeptide-reverse transcriptase fusions, are smaller than Cas9-reverse transcriptase fusions. Both of these aspects are preferable in terms of delivery and cost of synthesis.

2. Editing template RNAs described herein can be designed to have a longer primer binding site (PBS) than the PBS of pegRNAs. This feature could increase efficiency of edit incorporation into a target nucleic acid.

3. Gene editing systems comprising an editing template RNA designed to bind the non-PAM strand only (i.e., the complementary strand of the strand on which the PAM motif resides; also described herein as the target strand), as described herein, are capable of incorporating edits over a broader window compared to prime editing systems. In particular, Cas12i polypeptide-reverse transcriptase systems are capable of rewriting the full recognition sequence of the Cas12i polypeptide and an RNA guide. Therefore, these gene editing systems may be more efficient at evading retargeting of the target nucleic acid by the CRISPR nuclease-reverse transcriptase fusion and an editing template RNA.

Accordingly, provided herein are gene editing systems, pharmaceutical compositions or kits comprising such, methods of using the gene editing system to produce genetically modified cells, and the resultant cells thus produced.

In some aspects, the present disclosure features a gene editing system comprising: (a) a Type V CRISPR nuclease polypeptide or a first nucleic acid encoding the Type V CRISPR nuclease polypeptide; (b) a reverse transcriptase (RT) polypeptide or a second nucleic acid encoding the RT polypeptide; (c) a guide RNA (gRNA) or a third nucleic acid encoding the gRNA, wherein the gRNA comprises one or more binding sites recognizable by the Type V CRISPR nuclease (CRISPR nuclease binding sites) and a spacer sequence specific to a target sequence within a genomic site of interest, the target sequence being adjacent to a protospacer adjacent motif (PAM); and (d) a reverse transcription donor RNA (RT donor RNA) or a fourth nucleic acid encoding the RT donor RNA, wherein the RT donor RNA comprises a primer binding site (PBS) and a template sequence.

In some embodiments, the Type V CRISPR nuclease polypeptide in any of the gene editing systems disclosed herein is a Cas12 polypeptide. In some examples, the Cas12 polypeptide is a Cas12i polypeptide, for example, a Cas12i2 polypeptide. In some instances, the Cas12i polypeptide is a Cas12i2 polypeptide, which comprises an amino acid sequence at least 95% identical to SEQ ID NO: 2.

In some instances, the Cas12i2 polypeptide comprises one or more mutations at positions D581, G624, F626, P868, I926, V1030, E1035, and/or S1046 of SEQ ID NO: 2. For example, the one or more mutations are amino acid substitutions, which optionally is D581R, G624R, F626R, P868T, I926R, V1030G, E1035R, S1046G, or a combination thereof. In one example, the Cas12i2 polypeptide comprises mutations at positions D581, D911, I926, and V1030 (e.g., amino acid substitutions of D581R, D911R, 1926R, and V1030G). In another example, the Cas12i2 polypeptide comprises mutations at positions D581, I926, and V1030 (e.g., amino acid substitutions of D581R, 1926R, and V1030G). In yet another example, the Cas12i2 polypeptide comprises mutations at positions D581, I926, V1030, and S1046 (e.g., amino acid substitutions of D581R, I926R, V1030G, and S1046G). In still another example, the Cas12i2 polypeptide comprises mutations at positions D581, G624, F626, I926, V1030, E1035, and S1046 (e.g., amino acid substitutions of D581R, G624R, F626R, I926R, V1030G, E1035R, and S1046G). In another example, the Cas12i2 polypeptide comprises mutations at positions D581, G624, F626, P868, I926, V1030, E1035, and S1046 (e.g., amino acid substitutions of D581R, G624R, F626R, P868T, I926R, V1030G, E1035R, and S1046G). Exemplary Cas12i2 polypeptides for use in any of the gene editing systems disclosed herein may comprise the amino acid sequence of any one of SEQ ID NOs: 3-7. In some examples, the exemplary Cas12i2 polypeptide can comprise the amino acid sequence of SEQ ID NO: 4. In other examples, the exemplary Cas12i2 polypeptide can comprise the amino acid sequence of SEQ ID NO: 7.

In other instances, the Cas12i polypeptide has diminished crRNA processing activity, optionally wherein the Cas12i polypeptide comprises mutations at position H485 and/or position H486 of SEQ ID NO: 2.

In some embodiments, any of the gene editing systems disclosed herein may comprise the Type V CRISPR nuclease polypeptide. Alternatively, the gene editing system may comprise the first nucleic acid encoding the Type V CRISPR nuclease polypeptide. In some instances, the first nucleic acid is located in a first vector (e.g., a viral vector such as an adeno-associated viral vector or AAV vector). In other instances, the first nucleic acid is a first messenger RNA (mRNA).

In any of the gene editing systems disclosed herein, the RT polypeptide may be Moloney Murine Leukemia Virus (MMLV)-RT, mouse mammary tumor virus (MMTV)-RT, Marathon-RT, or RTx-RT (e.g., the MMLV RT, which may comprise the amino acid sequence of SEQ ID NO: 29). In some instances, the gene editing system comprises the RT polypeptide. Alternatively, the system comprises the second nucleic acid encoding the RT polypeptide. In some instances, the second nucleic acid is located in a second vector (e.g., a viral vector such as an adeno-associated viral vector or AAV vector). In one example, the gene editing system comprises a vector (e.g., a viral vector) that comprises both the first nucleic acid encoding the Type V CRISPR polypeptide and the second nucleic acid encoding the RT polypeptide. In other examples, the second nucleic acid encoding the RT is a second mRNA. In one example, the gene editing system comprises a single RNA molecule comprising both the first mRNA encoding the Type V CRISPR polypeptide and the second mRNA encoding the RT.

In some embodiments, the gene editing system disclosed herein comprises a fusion polypeptide, which comprises the Type V CRISPR nuclease polypeptide and the RT polypeptide, or a nucleic acid (e.g., vector such as a viral vector) encoding the fusion polypeptide. Alternatively, the gene editing system comprises the Type V CRISPR nuclease polypeptide and the RT polypeptide as two separate polypeptides.

In any of the gene editing systems disclosed herein, the spacer sequence can be 20-30-nucleotide in length. In some examples, the spacer sequence is 20-nucleotide in length.

In some embodiments, the PAM comprises the motif of 5′-TTN-3.′ In some instances (e.g., in association with a Cas12i2 polypeptide), the PAM may be located 5′ to the target sequence.

In some embodiments, the one or more CRISPR nuclease binding sites are direct repeat sequence(s). In some instances, each direct repeat sequence is 23-36-nucleotide in length. In one example, the direct repeat sequence is 23-nucleotide in length. In some examples, the direct repeat sequence is at least 90% identical to any one of SEQ ID NOs: 15-17 and 241-247 (e.g., SEQ ID NO: 17) or a fragment thereof that is at least 23-nucleotide in length. In specific examples, the direct repeat sequence is any one of SEQ ID NOs: 15-17 and 241-247 (e.g., SEQ ID NO: 17), or a fragment thereof that is at least 23-nucleotide in length.

In some embodiments, the gene editing system disclosed herein comprises the gRNA. Alternatively, the gene editing system comprises the third nucleic acid encoding the gRNA. In some examples, the third nucleic acid is located in a third vector, which optionally is a viral vector. In some examples, the gene editing system may comprise a vector such as a viral vector that comprises the third nucleic acid encoding the gRNA and the first and/or second nucleic acids encoding the Type V CRISPR nuclease polypeptide and/or the RT polypeptide.

In some embodiments, the PBS in the RT donor RNA of any of the gene editing systems disclosed herein can be 5-100-nucleotide in length. In some examples, the PBS is 10-60-nucleotide in length. In specific examples, the PBS is 10-30-nucleotide in length. In some instances, the PBS binds a PBS-targeting site that is adjacent to the complementary region of the target sequence. The PBS-targeting site is upstream to the complementary region of the target sequence. For example, the PBS-targeting site may be 3-10-nucleotide (e.g., 4-10-nucleotide) upstream to the complementary region of the target sequence. Alternatively, the PBS-targeting site may overlap with the complementary region of the target sequence. In other instances, the PBS-targeting site is adjacent to or overlap with the target sequence.

In some embodiments, the template sequence in the RT donor RNA of any of the gene editing systems disclosed herein can be 5-100-nucleotide in length. For example, the template sequence may be 30-50-nucleotide in length. In some instances, the template sequence may be homologous to the genomic site of interest and comprises one or more nucleotide variations relative to the genomic site of interest. In some examples, at least one nucleotide variation is located within the target sequence. Alternatively or in addition, at least one nucleotide variation is located in the PAM.

In some embodiments, any of the gene editing system disclosed herein comprises the RT donor RNA. Alternatively, the gene editing system comprises the fourth nucleic acid encoding the RT donor RNA. In some examples, the fourth nucleic acid is located in a fourth vector, which optionally is a fourth viral vector. In some instances, the gene editing system comprises a vector such as a viral vector comprising the nucleic acid encoding the RT donor RNA, and one or more additional nucleic acids encoding the guide RNA, the Type V CRISPR nuclease polypeptide, and the RT polypeptide.

In some embodiments, the gene editing system disclosed herein comprises a single RNA molecule comprising the gRNA and the RT donor RNA. Such a single RNA comprises the CRISPR nuclease binding site, the spacer sequence, the PBS, and the template sequence, which may be arranged in any suitable order. In some examples, the single RNA molecule further comprises a linker between the gRNA and the RT donor RNA. Such a linker may comprise a hairpin structure. In one example, the single RNA molecule comprises, from 5′ to 3′: the CRISPR nuclease binding site, the spacer sequence, the template sequence, and the PBS. In another example, the single RNA molecule comprises, from 5′ to 3′: the CRISPR nuclease binding site, the spacer sequence, the linker, the template sequence, and the PBS. In yet another example, the single RNA molecule comprises, from 5′ to 3′: the template sequence, the PBS, the CRISPR nuclease binding site, and the spacer sequence. In yet another example, the single RNA molecule comprises, from 5′ to 3′: the template sequence, the PBS, the linker, the CRISPR nuclease binding site, and the spacer sequence.

In some instances, any of the single RNA molecule disclosed herein may further comprise a 5′ end protection fragment, a 3′ end protection fragment, or both. Each of the 5′ end protection fragment and the 3′ end protection fragment may form a secondary structure, for example, a hairpin, a pseudoknot, or a triplex structure. In some examples, the 5′ end protection fragment and/or the 3′ end protection fragment is an exoribonuclease-resistant RNA (xrRNA), a transfer RNA (tRNA), or a truncated tRNA. In specific examples, the 5′ end protection fragment, the 3′ end protection fragment, or both may comprise one or more of the CRISPR nuclease binding site. The 5′ end protection fragment, the 3′ end protection fragment, or both may further comprise one or more segments that are not homologous to any human sequence (cannot bind to any human sequences via base pairing).

In some embodiments, the gene editing system disclosed herein comprises any of the gRNAs and any of the RT donor RNAs as two separate RNA molecules. In some examples, the gRNA, the RT donor RNA, or both may further comprise a 5′ end protection fragment and/or a 3′ end protection fragment. Each of the protection fragment may form a secondary structure, for example, a hairpin, a pseudoknot, or a triplex structure. In some examples, the 5′ end protection fragment and/or the 3′ end protection fragment is an exoribonuclease-resistant RNA (xrRNA), a transfer RNA (tRNA), or a truncated tRNA. In other examples, the 5′ end protection fragment and/or the 3′ end protection fragment comprises one or more of the CRISPR nuclease binding site, and optionally one or more segments that are not homologous to any human sequence.

Any of the gene editing systems disclosed herein may comprise one or more lipid nanoparticles (LNPs), which encompass the Type V CRISPR nuclease polypeptide or the encoding nucleic acid, the RT polypeptide or the encoding nucleic acid, the guide RNA or the encoding nucleic acid, the RT donor RNA or the encoding nucleic acid, or any combination thereof. Alternatively, the gene editing system may comprise (i) one or more lipid nanoparticles (LNPs), which collectively encompass up to three components selected from of the Type V CRISPR nuclease polypeptide or the encoding nucleic acid, the RT polypeptide or the encoding nucleic acid, the guide RNA or the encoding nucleic acid, the RT donor RNA or the encoding nucleic acid; and (ii) one or more vectors encoding the remaining components in the gene editing system. In some instances, the one or more vectors can be one or more viral vectors, for example, one or more adeno-associated viral (AAV) vectors.

In some examples, the gene editing system disclosed herein comprises the Type V CRISPR nuclease polypeptide, the RT polypeptide, the gRNA, and the RT donor RNA. In some instances, the Type V CRISPR nuclease polypeptide and/or the RT polypeptide forms a complex (e.g., a ribonucleoprotein (RNP) complex) with the gRNA and/or the RT donor RNA.

In some aspects, the present disclosure also provides a pharmaceutical composition comprising any of the gene editing systems disclosed herein and a pharmaceutically acceptable carrier, and a kit comprising the components of the gene editing system.

In other aspects, the present disclosure also features a method for genetically editing a cell, the method comprising contacting a host cell any of the gene editing systems disclosed herein or the pharmaceutical composition comprising such to genetically edit the host cell. In some examples, the host cell is cultured in vitro. In other examples, the contacting step is performed by administering the gene editing system to a subject comprising the host cell.

Also within the scope of the present disclosure is a population of genetically modified cells, which can be produced by the gene editing system disclosed herein. In some examples, the genetically modified cells may comprise cells not editable by the gene editing system, for example, comprise one or more modifications in the PAM, in the target sequence, or in both.

In yet other aspects, the present disclosure features a gene editing RNA molecule, comprising: (i) one or more binding sites recognizable by a Type V CRISPR nuclease (CRISPR nuclease binding sites); (ii) a spacer sequence specific to a target sequence within a genetic site, the target sequence being adjacent to a protospacer adjacent motif (PAM); (iii) a primer binding site (PBS); and (iv) a template sequence. In some embodiments, the gene editing RNA molecule may further comprise one or more linkers such as those disclosed herein.

In some examples, the RNA molecule comprises, from 5′ to 3′: the CRISPR nuclease binding site, the spacer sequence, the template sequence, and the PBS. In other examples, the RNA molecule comprises, from 5′ to 3′: the CRISPR nuclease binding site, the spacer sequence, the linker, the template sequence, and the PBS. In yet other examples, the RNA molecule comprises, from 5′ to 3′: the template sequence, the PBS, the CRISPR nuclease binding site, and the spacer sequence. In still other examples, the RNA molecule comprises, from 5′ to 3′: the template sequence, the PBS, the linker, the CRISPR nuclease binding site, and the spacer sequence.

Any of the gene editing RNA molecules disclosed herein may further comprise a 5′ end protection fragment, a 3′ end protection fragment, or both. Each of the protection fragment may form a secondary structure, for example, a hairpin, a pseudoknot, or a triplex structure. In some examples, the 5′ end protection fragment and/or the 3′ end protection fragment is an exoribonuclease-resistant RNA (xrRNA), a transfer RNA (tRNA), or a truncated tRNA. In other examples, the 5′ end protection fragment and/or the 3′ end protection fragment comprises one or more of the CRISPR nuclease binding site, and optionally one or more segments that are not homologous to any human sequence.

In addition, the present disclosure features a set of gene editing RNA molecules (two separate RNA molecules), comprising: (i) a guide RNA comprising one or more binding sites recognizable by the Type V CRISPR nuclease (CRISPR nuclease binding sites), and a spacer sequence specific to a target sequence within a genetic site, the target sequence being adjacent to a protospacer adjacent motif (PAM); and (ii) a reverse transcription donor RNA (RT donor RNA) or a fourth nucleic acid encoding the RT donor RNA, wherein the RT donor RNA comprises a primer binding site (PBS) and a template sequence. In some examples, the gRNA, the RT donor RNA, or both further comprise a 5′ end protection fragment and/or a 3′ end protection fragment. Each of the protection fragment may form a secondary structure, for example, a hairpin, a pseudoknot, or a triplex structure. In some examples, the 5′ end protection fragment and/or the 3′ end protection fragment is an exoribonuclease-resistant RNA (xrRNA), a transfer RNA (tRNA), or a truncated tRNA. In other examples, the 5′ end protection fragment and/or the 3′ end protection fragment comprises one or more of the CRISPR nuclease binding site, and optionally one or more segments that are not homologous to any human sequence.

Also provided herein is a DNA molecule or a set of DNA molecules, which encode the gene editing RNA molecule or the set of gene editing RNA molecules as disclosed herein. In some examples, the DNA molecule or the set of DNA molecules of claim 76, which is included in a vector or a set of vectors, optionally wherein the vector or set of vectors are viral vectors.

In addition, provided herein is a fusion polypeptide comprising a CRISPR nuclease and a reverse transcriptase. Any of such CRISPR nuclease-RT fusion polypeptides can be used in the gene editing system disclosed herein. In some embodiments, the CRISPR nuclease is a Type V CRISPR nuclease, for example, a Cas12i polypeptide. In some examples, the Cas12i polypeptide is a Cas12i2 polypeptide, e.g., those disclosed herein. In specific examples, the fusion polypeptide may comprise the amino acid sequence of 25-26 and 219-223.

In some embodiments, the Cas12i polypeptide is a Cas12i4 polypeptide. In some examples, the Cas12i4 polypeptide may be fused with a reverse transcriptase, such as an MMLV RT. Such a fusion Cas12i4-RT fusion polypeptide may comprise the amino acid sequence of SEQ ID NO: 53.

Any of the nucleic acids encoding any of the CRISPR nuclease-RT fusion polypeptides, including vectors such as expression vectors (e.g., viral vectors), is also within the scope of the present disclosure.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B include schematics showing exemplary gene editing systems disclosed herein. FIG. 1A is a schematic showing a gene editing system comprising a CRISPR nuclease (e.g., a Cas12i polypeptide) fused to a reverse transcriptase polypeptide and an RNA guide fused to an RT donor RNA at the 3′ end of the RNA guide. The RT donor RNA comprises a reverse transcription template sequence and a PBS. The PBS comprises substantial complementarity to the PAM-strand (a.k.a., the non-target strand) of a target nucleic acid. FIG. 1B shows a Cas9 nickase fused to a reverse transcriptase (left) and a Cas12i nickase fused to a reverse transcriptase (right). Using an RT donor RNA fused to the 3′ end of an RNA guide, an edit is incorporated into the PAM strand of a target nucleic acid.

FIG. 2 is a schematic showing an exemplary gene editing system comprising a CRISPR nuclease (e.g., a Cas12i polypeptide) fused to a reverse transcriptase polypeptide and an RNA guide fused to an RT donor RNA at the 5′ end of the RNA guide. The RT donor RNA comprises a PBS and a reverse transcription template sequence. The PBS comprises complementarity to the PAM strand of a target nucleic acid.

FIG. 3 is a schematic showing a CRISPR nuclease (e.g., a Cas12i polypeptide), a reverse transcriptase polypeptide, an RNA guide, and an RT donor RNA. The RT donor RNA comprises a reverse transcription template sequence and a PBS. An edit is incorporated into the genome following cleavage by the CRISPR nuclease.

FIG. 4 is a schematic showing a CRISPR nuclease (e.g., a Cas12i polypeptide), a reverse transcriptase polypeptide, an RNA guide, and an RNA reverse transcription template sequence. The RT donor RNA comprises a PBS and a reverse transcription template sequence. An edit is incorporated into the genome in the presence of the CRISPR nuclease.

FIG. 5 is a schematic showing an exemplary gene editing system comprising a CRISPR nuclease (e.g., a Cas12i polypeptide) fused to a reverse transcriptase polypeptide and an RNA guide containing mismatches to the target nucleic acid, fused to an RT donor RNA at the 3′ end of the RNA guide. The RT donor RNA comprises a PBS. The PBS comprises complementarity to the non-PAM strand (a.k.a., target strand or TS) of a target nucleic acid.

FIGS. 6A-6B include schematics showing exemplary gene editing systems disclosed herein. FIG. 6A is a schematic showing an exemplary gene editing system comprising a CRISPR nuclease (e.g., a Cas12i polypeptide) fused to a reverse transcriptase polypeptide and an RNA guide fused to an RT donor RNA at the 3′ end of the RNA guide. The RT donor RNA comprises a reverse transcription template sequence and a PBS. When the spacer sequence of the RNA guide and the PBS are bound to the target nucleic acid, the reverse transcription template sequence forms a loop of unpaired nucleotides. The PBS comprises complementarity to the non-PAM strand of a target nucleic acid. The variant Cas12i2 cleavage sites in the PAM strand and non-PAM strand are indicated by the triangles. Using an RT donor RNA fused to the 3′ end of an RNA guide, an edit is incorporated into the non-PAM strand of a target nucleic acid. FIG. 6B shows the positioning of an edit, reverse transcription template sequence, and PBS, wherein the length of the reverse transcription template sequence and PBS can be varied.

FIG. 7 is a schematic showing an exemplary gene editing system comprising a CRISPR nuclease (e.g., a Cas12i polypeptide) fused to a reverse transcriptase polypeptide and an RNA guide fused to an RT donor RNA at the 5′ end of the RNA guide. The RT donor RNA comprises a PBS and a reverse transcription template sequence. The PBS comprises complementarity to the non-PAM strand of a target nucleic acid.

FIGS. 8A-8C include schematics showing exemplary Cas12i2 RNA guide-RT donor RNA fusions. FIG. 8A is a schematic of a variant Cas12i2 RNA guide fused to an RT donor RNA, which was tested in Example 1. The spacer of the RNA guide binds to the non-PAM strand adjacent to a 5′-TTT-3′ PAM. The RT donor RNA comprises a reverse transcription template sequence and a PBS. When the spacer sequence and the PBS are bound to the target nucleic acid, the reverse transcription template sequence forms a loop of unpaired nucleotides. The PBS comprises complementarity to the non-PAM strand of a target nucleic acid. In this schematic, the PBS is 13 nucleotides in length and the reverse transcription template sequence is 34 nucleotides in length. The PBS is designed such that complementarity to non-PAM strand begins at a cleavage site (triangle). FIG. 8B shows exemplary RNA guide-RT donor RNA fusions targeting an AAVS1_T7 genomic site, as tested in Example 1. Various PBS lengths were tested (13, 30, and 60 nucleotides). The RNA guide-RT donor RNA fusions were designed to introduce substitutions (S), an insertion (I), a deletion (D), or a hairpin (H) into the target sequence. FIG. 8C shows encoded edits (substitutions, insertions, and deletions) introduced into an AAVS1_T7 genomic site (top panel), an EMX1_T6 genomic site (middle panel), and a VEGFA_T5 genomic site (bottom panel) as described in Example 1. Sequences in FIG. 8A, from top to bottom, are SEQ ID NOs: 65-67. Sequences in FIG. 8B, from top to bottom, are SEQ ID NOs: 74-80, and 87-89. Sequences in FIG. 8C, from top to bottom, are SEQ ID NOs: 248-259.

FIGS. 9A-9J include diagrams showing gene editing efficiencies resulting from exemplary gene editing systems disclosed herein. FIG. 9A shows percentage of NGS reads analyzed with indels and encoded edits induced by variant Cas12i2 of SEQ ID NO: 4 and C-terminal and N-terminal Cas12i2-MMLV RT fusions of SEQ ID NO: 25 and SEQ ID NO: 26 with an RNA guide targeting an AAVS1_T6 genomic site. FIG. 9B shows the percentage of NGS reads analyzed with indels and encoded edits induced by N-terminal and C-terminal Cas12i2-MMLV RT fusions of SEQ ID NO: 26 and SEQ ID NO: 25 and RNA guide-RT donor RNA fusions targeting an AAVS1_T6 genomic site. The RNA guide-RT donor RNA fusions had a PBS length of 13, 30, or 60 nucleotides and were designed to introduce substitutions (S), an insertion (I), a deletion (D), or a hairpin (H) into the AAVS1_T6 genomic site. FIG. 9C shows the percentage of NGS reads analyzed with indels and encoded edits induced by variant Cas12i2 of SEQ ID NO: 4 and C-terminal and N-terminal Cas12i2-MMLV RT fusions of SEQ ID NO: 25 and SEQ ID NO: 26 with an RNA guide targeting an AAVS1_T7 genomic site. FIG. 9D shows the percentage of NGS reads analyzed with indels and encoded edits induced by N-terminal and C-terminal Cas12i2-MMLV RT fusions of SEQ ID NO: 26 and SEQ ID NO: 25 and RNA guide-RT donor RNA fusions targeting an AAVS1_T7 genomic site. The RNA guide-RT donor RNA fusions had a PBS length of 13, 30, or 60 nucleotides and were designed to introduce substitutions (S), an insertion (I), a deletion (D), or a hairpin (H) into the AAVS1_T7 genomic site. FIG. 9E shows the percentage of NGS reads analyzed with indels and encoded edits induced by variant Cas12i2 of SEQ ID NO: 4 and C-terminal and N-terminal Cas12i2-MMLV RT fusions of SEQ ID NO: 25 and SEQ ID NO: 26 with an RNA guide targeting an EMX1_T6 genomic site. FIG. 9F shows the percentage of NGS reads analyzed with indels and edits induced by N-terminal and C-terminal Cas12i2-MMLV RT fusions of SEQ ID NO: 26 and SEQ ID NO: 25 and RNA guide-RT donor RNA fusions targeting an EMX1_T6 genomic site. The RNA guide-RT donor RNA fusions had a PBS length of 13, 30, or 60 nucleotides and were designed to introduce substitutions (S), an insertion (I), a deletion (D), or a hairpin (H) into the EMX1_T6 genomic site. FIG. 9G shows the percentage of NGS reads analyzed with indels and encoded edits induced by variant Cas12i2 of SEQ ID NO: 4 and C-terminal and N-terminal Cas12i2-MMLV RT fusions of SEQ ID NO: 25 and SEQ ID NO: 26 with an RNA guide targeting a VEGFA_T2 genomic site. FIG. 9H shows the percentage of NGS reads analyzed with indels and encoded edits induced by N-terminal and C-terminal Cas12i2-MMLV RT fusions of SEQ ID NO: 26 and SEQ ID NO: 25 and RNA guide-RT donor RNA fusions targeting a VEGFA_T2 genomic site. The RNA guide-RT donor RNA fusions had a PBS length of 13, 30, or 60 nucleotides and were designed to introduce substitutions (S), an insertion (I), a deletion (D), or a hairpin (H) into the VEGFA_T2 genomic site. FIG. 9I shows the percentage of NGS reads analyzed with indels and encoded edits induced by variant Cas12i2 of SEQ ID NO: 4 and C-terminal and N-terminal Cas12i2-MMLV RT fusions of SEQ ID NO: 25 and SEQ ID NO: 26 with an RNA guide targeting a VEGFA_T5 genomic site. FIG. 9J shows the percentage of NGS reads analyzed with indels and encoded edits induced by N-terminal and C-terminal Cas12i2-MMLV RT fusions of SEQ ID NO: 26 and SEQ ID NO: 25 and RNA guide-RT donor RNA fusions targeting a VEGFA_T5 genomic site. The RNA guide-RT donor RNA fusions had a PBS length of 13, 30, or 60 nucleotides and were designed to introduce substitutions (S), an insertion (I), a deletion (D), or a hairpin (H) into the VEGFA_T5 genomic site.

FIG. 10 is a schematic showing a Cas12i polypeptide (e.g., a Cas12i2 nickase) fused to a reverse transcriptase. Using an RT donor RNA fused to the 5′ end or the 3′ end of an RNA guide, an encoded edit is incorporated into the PAM strand of a target nucleic acid. The ends of the RNA guide-RT donor RNA can be protected to prevent exonuclease or endonuclease activity. The PBS length can vary between about 3-100 nucleotides and comprise substantial complementarity to the PAM strand. Structured RNA such as hairpins can be introduced between the spacer and the reverse transcription template sequence.

FIG. 11 is a schematic showing an RNA guide-RT donor RNA further fused to a second direct repeat (DR)-spacer sequence. The additional DR-spacer inhibits exonuclease activity.

FIGS. 12A-12B include schematics showing exemplary designs of editing template RNAs (gene editing RNAs). FIG. 12A is a schematic depicting editing template RNAs (5′-nuclease binding sequence—DNA-binding sequence—reverse transcription template—PBS3′) further comprising 3′ end protection. The 3′ end protection can be a chemical end protection (top portion of the figure) or a hairpin (bottom portion of the figure). The hairpin can be a nuclease binding sequence such as a direct repeat sequence. FIG. 12B is a schematic depicting editing template RNAs (5′-reverse transcription template—PBS nuclease binding sequence—DNA-binding sequence-3′) with and without 5′ end protection. The 5′ end protection can be a hairpin (e.g., a nuclease binding sequence such as a direct repeat sequence), as shown in the bottom portion of the figure.

FIGS. 13A-13D include diagrams showing gene editing efficiencies resulting from exemplary gene editing systems disclosed herein. FIG. 13A shows activity of Cas12i2 (SEQ ID NO: 4) and Cas12i2-RT (SEQ ID NO: 25) with the RNA guide of SEQ ID NO: 112 or the editing template RNAs of SEQ ID NOs: 123-137 at an AAVS1_T7 genomic site (SEQ ID NO: 30). % NGS reads analyzed as having an indel are shown in the white bars for Cas12i2 and grey bars for Cas12i2-RT. % NGS reads analyzed as having the encoded edit are shown in the checkered bars for Cas12i2 and black bars for Cas12i2-RT. FIG. 13B shows activity of Cas12i2 (SEQ ID NO: 4) and Cas12i2-RT (SEQ ID NO: 25) with the RNA guide of SEQ ID NO: 114 or the editing template RNAs of SEQ ID NOs: 138-152 at an EMX1_T6 genomic site (SEQ ID NO: 34). % NGS reads analyzed as having an indel are shown in the white bars for Cas12i2 and grey bars for Cas12i2-RT. % reads analyzed as having the encoded edit are shown in the checkered bars for Cas12i2 and black bars for Cas12i2-RT. FIG. 13C shows activity of Cas12i2 (SEQ ID NO: 4) and Cas12i2-RT (SEQ ID NO: 25) with the RNA guide of SEQ ID NO: 116 or the editing template RNAs of SEQ ID NOs: 153-167 at VEGFA_T2 (SEQ ID NO: 36). % NGS reads analyzed as having an indel are shown in the white bars for Cas12i2 and grey bars for Cas12i2-RT. % NGS reads analyzed as having the encoded edit are shown in the checkered bars for Cas12i2 and black bars for Cas12i2-RT. FIG. 13D shows activity of Cas12i2 (SEQ ID NO: 4) and Cas12i2-RT (SEQ ID NO: 25) with the RNA guide of SEQ ID NO: 118 or the editing template RNAs of SEQ ID NOs: 168-182 at a VEGFA_T5 genomic site (SEQ ID NO: 38). % NGS reads analyzed as having an indel are shown in the white bars for Cas12i2 and grey bars for Cas12i2-RT. % NGS reads analyzed as having the encoded edit are shown in the checkered bars for Cas12i2 and black bars for Cas12i2-RT.

FIG. 14A-14C include schematics depicting the steps of an assay used to identify cleavage patterns of Cas12i2 with an RNA guide or an editing template RNA. FIG. 14A shows an oligo configuration comprising a target sequence and a barcode. FIG. 14B shows treatment of cleavage products to blunt 5′ and 3′ overhangs or end repair to fill in the 5′ overhangs. FIG. 14C shows amplification of cleavage products.

FIGS. 15A-15E include diagrams showing gene editing using exemplary gene editing systems disclosed herein. FIG. 15A is a schematic depicting in vitro cleavage sites (triangles) induced by Cas12i2 of SEQ ID NO: 2 on the PAM strand and non-PAM strand of an AAVS1_T2 genomic site. FIG. 15B is a histogram of read lengths obtained from amplification of 5′ cleavage products following fill-in treatment. FIG. 15C is a histogram of read lengths obtained from amplification of 3′ cleavage products following fill-in treatment. FIG. 15D is a histogram of read lengths obtained from amplification of 5′ cleavage products following blunting treatment. FIG. 15E is a histogram of read lengths obtained from amplification of 3′ cleavage products following blunting treatment. Each read length histogram is mapped to the target sequence as shown on the x-axis of FIGS. 15B-15E.

FIGS. 16A-16B show in vitro cleavage sites (triangles) induced by Cas12i2 of SEQ ID NO: 2 or variant Cas12i2 of SEQ ID NO: 4 on the PAM strand or the non-PAM strand of an EMX1_T6 genomic site (FIG. 16A) and a VEGFA_T5 genomic site (FIG. 16B). The scale bar (right) represents the cleavage frequency as measured by the number of sequencing reads.

FIGS. 17A-17B include diagrams showing gene editing results at exemplary genomic sizes. FIG. 17A shows activity by editing template RNAs introducing 4-nucleotide insertions into an AAVS1_T7 genomic site (SEQ ID NO: 30), an EMX1_T6 genomic site (SEQ ID NO: 34), or a VEGFA_T5 genomic site (SEQ ID NO: 38). The editing template RNAs comprised a 34-nucleotide reverse transcription template sequence and a 3, 8, 13, 30, or 60-nucleotide PBS. Ratio of encoded edits to total edits is shown on the y-axis. FIG. 17B shows activity by editing template RNAs in introducing 4-nucleotide insertions into the AAVS1_T7 genomic site (SEQ ID NO: 30), the EMX1_T6 genomic site (SEQ ID NO: 34), or the VEGFA_T5 genomic site (SEQ ID NO: 38). The editing template RNAs comprised a 13-nucleotide PBS and a 14, 24, 34, 44, or 54-nucleotide reverse transcription template sequence. Ratio of encoded edits to total edits is shown on the y-axis. Sequences in FIG. 17A, from top to bottom, are SEQ ID NOs: 90-92. Sequences in FIG. 17B, from top to bottom, are SEQ ID NOs: 90-92.

FIG. 18 shows encoded edits incorporated into an AAVS1_T7 genomic site (SEQ ID NO: 32) and an EMX1_T6 genomic site (SEQ ID NO: 34) in U2OS cells.

FIGS. 19A-19B include schematics illustrating gene editing procedures using exemplary gene editing systems disclosed herein. FIG. 19A is a schematic depicting a Cas9 prime editor comprising a Cas9 fused to a reverse transcriptase and a pegRNA. A primer on the target DNA is generated following cleavage of the PAM strand by Cas9. Hybridization of the primer with the pegRNA initiates reverse transcription. FIG. 19B is a schematic depicting a Type V CRISPR nuclease fused to a reverse transcriptase and an editing template RNA. A primer on the target DNA is generated following cleavage of the non-PAM strand by the Type V CRISPR nuclease. Hybridization of the primer with the editing template RNA initiates reverse transcription.

FIGS. 20A-20C include diagrams showing edits at various genomic sites with Cas12i2-RT fusion polypeptides as indicated. FIG. 20A is a plot showing % of NGS reads comprising an indel edit (white bars) or an encoded edit (grey bar) introduced by a variant Cas12i2-RT fusion of SEQ ID NOs: 219-223 at an AAVS1 genomic site. FIG. 20B is a plot showing % of NGS reads comprising an indel edit (white bars) or an encoded edit (grey bar) introduced by a variant Cas12i2-RT fusion of SEQ ID NOs: 219-223 at an EMX1 genomic site. FIG. 20C is a plot showing % of NGS reads comprising an indel edit (white bars) or an encoded edit (grey bar) introduced by a variant Cas12i2-RT fusion of SEQ ID NOs: 219-223 at a VEGFA genomic site.

FIG. 21 is a plot showing % of NGS reads comprising an indel edit or an encoded edit introduced by a variant Cas12i2 (SEQ ID NO: 4) or variant Cas12i2-RT fusion (SEQ ID NO: 219) and an RNA guide or an editing template RNA. The RNA guides and editing template RNAs were either unmodified or comprised terminal phosphorothioate backbone linkages and/or 2′O-methyl nucleotides.

FIG. 22 is a plot showing % of NGS reads comprising an indel edit (white bars) or an encoded edit (grey bar) introduced by a variant Cas12i4-RT fusion at an AAVS1 genomic site.

FIG. 23 is a plot showing % of NGS reads comprising an indel edit (white bars) or an encoded edit (grey bar) introduced by a variant Cas12i2 or a variant Cas12i2-RT fusion, an RNA guide, and an RT donor RNA at an AAVS1, EMX1, or VEGFA genomic site.

### DETAILED DESCRIPTION

The present disclosure relates to gene editing systems comprising a Type V nuclease or a nucleic acid encoding such, an RNA guide or a nucleic acid encoding such, a reverse transcriptase or a nucleic acid encoding such, and an RT donor RNA or a nucleic acid encoding such. Also provided herein are pharmaceutical compositions and kits comprising any of the gene editing systems disclosed herein, methods for genetically editing a cell using any of the gene editing systems disclosed herein, genetically engineered cells thus produced, and gene editing RNA molecules or a set of RNA molecules involved in the gene editing system, as well as DNA molecule(s) for producing such.

### Definitions

The present disclosure will be described with respect to particular embodiments and with reference to certain Figures, but the disclosure is not limited thereto but only by the claims. Terms as set forth hereinafter are generally to be understood in their common sense unless indicated otherwise.

As used herein, the term “activity” refers to a biological activity. In some embodiments, the activity refers to effector activity. In some embodiments, activity includes enzymatic activity, e.g., catalytic ability of an effector. For example, activity can include nuclease activity. In another example, activity refers to the ability of an enzyme to generate DNA from RNA or to introduce an edit into a target sequence.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the term “CRISPR nuclease” refers to an RNA-guided effector that is capable of binding a nucleic acid and introducing a single-stranded break or double-stranded break. In some embodiments, a CRISPR nuclease is a Type II CRISPR nuclease or a Type V CRISPR nuclease. In some embodiments, a CRISPR nuclease is an effector as described in Makarova et al. “Classification and Nomenclature of CRISPR-Cas Systems: Where from Here?” CRISPRJ. 1(5):325-36 (2018).

As used herein, the term “Type II” and “Type II nuclease” refers to a nuclease comprising a RuvC domain and an HNH domain. The Type II nuclease can be a Type II-A nuclease, a Type II-B nuclease, or a Type II-C nuclease. In some embodiments, the Type II nuclease requires a tracrRNA. In some embodiments, the Type II nuclease is a Cas9 polypeptide. The Cas9 polypeptide can cleave a double-stranded DNA target or be a nickase.

As used herein, the terms “Type V” and “Type V nuclease” refer to an RNA-guided CRISPR nuclease with a RuvC domain. In some embodiments, a Type V nuclease does not require a tracrRNA. In some embodiments, a Type V nuclease requires a tracrRNA. In some embodiments, the Type V nuclease is a Cas12 polypeptide, such as a Cas12a (Cpf1), Cas12b (C2c1), Cas12c, Cas12d, Cas12e, Cas12f, Cas12h, Cas12i, or Cas12j (CasPhi) polypeptide.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference for the subject matter and purpose referenced herein. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 8, 2, 11, and 9 of the present application. In some embodiments, a Cas12i polypeptide of the disclosure is a Cas12i2 polypeptide as described in WO/2021/202800, the relevant disclosures of which are incorporated by reference for the subject matter and purpose referenced herein. In some embodiments, the Cas12i polypeptide cleaves a target nucleic acid (e.g., as a nick or a double strand break).

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the term “complex” refers to a grouping of two or more molecules. In some embodiments, the complex comprises a polypeptide and a nucleic acid molecule interacting with (e.g., binding to, coming into contact with, adhering to) one another. In some embodiments, the term “complex” is used to refer to association of a CRISPR nuclease (e.g., a Type V nuclease such as a Cas12i polypeptide) and a reverse transcriptase polypeptide. For example, a complex of a CRISPR nuclease (e.g., a Cas12i2 polypeptide as disclosed herein) and a reverse transcriptase polypeptide may be a heterodimer of the two polypeptides, e.g., via a dimerization domain (e.g., a leucine zipper), an antibody, a nanobody, or an aptamer. In some embodiments, the term “complex” is used to refer to association of an RNA guide and an RT donor RNA. In some embodiments, the term “complex” is used to refer to association of a CRISPR nuclease (e.g., a Type V nuclease such as a Cas12i polypeptide), a reverse transcriptase polypeptide, an RNA guide, and an RT donor RNA. In some embodiments, the term “complex” is used to refer to association of a reverse transcriptase polypeptide and an RT donor RNA.

As used herein, the term “binding site recognizable by a nuclease” or “nuclease binding sequence” refers to a sequence that is capable of binding to a CRISPR nuclease. In some embodiments, the nuclease binding sequence is an RNA sequence. In some embodiments, the nuclease binding sequence is a direct repeat sequence. In some embodiments, a nuclease binding sequence is capable of binding to a Type II CRISPR nuclease or a Type V CRISPR nuclease (e.g., binding site recognizable by a Type II CRISPR nuclease, or binding site recognizable by a Type V CRISPR nuclease).

As used herein, the term “deletion” refers to a loss of a nucleotide or nucleotides in a nucleic acid sequence, relative to a reference sequence. No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to an insertion of up to several kilobases.

As used herein, the term “edit” refers to one or more modifications introduced into a nucleotide sequence in a target nucleic acid such as in a genomic site of interest. The edit may occur within a target sequence as defined herein. Alternatively, the edit may occur outside the target sequence (e.g., adjacent to the target sequence). The edit can be one or more substitutions, one or more insertions, one or more deletions, or a combination thereof.

As used herein, the terms “fusion” and “fused” refer to the joining of at least two nucleotide or protein molecules. For example, “fusion” and “fused” can refer to the joining of at least two polypeptide domains that are encoded by separate genes (e.g., a Type V nuclease and a reverse transcriptase polypeptide) in nature. The fusion can be an N-terminal fusion, a C-terminal fusion, or an intramolecular fusion. In some aspects, the domains are transcribed and translated to produce a single polypeptide. Also as used herein, the terms “fusion” and “fused” are used to refer to the joining of two nucleic acid molecules, such as two RNA molecules (e.g., an RNA guide and an RT donor RNA). The fusion can be a 5′ fusion, a 3′ fusion, or an intramolecular fusion.

As used herein, the term “insertion” refers to a gain of a nucleotide or nucleotides in a nucleic acid sequence, relative to a reference sequence. No particular process is implied in how to make a sequence comprising an insertion. For instance, a sequence comprising an insertion can be synthesized directly from individual nucleotides. In other embodiments, an insertion is made by providing and then altering a reference sequence. The nucleic acid sequence can be in a genome of an organism. The nucleic acid sequence can be in a cell. The nucleic acid sequence can be a DNA sequence. The insertion can be a frameshift mutation or a non-frameshift mutation. An insertion described herein refers to an insertion of up to several kilobases.

As used herein, the term “protospacer adjacent motif” or “PAM sequence” refers to a DNA sequence adjacent to a target sequence. In some embodiments, a PAM sequence is required for enzyme activity. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein, and the PAM sequence as described herein is present in the PAM-strand.

As used herein, the term “PAM strand” refers to the strand of a target nucleic acid (double-stranded) that comprises a PAM motif. In some embodiments, the PAM strand is a coding (e.g., sense) strand. In other embodiments, the PAM strand is a non-coding (e.g., antisense strand). The term “non-PAM strand” refers to the complementary strand of the PAM strand. Since a gRNA binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein).

As used herein, the terms “RNA guide” or “RNA guide sequence” refer to an RNA molecule or a modified RNA molecule that facilitates the targeting of a CRISPR nuclease described herein to a genomic site of interest. For example, an RNA guide can be a molecule that recognizes (e.g., binds to) a site in a non-PAM strand that is complementary to a target sequence in the PAM strand, e.g., designed to be complementary to a specific nucleic acid sequence. An RNA guide comprises a spacer and a nuclease binding sequence (e.g., a direct repeat (DR) sequence). The terms CRISPR RNA (crRNA), pre-crRNA and mature crRNA are also used herein to refer to an RNA guide. The 5′ end or 3′ end of an RNA guide may be fused to an RT donor RNA as disclosed herein. In some instances, the RNA guide can be a modified RNA molecule comprising one or more deoxyribonucleotides, for example, in a DNA-binding sequence contained in the RNA guide, which binds the complementary sequence of the target sequence. In some examples, the DNA-binding sequence may contain a DNA sequence or a DNA/RNA hybrid sequence.

As used herein, the term “spacer” and “spacer sequence” (a.k.a., a DNA-binding sequence) is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide (e.g., a Cas12i2 polypeptide, a Cas12i2-reverse transcriptase fusion polypeptide, or a variant thereof) that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

As used herein, the terms “reverse transcriptase” and “RT” refer to a multi-functional enzyme that typically has three enzymatic activities including RNA- and DNA-dependent DNA polymerization activity and an RNase H activity that catalyzes the cleavage of RNA in RNA-DNA hybrids. A reverse transcriptase can generate DNA from an RNA template.

As used herein, the terms “reverse transcription donor RNA” and “RT donor RNA” refer to an RNA molecule comprising a reverse transcription template sequence (template sequence) and a primer binding site (PBS). An RT donor RNA may be fused to an RNA guide at either the 5′ end or 3′ end of the RNA guide.

As used herein, the term “PBS-targeting site” refers to the region to which a PBS binds. The PBS-targeting site may be adjacent to (e.g., upstream to) a region of the non-PAM strand that is complementary to the target sequence. For example, the PBS-targeting site can be 3-10 nucleotides (e.g., 3-nucleotide or 4-nucleotide) upstream to the region that is complementary to the target sequence. In some instances, the PBS-targeting site may be immediately adjacent to the region of the non-PAM stand that is complementary to the target sequence. In other examples, the PBS-targeting site may overlap with the region of the non-PAM strand that is complementary to the target sequence. Alternatively, the PBS-targeting site may be adjacent to, upstream to, or overlap with the target sequence on the PAM strand.

As used herein, the term “reverse transcription template sequence” or “template sequence” refers to an RNA molecule or a fragment of an RT donor RNA that serves as a template for DNA synthesis by a reverse transcriptase. In some embodiments, the reverse transcription template sequence comprises an edit to be incorporated into a genomic site where gene editing is needed. In some instances, an edit mediated by the reverse transcription template sequence in the RT donor RNA disrupts or removes the PAM sequence, the target sequence, or both.

As used herein, the term “editing template RNA” or “gene editing RNA” (used herein interchangeably) refers to an RNA molecule or a set of RNA molecules comprising an RNA guide (comprising a spacer and one or more binding site recognizable by a CRISPR nuclease such as those disclosed herein) and a RT doner RNA (comprising a PBS and a reverse transcription template sequence). A gene editing RNA is capable of mediating cleavage at a target sequence within a genomic site of interest by a CRISPR nuclease and synthesis of a DNA fragment from a free 3′end of a free DNA strand generated by the CRISPR nuclease cleavage based on the template sequence in the gene editing RNA. In some embodiments, an editing template RNA or gene editing RNA is a single RNA molecule comprising the RNA guide linked (e.g., fused) to the RT donor RNA. In some embodiments, an editing template RNA from 5′ to 3′ comprises one or more binding site recognizable by a CRISPR nuclease, a spacer sequence, a PBS, and an RT donor RNA. In some embodiments, an editing template RNA or gene editing RNA from 5′ to 3′ comprises one or more binding site recognizable by a CRISPR nuclease, a spacer, a template sequence, and a PBS. In some embodiments, an editing template RNA or gene editing RNA from 5′ to 3′ comprises a template sequence, a PBS, one or more binding site recognizable by a CRISPR nuclease, and a spacer sequence. In some embodiments, an editing template RNA further comprises a linker. For example, in some embodiments, an editing template RNA comprises a linker between the one or more binding site recognizable by a CRISPR nuclease and the PBS or between the spacer sequence and the RT donor RNA.

As used herein, the term “substitution” refers to a replacement of a nucleotide or nucleotides with a different nucleotide or nucleotides, relative to a reference sequence. No particular process is implied in how to make a sequence comprising a substitution. For instance, a sequence comprising a substitution can be synthesized directly from individual nucleotides. In other embodiments, a substitution is made by providing and then altering a reference sequence. The nucleic acid sequence can be in a genome of an organism. The nucleic acid sequence can be in a cell. The nucleic acid sequence can be a DNA sequence. The substitution described herein refers to a substitution of up to several kilobases.

As used herein, the terms “upstream” and “downstream” refer to relative positions within a single nucleic acid (e.g., DNA) sequence. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the terms “upstream” and downstream” are used in reference to a non-PAM strand. For example, in some embodiments, a PBS is complementary to a non-PAM strand sequence that is upstream of a target sequence. As such, in some embodiments, a PBS binds to a sequence upstream of a sequence to which a spacer sequence binds, and the spacer sequence binds downstream of a sequence to which the PBS binds.

### I. Gene Editing Systems

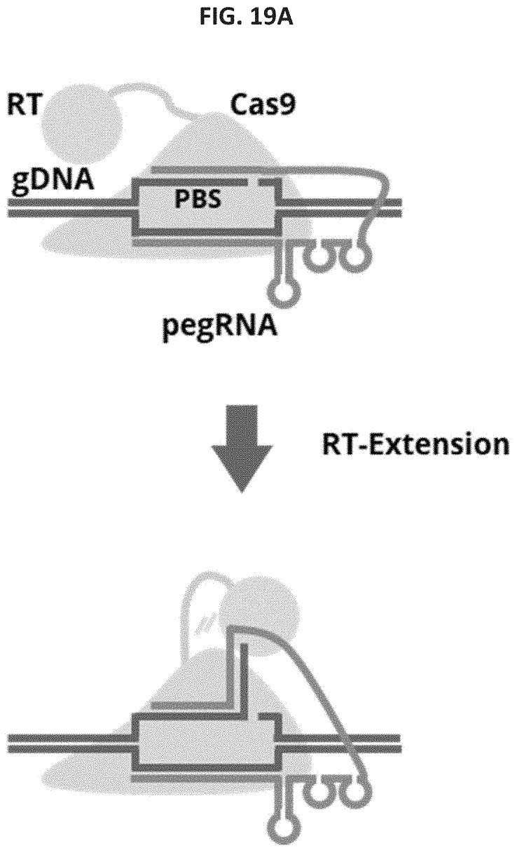

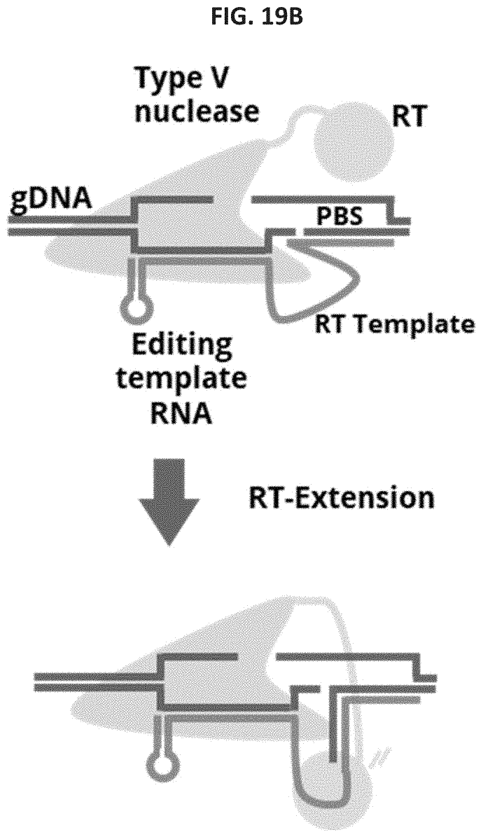

Prime editing was developed to introduce substitutions, small insertions, or small deletions into target sequences. The prime editing approach relies on a Cas9 nickase fused to a reverse transcriptase and a prime editing guide RNA (pegRNA). The pegRNA comprises a spacer sequence capable of binding to the non-PAM strand of a target locus (strand opposite of the PAM sequence), a primer binding site (PBS) capable of binding to the PAM strand of the target locus (strand comprising the PAM sequence), and a reverse transcription template sequence comprising an edit. The spacer sequence of the pegRNA binds to the target sequence on the non-PAM strand, and the nickase Cas9 nicks the PAM strand. This exposes a 3′ flap on the PAM strand of the target locus that can hybridize to the PBS. The reverse transcriptase then copies the reverse transcription template, thereby extending the 3′ flap. See, e.g., FIG. 19A. Through DNA repair mechanisms, the edit is incorporated into the target locus.

Provided herein, in some aspects, is a gene editing system capable of editing a target nucleic acid (e.g., at a genomic site of interest), e.g., introducing insertion, deletion, substitution, or a combination thereof, at the genomic site. The edit may occur on either strand of the target nucleic acid. The gene editing system disclosed herein comprises at least one protein component or a nucleotide sequence encoding such, and at least one RNA component or a nucleotide sequence encoding such. The protein component has the activity of cleaving the target nucleic acid at a desired site guided by the RNA component and the activity of synthesizing new DNA sequences, starting from the free 3′end of a DNA strand generated due to the cleavage, using portion of the RNA component as a template. The newly synthesized DNA fragment can then be incorporated into the target nucleic acid via, e.g., the DNA repair mechanisms in a host cell, leading to the genetic editing of the target nucleic acid.

The protein component in the gene editing system disclosed herein may comprise a CRISPR nuclease (e.g., a Type V nuclease such as a variant Cas12i polypeptide) and a reverse transcriptase (RT) polypeptide. In some examples, the CRISPR nuclease and the RT polypeptide are two separate polypeptides. In other examples, the CRISPR nuclease and the RT polypeptide are parts of a fusion polypeptide.

The RNA component in the gene editing system disclosed herein may comprise a guide RNA (gRNA) (also described as an RNA guide or CRISPR RNA (crRNA) herein), which mediates CRISPR nuclease cleavage at a particular site in a target nucleic acid as designed, and a reverse transcription donor RNA (RT donor RNA), which mediates reverse transcription by the RT polypeptide and provides a template sequence for the reverse transcription. In some examples, the gRNA and the RT donor RNA are two separate RNA molecules. In other examples, the gRNA and the RT donor RNA are parts of a single RNA molecule.

As shown herein and without being bound by theory, the gene editing systems described herein provide several advantages over the art. For example, RNA-templated editing has not been demonstrated with a Type V CRISPR nuclease, such as a Cas12i CRISPR nuclease. There is a wealth of Type V nucleases that are smaller than Cas9 nucleases. For example, Cas12i2 is 1,054 amino acids in length, whereas S. pyogenes Cas9 (SpCas9) is 1,368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Additionally, many Type V nucleases utilize RNA guides that do not require a trans-activating CRISPR RNA (tracrRNA) and are thus smaller than Cas9 RNA guides. See, e.g., Table 4 below. The smaller Cas12i polypeptide and RNA guide sizes are beneficial for delivery. Additionally, RNA-templated editing has not been demonstrated with any CRISPR nuclease utilizing a single editing template RNA that binds a single strand of the target locus, such as the target strand (non-PAM strand). As shown herein, gene editing systems comprising a Cas12i polypeptide also demonstrate decreased off-target activity compared to gene editing systems comprising an SpCas9 polypeptide. See PCT/US2021/025257, which is incorporated by reference in its entirety.

A. CRISPR Nuclease

Any of the gene editing systems disclosed herein may comprises a CRISPR nuclease. In some embodiments, a CRISPR nuclease is capable of binding and/or binds to a nuclease binding sequence as described elsewhere herein. In some embodiments, a CRISPR nuclease cleaves DNA at a target sequence. In some embodiments, a CRISPR nuclease is recruited to a target sequence via a DNA-binding sequence described elsewhere herein that specifically recognizes and/or binds at the target sequence. In some embodiments, a CRISPR nuclease cleaves one or both strands of DNA at a target sequence. In some embodiments, more than one CRISPR nuclease is recruited to a target sequence and one or more CRISPR nucleases cleaves one or both strands of DNA at or near the target sequence. In such embodiments, the CRISPR nuclease may possess or be capable of nuclease activity. In some embodiments, the CRISPR nuclease may possess reduced or limited nuclease activity. In some embodiments, a CRISPR nuclease-reverse transcriptase fusion polypeptide as described elsewhere herein is capable of binding and binds to at least one nuclease binding sequence in an editing template RNA as described elsewhere herein. In some embodiments, the CRISPR nuclease-reverse transcriptase fusion is capable of binding and binds to a target sequence through at least one DNA-binding sequence in an editing template RNA. In such embodiments, the CRISPR nuclease is recruited to or brought in close proximity to a target sequence by binding to the nuclease binding sequence and the DNA-binding sequence of the editing template RNA. Further in such embodiments, the reverse transcriptase is capable of transcribing and transcribes a reverse transcription template sequence as described elsewhere herein into DNA.

In some embodiments, a CRISPR nuclease-reverse transcriptase fusion polypeptide transcribes the reverse transcription template sequence into the non-PAM strand of a target nucleic acid. In some embodiments, a CRISPR nuclease-reverse transcriptase fusion polypeptide transcribes the reverse transcription template sequence into the PAM strand of a target nucleic acid. In some embodiments, a CRISPR nuclease-reverse transcriptase fusion polypeptide transcribes the reverse transcription template sequence from 5′ to 3′ starting from the PBS (e.g., the 5′ or 3′ end of the PBS). In some embodiments, following hybridization of a PBS to a free 3′ end of a non-PAM strand of a target nucleic acid, a CRISPR nuclease-reverse transcriptase fusion transcribes the reverse transcription template sequence from the 3′ end of the non-PAM strand. In some embodiments, following hybridization of a PBS to a free 3′ end of a PAM strand of a target nucleic acid, a CRISPR nuclease-reverse transcriptase fusion transcribes the reverse transcription template sequence from the 3′ end of the PAM strand.

In some embodiments, the CRISPR nuclease is an RNA-guided CRISPR nuclease. In some embodiments, the CRISPR nuclease is a DNA-targeting nuclease.

In some embodiments, the CRISPR nuclease is Cas9 (e.g., Cas9 and nCas9), Cas12a/Cpf1, Cas12b/C2c1, Cas12c/C2c3, Cas12d/CasY, Cas12e/CasX, Cas12 g, Cas12h, Cas12i, and Cas12j/CasPhi). Non-limiting examples of Cas enzymes include Cas1, CasIB, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12 g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, a Type II CRISPR nuclease, a Type V CRISPR nuclease, a Type VI CRISPR nuclease, CARF, DinG, homologue thereof, or modified or engineered version thereof. Other CRISPR nucleases are also within the scope of this disclosure, although they may not be specifically listed in this disclosure. See, e.g., Makarova et al. “Classification and Nomenclature of CRISPR-Cas Systems: Where from Here?” CRISPRJ. 1(5):325-36 (2018).

In some embodiments, the CRISPR nuclease is a nuclease disclosed in WO2021055874, WO2020206036, WO2020191102, WO2020186213, WO2020028555, WO2020033601, WO2019126762, WO2019126774, WO2019071048, WO2019018423, WO2019005866, WO2018191388, WO2018170333, WO2018035388, WO2018035387, WO2017219027, WO2017189308, WO2017184768, WO2017106657, WO2016205749, WO2017070605, WO2016205764, WO2016205711, WO2016028682, WO2015089473, WO2014093595, WO2015089427, WO2014204725, WO2015070083, WO2014093655, WO2014093694, WO2014093712, WO2014093635, WO2021133829, WO2021007177, WO2020197934, WO2020181102, WO2020181101, WO2020041456, WO2020023529, WO2020005980, WO2019104058, WO2019089820, WO2019089808, WO2019089804, WO2019089796, WO2019036185, WO2018226855, WO2018213351, WO2018089664, WO2018064371, WO2018064352, WO2017106569, WO2017048969, WO2016196655, WO2016106239, WO2016036754, WO2015103153, WO2015089277, WO2014150624, WO2013176772, WO2021119563, WO2021118626, WO2020247883, WO2020247882, WO2020223634, WO2020142754, WO2020086475, WO2020028729, WO2019241452, WO2019173248, WO2018236548, WO2018183403, WO2017027423, WO2018106727, WO2018071672, WO2017096328, WO2017070598, WO2016201155, WO2014150624, WO2013098244, WO2021113522, WO2021050534, WO2021046442, WO2021041569, WO2021007563, WO2020252378, WO2020180699, WO2020018142, WO2019222555, WO2019178428, WO2019178427, or WO2019006471, which are incorporated by reference for the subject matter and purpose referenced herein.

In some embodiments, a composition of the present invention comprises a Type V CRISPR nuclease (e.g., a Type V nuclease). In some embodiments, the Type V nuclease is a Cas12 CRISPR nuclease. In some embodiments, the Type V nuclease is a Cas12a (Cpf1), Cas12b (C2c1), Cas12c, Cas12d, Cas12e, Cas12f, Cas12h, Cas12i, or Cas12j (CasPhi) CRISPR nuclease. In some embodiments, the Type V nuclease is a variant (e.g., a functional variant) of a Cas12a (Cpf1), Cas12b (C2c1), Cas12c, Cas12d, Cas12e, Cas12f, Cas12h, Cas12i, or Cas12j (CasPhi) CRISPR nuclease. In some embodiments, the Type V nuclease comprises an amino acid sequence with at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to a wild-type Type V nuclease sequence (e.g., a wild-type amino acid sequence of Cas12a (Cpf1), Cas12b (C2c1), Cas12c, Cas12d, Cas12e, Cas12f, Cas12h, Cas12i, or Cas12j (CasPhi).

In some embodiments, the Type V nuclease of the present invention is a Cas12i CRISPR nuclease. In some embodiments, the Cas12i CRISPR nuclease is a Cas12i2 CRISPR nuclease comprising a nucleotide sequence such as SEQ ID NO: 1 or is encoded by polypeptide comprising an amino acid sequence such as SEQ ID NO: 2. In some embodiments, the CRISPR nuclease of the present invention is a variant of a wildtype CRISPR nuclease, wherein the wildtype comprises a nucleotide sequence such as SEQ ID NO: 1 or is encoded by a polypeptide that comprises an amino acid sequence such as SEQ ID NO: 2. See Table 1.

In some embodiments, the Type II nuclease of the present invention is a Cas9 CRISPR nuclease. In some embodiments, the Cas9 CRISPR nuclease is an SpCas9 CRISPR nuclease comprising an amino acid sequence such as SEQ ID NO: 120. In some embodiments, the Cas9 CRISPR nuclease is a nickase, e.g., an nSpCas9 comprising an amino acid sequence such as SEQ ID NO: 121. In some embodiments, the CRISPR nuclease of the present invention is a different species of a Cas9 CRISPR nuclease. In some embodiments, the Cas9 CRISPR nuclease is an SaCas9 CRISPR nuclease comprising an amino acid sequence such as SEQ ID NO: 122. See Table 1.

TABLE 1
Cas12i and Cas9 Sequences.

| SEQ ID NO | Sequence | Description |
|---|---|---|
| 1 | ATGAGCAGCGCGATCAAAAGCTACAAGAGCGTTCTGCGTCCGAACGAGCG | Nucleotide |
| | TAAGAACCAACTGCTGAAAAGCACCATTCAGTGCCTGGAAGACGGTAGCG | sequence |
| | CGTTCTTTTTCAAGATGCTGCAAGGCCTGTTTGGTGGCATCACCCCGGAG | encoding |
| | ATTGTTCGTTTCAGCACCGAACAGGAGAAACAGCAACAGGATATCGCGCT | Cas12i2 |
| | GTGGTGCGCGGTTAACTGGTTCCGTCCGGTGAGCCAAGACAGCCTGACCC | |
| | ACACCATTGCGAGCGATAACCTGGTGGAGAAGTTTGAGGAATACTATGGT | |
| | GGCACCGCGAGCGACGCGATCAAACAGTACTTCAGCGCGAGCATTGGCGA | |
| | AAGCTACTATTGGAACGACTGCCGTCAACAGTACTATGATCTGTGCCGTG | |
| | AGCTGGGTGTTGAGGTGAGCGACCTGACCCATGATCTGGAGATCCTGTGC | |
| | CGTGAAAAGTGCCTGGCGGTTGCGACCGAGAGCAACCAGAACAACAGCAT | |
| | CATTAGCGTTCTGTTTGGCACCGGCGAAAAAGAGGACCGTAGCGTGAAAC | |
| | TGCGTATCACCAAGAAAATTCTGGAGGCGATCAGCAACCTGAAAGAAATC | |
| | CCGAAGAACGTTGCGCCGATTCAAGAGATCATTCTGAACGTGGCGAAAGC | |
| | GACCAAGGAAACCTTCCGTCAGGTGTATGCGGGTAACCTGGGTGCGCCGA | |
| | GCACCCTGGAGAAATTTATCGCGAAGGACGGCCAAAAAGAGTTCGATCTG | |
| | AAGAAACTGCAGACCGACCTGAAGAAAGTTATTCGTGGTAAAAGCAAGGA | |
| | GCGTGATTGGTGCTGCCAGGAAGAGCTGCGTAGCTACGTGGAGCAAAACA | |
| | CCATCCAGTATGACCTGTGGGCGTGGGGCGAAATGTTCAACAAAGCGCAC | |
| | ACCGCGCTGAAAATCAAGAGCACCCGTAACTACAACTTTGCGAAGCAACG | |
| | TCTGGAACAGTTCAAAGAGATTCAGAGCCTGAACAACCTGCTGGTTGTGA | |
| | AGAAGCTGAACGACTTTTTCGATAGCGAATTTTTCAGCGGCGAGGAAACC | |
| | TACACCATCTGCGTTCACCATCTGGGTGGCAAGGACCTGAGCAAACTGTA | |
| | TAAGGCGTGGGAGGATGATCCGGCGGACCCGGAAAACGCGATTGTGGTTC | |
| | TGTGCGACGATCTGAAAAACAACTTTAAGAAAGAGCCGATCCGTAACATT | |
| | CTGCGTTACATCTTCACCATTCGTCAAGAATGCAGCGCGCAGGACATCCT | |
| | GGCGGCGGCGAAGTACAACCAACAGCTGGATCGTTATAAAAGCCAAAAGG | |
| | CGAACCCGAGCGTTCTGGGTAACCAGGGCTTTACCTGGACCAACGCGGTG | |
| | ATCCTGCCGGAGAAGGCGCAGCGTAACGACCGTCCGAACAGCCTGGATCT | |
| | GCGTATTTGGCTGTACCTGAAACTGCGTCACCCGGACGGTCGTTGGAAGA | |
| | AACACCATATCCCGTTCTACGATACCCGTTTCTTCCAAGAAATTTATGCG | |
| | GCGGGCAACAGCCCGGTTGACACCTGCCAGTTTCGTACCCCGCGTTTCGG | |
| | TTATCACCTGCCGAAACTGACCGATCAGACCGCGATCCGTGTTAACAAGA | |
| | AACATGTGAAAGCGGCGAAGACCGAGGCGCGTATTCGTCTGGCGATCCAA | |
| | CAGGGCACCCTGCCGGTGAGCAACCTGAAGATCACCGAAATTAGCGCGAC | |
| | CATCAACAGCAAAGGTCAAGTGCGTATTCCGGTTAAGTTTGACGTGGGTC | |
| | GTCAAAAAGGCACCCTGCAGATCGGTGACCGTTTCTGCGGCTACGATCAA | |
| | AACCAGACCGCGAGCCACGCGTATAGCCTGTGGGAAGTGGTTAAAGAGGG | |
| | TCAATACCATAAAGAGCTGGGCTGCTTTGTTCGTTTCATCAGCAGCGGTG | |
| | ACATCGTGAGCATTACCGAGAACCGTGGCAACCAATTTGATCAGCTGAGC | |
| | TATGAAGGTCTGGCGTACCCGCAATATGCGGACTGGCGTAAGAAAGCGAG | |
| | CAAGTTCGTGAGCCTGTGGCAGATCACCAAGAAAAACAAGAAAAAGGAAA | |
| | TCGTGACCGTTGAAGCGAAAGAGAAGTTTGACGCGATCTGCAAGTACCAG | |
| | CCGCGTCTGTATAAATTCAACAAGGAGTACGCGTATCTGCTGCGTGATAT | |
| | TGTTCGTGGCAAAAGCCTGGTGGAACTGCAACAGATTCGTCAAGAGATCT | |
| | TTCGTTTCATTGAACAGGACTGCGGTGTTACCCGTCTGGGCAGCCTGAGC | |
| | CTGAGCACCCTGGAAACCGTGAAAGCGGTTAAGGGTATCATTTACAGCTA | |
| | TTTTAGCACCGCGCTGAACGCGAGCAAGAACAACCCGATCAGCGACGAAC | |
| | AGCGTAAAGAGTTTGATCCGGAACTGTTCGCGCTGCTGGAAAAGCTGGAG | |
| | CTGATTCGTACCCGTAAAAAGAAACAAAAAGTGGAACGTATCGCGAACAG | |
| | CCTGATTCAGACCTGCCTGGAGAACAACATCAAGTTCATTCGTGGTGAAG | |
| | GCGACCTGAGCACCACCAACAACGCGACCAAGAAAAAGGCGAACAGCCGT | |
| | AGCATGGATTGGTTGGCGCGTGGTGTTTTTAACAAAATCCGTCAACTGGC | |
| | GCCGATGCACAACATTACCCTGTTCGGTTGCGGCAGCCTGTACACCAGCC | |
| | ACCAGGACCCGCTGGTGCATCGTAACCCGGATAAAGCGATGAAGTGCCGT | |
| | TGGGCGGCGATCCCGGTTAAGGACATTGGCGATTGGGTGCTGCGTAAGCT | |
| | GAGCCAAAACCTGCGTGCGAAAAACATCGGCACCGGCGAGTACTATCACC | |
| | AAGGTGTTAAAGAGTTCCTGAGCCATTATGAACTGCAGGACCTGGAGGAA | |
| | GAGCTGCTGAAGTGGCGTAGCGATCGTAAAAGCAACATTCCGTGCTGGGT | |
| | GCTGCAGAACCGTCTGGCGGAGAAGCTGGGCAACAAAGAAGCGGTGGTTT | |
| | ACATCCCGGTTCGTGGTGGCCGTATTTATTTTGCGACCCACAAGGTGGCG | |
| | ACCGGTGCGGTGAGCATCGTTTTCGACCAAAAACAAGTGTGGGTTTGCAA | |
| | CGCGGATCATGTTGCGGCGGCGAACATCGCGCTGACCGTGAAGGGTATTG | |
| | GCGAACAAAGCAGCGACGAAGAGAACCCGGATGGTAGCCGTATCAAACTG | |
| | CAGCTGACCAGC | |
| 2 | MSSAIKSYKSVLRPNERKNQLLKSTIQCLEDGSAFFFKMLQGLFGGITPE | Cas12i2 |
| | IVRFSTEQEKQQQDIALWCAVNWFRPVSQDSLTHTIASDNLVEKFEEYYG | amino acid |
| | GTASDAIKQYFSASIGESYYWNDCRQQYYDLCRELGVEVSDLTHDLEILC | sequence |
| | REKCLAVATESNQNNSIISVLFGTGEKEDRSVKLRITKKILEAISNLKEI | |
| | PKNVAPIQEIILNVAKATKETFRQVYAGNLGAPSTLEKFIAKDGQKEFDL | |
| | KKLQTDLKKVIRGKSKERDWCCQEELRSYVEQNTIQYDLWAWGEMFNKAH | |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFDVGRQKGTLQIGDRFCGYDQ | |
| | NQTASHAYSLWEVVKEGQYHKELGCFVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLAPMHNITLFGCGSLYTSHQDPLVHRNPDKAMKCR | |
| | WAAIPVKDIGDWVLRKLSQNLRAKNIGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTVKGIGEQSSDEENPDGSRIKL | |
| | QLTS | |
| 3 | MSSAIKSYKSVLRPNERKNQLLKSTIQCLEDGSAFFFKMLQGLFGGITPE | Variant |
| | IVRFSTEQEKQQQDIALWCAVNWFRPVSQDSLTHTIASDNLVEKFEEYYG | Cas12i2 of |
| | GTASDAIKQYFSASIGESYYWNDCRQQYYDLCRELGVEVSDLTHDLEILC | SEQ ID |
| | REKCLAVATESNQNNSIISVLFGTGEKEDRSVKLRITKKILEAISNLKEI | NO: 3 of |
| | PKNVAPIQEIILNVAKATKETFRQVYAGNLGAPSTLEKFIAKDGQKEFDL | PCT/US20 |
| | KKLQTDLKKVIRGKSKERDWCCQEELRSYVEQNTIQYDLWAWGEMFNKAH | 21/025257 |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFRVGRQKGTLQIGDRFCGYDQ | |
| | NQTASHAYSLWEVVKEGQYHKELGCFVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLAPMHNITLFGCGSLYTSHQDPLVHRNPDKAMKCR | |
| | WAAIPVKDIGRWVLRKLSQNLRAKNRGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTGKGIGEQSSDEENPDGSRIKL | |
| | QLTS | |
| 4 | MSSAIKSYKSVLRPNERKNQLLKSTIQCLEDGSAFFFKMLQGLFGGITPE | Variant |
| | IVRFSTEQEKQQQDIALWCAVNWFRPVSQDSLTHTIASDNLVEKFEEYYG | Cas12i2 of |
| | GTASDAIKQYFSASIGESYYWNDCRQQYYDLCRELGVEVSDLTHDLEILC | SEQ ID |
| | REKCLAVATESNQNNSIISVLFGTGEKEDRSVKLRITKKILEAISNLKEI | NO: 4 of |
| | PKNVAPIQEIILNVAKATKETFRQVYAGNLGAPSTLEKFIAKDGQKEFDL | PCT/US20 |
| | KKLQTDLKKVIRGKSKERDWCCQEELRSYVEQNTIQYDLWAWGEMFNKAH | 21/025257 |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFRVGRQKGTLQIGDRFCGYDQ | |
| | NQTASHAYSLWEVVKEGQYHKELGCFVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLAPMHNITLFGCGSLYTSHQDPLVHRNPDKAMKCR | |
| | WAAIPVKDIGDWVLRKLSQNLRAKNRGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTGKGIGEQSSDEENPDGSRIKL | |
| | QLTS | |
| 5 | MSSAIKSYKSVLRPNERKNQLLKSTIQCLEDGSAFFFKMLQGLFGGITPE | Variant |
| | IVRFSTEQEKQQQDIALWCAVNWFRPVSQDSLTHTIASDNLVEKFEEYYG | Cas12i2 of |
| | GTASDAIKQYFSASIGESYYWNDCRQQYYDLCRELGVEVSDLTHDLEILC | SEQ ID |
| | REKCLAVATESNQNNSIISVLFGTGEKEDRSVKLRITKKILEAISNLKEI | NO: 5 of |
| | PKNVAPIQEIILNVAKATKETFRQVYAGNLGAPSTLEKFIAKDGQKEFDL | PCT/US20 |
| | KKLQTDLKKVIRGKSKERDWCCQEELRSYVEQNTIQYDLWAWGEMFNKAH | 21/025257 |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFRVGRQKGTLQIGDRFCGYDQ | |
| | NQTASHAYSLWEVVKEGQYHKELGCFVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLAPMHNITLFGCGSLYTSHQDPLVHRNPDKAMKCR | |
| | WAAIPVKDIGDWVLRKLSQNLRAKNRGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTGKGIGEQSSDEENPDGGRIKL | |
| | QLTS | |
| 6 | MSSAIKSYKSVLRPNERKNQLLKSTIQCLEDGSAFFFKMLQGLFGGITPE | Variant |
| | IVRFSTEQEKQQQDIALWCAVNWFRPVSQDSLTHTIASDNLVEKFEEYYG | Cas12i2 of |
| | GTASDAIKQYFSASIGESYYWNDCRQQYYDLCRELGVEVSDLTHDLEILC | SEQ ID |
| | REKCLAVATESNQNNSIISVLFGTGEKEDRSVKLRITKKILEAISNLKEI | NO: 495 of |
| | PKNVAPIQEIILNVAKATKETFRQVYAGNLGAPSTLEKFIAKDGQKEFDL | PCT/US20 |
| | KKLQTDLKKVIRGKSKERDWCCQEELRSYVEQNTIQYDLWAWGEMFNKAH | 21/025257 |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFRVGRQKGTLQIGDRFCGYDQ | |
| | NQTASHAYSLWEVVKEGQYHKELRCRVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLAPMHNITLFGCGSLYTSHQDPLVHRNPDKAMKCR | |
| | WAAIPVKDIGDWVLRKLSQNLRAKNRGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTGKGIGRQSSDEENPDGGRIKL | |
| | QLTS | |
| 7 | MSSAIKSYKSVLRPNERKNQLLKSTIQCLEDGSAFFFKMLQGLFGGITPE | Variant |
| | IVRFSTEQEKQQQDIALWCAVNWFRPVSQDSLTHTIASDNLVEKFEEYYG | Cas12i2 of |
| | GTASDAIKQYFSASIGESYYWNDCRQQYYDLCRELGVEVSDLTHDLEILC | SEQ ID |
| | REKCLAVATESNQNNSIISVLFGTGEKEDRSVKLRITKKILEAISNLKEI | NO: 496 of |
| | PKNVAPIQEIILNVAKATKETFRQVYAGNLGAPSTLEKFIAKDGQKEFDL | PCT/US20 |
| | KKLQTDLKKVIRGKSKERDWCCQEELRSYVEQNTIQYDLWAWGEMFNKAH | 21/025257 |
| | TALKIKSTRNYNFAKQRLEQFKEIQSLNNLLVVKKLNDFFDSEFFSGEET | |
| | YTICVHHLGGKDLSKLYKAWEDDPADPENAIVVLCDDLKNNFKKEPIRNI | |
| | LRYIFTIRQECSAQDILAAAKYNQQLDRYKSQKANPSVLGNQGFTWTNAV | |
| | ILPEKAQRNDRPNSLDLRIWLYLKLRHPDGRWKKHHIPFYDTRFFQEIYA | |
| | AGNSPVDTCQFRTPRFGYHLPKLTDQTAIRVNKKHVKAAKTEARIRLAIQ | |
| | QGTLPVSNLKITEISATINSKGQVRIPVKFRVGRQKGTLQIGDRFCGYDQ | |
| | NQTASHAYSLWEVVKEGQYHKELRCRVRFISSGDIVSITENRGNQFDQLS | |
| | YEGLAYPQYADWRKKASKFVSLWQITKKNKKKEIVTVEAKEKFDAICKYQ | |
| | PRLYKFNKEYAYLLRDIVRGKSLVELQQIRQEIFRFIEQDCGVTRLGSLS | |
| | LSTLETVKAVKGIIYSYFSTALNASKNNPISDEQRKEFDPELFALLEKLE | |
| | LIRTRKKKQKVERIANSLIQTCLENNIKFIRGEGDLSTTNNATKKKANSR | |
| | SMDWLARGVFNKIRQLATMHNITLFGCGSLYTSHQDPLVHRNPDKAMKCR | |
| | WAAIPVKDIGDWVLRKLSQNLRAKNRGTGEYYHQGVKEFLSHYELQDLEE | |
| | ELLKWRSDRKSNIPCWVLQNRLAEKLGNKEAVVYIPVRGGRIYFATHKVA | |
| | TGAVSIVFDQKQVWVCNADHVAAANIALTGKGIGRQSSDEENPDGGRIKL | |
| | QLTS | |
| 8 | MSNKEKNASETRKAYTTKMIPRSHDRMKLLGNFMDYLMDGTPIFFELWNQ | Cas12i1 |
| | FGGGIDRD11SGTANKDKISDDLLLAVNWFKVMPINSKPQGVSPSNLANL | (SEQ ID |
| | FQQYSGSEPDIQAQEYFASNFDTEKHQWKDMRVEYERLLAELQLSRSDMH | NO: 3 of |
| | HDLKLMYKEKCIGLSLSTAHYITSVMFGTGAKNNRQTKHQFYSKVIQLLE | U.S. Pat. No. |
| | ESTQINSVEQLASIILKAGDCDSYRKLRIRCSRKGATPSILKIVQDYELG | 10,808,245) |
| | TNHDDEVNVPSLIANLKEKLGRFEYECEWKCMEKIKAFLASKVGPYYLGS | |
| | YSAMLENALSPIKGMTTKNCKFVLKQIDAKNDIKYENEPFGKIVEGFFDS | |
| | PYFESDTNVKWVLHPHHIGESNIKTLWEDLNAIHSKYEEDIASLSEDKKE | |
| | KRIKVYQGDVCQTINTYCEEVGKEAKTPLVQLLRYLYSRKDDIAVDKIID | |
| | GITFLSKKHKVEKQKINPVIQKYPSFNFGNNSKLLGKIISPKDKLKHNLK | |
| | CNRNQVDNYIWIEIKVLNTKTMRWEKHHYALSSTRFLEEVYYPATSENPP | |
| | DALAARFRTKTNGYEGKPALSAEQIEQIRSAPVGLRKVKKRQMRLEAARQ | |
| | QNLLPRYTWGKDFNINICKRGNNFEVTLATKVKKKKEKNYKVVLGYDANI | |
| | VRKNTYAAIEAHANGDGVIDYNDLPVKPIESGFVTVESQVRDKSYDQLSY | |
| | NGVKLLYCKPHVESRRSFLEKYRNGTMKDNRGNNIQIDFMKDFEAIADDE | |
| | TSLYYFNMKYCKLLQSSIRNHSSQAKEYREEIFELLRDGKLSVLKLSSLS | |
| | NLSFVMFKVAKSLIGTYFGHLLKKPKNSKSDVKAPPITDEDKQKADPEMF | |
| | ALRLALEEKRLNKVKSKKEVIANKIVAKALELRDKYGPVLIKGENISDTT | |
| | KKGKKSSTNSFLMDWLARGVANKVKEMVMMHQGLEFVEVNPNFTSHQDPF | |
| | VHKNPENTFRARYSRCTPSELTEKNRKEILSFLSDKPSKRPTNAYYNEGA | |
| | MAFLATYGLKKNDVLGVSLEKFKQIMANILHQRSEDQLLFPSRGGMFYLA | |
| | TYKLDADATSVNWNGKQFWVCNADLVAAYNVGLVDIQKDFKKK | |
| 9 | MASISRPYGTKLRPDARKKEMLDKFFNTLTKGQRVFADLALCIYGSLTLE | Cas12i4 |
| | MAKSLEPESDSELVCAIGWFRLVDKTIWSKDGIKQENLVKQYEAYSGKEA | (SEQ ID |
| | SEVVKTYLNSPSSDKYVWIDCRQKFLRFQRELGTRNLSEDFECMLFEQYI | NO: 16 of |
| | RLTKGEIEGYAAISNMFGNGEKEDRSKKRMYATRMKDWLEANENITWEQY | U.S. Pat. No. |
| | REALKNQLNAKNLEQVVANYKGNAGGADPFFKYSFSKEGMVSKKEHAQQL | 10,808,245) |
| | DKFKTVLKNKARDLNFPNKEKLKQYLEAEIGIPVDANVYSQMFSNGVSEV | |
| | QPKTTRNMSFSNEKLDLLTELKDLNKGDGFEYAREVLNGFFDSELHTTED | |
| | KFNITSRYLGGDKSNRLSKLYKIWKKEGVDCEEGIQQFCEAVKDKMGQIP | |
| | IRNVLKYLWQFRETVSAEDFEAAAKANHLEEKISRVKAHPIVISNRYWAF | |
| | GTSALVGNIMPADKRHQGEYAGQNFKMWLEAELHYDGKKAKHHLPFYNAR | |
| | FFEEVYCYHPSVAEITPFKTKQFGCEIGKDIPDYVSVALKDNPYKKATKR | |
| | ILRAIYNPVANTTGVDKTTNCSFMIKRENDEYKLVINRKISVDRPKRIEV | |
| | GRTIMGYDRNQTASDTYWIGRLVPPGTRGAYRIGEWSVQYIKSGPVLSST | |
| | QGVNNSTTDQLVYNGMPSSSERFKAWKKARMAFIRKLIRQLNDEGLESKG | |
| | QDYIPENPSSFDVRGETLYVFNSNYLKALVSKHRKAKKPVEGILDEIEAW | |
| | TSKDKDSCSLMRLSSLSDASMQGIASLKSLINSYFNKNGCKTIEDKEKFN | |
| | PVLYAKLVEVEQRRTNKRSEKVGRIAGSLEQLALLNGVEVVIGEADLGEV | |
| | EKGKSKKQNSRNMDWCAKQVAQRLEYKLAFHGIGYFGVNPMYTSHQDPFE | |
| | HRRVADHIVMRARFEEVNVENIAEWHVRNFSNYLRADSGTGLYYKQATMD | |
| | FLKHYGLEEHAEGLENKKIKFYDFRKILEDKNLTSVIIPKRGGRIYMATN | |
| | PVTSDSTPITYAGKTYNRCNADEVAAANIVISVLAPRSKKNEEQDDIPLI | |
| | TKKAESKSPPKDRKRSKTSQLPQK | |
| 10 | MASISRPYGTKLRPDARKKEMLDKFFNTLTKGQRVFADLALCIYGSLTLE | Variant |
| | MAKSLEPESDSELVCAIGWFRLVDKTIWSKDGIKQENLVKQYEAYSGKEA | Cas12i4 |
| | SEVVKTYLNSPSSDKYVWIDCRQKFLRFQRELGTRNLSEDFECMLFEQYI | |
| | RLTKGEIEGYAAISNMFGNGEKEDRSKKRMYATRMKDWLEANENITWEQY | |
| | REALKNQLNAKNLEQVVANYKGNAGGADPFFKYSFSKEGMVSKKEHAQQL | |
| | DKFKTVLKNKARDLNFPNKEKLKQYLEAEIGIPVDANVYSQMFSNGVSEV | |
| | QPKTTRNMSFSNEKLDLLTELKDLNKGDGFEYAREVLNGFFDSELHTTED | |
| | KFNITSRYLGGDKSNRLSKLYKIWKKEGVDCEEGIQQFCEAVKDKMGQIP | |
| | IRNVLKYLWQFRETVSAEDFEAAAKANHLEEKISRVKAHPIVISNRYWAF | |
| | GTSALVGNIMPADKRHQGEYAGQNFKMWLRAELHYDGKKAKHHLPFYNAR | |
| | FFEEVYCYHPSVAEITPFKTKQFGCEIGKDIPDYVSVALKDNPYKKATKR | |
| | ILRAIYNPVANTTRVDKTTNCSFMIKRENDEYKLVINRKISRDRPKRIEV | |
| | GRTIMGYDRNQTASDTYWIGRLVPPGTRGAYRIGEWSVQYIKSGPVLSST | |
| | QGVNNSTTDQLVYNGMPSSSERFKAWKKARMAFIRKLIRQLNDEGLESKG | |
| | QDYIPENPSSFDVRGETLYVFNSNYLKALVSKHRKAKKPVEGILDEIEAW | |
| | TSKDKDSCSLMRLSSLSDASMQGIASLKSLINSYFNKNGCKTIEDKEKFN | |
| | PVLYAKLVEVEQRRTNKRSEKVGRIAGSLEQLALLNGVEVVIGEADLGEV | |
| | EKGKSKKQNSRNMDWCAKQVAQRLEYKLAFHGIGYFGVNPMYTSHQDPFE | |
| | HRRVADHIVMRARFEEVNVENIAEWHVRNFSNYLRADSGTGLYYKQATMD | |
| | FLKHYGLEEHAEGLENKKIKFYDFRKILEDKNLTSVIIPKRGGRIYMATN | |
| | PVTSDSTPITYAGKTYNRCNADEVAAANIVISVLAPRSKKNREQDDIPLI | |
| | TKKAESKSPPKDRKRSKTSQLPQK | |
| 11 | MSISNNNILPYNPKLLPDDRKHKMLVDTFNQLDLIRNNLHDMIIALYGAL | Cas12i3 |
| | KYDNIKQFASKEKPHISADALCSINWFRLVKTNERKPAIESNQIISKFIQ | (SEQ ID |
| | YSGHTPDKYALSHITGNHEPSHKWIDCREYAINYARIMHLSFSQFQDLAT | NO: 14 of |
| | ACLNCKILILNGTLTSSWAWGANSALFGGSDKENFSVKAKILNSFIENLK | U.S. Pat. No. |
| | DEMNTTKFQQQEKVCQQIGSSDAADLFDLYRSTVKDGNRGPATGRNPKVM | 10,808,245) |
| | NLFSQDGEISSEQREDFIESFQKVMQEKNSKQIIPHLDKLKYHLVKQSGL | |
| | YDIYSWAAAIKNANSTIVASNSSNLNTILNKTEKQQTFEELRKDEKIVAC | |
| | SKILLSVNDTLPEDLHYNPSTSNLGKNLDVFFDLLNENSVHTIENKEEKN | |
| | KIVKECVNQYMEECKGLNKPPMPVLLTFISDYAHKHQAQDFLSAAKMNFI | |
| | DLKIKSIKVVPTVHGSSPYTWISNLSKKNKDGKMIRTPNSSLIGWIIPPE | |
| | EIHDQKFAGQNPIIWAVLRVYCNNKWEMHHFPFSDSRFFTEVYAYKPNLP | |
| | YLPGGENRSKRFGYRHSTNLSNESRQILLDKSKYAKANKSVLRCMENMTH | |
| | NVVFDPKTSLNIRIKTDKNNSPVLDDKGRITFVMQINHRILEKYNNTKIE | |
| | IGDRILAYDQNQSENHTYAILQRTEEGSHAHQFNGWYVRVLETGKVTSIV | |
| | QGLSGPIDQLNYDGMPVTSHKFNCWQADRSAFVSQFASLKISETETFDEA | |
| | YQAINAQGAYTWNLFYLRILRKALRVCHMENINQFREEILAISKNRLSPM | |
| | SLGSLSQNSLKMIRAFKSIINCYMSRMSFVDELQKKEGDLELHTIMRLTD | |
| | NKLNDKRVEKINRASSFLTNKAHSMGCKMIVGESDLPVADSKTSKKQNVD | |
| | RMDWCARALSHKVEYACKLMGLAYRGIPAYMSSHQDPLVHLVESKRSVLR | |
| | PRFVVADKSDVKQHHLDNLRRMLNSKTKVGTAVYYREAVELMCEELGIHK | |
| | TDMAKGKVSLSDFVDKFIGEKAIFPQRGGRFYMSTKRLTTGAKLICYSGS | |
| | DVWLSDADEIAAINIGMFVVCDQTGAFKKKKKEKLDDEECDILPFRPM | |
| 120 | MDKKYSIGLDIGTNSVGWAVITDEYKVPSKKFKVLGNTDRHSIKKNLIGA | SpCas9 |
| | LLFDSGETAEATRLKRTARRRYTRRKNRICYLQEIFSNEMAKVDDSFFHR | |
| | LEESFLVEEDKKHERHPIFGNIVDEVAYHEKYPTIYHLRKKLVDSTDKAD | |
| | LRLIYLALAHMIKFRGHFLIEGDLNPDNSDVDKLFIQLVQTYNQLFEENP | |
| | INASGVDAKAILSARLSKSRRLENLIAQLPGEKKNGLFGNLIALSLGLTP | |
| | NFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLR | |
| | KQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDK | |
| | NLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQ | |
| | LKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHP | |
| | VENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLI | |
| | REVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGD | |
| 121 | MDKKYSIGLDIGTNSVGWAVITDEYKVPSKKFKVLGNTDRHSIKKNLIGA | nSpCas9 |
| | LLFDSGETAEATRLKRTARRRYTRRKNRICYLQEIFSNEMAKVDDSFFHR | |
| | LEESFLVEEDKKHERHPIFGNIVDEVAYHEKYPTIYHLRKKLVDSTDKAD | |
| | LRLIYLALAHMIKFRGHFLIEGDLNPDNSDVDKLFIQLVQTYNQLFEENP | |
| | INASGVDAKAILSARLSKSRRLENLIAQLPGEKKNGLFGNLIALSLGLTP | |
| | NFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLR | |
| | KQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDK | |
| | NLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQ | |
| | LKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHP | |
| | VENTQLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLI | |
| | REVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGD | |
| 122 | MKRNYILGLDIGITSVGYGIIDYETRDVIDAGVRLFKEANVENNEGRRSK | SaCas9 |
| | RGARRLKRRRRHRIQRVKKLLFDYNLLTDHSELSGINPYEARVKGLSQKL | |
| | SEEEFSAALLHLAKRRGVHNVNEVEEDTGNELSTKEQISRNSKALEEKYV | |
| | AELQLERLKKDGEVRGSINRFKTSDYVKEAKQLLKVQKAYHQLDQSFIDT | |
| | YIDLLETRRTYYEGPGEGSPFGWKDIKEWYEMLMGHCTYFPEELRSVKYA | |
| | YNADLYNALNDLNNLVITRDENEKLEYYEKFQIIENVFKQKKKPTLKQIA | |
| | KEILVNEEDIKGYRVTSTGKPEFTNLKVYHDIKDITARKEIIENAELLDQ | |
| | IAKILTIYQSSEDIQEELTNLNSELTQEEIEQISNLKGYTGTHNLSLKAI | |
| | NLILDELWHTNDNQIAIFNRLKLVPKKVDLSQQKEIPTTLVDDFILSPVV | |
| | KRSFIQSIKVINAIIKKYGLPNDIIIELAREKNSKDAQKMINEMQKRNRQ | |
| | TNERIEEIIRTTGKENAKYLIEKIKLHDMQEGKCLYSLEAIPLEDLLNNP | |
| | FNYEVDHIIPRSVSFDNSFNNKVLVKQEENSKKGNRTPFQYLSSSDSKIS | |
| | YETFKKHILNLAKGKGRISKTKKEYLLEERDINRFSVQKDFINRNLVDTR | |
| | YATRGLMNLLRSYFRVNNLDVKVKSINGGFTSFLRRKWKFKKERNKGYKH | |
| | HAEDALIIANADFIFKEWKKLDKAKKVMENQMFEEKQAESMPEIETEQEY | |
| | KEIFITPHQIKHIKDFKDYKYSHRVDKKPNRELINDTLYSTRKDDKGNTL | |
| | IVNNLNGLYDKDNDKLKKLINKSPEKLLMYHHDPQTYQKLKLIMEQYGDE | |
| | KNPLYKYYEETGNYLTKYSKKDNGPVIKKIKYYGNKLNAHLDITDDYPNS | |
| | RNKVVKLSLKPYRFDVYLDNGVYKFVTVKNLDVIKKENYYEVNSKCYEEA | |
| | KKLKKISNQAEFIASFYNNDLIKINGELYRVIGVNNDLLNRIEVNMIDIT | |
| | YREYLENMNDKRPPRIIKTIASKTQSIKKYSTDILGNLYEVKSKKHPQII | |
| | KKG | |

A nucleic acid sequence encoding the CRISPR nuclease described herein may be substantially identical to a reference nucleic acid sequence, e.g., SEQ ID NO: 1. In some embodiments, the CRISPR nuclease is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., nucleic acid sequence encoding the wildtype polypeptide, e.g., SEQ ID NO: 1. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the CRISPR nuclease is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., nucleic acid sequence encoding the CRISPR nuclease, e.g., SEQ ID NO: 1.

In some embodiments, the CRISPR nuclease of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to SEQ ID NO: 2. In some embodiments, the CRISPR nuclease of the present invention comprises a sequence having greater than 50%, 60%, 65%, 70%, 75%, 80%, 85%, 90%, 91%, 92%, 93%, 94%, 95%, 96%, 97%, 98%, or 99%, but not 100%, identity to SEQ ID NO: 2.

In some embodiments, the present invention describes a CRISPR nuclease having a specified degree of amino acid sequence identity to one or more reference polypeptides, e.g., a wildtype polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2. Homology or identity can be determined by amino acid sequence alignment, e.g., using a program such as BLAST, ALIGN, or CLUSTAL, as described herein.

In some embodiments, the CRISPR nuclease is a variant Cas12i2 polypeptide described in WO/2021/202800, the relevant disclosures of which are incorporated by reference for the subject matter and purpose referenced herein. In some embodiments, the variant Cas12i2 polypeptide comprises one or more of the amino acid substitutions listed in Table 2 of WO/2021/202800. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3 of PCT/US2021/025257. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4 of PCT/US2021/025257. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 5 of PCT/US2021/025257. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 495 of PCT/US2021/025257. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 496 of PCT/US2021/025257. In some embodiments, the CRISPR nuclease is a variant Cas12i2 polypeptide having at least 90%, at least 91%, at least 92%, at least 93%, at least 94%, at least 95%, at least 96%, at least 97%, at least 98%, at least 99%, or 100% sequence identity to any one of SEQ ID NOs: 3-146 and 495-512 of WO/2021/202800, which are incorporated by reference.

In some embodiments, the CRISPR nuclease is a Cas12i polypeptide. In some embodiments, the CRISPR nuclease is a Cas12i1 polypeptide. In some embodiments, the Cas12i1 polypeptide is a variant Cas12i1 polypeptide. In some embodiments, the variant Cas12i1 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to SEQ ID NO: 8. In some embodiments, the variant Cas12i1 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, 99%, or 100% identity to SEQ ID NO: 8.

In some embodiments, the CRISPR nuclease has a specified degree of amino acid sequence identity to one or more reference polypeptides, e.g., a wildtype Casi1 polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 8. Homology or identity can be determined by amino acid sequence alignment, e.g., using a program such as BLAST, ALIGN, or CLUSTAL, as described herein.

In some embodiments, a nucleic acid encoding the variant Cas12i1 polypeptide as described herein encodes an amino acid sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 8. In some embodiments, the variant Cas12i1 polypeptide has a sequence greater than 50%, 60%, 65%, 70%, 75%, 80%, 85%, 90%, 91%, 92%, 93%, 94%, 95%, 96%, 97%, 98%, 99%, or 100% identity to SEQ ID NO: 8.

In some embodiments, a variant Cas12i1 polypeptide described herein having enzymatic activity, e.g., nuclease or endonuclease activity, comprises an amino acid sequence which differs from the amino acid sequences of any one of a CRISPR nuclease and SEQ ID NO: 8 by 100, 95, 90, 85, 80, 75, 70, 65, 60, 55, 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some embodiments, the Cas12i polypeptide is a Cas12i3 polypeptide. In some embodiments, the Cas12i3 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to SEQ ID NO: 11. In some embodiments, the Cas12i3 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, 99%, or 100% identity to SEQ ID NO: 11.

In some embodiments, the Cas12i3 polypeptide is a variant Cas12i3 polypeptide. In some embodiments, the variant Cas12i3 polypeptide has a specified degree of amino acid sequence identity to one or more reference polypeptides, e.g., a wildtype polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11. Homology or identity can be determined by amino acid sequence alignment, e.g., using a program such as BLAST, ALIGN, or CLUSTAL, as described herein.

In some embodiments, a nucleic acid encoding the variant Cas12i3 polypeptide as described herein encodes an amino acid sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 11. In some embodiments, the variant Cas12i3 polypeptide has a sequence greater than 50%, 60%, 65%, 70%, 75%, 80%, 85%, 90%, 91%, 92%, 93%, 94%, 95%, 96%, 97%, 98%, 99%, or 100% identity to SEQ ID NO: 11.

In some embodiments, a variant Cas12i3 polypeptide described herein having enzymatic activity, e.g., nuclease or endonuclease activity, comprises an amino acid sequence which differs from the amino acid sequences of any one of a CRISPR nuclease and SEQ ID NO: 11 by 100, 95, 90, 85, 80, 75, 70, 65, 60, 55, 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some embodiments, the Cas12i polypeptide is a Cas12i4 polypeptide. In some embodiments, the Cas12i4 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to SEQ ID NO: 9 or SEQ ID NO: 10. In some embodiments, the Cas12i4 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, 99%, or 100% identity to SEQ ID NO: 9 or SEQ ID NO: 10.

In some embodiments, the Cas12i4 polypeptide is a variant Cas12i4 polypeptide. In some embodiments, the variant Cas12i4 polypeptide has a specified degree of amino acid sequence identity to one or more reference polypeptides, e.g., a wildtype polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9 or SEQ ID NO: 10. Homology or identity can be determined by amino acid sequence alignment, e.g., using a program such as BLAST, ALIGN, or CLUSTAL, as described herein.

In some embodiments, a nucleic acid encoding the variant Cas12i4 polypeptide as described herein encodes an amino acid sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 9 or SEQ ID NO: 10. In some embodiments, the variant Cas12i4 polypeptide has a sequence greater than 50%, 60%, 65%, 70%, 75%, 80%, 85%, 90%, 91%, 92%, 93%, 94%, 95%, 96%, 97%, 98%, 99%, or 100% identity to SEQ ID NO: 9 or SEQ ID NO: 10.

In some embodiments, a variant Cas12i4 polypeptide described herein having enzymatic activity, e.g., nuclease or endonuclease activity, comprises an amino acid sequence which differs from the amino acid sequences of any one of a CRISPR nuclease and SEQ ID NO: 9 or SEQ ID NO: 10 by 100, 95, 90, 85, 80, 75, 70, 65, 60, 55, 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some embodiments, the CRISPR nuclease is a Type II CRISPR nuclease, e.g., a Cas9 nuclease. In some embodiments, the Cas9 nuclease is a Cas9 from S. pyogenes or S. aureus or a variant thereof. See, e.g., U.S. 20190136248, which is incorporated by reference in its entirety. In some embodiments, the Cas9 polypeptide is a nickase.

In some embodiments, the Cas9 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to any one of SEQ ID NOs: 120-122. In some embodiments, the Cas9 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, 99%, or 100% identity to any one of SEQ ID NOs: 120-122.

In some embodiments, the Cas9 polypeptide is a variant Cas9 polypeptide. In some embodiments, the variant Cas9 polypeptide has a specified degree of amino acid sequence identity to one or more reference polypeptides, e.g., a wildtype polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 120-122. Homology or identity can be determined by amino acid sequence alignment, e.g., using a program such as BLAST, ALIGN, or CLUSTAL, as described herein.

In some embodiments, a nucleic acid encoding the variant Cas9 polypeptide as described herein encodes an amino acid sequence having at least 50%, 60%, 65%, 70%, 75%, 80%, 85%, 90%, 91%, 92%, 93%, 94%, 95%, 96%, 97%, 98%, 99%, or 100% identity to any one of SEQ ID NOs: 120-122. In some embodiments, the variant Cas9 polypeptide has a sequence greater than 50%, 60%, 65%, 70%, 75%, 80%, 85%, 90%, 91%, 92%, 93%, 94%, 95%, 96%, 97%, 98%, 99%, or 100% identity to any one of SEQ ID NOs: 120-122.

In some embodiments, a variant Cas9 polypeptide described herein having enzymatic activity, e.g., nuclease or endonuclease activity, comprises an amino acid sequence which differs from the amino acid sequences of any one of a CRISPR nuclease and SEQ ID NO: 120 or SEQ ID NO: 121 by 100, 95, 90, 85, 80, 75, 70, 65, 60, 55, 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some embodiments, the CRISPR nuclease (e.g., a Type V nuclease such as a Cas12i polypeptide or a Type II nuclease) comprises an alteration at one or more (e.g., several) amino acids of a wildtype polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more are altered.

In some embodiments, the CRISPR nuclease as in any one of the embodiments described herein comprises crRNA processing activity. In some embodiments, the Type V nuclease (e.g., the Cas12i polypeptide) is a variant that lacks crRNA processing activity. For example, in some embodiments wherein the Type V nuclease is a variant Cas12i2 polypeptide, the variant Cas12i2 polypeptide comprises an H485 or H486 substitution. In some embodiments, a variant Cas12i2 polypeptide having at least 90% identity (e.g., 90%, 91%, 92%, 93%, 94%, 95%, 96%, 97%, 98%, 99%, or 100%) to any one of SEQ ID NOs: 2-7 further comprises an H485 or H486 mutation. In some embodiments, a variant Cas12i2 polypeptide comprising an H485 or H486 mutation comprises diminished crRNA processing activity or lacks crRNA processing activity.

In some embodiments, the nucleotide sequence encoding the CRISPR nuclease described herein can be codon-optimized for use in a particular host cell or organism, or for particular purposes, e.g., expression. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/ and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, Pa.). In some examples, the nucleic acid encoding the CRISPR nuclease (e.g., any of the Cas12i polypeptides such as Cas12i2 or a Cas12i4 polypeptides disclosed herein), the reverse transcriptase, or any of the fusion polypeptides thereof can be mRNA molecules, which can be codon optimized. In some examples, the RT template sequence in any of the editing template RNAs disclosed herein or a portion thereof may also be codon-optimized.

Although the changes described herein may be one or more amino acid changes, changes to the CRISPR nuclease may also be of a structural or substantive nature, such as fusion of polypeptides as amino- and/or carboxyl-terminal extensions. For example, the CRISPR nuclease may contain additional peptides, e.g., one or more peptides. Examples of additional peptides may include epitope peptides for labelling, such as a polyhistidine tag (His-tag), Myc, and FLAG. In some embodiments, the CRISPR nuclease described herein can be fused to a detectable moiety such as a fluorescent protein (e.g., green fluorescent protein (GFP) or yellow fluorescent protein (YFP)).

In some embodiments, the CRISPR nuclease as in any one of the embodiments described herein comprises at least one (e.g., two, three, four, five, six, or more) nuclear localization signal (NLS). In some embodiments, the CRISPR nuclease comprises at least one (e.g., two, three, four, five, six, or more) nuclear export signal (NES). In some embodiments, the CRISPR nuclease comprises at least one (e.g., two, three, four, five, six, or more) NLS and at least one (e.g., two, three, four, five, six, or more) NES.

In some embodiments, the CRISPR nuclease comprises at least a RuvC domain but less than the whole CRISPR nuclease. In some embodiments, the CRISPR nuclease is a truncated CRISPR nuclease relative to a wild-type CRISPR nuclease. In some embodiments, the truncated CRISPR nuclease comprises a RuvC domain. In some embodiments, the CRISPR nuclease comprises at least one functional domain of the whole CRISPR nuclease. In some embodiments, the CRISPR nuclease comprises at least two RuvC domains or at least two RuvC motifs. In some embodiments, the CRISPR nuclease comprises at least three RuvC domains or at least three RuvC motifs. In some embodiments, the CRISPR nuclease comprises at least one catalytically dead RuvC domain and at least one catalytically active RuvC domain. In some embodiments, the CRISPR nuclease comprises two RuvC domains from one or more Type V or Type II nucleases. In some embodiments, the CRISPR nuclease comprises at least a RuvC domain and a dimerization domain.

In some embodiments, the CRISPR nuclease as in any one of the embodiments described herein is fused to a polymerase. In some embodiments, the CRISPR nuclease as described in any one of the previous embodiments is fused to a reverse transcriptase polypeptide. In some embodiments, the CRISPR nuclease comprises an N-terminal reverse transcriptase polypeptide. In some embodiments, the CRISPR nuclease comprises a C-terminal reverse transcriptase polypeptide. In some embodiments, the CRISPR nuclease comprises a reverse transcriptase polypeptide at an intramolecular position within the CRISPR nuclease (e.g., the reverse transcriptase is within a loop of the CRISPR nuclease).

In some embodiments, the CRISPR nuclease as in any one of the embodiments described herein interacts with a reverse transcriptase polypeptide (e.g., through electrostatic interactions). In some embodiments, the CRISPR nuclease comprises a dimerization domain. As used herein, the term “dimerization domain,” refers to a polypeptide domain capable of specifically binding a separate, and compatible, polypeptide domain (e.g., a second compatible dimerization domain). In some embodiments, the dimer is formed by a noncovalent bond between the first dimerization domain and the second compatible dimerization domain. In some embodiments, a dimerization domain is a leucine zipper, nanobody, or antibody. In some embodiments, the dimerization domain recruits a reverse transcriptase polypeptide. In some embodiments, the CRISPR nuclease and the reverse transcriptase polypeptide interact through coiled-coil peptide heterodimers.

In some embodiments, the CRISPR nuclease as in any one of the embodiments described herein interacts with a ligase, an integrase, and/or a recombinase. In some embodiments, the CRISPR nuclease as in any one of the embodiments described herein is fused to a ligase, an integrase, and/or a recombinase. In some embodiments, the ligase, integrase, and/or recombinase is fused to the N-terminus or C-terminus of the CRISPR nuclease. In some embodiments, the ligase, integrase, and/or recombinase is fused internally to the CRISPR nuclease. In some embodiments, the integrase is a serine integrase. In some embodiments, the integrase is a Bxb1, TP901, or PhiBT1 integrase. In some embodiments, the recombinase is a serine recombinase or a tyrosine recombinase. In some embodiments, the recombinase is a CRE recombinase. In some embodiments, a CRISPR nuclease that interacts with or is fused to a ligase, integrase, and/or recombinase further interacts with or is fused to a reverse transcriptase.

B. Reverse Transcriptase

In various embodiments, the composition disclosed herein includes a polymerase (e.g., DNA-dependent DNA polymerase or RNA-dependent DNA polymerase), or a variant thereof, which can be provided as a fusion to the CRISPR nuclease. The polymerase may be a wild-type polymerase, functional fragment, variant, truncated variant, or the like. The polymerase may include a wild-type polymerase from eukaryotic, prokaryotic, archaeal, or viral organisms, and/or the polymerases may be modified by genetic engineering, mutagenesis, directed evolution-based processes.

Any of the CRISPR nuclease-RT fusion polypeptides, such as those disclosed herein (e.g., those shown in Tables 7 and 17), their encoding nucleic acids, vectors comprising such and method of making such are also within the scope of the present disclosure.

In some embodiments, the polymerase is a reverse transcriptase. In some embodiments, the reverse transcriptase polypeptide is any wild-type reverse transcriptase obtained from any naturally-occurring organism or virus, or obtained from a commercial or non-commercial source. The reverse transcriptase polypeptide may also be a variant reverse transcriptase polypeptide.

The reverse transcriptase polypeptide can be obtained from a number of different sources. For instance, the gene may be obtained from eukaryotic cells which are infected with retrovirus or from a plasmid that comprises either a portion of or the entire retrovirus genome. In addition, RNA that comprises the reverse transcriptase gene can be obtained from retroviruses. In some embodiments, the reverse transcriptase is expressed or otherwise provided as an individual component, i.e., not as a fusion protein with a CRISPR nuclease (e.g., a Cas12i) polypeptide.

A person of ordinary skill in the art will recognize that reverse transcriptases are known in the art, including, but not limited to, Moloney Murine Leukemia Virus (MMLV) reverse transcriptase, Human Immunodeficiency Virus (HIV) reverse transcriptase, and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the composition described herein.

In some embodiments, the reverse transcriptase is MMLV-RT, MarathonRT from Eubacterium rectale, or RTX reverse transcriptase or a variant of MMLV-RT, MarathonRT, or RTX reverse transcriptase. In some embodiments, the reverse transcriptase is a sequence shown in Table 2, a variant thereof, or an ortholog thereof.

TABLE 2
Reverse Transcriptase Sequences.

| Reverse | |
|---|---|
| Transcriptase | Sequence |
| MMLV-RT | MTLNIEDEYRLHETSKEPDVSLGSTWLSDFPQAWAETGGMGLAVRQAPLIIPLKATSTPV |
| | SIKQYPMSQEARLGIKPHIQRLLDQGILVPCQSPWNTPLLPVKKPGTNDYRPVQDLREVN |
| | KRVEDIHPTVPNPYNLLSGLPPSHQWYTVLDLKDAFFCLRLHPTSQPLFAFEWRDPEMGI |
| | SGQLTWTRLPQGFKNSPTLFNEALHRDLADFRIQHPDLILLQYVDDLLLAATSELDCQQG |
| | TRALLQTLGNLGYRASAKKAQICQKQVKYLGYLLKEGQRWLTEARKETVMGQPTPKTPRQ |
| | LREFLGKAGFCRLFIPGFAEMAAPLYPLTKPGTLFNWGPDQQKAYQEIKQALLTAPALGL |
| | PDLTKPFELFVDEKQGYAKGVLTQKLGPWRRPVAYLSKKLDPVAAGWPPCLRMVAAIAVL |
| | TKDAGKLTMGQPLVILAPHAVEALVKQPPDRWLSNARMTHYQALLLDTDRVQFGPVVALN |
| | PATLLPLPEEGLQHNCLDILAEAHGTRPDLTDQPLPDADHTWYTDGSSLLQEGQRKAGAA |
| | VTTETEVIWAKALPAGTSAQRAELIALTQALKMAEGKKLNVYTDSRYAFATAHIHGEIYR |
| | RRGWLTSEGKEIKNKDEILALLKALFLPKRLSIIHCPGHQKGHSAEARGNRMADQAARKA |
| | AITETPDTSTLLIENSSP (SEQ ID NO: 29) |
| MMLV-RT_2 | MTWLSDFPQAWAETGGMGLAVRQAPLIIPLKATSTPVSIKQYPMSQEARLGIKPHIQRLL |
| | DQGILVPCQSPWNTPLLPVKKPGTNDYRPVQDLREVNKRVEDIHPTVPNPYNLLSGLPPS |
| | HQWYTVLDLKDAFFCLRLHPTSQPLFAFEWRDPEMGISGQLTWTRLPQGFKNSPTLFDEA |
| | LHRDLADFRIQHPDLILLQYVDDLLLAATSELDCQQGTRALLQTLGNLGYRASAKKAQIC |
| | QKQVKYLGYLLKEGQRWLTEARKETVMGQPTPKTPRQLREFLGTAGFCRLWIPGFAEMAA |
| | PLYPLTKTGTLFNWGPDQQKAYQEILQALLTAPALGLPDLTKPFELFVDEKQGYAKGVLT |
| | QKLGPWRRPVAYLSKKLDPVAAGWPPCLRMVAAIAVLTKDAGKLTMGQPLVILAPHAVEA |
| | LVKQPPDRWLSNARMTHYQALLLDTDRVQFGPVVALNPATLLPLPEEGLQHNCLDILAEA |
| | HGTRPDL (SEQ ID NO: 230) |
| MMTV-RT | MLQLGHLEESNSPWNTPVFVIKKKSGKWRLLQDLRAVNATMHDMGALQPGLPSPVAVPKG |
| | WEIIIIDLQDCFFNIKLHPEDCKRFAFSVPSPNFKRPYQRFQWKVLPQGMKNSPTLCQKF |
| | VDKAILTVRDKYQDSYIVHYMDDILLAHPSRSIVDEILTSMIQALNKHGLVVSTEKIQKY |
| | DNLKYLGTHIQGDSVSYQKLQIRTDKLRTLNDFQKLLGNINWIRPFLKLTT (SEQ ID |
| | NO: 231) |
| MarathonRT | MDTSNLMEQILSSDNLNRAYLQVVRNKGAEGVDGMKYTELKEHLAKNGETIKGQLRTRKY |
| | KPQPARRVEIPKPDGGVRNLGVPTVTDRFIQQAIAQVLTPIYEEQFHDHSYGFRPNRCAQ |
| | QAILTALNIMNDGNDWIVDIDLEKFFDTVNHDKLMTLIGRTIKDGDVISIVRKYLVSGIM |
| | IDDEYEDSIVGTPQGGNLSPLLANIMLNELDKEMEKRGLNFVRYADDCIIMVGSEMSANR |
| | VMRNISRFIEEKLGLKVNMTKSKVDRPSGLKYLGFGFYFDPRAHQFKAKPHAKSVAKFKK |
| | RMKELTCRSWGVSNSYKVEKLNQLIRGWINYFKIGSMKTLCKELDSRIRYRLRMCIWKQW |
| | KTPQNQEKNLVKLGIDRNTARRVAYTGKRIAYVCNKGAVNVAISNKRLASFGLISMLDYY |
| | IEKCVTC (SEQ ID NO: 232) |
| RTX reverse | MILDTDYITEDGKPVIRIFKKENGEFKIEYDRTFEPYLYALLKDDSAIEEVKKITAERHG |
| transcriptase | TVVTVKRVEKVQKKFLGRPVEVWKLYFTHPQDVPAIMDKIREHPAVIDIYEYDIPFAIRY |
| | LIDKGLVPMEGDEELKLLAFDIETLYHEGEEFAEGPILMISYADEEGARVITWKNVDLPY |
| | VDVVSTEREMIKRFLRVVKEKDPDVLITYNGDNFDFAYLKKRCEKLGINFALGRDGSEPK |
| | IQRMGDRFAVEVKGRIHFDLYPVIRRTINLPTYTLEAVYEAVFGQPKEKVYAEEITTAWE |
| | TGENLERVARYSMEDAKVTYELGKEFLPMEAQLSRLIGQSLWDVSRSSTGNLVEWFLLRK |
| | AYERNELAPNKPDEKELARRHQSHEGGYIKEPERGLWENIVYLDFRSLYPSIIITHNVSP |
| | DTLNREGCKEYDVAPQVGHRFCKDFPGFIPSLLGDLLEERQKIKKRMKATIDPIERKLLD |
| | YRQRAIKILANSLYGYYGYARARWYCKECAESVIAWGREYLTMTIKEIEEKYGFKVIYSD |
| | TDGFFATIPGADAETVKKKAMEFLKYINAKLPGALELEYEGFYKRGLFVTKKKYAVIDEE |
| | GKITTRGLEIVRRDWSEIAKETQARVLEALLKDGDVEKAVRIVKEVTEKLSKYEVPPEKL |
| | VIHKQITRDLKDYKATGPHVAVAKRLAARGVKIRPGTVISYIVLKGSGRIVDRAIPFDEF |
| | DPTKHKYDAEYYIEKQVLPAVERILRAFGYRKEDLRYQKTRQVGLSARLKPKGT (SEQ |
| | ID NO: 233) |
| RTX-exoMinus | MILDTDYITEDGKPVIRIFKKENGEFKIEYDRTFEPYLYALLKDDSAIEEVKKITAERHG |
| | TVVTVKRVEKVQKKFLGRPVEVWKLYFTHPQDVPAIMDKIREHPAVIDIYEYDIPFAIRY |
| | LIDKGLVPMEGDEELKLLAFDIETLYHEGEEFAEGPILMISYADEEGARVITWKNVDLPY |
| | VDVVSTEREMIKRFLRVVKEKDPDVLITYDGDNFDFAYLKKRCEKLGINFALGRDGSEPK |
| | IQRMGDRFAVEVKGRIHFDLYPVIRRTINLPTYTLEAVYEAVFGQPKEKVYAEEITTAWE |
| | TGENLERVARYSMEDAKVTYELGKEFLPMEAQLSRLIGQSLWDVSRSSTGNLVEWFLLRK |
| | AYERNELAPNKPDEKELARRHQSHEGGYIKEPERGLWENIVYLDFRSLYPSIIITHNVSP |
| | DTLNREGCKEYDVAPQVGHRFCKDFPGFIPSLLGDLLEERQKIKKRMKATIDPIERKLLD |
| | YRQRAIKILANSLYGYYGYARARWYCKECAESVIAWGREYLTMTIKEIEEKYGFKVIYSD |
| | TDGFFATIPGADAETVKKKAMEFLKYINAKLPGALELEYEGFYKRGLFVTKKKYAVIDEE |
| | GKITTRGLEIVRRDWSEIAKETQARVLEALLKDGDVEKAVRIVKEVTEKLSKYEVPPEKL |
| | VIHKQITRDLKDYKATGPHVAVAKRLAARGVKIRPGTVISYIVLKGSGRIVDRAIPFDEF |
| | DPTKHKYDAEYYIEKQVLPAVERILRAFGYRKEDLRYQKTRQVGLSARLKPKGT (SEQ |
| | ID NO: 234) |
| B11 RT | MILDTDYITEDGKPVIRIFKKENGEFKIEYDRTFEPYLYALLKDDSAIEEVKKITAERHS |
| | TVVTVKRVEKVQKKFLGRSVEVWKLYFTHPQDVPAIMDKIREHPAVIDIYEYDIPFAIRY |
| | LIDKGLVPMEGDEELKLLALDIGTPCHEGEVFAEGPILMISYADEEGTRVITWRNVDLPY |
| | VDVLSTEREMIQRFLRVVKEKDPDVLITYNGDNFDFAYLKKRCEKLGINFTLGREGSEPK |
| | IQRMGDRFAVEVKGRIHFDLYPVIRRTVNLPIYTLEAVYEAVFGQPKEKVYAEEITTAWE |
| | TGENLERVARYSMEDAKVTYELGKEFMPMEAQLSRLIGQSLWDVSRSSTGNLVEWFLLRK |
| | AYERNELAPNKPDEKELARRHQSHEGGYIKEPERGLWENIVYLDFRSLYPSIIITHNVSP |
| | DTLNREGCKEYDVAPQVGHRFCKDFPGFIPSLLGDLLEERQKIKKRMKATIDPIERKLLD |
| | YRQRAIKILANSLYGYYGYARARWYCKECAESVIAWGREYITMTIKEIEEKYGFKLIYSD |
| | TDGFFATIPGAEAETVKKKAMEFLKYINAKLPGALELEYEGFYKRGLFVTKKKYAVIDEE |
| | GKITTRGLEIVRRDWSEIAKETQARVLEALLKDGDVEKAVRIVKEVTEKLSKYEVPPEKL |
| | VIHKQITRDLKDYKATGPHVAVAKRLAARGVKIRPGTVISYIVLKGSGRIVDRAIPFDEF |
| | DPTKHKYDAEYYIENQVLPAVERILRAYGYRKEDLWYQKTRQVGLSARLKPKGT (SEQ |
| | ID NO: 235) |
| RT CaMV | MDHLLLKTQTQTEQVMNVTNPNSIYIKGRLYFKGYKKIELHCFVDTGASLCIASKFVIPE |
| | EHWVNAERPIMVKIADGSSITISKVCKDIDLIIAGEIFRIPTVYQQESGIDFIIGNNFCQ |
| | LYEPFIQFTDRVIFTKNKSYPVHIAKLTRAVRVGTEGFLESMKKRSKTQQPEPVNISTNK |
| | IENPLEEIAILSEGRRLSEEKLFITQQRMQKIEELLEKVCSENPLDPNKTKQWMKASIKL |
| | SDPSKAIKVKPMKYSPMDREEFDKQIKELLDLKVIKPSKSPHMAPAFLVNNEAEKRRGKK |
| | RMVVNYKAMNKATVGDAYNLPNKDELLTLIRGKKIFSSFDCKSGFWQVLLDQESRPLTAF |
| | TCPQGHYEWNVVPFGLKQAPSIFQRHMDEAFRVFRKFCCVYVDDILVFSNNEEDHLLHVA |
| | MILQKCNQHGIILSKKKAQLFKKKINFLGLEIDEGTHKPQGHILEHINKFPDTLEDKKQL |
| | QRFLGILTYASDYIPKLAQIRKPLQAKLKENVPWRWTKEDTLYMQKVKKNLQGFPPLHHP |
| | LPEEKLIIETDASDDYWGGMLKAIKINEGTNTELICRYASGSFKAAEKNYHSNDKETLAV |
| | INTIKKFSIYLTPVHFLIRTDNTHFKSFVNLNYKGDSKLGRNIRWQAWLSHYSFDVEHIK |
| | GTDNHFADFLSREFNKVNSSGGS (SEQ ID NO: 236) |
| RT retron | MGIHGVPAAMKSAEYLNTFRLRNLGLPVMNNLHDMSKATRISVETLRLLIYTADFRYRIY |
| | TVEKKGPEKRMRTIYQPSRELKALQGWVLRNILDKLSSSPFSIGFEKHQSILNNATPHIG |
| | ANFILNIDLEDFFPSLTANKVFGVFHSLGYNRLISSVLTKICCYKNLLPQGAPSSPKLAN |
| | LICSKLDYRIQGYAGSRGLIYTRYADDLTLSAQSMKKVVKARDFLFSIIPSEGLVINSKK |
| | TCISGPRSQRKVTGLVISQEKVGIGREKYKEIRAKIHHIFCGKSSEIEHVRGWLSFILSV |
| | DSKSHRRLITYISKLEKKYGKNPLNKAKT (SEQ ID NO: 237) |
| HIV2 | MEKEGQLEEAPPTNPYNTPTFAIKKKDKNKWRMLIDFRELNKVTQDFTEIQLGIPHPAGL |
| | AKKRRITVLDVGDAYFSIPLHEDFRPYTAFTLPSVNNAEPGKRYIYKVLPQGWKGSPAIF |
| | QHTMRQVLEPFRKANKDVIIIQYMDDILIASDRTDLEHDRVVLQLKELLNGLGFSTPDEK |
| | FQKDPPIHWMGYELWPTKWKLQKIQLPQKEIWTVNDIQKLVGVLNWAAQLYPGIK (SEQ |
| | ID NO: 238) |
| HIV1 | MEEEGKISKIGPENPYNTPVFAIKKKDSTKWRKLVDFRELNKRTQDFWEVQLGIPHPAGL |
| | KKKKSVTVLDVGDAYFSVPLDESFRKYTAFTIPSINNETPGIRYQYNVLPQGWKGSPAIF |
| | QSSMTKILEPFRIKNPEIVIYQYMDDLYVGSDLEIGQHRTKIEELRAHLLSWGFTTPDKK |
| | HQKEPPFLWMGYELHPDRWTVQPIDLPEKDSWTVNDIQKLVGKLNWASQIYAGIK (SEQ |
| | ID NO: 239) |
| HIV-1 RT p51 | MPISPIETVPVKLKPGMDGPKVKQWPLTEEKIKALVEICTEMEKEGKISKIGPENPYNTP |
| | VFAIKKKDSTKWRKLVDFRELNKRTQDFWEVQLGIPHPAGLKKKKSVTVLDVGDAYFSVP |
| | LDEDFRKYTAFTIPSINNETPGIRYQYNVLPQGWKGSPAIFQSSMTKILEPFRKQNPDIV |
| | IYQYMDDLYVGSDLEIGQHRTKIEELRQHLLRWGLTTPDKKHQKEPPFLWMGYELHPDKW |
| | TVQPIVLPEKDSWTVNDIQKLVGKLNWASQIYPGIKVRQLCKLLRGTKALTEVIPLTEEA |
| | ELELAENREILKEPVHGVYYDPSKDLIAEIQKQGQGQWTYQIYQEPFKNLKTGKYARMRG |
| | AHTNDVKQLTEAVQKITTESIVIWGKTPKFKLPIQKETWETWWTEYWQATWIPEWEFVNT |
| | PPLVKLWYQLEKEPIVGAETF (SEQ ID NO: 240) |
| MMLV RT | STLNIEDEYRLHETSKEPDVSLGSTWLSDFPQAWAETGGMGLAVRQAPLIIPLKATSTPV |
| lacking the | SIKQYPMSQEARLGIKPHIQRLLDQGILVPCQSPWNTPLLPVKKPGTNDYRPVQDLREVN |
| RNase H domain | KRVEDIHPTVPNPYNLLSGLPPSHQWYTVLDLKDAFFCLRLHPTSQPLFAFEWRDPEMGI |
| | SGQLTWTRLPQGFKNSPTLFNEALHRDLADFRIQHPDLILLQYVDDLLLAATSELDCQQG |
| | TRALLQTLGNLGYRASAKKAQICQKQVKYLGYLLKEGQRWLTEARKETVMGQPTPKTPRQ |
| | LREFLGKAGFCRLFIPGFAEMAAPLYPLTKPGTLFNWGPDQQKAYQEIKQALLTAPALGL |
| | PDLTKPFELFVDEKQGYAKGVLTQKLGPWRRPVAYLSKKLDPVAAGWPPCLRMVAAIAVL |
| | TKDAGKLTMGQPLVILAPHAVEALVKQPPDRWLSNARMTHYQALLLDTDRVQFGPVVALN |
| | PATLLP (SEQ ID NO: 224) |

In some embodiments, the reverse transcriptase polypeptide is fused to a CRISPR nuclease as in any one of the embodiments described herein. In some embodiments, the reverse transcriptase polypeptide comprises an N-terminal CRISPR nuclease. In some embodiments, the reverse transcriptase polypeptide comprises a C-terminal CRISPR nuclease. In some embodiments, the reverse transcriptase polypeptide comprises a CRISPR nuclease at an intramolecular position within the reverse transcriptase polypeptide (e.g., the CRISPR nuclease) is within a loop of the reverse transcriptase polypeptide.

In some embodiments, the reverse transcriptase polypeptide comprises a dimerization domain. In some embodiments, a dimerization domain is a leucine zipper, nanobody, or antibody. In some embodiments, the dimerization domain recruits a CRISPR nuclease (e.g., a Type V nuclease such as a Cas12i polypeptide).

In some embodiments, the reverse transcriptase polypeptide is an “error-prone” reverse transcriptase variant. Error-prone reverse transcriptases that are known and/or available in the art may be used. It will be appreciated that reverse transcriptases naturally do not have any proofreading function; thus, the error rate of reverse transcriptases is generally higher than DNA polymerases comprising a proofreading activity. In some embodiments, the reverse transcriptase is considered to be “error-prone” if it has an error rate that is less than one error in about 15,000 nucleotides synthesized.

In some embodiments, the reverse transcriptase polypeptide has a mutation or mutations in the RNase H domain. In some embodiments, the reverse transcriptase polypeptide does not comprise an RNase H domain (e.g., the RNase H domain has been removed from the reverse transcriptase polypeptide). In some embodiments, the RNase H domain is truncated in a reverse transcriptase polypeptide. In some embodiments, the reverse transcriptase polypeptide has a mutation or mutations in the RNA-dependent DNA polymerase domain. In some embodiments, the reverse transcriptase polypeptide is a variant that has altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields. Wild-type MMLV reverse transcriptase typically has an optimal temperature in the range of 37-48° C.; however, mutations may be introduced that allow for the reverse transcription activity at higher temperatures of over 48° C., including 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., and higher.

Variant reverse transcriptase polypeptides used herein may be at least about 20% identical, at least about 25% identical, at least about 30% identical, at least about 35% identical, at least about 40% identical, at least about 45% identical, at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to any reference reverse transcriptase polypeptide, including any wild-type reverse transcriptase, mutant reverse transcriptase, or fragment of a reverse transcriptase, or other reverse transcriptase variant disclosed or contemplated herein or known in the art. In some embodiments, a reverse transcriptase variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference reverse transcriptase. In some embodiments, the reverse transcriptase variant comprises a fragment of a reference reverse transcriptase, such that the fragment is at least about 20% identical, at least about 25% identical, at least about 30% identical, at least about 35% identical, at least about 40% identical, at least about 45% identical, at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference reverse transcriptase.

Variant reverse transcriptases, including error-prone reverse transcriptases, thermostable reverse transcriptases, and reverse transcriptases with increased processivity, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

In some embodiments, the reverse transcriptase polypeptides comprise or is fused to a domain to improve extension rates and/or efficiency of the reverse transcriptase. In some embodiments, the reverse transcriptase polypeptide is fused to an Sso7d polypeptide such as an Sso7d polypeptide from Sulfolobus solfataricus. See, e.g., Wang et al., Nucleic Acids Res. 32(3): 1197-207 (2004).

In some embodiments, a CRISPR nuclease-reverse transcriptase fusion polypeptide as described elsewhere herein is capable of binding and binds to at least one nuclease binding sequence in the editing template RNA. In some embodiments, the CRISPR nuclease-reverse transcriptase fusion polypeptide is capable of binding and binds to a target sequence through at least one DNA-binding sequence in the editing template RNA. In such embodiments, the CRISPR nuclease-reverse transcriptase fusion polypeptide is recruited to or brought in close proximity to the target sequence through binding of the CRISPR nuclease via the nuclease binding sequence and the DNA-binding sequence of the editing template RNA.

In some embodiments, the reverse transcriptase transcribes the reverse transcription template sequence into the non-PAM strand of a target nucleic acid starting at the 5′ end of a PBS. In some embodiments, the reverse transcriptase transcribes the reverse transcription template sequence into the non-PAM strand of a target nucleic acid starting at the 3′ end of a PBS. In some embodiments, the reverse transcriptase transcribes the reverse transcription template sequence into the PAM strand of a target nucleic acid starting at the 5′ end of a PBS. In some embodiments, the reverse transcriptase transcribes the reverse transcription template sequence into the PAM strand of a target nucleic acid starting at the 3′ end of a PBS. In some embodiments, following binding of a PBS to a non-PAM strand of a target nucleic acid, the reverse transcriptase transcribes the reverse transcription template sequence from a free 3′ end of the non-PAM strand. In some embodiments, following hybridization of a PBS to a PAM strand of a target nucleic acid, the reverse transcriptase transcribes the reverse transcription template sequence from a free 3′ end of the PAM strand.

In some embodiments, the reverse transcriptase as in any one of the embodiments described herein interacts with a ligase, an integrase, and/or a recombinase. In some embodiments, the reverse transcriptase as in any one of the embodiments described herein is fused to a ligase, an integrase, and/or a recombinase. In some embodiments, the ligase, integrase, and/or recombinase is fused to the N-terminus or C-terminus of the reverse transcriptase. In some embodiments, the ligase, integrase, and/or recombinase is fused internally to the reverse transcriptase. In some embodiments, the integrase is a serine integrase. In some embodiments, the integrase is a Bxb1, TP901, or PhiBT1 integrase. In some embodiments, the recombinase is a serine recombinase or a tyrosine recombinase. In some embodiments, the recombinase is a CRE recombinase. In some embodiments, a reverse transcriptase that interacts with or is fused to a ligase, integrase, and/or recombinase further interacts with or is fused to a CRISPR nuclease.

C. Gene Editing RNA Molecules

Any of the gene editing systems disclosed herein may comprise an editing template RNA(s) (gene editing RNAs), which comprises an RNA guide and an RNA reverse transcriptase (RT) donor (RT donor RNA). The editing template RNA(s) aids in editing sequences in a target nucleic acid such as a desired genomic site. In some embodiments, the editing template RNA can be a single RNA molecule comprising both the RNA guide (e.g., comprises a nuclease binding sequence and a DNA-binding sequence) and an RT donor RNA. In other embodiments, the editing template RNA comprises the RNA guide and the RT donor RNA as separate RNA molecules.

In some embodiments, the editing template RNA or any portion thereof is encoded in a vector. In some embodiments, the vector comprises a Pol II promoter or a Pol III promoter. In some embodiments, the editing template RNA disclosed herein does not comprise a tracrRNA component. Alternatively, the editing template RNA disclosed herein may comprise a tracrRNA component.

i. RNA Guide

In any of the gene editing systems disclosed herein, the editing template RNA comprises an RNA guide, which medicates cleavage of a target nucleic acid via the CRISPR nuclease also contained in the gene editing system. The RNA guide (or a gRNA) comprises a nuclease binding sequence and a DNA-binding sequence (a spacer). The nuclease binding sequence may comprise one or more binding sites that can be recognized by the CRISPR nuclease for binding. In some instances, the gRNA is a single RNA molecule comprising both the nuclease binding sequence and a spacer sequence. Alternatively, the gRNA may comprise the nuclease binding sequence and the spacer as two separate RNA molecules.

In some embodiments, an RNA guide comprises an RNA extension at the 5′ end of the RNA guide, at the 3′ end of the RNA guide, or at an intramolecular position within the RNA guide. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides in length. In some embodiments, the RNA extension is a reverse transcription donor RNA (“RT donor RNA”) (e.g., the RNA guide is fused to an RT donor RNA). In some embodiments, the RT donor RNA comprises a primer binding site (PBS) and a reverse transcription template sequence, as described herein.

Nuclease Binding Sequences

In some embodiments, a composition as described herein comprises a nuclease binding sequence. In some embodiments, the nuclease binding sequence is a CRISPR nuclease binding sequence (e.g., the nuclease binding sequence is capable of binding to a Type V nuclease or a Type II nuclease). In some embodiments, the nuclease binding sequence is further a nucleic acid binding sequence (e.g., a DNA binding sequence).

In some embodiments, the nuclease binding sequence comprises an RNA guide. The RNA guide can bind any one of the CRISPR nucleases described herein (e.g., a Type V nuclease or a Type II nuclease) with specific binding affinity. In some embodiments, the RNA guide further comprises specific binding affinity to a target sequence. In some embodiments, a composition described herein comprises two or more RNA guides (e.g., 2, 3, 4, 5, 6, 7, 8, 9, or more). In some embodiments, the nuclease binding sequence is encoded in a vector. In some embodiments, the vector comprises a Pol II promoter or a Pol III promoter.

In some embodiments, the nuclease binding sequence comprises a direct repeat sequence. In certain embodiments, the nuclease binding sequence includes a direct repeat sequence linked to a DNA-binding sequence (e.g., a DNA-targeting sequence or spacer). In some embodiments, the nuclease binding sequence includes a direct repeat sequence and a DNA-binding sequence or a direct repeat—DNA-binding sequence—direct repeat sequence. In some embodiments, the nuclease binding sequence includes a truncated direct repeat sequence and a DNA-binding sequence, which is typical of processed or mature crRNA.

In some embodiments, the nuclease binding sequence (e.g., the direct repeat sequence) is capable of binding a Cas12a (Cpf1), Cas12b (C2c1), Cas12c, Cas12d, Cas12e, Cas12f, Cas12h, Cas12i, or Cas12j (CasPhi) polypeptide. In some embodiments, the direct repeat sequence is capable of binding a Cas9 polypeptide.

In the embodiments where the nuclease binding sequence is a direct repeat for a publicly available CRISPR nuclease, those direct repeat sequences are known in the art. In some embodiments, direct repeat sequences capable of binding a CRISPR nuclease are any of those disclosed in WO2021055874, WO2020206036, WO2020191102, WO2020186213, WO2020028555, WO2020033601, WO2019126762, WO2019126774, WO2019071048, WO2019018423, WO2019005866, WO2018191388, WO2018170333, WO2018035388, WO2018035387, WO2017219027, WO2017189308, WO2017184768, WO2017106657, WO2016205749, WO2017070605, WO2016205764, WO2016205711, WO2016028682, WO2015089473, WO2014093595, WO2015089427, WO2014204725, WO2015070083, WO2014093655, WO2014093694, WO2014093712, WO2014093635, WO2021133829, WO2021007177, WO2020197934, WO2020181102, WO2020181101, WO2020041456, WO2020023529, WO2020005980, WO2019104058, WO2019089820, WO2019089808, WO2019089804, WO2019089796, WO2019036185, WO2018226855, WO2018213351, WO2018089664, WO2018064371, WO2018064352, WO2017106569, WO2017048969, WO2016196655, WO2016106239, WO2016036754, WO2015103153, WO2015089277, WO2014150624, WO2013176772, WO2021119563, WO2021118626, WO2020247883, WO2020247882, WO2020223634, WO2020142754, WO2020086475, WO2020028729, WO2019241452, WO2019173248, WO2018236548, WO2018183403, WO2017027423, WO2018106727, WO2018071672, WO2017096328, WO2017070598, WO2016201155, WO2014150624, WO2013098244, WO2021113522, WO2021050534, WO2021046442, WO2021041569, WO2021007563, WO2020252378, WO2020180699, WO2020018142, WO2019222555, WO2019178428, WO2019178427, or WO2019006471, the relevant disclosures of which are incorporated by reference for the subject matter and purpose referenced herein.

In some embodiments wherein the CRISPR nuclease is a Cas12i polypeptide, the direct repeat sequence comprises at least 90% identity to any one of SEQ ID NOs: 12-24. In some embodiments wherein the CRISPR nuclease is a Cas12i polypeptide, the direct repeat sequence comprises at least 95% identity to any one of SEQ ID NOs: 1224. In some embodiments wherein the CRISPR nuclease is a Cas12i polypeptide, the direct repeat sequence comprises any one of SEQ ID NOs: 12-24. In some embodiments, the direct repeat sequence comprises a portion of any one of SEQ ID NOs: 12-24.

TABLE 3
Direct Repeat Sequences.

| Sequence | | |
|---|---|---|
| identifier | Direct Repeat Sequence | Cas12i Description |
| SEQ ID | GUUGGAAUGACUAAUUUUUGUGCCCACCGUUGGCAC | Cas12i1 (SEQ ID NO: 8 |
| NO: 12 | | of present application) |
| SEQ ID | AAUUUUUGUGCCCAUCGUUGGCAC | Cas12i1 (SEQ ID NO: 8 |
| NO: 13 | | of present application) |
| SEQ ID | AUUUUUGUGCCCAUCGUUGGCAC | Cas12i1 (SEQ ID NO: 8 |
| NO: 14 | | of present application) |
| SEQ ID | GUUGCAAAACCCAAGAAAUCCGUCUUUCAUUGACGG | Cas12i2 (SEQ ID NO: 5 |
| NO: 15 | | of U.S. Pat. No. |
| | | 10,808,245 or SEQ ID |
| | | NOs: 2-7 of present ap- |
| | | plication) |
| SEQ ID | GCAACACCUAAGAAAUCCGUCUUUCAUUGACGGG | Cas12i2 (SEQ ID NO: 5 |
| NO: 16 | | of U.S. Pat. No. |
| | | 10,808,245 or SEQ ID |
| | | NOs: 2-7 of present ap- |
| | | plication) |
| SEQ ID | AGAAAUCCGUCUUUCAUUGACGG | Cas12i2 (SEQ ID NO: 5 |
| NO: 17 | | of U.S. Pat. No. |
| | | 10,808,245 or SEQ ID |
| | | NOs: 2-7 of present ap- |
| | | plication) |
| SEQ ID | CUAGCAAUGACCUAAUAGUGUGUCCUUAGUUGACAU | Cas12i3 (SEQ ID NO: |
| NO: 18 | | 14 of U.S. Pat. No. |
| | | 10,808,245 or SEQ ID |
| | | NO: 11 or present appli- |
| | | cation) |
| SEQ ID | CCUACAAUACCUAAGAAAUCCGUCCUAAGUUGACGG | Cas12i3 (SEQ ID NO: |
| NO: 19 | | 14 of U.S. Pat. No. |
| | | 10,808,245 or SEQ ID |
| | | NO: 11 or present appli- |
| | | cation) |
| SEQ ID | AUAGUGUGUCCUUAGUUGACAU | Cas12i3 (SEQ ID NO: |
| NO: 20 | | 14 of U.S. Pat. No. |
| | | 10,808,245 or SEQ ID |
| | | NO: 11 or present appli- |
| | | cation) |
| SEQ ID | GUUGGAAUGACUAAUUUUUGUGCCCACCGUUGGCAC | Cas12i4 (SEQ ID NO: |
| NO: 21 | | 16 of U.S. Pat. No. |
| | | 10,808,245, SEQ ID |
| | | NOs: 9 or 10 of present |
| | | application) |
| SEQ ID | CCCACAAUACCUGAGAAAUCCGUCCUACGUUGACGG | Cas12i4 (SEQ ID NO: |
| NO: 22 | | 16 of U.S. Pat. No. |
| | | 10,808,245, SEQ ID |
| | | NOs: 9 or 10 of present |
| | | application) |
| SEQ ID | UCUCAACGAUAGUCAGACAUGUGUCCUCAGUGACAC | Cas12i4 (SEQ ID NO: |
| NO: 23 | | 16 of U.S. Pat. No. |
| | | 10,808,245, SEQ ID |
| | | NOs: 9 or 10 of present |
| | | application) |
| SEQ ID | AGACAUGUGUCCUCAGUGACAC | Cas12i4 (SEQ ID NO: |
| NO: 24 | | 16 of U.S. Pat. No. |
| | | 10,808,245, SEQ ID |
| | | NOs: 9 or 10 of present |
| | | application) |
| SEQ ID | AAUAGCGGCCCUAAGAAAUCCGUCUUUCAUUGACGG | Cas12i2 (SEQ ID NO: 5 |
| NO: 241 | | of U.S. Pat. No. |
| | | 10,808,245 or SEQ ID |
| | | NOs: 2-7 of present ap- |
| | | plication) |
| SEQ ID | AUUGGAACUGGCGAGAAAUCCGUCUUUCAUUGACGG | Cas12i2 (SEQ ID NO: 5 |
| NO: 242 | | of U.S. Pat. No. |
| | | 10,808,245 or SEQ ID |
| | | NOs: 2-7 of present ap- |
| | | plication) |
| SEQ ID | CCAGCAACACCUAAGAAAUCCGUCUUUCAUUGACGG | Cas12i2 (SEQ ID NO: 5 |
| NO: 243 | | of U.S. Pat. No. |
| | | 10,808,245 or SEQ ID |
| | | NOs: 2-7 of present ap- |
| | | plication) |
| SEQ ID | CGGCGCUCGAAUAGGAAAUCCGUCUUUCAUUGACGG | Cas12i2 (SEQ ID NO: 5 |
| NO: 244 | | of U.S. Pat. No. |
| | | 10,808,245 or SEQ ID |
| | | NOs: 2-7 of present ap- |
| | | plication) |
| SEQ ID | GUGGCAACACCUAAGAAAUCCGUCUUUCAUUGACGG | Cas12i2 (SEQ ID NO: 5 |
| NO: 245 | | of U.S. Pat. No. |
| | | 10,808,245 or SEQ ID |
| | | NOs: 2-7 of present ap- |
| | | plication) |
| SEQ ID | GUUGCAACACCUAAGAAAUCCGUCUUUCAUUGACGG | Cas12i2 (SEQ ID NO: 5 |
| NO: 246 | | of U.S. Pat. No. |
| | | 10,808,245 or SEQ ID |
| | | NOs: 2-7 of present ap- |
| | | plication) |
| SEQ ID | GUUGCAAUGCCUAAGAAAUCCGUCUUUCAUUGACGG | Cas12i2 (SEQ ID NO: 5 |
| NO: 247 | | of U.S. Pat. No. |
| | | 10,808,245 or SEQ ID |
| | | NOs: 2-7 of present ap- |
| | | plication) |

Nuclease binding sequences for other CRISPR nucleases such as other Type V CRISPR nucleases are known in the art and/or provided in Tables 4-6 below.

DNA-Binding Sequence

The RNA guide may also comprise a DNA-binding sequence. In some embodiments, the DNA-binding sequence is a DNA-targeting sequence (e.g., spacer). A spacer may have a length of from about 7 nucleotides to about 100 nucleotides. For example, the spacer can have a length of from about 7 nucleotides to about 80 nucleotides, from about 7 nucleotides to about 50 nucleotides, from about 7 nucleotides to about 40 nucleotides, from about 7 nucleotides to about 30 nucleotides, from about 7 nucleotides to about 25 nucleotides, from about 7 nucleotides to about 20 nucleotides, or from about 7 nucleotides to about 19 nucleotides. For example, the spacer can have a length of from about 7 nucleotides to about 20 nucleotides, from about 7 nucleotides to about 25 nucleotides, from about 7 nucleotides to about nucleotides, from about 7 nucleotides to about 35 nucleotides, from about 7 nucleotides to about 40 nucleotides, from about 7 nucleotides to about 45 nucleotides, from about 7 nucleotides to about 50 nucleotides, from about 7 nucleotides to about 60 nucleotides, from about 7 nucleotides to about 70 nucleotides, from about 7 nucleotides to about 80 nucleotides, from about 7 nucleotides to about 90 nucleotides, from about 7 nucleotides to about 100 nucleotides, from about 10 nucleotides to about 25 nucleotides, from about 10 nucleotides to about nucleotides, from about 10 nucleotides to about 35 nucleotides, from about 10 nucleotides to about 40 nucleotides, from about 10 nucleotides to about 45 nucleotides, from about 10 nucleotides to about 50 nucleotides, from about 10 nucleotides to about 60 nucleotides, from about 10 nucleotides to about 70 nucleotides, from about 10 nucleotides to about 80 nucleotides, from about 10 nucleotides to about 90 nucleotides, or from about 10 nucleotides to about 100 nucleotides.

In some embodiments, the spacer in the RNA guide may be generally designed to have a length of between 7 and 50 nucleotides or between 15 and 35 nucleotides (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides) and be complementary to a specific target sequence. In some embodiments, the RNA guide may be designed to have a length of between 18-22 nucleotides.

In some embodiments, the DNA-binding sequence has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a target sequence as described herein and is capable of binding to the complementary region of the target sequence via base-pairing.

In some embodiments, the DNA-binding sequence comprises only RNA bases. In some embodiments, the DNA-binding sequence comprises a DNA base (e.g., the spacer comprises at least one thymine). In some embodiments, the DNA-binding sequence comprises RNA bases and DNA bases (e.g., the DNA-binding sequence comprises at least one thymine and at least one uracil).

In some instances, the RNA guide disclosed herein may further comprise a linker sequence, a 5′ end and/or 3′ end protection fragment (see disclosures herein), or a combination thereof.

The spacer in any of the RNA guides disclosed herein can be specific to a target sequence, i.e., capable of binding to the complementary region of the target sequence via base-pairing. In some instances, the target sequence may be within a genomic site of interest, e.g., where gene editing is needed.

In some embodiments, the target sequence is adjacent to a PAM sequence. PAM sequences are known in the art. In some embodiments, PAM sequences capable of being recognized by a CRISPR nuclease are disclosed in WO2021055874, WO2020206036, WO2020191102, WO2020186213, WO2020028555, WO2020033601, WO2019126762, WO2019126774, WO2019071048, WO2019018423, WO2019005866, WO2018191388, WO2018170333, WO2018035388, WO2018035387, WO2017219027, WO2017189308, WO2017184768, WO2017106657, WO2016205749, WO2017070605, WO2016205764, WO2016205711, WO2016028682, WO2015089473, WO2014093595, WO2015089427, WO2014204725, WO2015070083, WO2014093655, WO2014093694, WO2014093712, WO2014093635, WO2021133829, WO2021007177, WO2020197934, WO2020181102, WO2020181101, WO2020041456, WO2020023529, WO2020005980, WO2019104058, WO2019089820, WO2019089808, WO2019089804, WO2019089796, WO2019036185, WO2018226855, WO2018213351, WO2018089664, WO2018064371, WO2018064352, WO2017106569, WO2017048969, WO2016196655, WO2016106239, WO2016036754, WO2015103153, WO2015089277, WO2014150624, WO2013176772, WO2021119563, WO2021118626, WO2020247883, WO2020247882, WO2020223634, WO2020142754, WO2020086475, WO2020028729, WO2019241452, WO2019173248, WO2018236548, WO2018183403, WO2017027423, WO2018106727, WO2018071672, WO2017096328, WO2017070598, WO2016201155, WO2014150624, WO2013098244, WO2021113522, WO2021050534, WO2021046442, WO2021041569, WO2021007563, WO2020252378, WO2020180699, WO2020018142, WO2019222555, WO2019178428, WO2019178427, or WO2019006471, the relevant disclosures of each of which are incorporated for the subject matter and purpose referenced herein.

When the gene editing system comprises a Cas12i polypeptide, the PAM sequence comprises 5′-NTTN-3′ (or 5′-TTN-3′) wherein N is any nucleotide (e.g., A, G, T, or C). The PAM sequence is upstream to the target sequence. The PAM sequence in association with other CRISPR nucleases may comprises the sequence 5′-TTY-3′ or 5′-TTB-3′, wherein Y is C or T, and B is G, T, or C. The PAM sequence may be immediately adjacent to the target sequence or, for example, within a small number (e.g., 1, 2, 3, 4, or 5) of nucleotides of the target sequence.

Tables 4-6 below provide exemplary Type V CRISPR nucleases and their corresponding nuclease binding sequences and PAM sequences as known in the art. These sequences allow one of skill in the art to design editing template RNAs as described herein with another Type V CRISPR nuclease.

TABLE 4
PAM Sequences of Exemplary Type V CRISPR Nucleases

| | | Commonly | | |
|---|---|---|---|---|
| | Protein | Associated | Tracr | |
| Subtype | Name | PAM* | Requirement | Reference^{a} |
| A | Cas12a | (T)TTV | No | Zetsche et al., Cell, 163: 759- |
| | (Cpf1) | | | 771; 2015 |
| B | Cas12b | TTN | Yes | Shmakov et al., Mol. Cell 60, |
| | | | | 385-397; 2015 |
| C | Cas12c | TG or TN | Yes | Yan et al., Science 363, 88- |
| | | | | 91; 2019; Harrington et al., |
| | | | | Mol. Cell 79, 416-424 e415; |
| | | | | 2020 |
| D | Cas12d | TA or TG | No | Chen L. X. et al., Front. |
| | | | | Microbiol. 10: 928; 2019; |
| | | | | Harrington et al., 2020 |
| E | Cas12e | TTCN | Yes | Liu J. J. et al., Nature 566, |
| | | | | 218-223; 2019 |
| F | Cas14 | — | Yes (Cas14a) | Harrington et al., Science 362, |
| | (Cas12f) | | No (Cas14b | 839-842; 2018 |
| | | | and Cas14c) | |
| G | Cas12g | na | Yes | Yan et al., Science 363, 88- |
| | | | | 91; 2019; |
| | | | | PCT/US2019/022376 |
| H | Cas12h | RTR | No | Yan et al., 2019; |
| | | | | PCT/US2020/063125 |
| I | Cas12i | TTN | No | Yan et al., 2019; |
| | | | | PCT/US2021/025257 |
| CRISPR- | Cas12j | TBN | No | Pausch et al. Science 369, |
| Casϕ | | | | 333-337; 2020 |

^{a}Relevant disclosures of the cited references are incorporated by reference for the subject matter and purpose referenced herein.
*V represents A, C or G; R represents A or G; B represents C, G or T; (T) optional; na represents no PAM

TABLE 5
Direct Repeat Sequences for Cas12 Family Proteins

| Cpf1 Protein | SEQ ID NO: | Direct Repeat |
|---|---|---|
| FnCpf1 | 93 | UAAUUUCUACUGUUGUAGAU |
| Lb3Cpf1 | 94 | AGAAAUGCAUGGUUCUCAUGC |
| BpCpf1 | 101 | AAAAUUACCUAGUAAUUAGGU |
| PeCpf1 | 102 | GGAUUUCUACUUUUGUAGAU |
| PbCpf1 | 103 | AAAUUUCUACUUUUGUAGAU |
| SsCpf1 | 104 | CGCGCCCACGCGGGGCGCGAC |
| AsCpf1 | 105 | UAAUUUCUACUGUUGUAGAU |
| Lb2Cpf1 | 106 | GAAUUUCUACUAUUGUAGAU |
| CMtCpf1 | 107 | GAAUCUCUACUCUUUGUAGAU |
| EeCpf1 | 108 | UAAUUUCUACUUUGUAGAU |
| MbCpf1 | 109 | AAAUUUCUACUGUUUGUAGAU |
| LiCpf1 | 111 | GAAUUUCUACUUUUGUAGAU |
| LbCpf1 | 113 | UAAUUUCUACUAAGUGUAGAU |
| PeCpf1 | 115 | UAAUUUCUACUAUUGUAGAU |
| PdCpf1 | 117 | UAAUUUCUACUUCGGUAGAU |
| PmCpf1 | 119 | UAAUUUCUACUAUUGUAGAU |

See also Zetsche et al., Cell 163:759-771 (2015), the relevant disclosures of which are incorporated by reference for the subject matter and purpose referenced herein.

Table 6 below provides information for additional Type V CRISPR nucleases as known in the art.

TABLE 6
Additional Type V CRISPR Nucleases

| Subtype | Protein name | Reference^{a} |
|---|---|---|
| B | Cas12b | Shmakov et al., 2015 |
| C | Cas12c | Yan et al., 2019; Harrington et al., 2020 |
| D | Cas12d | Harrington et al., 2020 |
| E | Cas12e | Liu J. J. et al., 2019 |
| F | Cas14 (cas12f) | Harrington et al., 2018 |

^{a}Relevant disclosures of the cited references are incorporated by reference for the subject matter and purpose referenced herein.

ii. RNA Reverse Transcriptase Donor or RT Donor RNA

The editing template RNA in any of the gene editing systems disclosed herein may also comprise an RNA reverse transcriptase (RT) donor (RT donor RNA). The RT donor RNA may comprise: (i) a primer binding site (PBS), and (ii) a reverse transcription template sequence. In some instances, the RT donor RNA may further comprise: (iii) a nucleotide linker sequence, (iv) a 5′ end and/or 3′ end protection fragment (see disclosures herein), or a combination thereof. In some embodiments, the editing template RNA comprises one or more RT donor RNAs. In some embodiments, the editing template RNA comprises one or more PBS, one or more reverse transcription template sequences, and/or one or more nucleotide linker sequences. In some embodiments, a first editing template RNA comprises one or more PBS and a second editing template RNA comprises one or more reverse transcription template sequences.

In some embodiments, a RT donor RNA comprises an aptamer. In some embodiments, the aptamer recruits a reverse transcriptase polypeptide.

Primer Binding Site (PBS)

In some embodiments, the PBS in an RT donor RNA as disclosed herein is an RNA sequence capable of binding to a DNA strand via base-paring. The DNA strand has been or can be nicked or cleaved by a CRISPR nuclease. In some embodiments, the PBS comprises an RNA sequence capable of binding to a DNA strand (a PBS-targeting site) via base-pairing. The DNA strand may have a free 3′ free end or a 3′ free end can be generated via cleavage by a CRISPR nuclease contained in the same gene editing system. In some examples, the PBS-targeting site may be located on the same DNA strand as the PAM sequence (the PAM strand). In some examples, the PBS-targeting site may be located on the complementary strand of the PAM strand (the non-PAM strand).

In some embodiments, the PBS is at least 3 nucleotides, at least 4 nucleotides, at least nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the PBS is about 3 nucleotides to about 200 nucleotides in length (e.g., about 3 nucleotides, 5 nucleotides, 8 nucleotides, 10 nucleotides, 13 nucleotides, nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides or any length in between). In some embodiments, the PBS is about 3 nucleotides to about 100 nucleotides in length (e.g., about 3 nucleotides, 5 nucleotides, 8 nucleotides, 10 nucleotides, 13 nucleotides, 15 nucleotides, 20 nucleotides, 30 nucleotides, 40 nucleotides, 50 nucleotides, 60 nucleotides, 70 nucleotides, 80 nucleotides, 90 nucleotides, or 100 nucleotides or any length in between).

In some embodiments, the PBS is about 10 nucleotides to about 50 nucleotides in length. In some embodiments, the PBS is about 10 nucleotides to about 40 nucleotides in length. In some embodiments, the PBS is about 10 nucleotides to about 30 nucleotides in length. In some embodiments, the PBS is about 10 nucleotides to about 20 nucleotides in length. In some embodiments, the PBS is about 10 nucleotides to about 15 nucleotides in length. In some embodiments, the PBS is about 11 nucleotides in length. In some embodiments, the PBS is about 12 nucleotides in length. In some embodiments, the PBS is about 13 nucleotides in length. In some embodiments, the PBS is about 14 nucleotides in length. In some embodiments, the PBS is about 30 nucleotides in length.

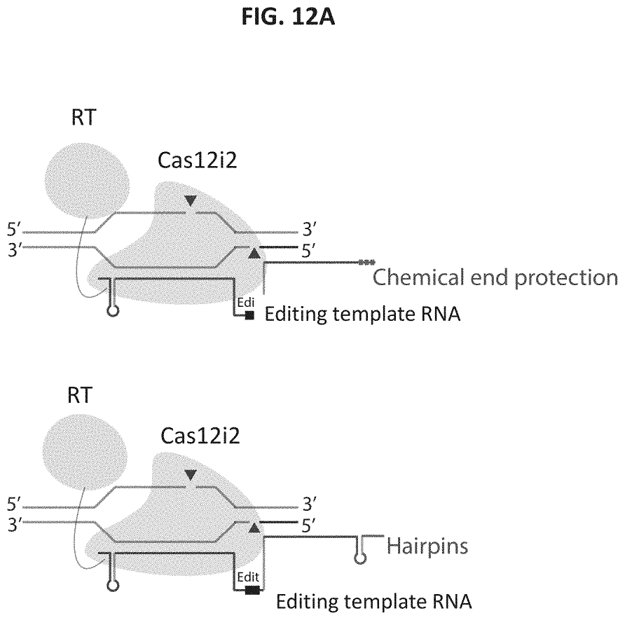

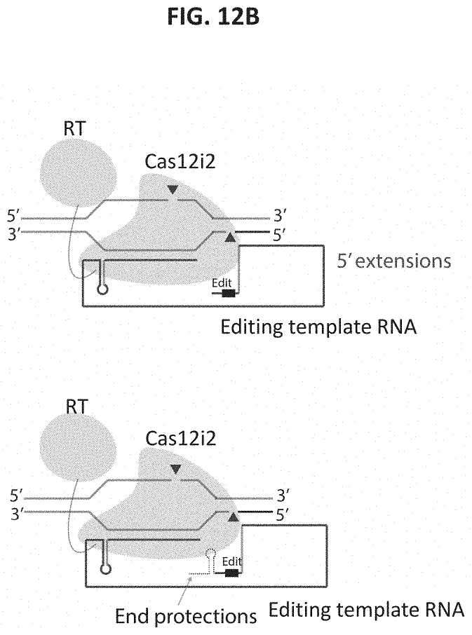

In a gene editing system comprising a Cas12i polypeptide (e.g., a Cas12i2 polypeptide as those disclosed herein), the PBS in the RT donor RNA may bind to a region (the PBS-targeting site) on the non-PAM strand. In some instances, the PBS-targeting site may be located upstream to the complementary region of a target sequence. For example, the PBS-targeting site may be up to 20 nucleotides upstream to the complementary region, for example, up to 15 nucleotides, up to 10 nucleotides, or up to 5 nucleotides. In specific examples, the PBS-targeting site may be about 3 nucleotides to about 10 nucleotides upstream of the complementary region. In specific examples, the PBS-targeting site may be 1 nucleotide, 1-2 nucleotides, 1-3 nucleotides, 1-4 nucleotides, 1-5 nucleotides, 1-6 nucleotides, 1-7 nucleotides, 1-8 nucleotides, 1-9 nucleotides, 1-10 nucleotides, 2-3 nucleotides, 2-4 nucleotides, 2-5 nucleotides, 2-6 nucleotides, 2-7 nucleotides, 2-8 nucleotides, 2-9 nucleotides, 2-10 nucleotides, 3-4 nucleotides, 3-5 nucleotides, 3-6 nucleotides, 3-7 nucleotides, 3-8 nucleotides, 3-9 nucleotides, 3-10 nucleotides, 4-5 nucleotides, 4-6 nucleotides, 4-7 nucleotides, 4-8 nucleotides, 4-9 nucleotides, 4-10 nucleotides, 5-6 nucleotides, 5-7 nucleotides, 5-8 nucleotides, 5-9 nucleotides, 5-10 nucleotides, 6-7 nucleotides, 6-8 nucleotides, 6-9 nucleotides, 6-10 nucleotides, 7-8 nucleotides, 7-9 nucleotides, 7-10 nucleotides, 8-9 nucleotides, 8-10 nucleotides, 9-10 nucleotides, or 10 nucleotides upstream of the complementary region. In other instances, the PBS-targeting site may overlap with the complementary region. When a free 3′ end is generated by the Cas12i polypeptide in the gene editing system within or nearby the target sequence and the complementary region, the PBS binding to the non-PAM strand at a site upstream to or overlapping with the complementary region could efficiently facilitate DNA synthesis by the RT polypeptide in the gene editing system, starting from the free 3′ end generated in the non-PAM strand. An exemplary illustration is provided in FIG. 12A and FIG. 12B.

Reverse Transcription Template Sequence

The reverse transcription template sequence (template sequence) serves as the template for the reverse transcription mediated by the RT polypeptide in the gene editing system disclosed herein. In some embodiments, the reverse transcription template sequence comprises a sequence with at least one encoded edit. In some embodiments, the reverse transcription template sequence comprises sequence homology to a target sequence or its complementary region with at least one encoded edit. In some embodiments, the reverse transcription template sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the reverse transcription template sequence is about 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, or 120 nucleotides in length or any length in between.

In some embodiments, the reverse transcription template sequence is about 25 nucleotides. In some embodiments, the reverse transcription template sequence is about 26 nucleotides. In some embodiments, the reverse transcription template sequence is about 27 nucleotides. In some embodiments, the reverse transcription template sequence is about 28 nucleotides. In some embodiments, the reverse transcription template sequence is about 29 nucleotides. In some embodiments, the reverse transcription template sequence is about 30 nucleotides. In some embodiments, the reverse transcription template sequence is about 31 nucleotides. In some embodiments, the reverse transcription template sequence is about 32 nucleotides. In some embodiments, the reverse transcription template sequence is about 33 nucleotides. In some embodiments, the reverse transcription template sequence is about 34 nucleotides. In some embodiments, the reverse transcription template sequence is about 35 nucleotides. In some embodiments, the reverse transcription template sequence is about 36 nucleotides. In some embodiments, the reverse transcription template sequence is about 37 nucleotides. In some embodiments, the reverse transcription template sequence is about 38 nucleotides. In some embodiments, the reverse transcription template sequence is about 39 nucleotides. In some embodiments, the reverse transcription template sequence is about 40 nucleotides. In some embodiments, the reverse transcription template sequence is about 41 nucleotides. In some embodiments, the reverse transcription template sequence is about 42 nucleotides. In some embodiments, the reverse transcription template sequence is about 43 nucleotides. In some embodiments, the reverse transcription template sequence is about 44 nucleotides. In some embodiments, the reverse transcription template sequence is about 45 nucleotides. In some embodiments, the reverse transcription template sequence is about 46 nucleotides. In some embodiments, the reverse transcription template sequence is about 47 nucleotides. In some embodiments, the reverse transcription template sequence is about 48 nucleotides. In some embodiments, the reverse transcription template sequence is about 49 nucleotides. In some embodiments, the reverse transcription template sequence is about 50 nucleotides.

In some embodiments, the reverse transcription template sequence comprises at least one encoded edit relative to a target sequence. In other embodiments, the reverse transcription template sequence comprises at least one encoded edit relative to the complementary region of a target sequence. In some embodiments, the at least one encoded edit comprises at least one substitution, insertion, and/or deletion. In some embodiments, the edit in the target sequence comprises a substitution, an insertion, and/or a deletion relative to the sequence of a target sequence. In some embodiments, the reverse transcription template sequence comprises at least one LoxP site.

In some embodiments, the edit can be a single or multi-nucleotide substitution, such as a G to T substitution, a G to A substitution, a G to C substitution, a T to G substitution, a T to A substitution, a T to C substitution, a C to G substitution, a C to T substitution, a C to A substitution, an A to T substitution, an A to G substitution, or an A to C substitution. In some embodiments, the change in sequence can convert a G:C base pair to a T:A base pair, a G:C base pair to an A:T base pair, a G:C base pair to C:G base pair, a T:A base pair to a G:C base pair, a T:A base pair to an A:T base pair, a T:A base pair to a C:G base pair, a C:G base pair to a G:C base pair, a C:G base pair to a T:A base pair, a C:G base pair to an A:T base pair, an A:T base pair to a T:A base pair, an A:T base pair to a G:C base pair, or an A:T base pair to a C:G base pair.

In some embodiments, the single or multi-nucleotide substitution comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In some embodiments, the substitution is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides in length. In some embodiments, the substitution is from 1 nucleotide to about 300 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to 35 nucleotides, from 35 nucleotides to nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides in length. In some embodiments, the substitution is up to about 10,000 bases (10 kb) in length. For example, in some embodiments, the substitution is 1 base, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, 5 kb, 6 kb, 7 kb, 8 kb, 9 kb, or 10 kb in length.

In some embodiments, the edit comprises a single or multi-nucleotide insertion that is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In some embodiments, the single or multi-nucleotide insertion is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides in length. In some embodiments, the single or multi-nucleotide insertion is from 1 nucleotide to about 300 nucleotides in length, e.g., 1 nucleotide to 5 nucleotides, from 5 nucleotides to 10 nucleotides, from 10 nucleotides to 15 nucleotides, from 15 nucleotides to nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides in length. In some embodiments, the single or multi-nucleotide insertion is up to about 10,000 bases (10 kb) in length. For example, in some embodiments, the insertion is 1 base, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, 5 kb, 6 kb, 7 kb, 8 kb, 9 kb, or 10 kb in length.

In some embodiments, the edit comprises a single or multi-nucleotide deletion that is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In some embodiments, the single or multi-nucleotide deletion is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides in length. In some embodiments, the single or multi-nucleotide deletion is from 1 nucleotide to about 300 nucleotides in length, e.g., 1 nucleotide to 5 nucleotides, from 5 nucleotides to 10 nucleotides, from 10 nucleotides to 15 nucleotides, from 15 nucleotides to nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides in length. In some embodiments, the deletion is up to about 10,000 bases (10 kb) in length. For example, in some embodiments, the deletion is 1 base, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, 5 kb, 6 kb, 7 kb, 8 kb, 9 kb, or 10 kb in length.

In some embodiments, the reverse transcription template sequence comprises at least one encoded edit and a length that is from about 5 nucleotides to about 10,000 nucleotides in length, e.g., from 5 nucleotides to 10 nucleotides, from 10 nucleotides to 15 nucleotides, from nucleotides to 20 nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides, or about 1 kilobase (kb), about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, 5 kb, 6 kb, 7 kb, 8 kb, 9 kb, or 10 kb in length.

The reverse transcription template sequence can be transcribed into DNA by the reverse transcriptase of the gene editing system described herein. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is 5′ of the PBS. In some embodiments, the reverse transcription template sequence is 3′ of the PBS. In some embodiments, the reverse transcription template sequence is transcribed into DNA of the PAM strand through 3′ extension from the PBS. In some embodiments, the reverse transcription template sequence is transcribed into DNA of the non-PAM strand through 3′ extension from the PBS.

iii. Additional Elements

In some embodiments, the editing template RNA may comprise one or more additional elements. For example, the editing template RNA, or the gRNA and/or the RT donor RNA thereof, may comprise one or more protection fragments at either or both ends of the RNA molecules. Alternatively or in addition, the editing template RNA, or the gRNA and/or the RT donor RNA thereof, may comprise additional elements internal to the RNA molecule (e.g., between one or more of the sequences in the editing template RNA, e.g., between a PBS and a reverse transcription template sequence, e.g., a linker). In some embodiments, the editing template RNA comprises additional elements between one or more sequence of the editing template RNA, e.g., such as an RNA guide (a nuclease binding sequence or a DNA-binding sequence) or an RT donor RNA (a PBS or a reverse transcription template sequence).

In some embodiments, the editing template RNA comprises additional elements, e.g., a direct repeat sequence, at one or more ends. In some embodiments, the direct repeat sequence may recruit a CRISPR nuclease (e.g., a Type V nuclease such as a variant Cas12i2 polypeptide or a variant Cas12i2-reverse transcriptase fusion polypeptide, or a Cas12i4-reverse transcriptase fusion polypeptide).

In some embodiment, the additional elements may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In some examples, the editing template RNA may comprise an optional nucleotide linker. Such an optional nucleotide linker sequence may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the optional nucleotide linker is between any of the nuclease binding sequence, the DNA-binding sequence, the PBS and/or reverse transcription template sequence.

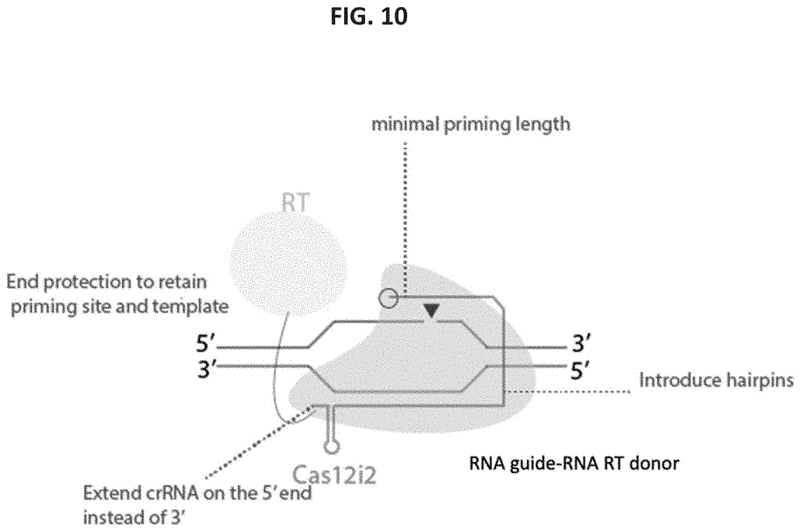

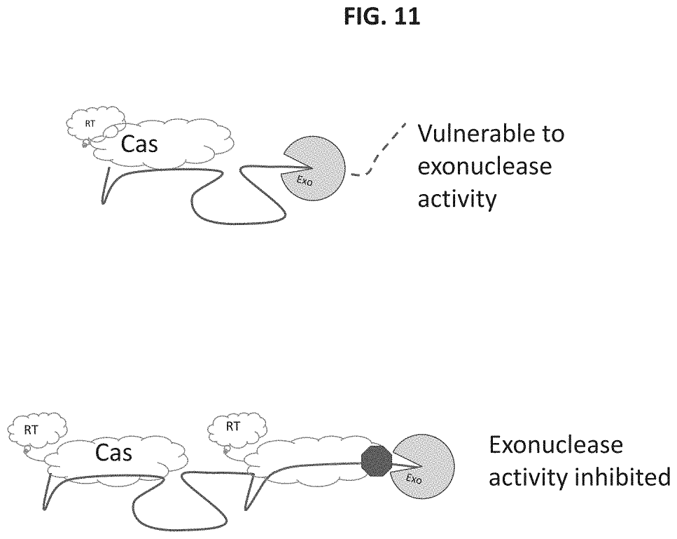

In some examples, the 5′ end and/or the 3′ end of the editing template RNA, or the gRNA and/or the RT donor RNA thereof, may contain a protection fragment, which may enhance resistance of the RNA molecule to exonuclease activity. See, e.g., FIG. 11. In some instances, the end protection fragment may comprise a nucleotide sequence capable of forming a secondary structure, such as hairpin, a pseudoknot, or a triplex structure. In other instances, the end protection fragment may comprise the sequence of an exoribonuclease-resistant RNA (xrRNA), a transfer RNA (tRNA), or a truncated tRNA. In some embodiments, the modification is a Zika-like pseudoknot, a murine leukemia virus pseudoknot (MLV-PK) sequence, a red clover necrotic mosaic virus (RCNMV) sequence, a sweet clover necrotic mosaic virus (SCNMV) sequence, a carnation ringspot virus (CRSV) sequence, preQ sequence, or an RNA bacteriophage MS2 sequence. In specific examples, the end protection fragment may comprise one or more CRISPR nuclease binding sites (e.g., bindings sites for a Cas12i polypeptide such as a Cas12i2 polypeptide), and optionally one or more segments (e.g., spacers) that share no homology with any human sequences. In some instances, the one or more segment bind to a sequence that is no more than 85% identical to any sequence of the human genome. See FIG. 10, FIG. 11, FIG. 12A, and FIG. 12B. Such an end protection fragment can recruit the CRISPR nuclease contained in the same gene editing system to inhibit exoribonuclease activity without inducing off-target gene edits.

In some embodiments, a gene editing system as disclosed herein comprises at least one editing template RNA (e.g., a gene editing RNA) or a nucleotide sequence encoding such. In some examples, the at least one editing template RNA is capable of binding to a CRISPR nuclease (e.g., a Type V CRISPR nuclease). In some examples, the at least one editing template RNA is further capable of binding to a nucleic acid (e.g., DNA or a target nucleic acid). In some examples, the at least one editing template RNA comprises a nuclease binding sequence (e.g., one or more binding sites recognizable by a CRISPR nuclease) and a DNA-binding sequence (e.g., a spacer). In some instances, the at least one editing template RNA comprises a gRNA (comprising a nuclease binding sequence and a spacer), and an RT donor RNA. In some embodiments, an editing template RNA comprises an RNA guide linked to an RT donor RNA. See, e.g., FIG. 19B.

iv. Modification of Nucleic Acids

Any of the RNA components in a gene editing system as disclosed herein, e.g., the editing template RNA, the RNA guide, the RT donor RNA, may include one or more modifications.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide or any of the nucleic acid sequences encoding components of the composition may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabinopentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethylpseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-azazebularine, 5-methyl-zebularine, 5-aza-2-thio-zebularine, 2-thio-zebularine, 2-methoxycytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cishydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, any RNA sequence described herein, such as an editing template RNA, may comprise an end modification (e.g., a 5′ end modification or a 3′ end modification). In some embodiments, the end modification is a chemical modification. In some embodiments, the end modification is a structural modification. See disclosures herein.

When a gene editing system disclosed herein comprises nucleic acids encoding the CRISPR nuclease and/or the RT polypeptide, e.g., mRNA molecules, such nucleic acid molecules may contain any of the modifications disclosed herein, where applicable.

D. Exemplary Gene Editing Systems

The exemplary gene editing systems described herein are meant to be illustrative only.

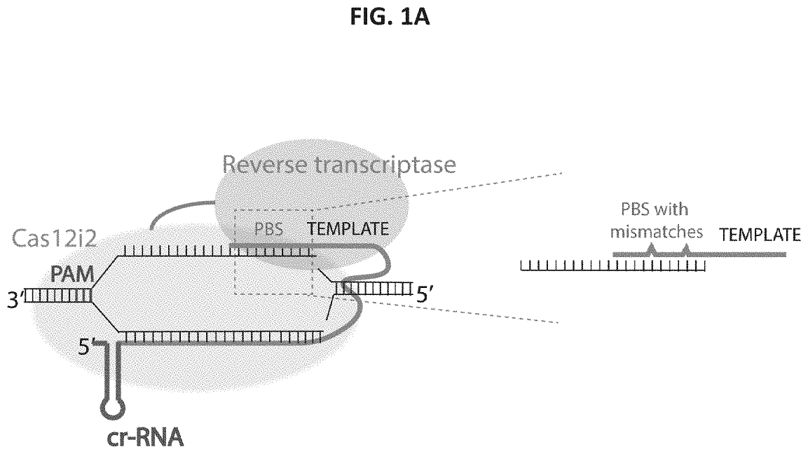

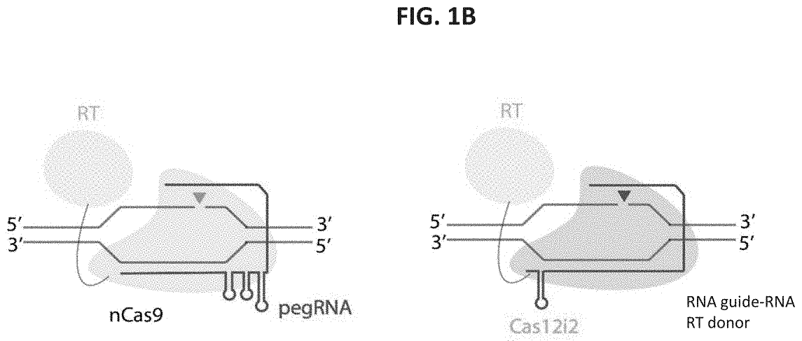

In some embodiments, exemplary gene editing systems are depicted in FIG. 1A and FIG. 1B. In these exemplary designs, an RNA guide may comprise a 3′ fusion partner, which may comprise an RT donor RNA (comprising a PBS and a reverse transcription template sequence), any of the additional elements disclosed herein, or a combination thereof. In some instances, the PBS is about 3 to about 24 nucleotides (e.g., about 3, 4, 5, 6, 7, 8, 9, 10, 11, 12, 13, 14, 15, 16, 17, 18, 19, 20, 21, 22, 23, or 24 nucleotides) in length. Alternatively or in addition, the PBS may have at least about 75% complementarity to the corresponding PBS-targeting site, which may be located on the PAM strand. In some embodiments, the reverse transcription template sequence is about 10 nucleotides to about 100 nucleotides (e.g., about 10, 15, 20, 25, 30, 35, 40, 45, 50, 55, 60, 65, 70, 75, 80, 85, 90, 95, or 100 nucleotides) in length. In some embodiments, a linker is present between the DNA-binding sequence (spacer) in the RNA guide and the reverse transcription template sequence. In some examples, the linker comprises one or more hairpins. For example, the hairpins can reduce annealing between the PBS and the DNA-binding sequence.

In some instances, the CRISPR nuclease (e.g., a Type V nuclease such as a Cas12i polypeptide) in the exemplary gene editing system may comprise an N-terminal or C-terminal fusion partner. In some embodiments, the N-terminal or C-terminal fusion partner comprises a reverse transcriptase polypeptide.

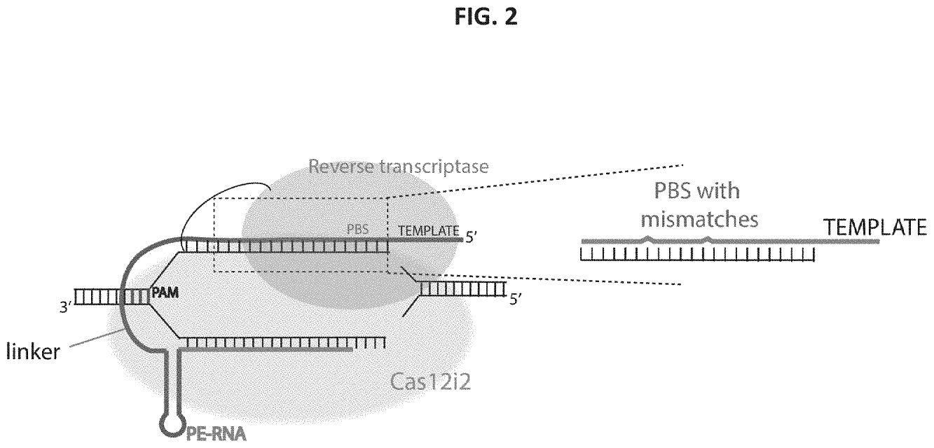

In other embodiments, exemplary gene editing systems as disclosed herein are depicted in FIG. 2. In these exemplary designs, an RNA guide may comprise a 5′ fusion partner, which may comprises an RT donor RNA (comprising a PBS and a reverse transcription template sequence), one or more of the additional elements, or a combination thereof. In some embodiments, the reverse transcription template sequence is about 10 nucleotides to about 100 nucleotides (e.g., about 10, 15, 20, 25, 30, 35, 40, 45, 50, 55, 60, 65, 70, 75, 80, 85, 90, 95, or 100 nucleotides) in length. In some embodiments, the PBS is about 3 nucleotides to about 24 nucleotides (e.g., about 3, 4, 5, 6, 7, 8, 9, 10, 11, 12, 13, 14, 15, 16, 17, 18, 19, 20, 21, 22, 23, or 24 nucleotides) in length. Alternatively or in addition, the PBS has at least about 75% complementarity to the corresponding PBS-targeting site, which may be located on the PAM strand. In some embodiments, a linker is present between the DNA-binding sequence of the RNA guide and the PBS. In some examples, the linker comprises one or more hairpins. For example, the hairpins can reduce annealing between the PBS and the DNA-binding sequence.

In some instances, the CRISPR nuclease (e.g., a Type V nuclease such as a Cas12i polypeptide) in the exemplary gene editing system may comprise an N-terminal or C-terminal fusion partner. In some embodiments, the N-terminal or C-terminal fusion partner comprises a reverse transcriptase polypeptide.

The exemplary gene editing systems depicted in FIG. 1A, FIG. 1B, and FIG. 2 can be used to edit the PAM-strand of a target nucleic acid (e.g., a genomic site of interest). Without wishing to be bound by theory, using these exemplary gene editing systems FIG. 1A, FIG. 1B, and FIG. 2, during cleavage by the CRISPR nuclease (e.g., a Type V nuclease such as a Cas12i polypeptide), the free 3′ end of the PAM strand can base-pair with the PBS, extend using the reverse transcription template sequence as the template, and strand exchange back to base-pairing with the complementary genomic strand, resulting in edit incorporation.

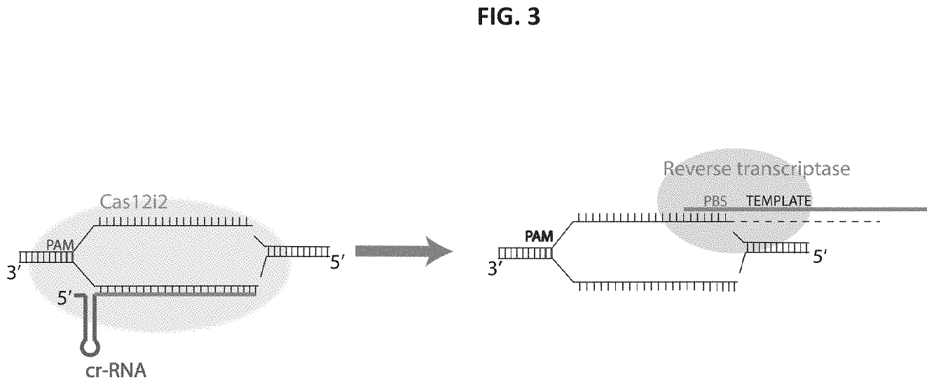

In yet other embodiments, exemplary gene editing systems disclosed herein are depicted in FIG. 3. Such an exemplary gene editing system comprises two RNA molecules: an RNA guide comprising a nuclease binding sequence and a DNA-binding sequence (a spacer) and an RT donor RNA. The RT donor RNA may comprise a PBS and a reverse transcription template sequence. In some examples, the reverse transcription template sequence does not encode an edit. In other examples, the RT donor RNA comprises a PBS and a reverse transcription template sequence encoding an edit. In some embodiments, the reverse transcription template sequence or a portion thereof can bind to the target nucleic acid via base pairing.

In some instances, the PBS is up to about 100 nucleotides in length. In some embodiments, the PBS is about 3 nucleotides to about 100 nucleotides in length. In some embodiments, the reverse transcription template sequence is about 10 nucleotides to about 100 nucleotides in length. In some embodiments, the reverse transcription template sequence of the RT donor RNA comprises an aptamer at the 5′ end. In some embodiments, the aptamer recruits a reverse transcriptase polypeptide. In some embodiments, the PBS of the RT donor RNA is not complementary to any other portion of the editing template RNA (e.g., the nuclease binding sequence and/or the DNA-binding sequence).

The exemplary gene editing system depicted in FIG. 3 can comprise either one or two protein components. For example, the exemplary gene editing system may comprise a CRISPR nuclease (e.g., a Type V nuclease such as a Cas12i polypeptide) having an N-terminal or C-terminal fusion partner, which may comprise a reverse transcriptase polypeptide. Alternatively, the gene editing system may comprise the CRISPR nuclease (e.g., a Type V nuclease such as a Cas12i polypeptide) and the reverse transcriptase polypeptide as two separate polypeptides.

The exemplary gene editing system depicted in FIG. 3 can be used to edit either the PAM strand or the non-PAM strand of a target nucleic acid (e.g., a genomic site of interest). Without wishing to be bound by theory, using such an exemplary gene editing system, after the CRISPR nuclease (e.g., a Type V nuclease such as a Cas12i polypeptide) is released from the target nucleic acid, the free 3′ end of the PAM strand or the non-PAM strand can base-pair with the PBS, extend using the reverse transcription template sequence as the template, and strand exchange back to hybridizing with the complementary genomic strand, resulting in incorporation of an edit from the RT donor RNA. The exemplary gene editing system can be used to edit at a PAM distal region of the target nucleic acid.

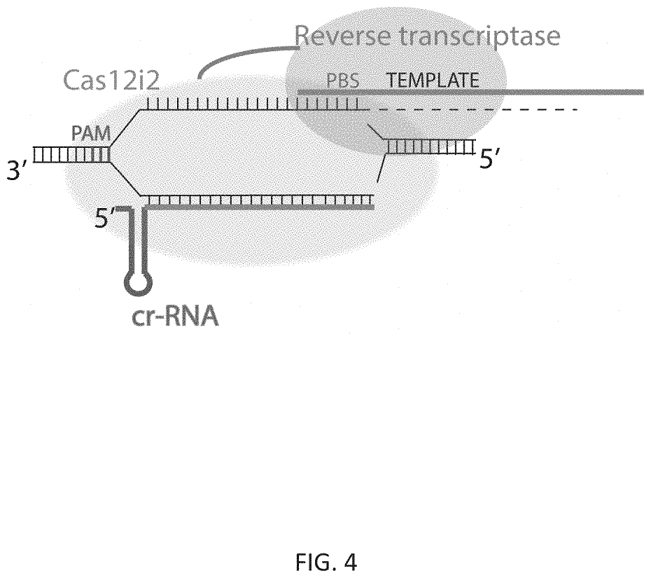

In still other embodiments, exemplary gene editing systems disclosed herein are depicted in FIG. 4. Such an exemplary gene editing system may comprise two RNA molecules: an RNA guide and an RT donor RNA as two separate RNA molecules. The exemplary gene editing system can comprise either one or two protein components as disclosed herein. For example, the exemplary gene editing system may comprise a CRISPR nuclease (e.g., a Type V nuclease such as a Cas12i polypeptide) having an N-terminal or C-terminal fusion partner, which may comprise a reverse transcriptase polypeptide. Alternatively, the CRISPR nuclease (e.g., a Type V nuclease such as a Cas12i polypeptide) and the reverse transcriptase polypeptide are not fused to one another (are two separate polypeptides).

The exemplary gene editing system depicted in FIG. 4 can be used to edit either the PAM strand or the non-PAM strand. Without wishing to be bound by theory, using the exemplary gene editing system, the free 3′ end of the PAM strand or the non-PAM strand can base-pair with the PBS of the RT donor RNA in the same gene editing system, extend using the reverse transcription template sequence as the template, and strand exchange back to hybridizing with the complementary genomic strand, resulting in incorporation of the edit from the RT donor RNA.

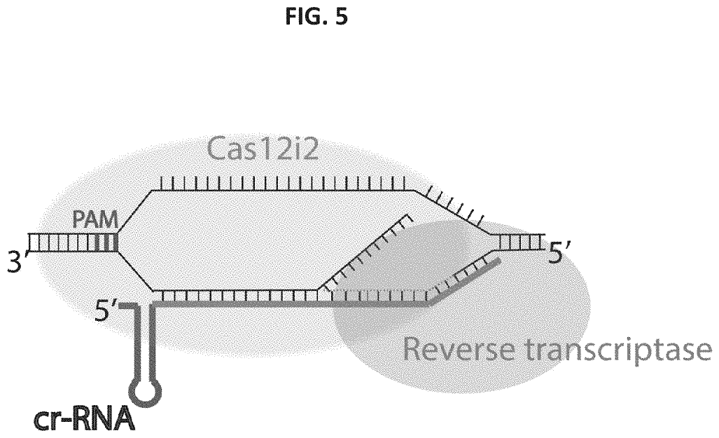

In some embodiments, exemplary gene editing systems disclosed herein are depicted in a FIG. 5. In such an exemplary gene editing system, the RNA guide may comprise a 3′ fusion partner, which may comprises an RT donor RNA (comprising a reverse transcription template sequence and a PBS). In some instances, the PBS binds a site on the non-PAM strand upstream to the complementary region of the target sequence.

In some examples, the PBS is about 3 nucleotides to about 100 nucleotides (e.g., about 3, 5, 10, 15, 20, 25, 30, 35, 40, 45, 50, 55, 60, 65, 70, 75, 80, 85, 90, 95, or 100 nucleotides) in length. In some embodiments, the DNA-binding sequence (spacer) is about 20 nucleotides to about 25 nucleotides in length. In some embodiments, the DNA-binding sequence comprises at least one edit that is incorporated about 10 nucleotides to about 25 nucleotides from the PAM sequence.

In some examples, the exemplary gene editing system may comprise the CRISPR nuclease (e.g., a Type V nuclease such as a Cas12i polypeptide), which comprises a 5′ fusion or 3′ fusion partner. The 5′ fusion or 3′ fusion partner may comprise a reverse transcriptase polypeptide. In some embodiments, the CRISPR nuclease (e.g., a Type V nuclease such as a Cas12i polypeptide) lacks crRNA processing activity.

The exemplary gene editing system depicted in FIG. 5 can be used to edit the non-PAM strand of a target nucleic acid (e.g., a genomic site of interest). Without wishing to be bound by theory, using such an exemplary gene editing system, during cleavage by the CRISPR nuclease (e.g., a Type V nuclease such as a Cas12i polypeptide), the free 3′ end of the non-PAM strand can base-pair with the PBS and extend using the DNA-binding sequence as a template. The RT extension on the non-PAM strand exchanges back to base-pairing with the complementary genomic strand, resulting in incorporation of the edit from the RT donor RNA.

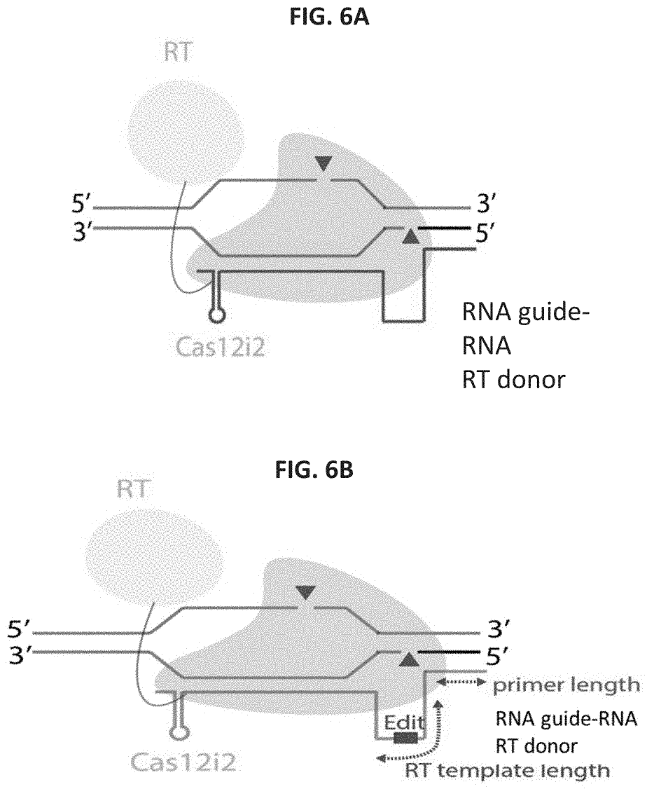

In some embodiments, exemplary gene editing systems are depicted FIG. 6A and FIG. 6B. In such an exemplary gene editing system, the RNA guide may comprise a 3′ fusion partner, which may comprise an RT donor RNA (comprising a reverse transcription template sequence and a PBS). In some embodiments, the PBS is complementary to a region in the non-PAM strand that is upstream to the complementary region of the target sequence on the PAM strand. In some examples, a hairpin is present between the DNA-binding sequence of the RNA guide and the reverse transcription template sequence. In some embodiments, a hairpin is present within the reverse transcription template sequence. In some embodiments, the edit in the template sequence may create a hairpin in the target nucleic acid where the edit is incorporated.

In some examples, the exemplary gene editing system may comprise the CRISPR nuclease (e.g., a Type V nuclease such as a Cas12i polypeptide), which comprises an N-terminal or C-terminal fusion partner. The N-terminal or C-terminal fusion partner may comprise a reverse transcriptase polypeptide.

In some embodiments, exemplary gene editing systems are depicted in FIG. 7. In such an exemplary gene editing system, the RNA guide may comprise a 5′ fusion partner, which may comprise an RT donor RNA (comprising a PBS and a reverse transcription template sequence). In some embodiments, the PBS is about 5 to about 20 nucleotides (e.g., about 5, 6, 7, 8, 9, 10, 11, 12, 13, 14, 15, 16, 17, 18, 19, or 20 nucleotides) in length. Alternatively or in addition, the PBS has at least about 75% complementarity to a region (the corresponding PBS-targeting site) on the non-PAM strand. In some instances, a linker is present between the nuclease binding sequence of the RNA guide and the PBS of the RT donor RNA. Alternatively or in addition, a hairpin may be present between the DNA-binding sequence of the RNA guide and the revere transcription template sequence of the RT donor RNA. In some embodiments, a hairpin is present within the reverse transcription template sequence. In some embodiments, the edit in the template sequence may create a hairpin in the target nucleic acid where the edit is incorporated.

In some instances, the CRISPR nuclease (e.g., a Type V nuclease such as a Cas12i polypeptide) in the exemplary gene editing system may comprise an N-terminal or C-terminal fusion partner, which may comprise a reverse transcriptase polypeptide. In some embodiments, the CRISPR nuclease (e.g., a Type V nuclease such as a Cas12i polypeptide) lacks crRNA processing activity (e.g., those disclosed herein).

The exemplary gene editing systems depicted in FIG. 6A, FIG. 6B, or FIG. 7 can be used to edit the non-PAM strand of a target nucleic acid (e.g., a genomic site of interest). Without wishing to be bound by theory, using the exemplary gene editing system, during cleavage by the CRISPR nuclease (e.g., a Type V nuclease such as a Cas12i polypeptide), the free 3′ end of the non-PAM strand can base-pair with the PBS and extend using the reverse transcription template sequence as the template. The RT extension on the non-PAM strand exchange back to base-pair with the complementary genomic strand, resulting in incorporation of at least one edit from the RT donor RNA.

The exemplary gene editing systems disclosed herein, e.g., those depicted in FIG. 6A, FIG. 6B, and FIG. 7, can be used to incorporate at least one PAM-proximal edit within the region on the non-PAM strand that is complementary to the target sequence on the PAM strand. In some examples, the exemplary gene editing system can be used to modify the PAM sequence and/or a sequence upstream of a PAM sequence (e.g., via introducing variations in the region complementary to the PAM sequence and/or the upstream sequence). Such exemplary gene editing systems can be used to prevent retargeting of the resultant modified genetic locus by the same CRISPR nuclease (e.g., a Type V nuclease such as a Cas12i polypeptide).

In some embodiments, exemplary gene editing systems disclosed herein are depicted in FIG. 10. In such an exemplary gene editing system, the RNA guide may comprises a 3′ fusion partner, which may comprise an RT donor RNA (comprising a PBS and a reverse transcription template sequence). Alternatively, the RNA guide may comprise a 5′ fusion partner, which may comprise the RT donor RNA (comprising a reverse transcription template sequence and a PBS). The length of the PBS can be variable. For example, the PBS length can be about 3 nucleotides to about 16 nucleotides in length. In some examples, the PBS is capable of binding to a region on the PAM strand, e.g., overlapping with the target sequence, of a target nucleic acid (e.g., a genomic site of interest). In some examples, a hairpin is present between the DNA-binding sequence of the RNA guide and the reverse transcription template sequence of the RT donor RNA. One or both ends of the RNA guide-reverse transcription template sequence can include a protection fragment, e.g., those disclosed herein, to prevent exonuclease or endonuclease activity.

The exemplary gene editing system may comprise a CRISPR nuclease (e.g., a Type V nuclease such as a Cas12i polypeptide), which may comprise an N-terminal or C-terminal fusion partner. In some examples, the N-terminal or C-terminal fusion partner comprises a reverse transcriptase polypeptide. In some examples, the CRISPR nuclease (e.g., a Type V nuclease such as a Cas12i polypeptide) lacks crRNA processing activity. In some examples, the CRISPR nuclease is a nickase. In some examples, an edit is incorporated into the PAM strand of a target nucleic acid using the exemplary gene editing system depicted in FIG. 10.

The exemplary editing template RNAs depicted in FIGS. 1-7, 8A-C, and 10, which comprise either an RT donor RNA sequence fused to the 3′ end of an RNA guide sequence or an RT donor RNA sequence fused to the 5′ end of an RNA guide sequence, can instead comprise an RT donor RNA sequence fused to an internal position of an RNA guide sequence, or vice versa. For example, an RT donor RNA can be fused to an internal position of an RNA guide, sgRNA, or an RNA guide-tracrRNA (e.g., an sgRNA).

Extended RNA guide ends (e.g., through 5′ extension or 3′ extension with an RT donor RNA) can be vulnerable to exonuclease and/or endonuclease activity, which reduces reverse transcription template sequence concentrations, along with efficiency of edit incorporation. In some embodiments, an RNA guide-RT donor RNA fusion further comprises added secondary structure to inhibit or prevent exonuclease activity. In some embodiments, the added secondary structure is a triplex structure, a pseudoknot, an xrRNA, a circular RNA, a tRNA, or a truncated tRNA. In some embodiments, the added secondary structure is a Zika-like pseudoknot, a murine leukemia virus pseudoknot (MLV-PK) sequence, a red clover necrotic mosaic virus (RCNMV) sequence, a sweet clover necrotic mosaic virus (SCNMV) sequence, a carnation ringspot virus (CRSV) sequence, preQ sequence, or an RNA bacteriophage MS2 sequence. In some embodiments, the added secondary structure is through base-stacking or 3′-end base pairing. In other embodiments, the added secondary structure is a nuclease binding sequence or a nuclease binding sequence and a DNA-binding sequence. See FIG. 10, FIG. 11, FIG. 12A, and FIG. 12B. In some embodiments, the added DNA-binding sequence is directed to a non-mammalian target. In some embodiments, the added DNA-binding sequence is directed to a non-human target. In some embodiments, the added DNA-binding sequence is not found in the human genome. In some embodiments, the added DNA-binding sequence is no more than 85% identical to any sequence of the human genome. See Example 2.

Without wishing to be bound by theory, the addition of a nuclease binding sequence and a DNA-binding sequence can recruit a CRISPR nuclease or a CRISPR nuclease-reverse transcriptase fusion. Through protein-RNA interactions, a bound CRISPR nuclease can provide resistance to endogenous exonucleases and endonucleases. In some embodiments, the additional nuclease binding sequence and DNA-binding sequence recruits a CRISPR nuclease that lacks RNA-processing activity. In some embodiments, the secondary structure is an aptamer (e.g., an RNA aptamer) and the composition further comprises a protein that interacts with the aptamer. In some embodiments, the composition comprising an aptamer and an aptamer-interacting protein inhibits endogenous exonuclease and/or endonuclease activity.

Additional exemplary gene editing systems as disclosed herein are provided below for illustrative purposes only.

In some embodiments, a gene editing system as disclosed herein comprises at least one RNA guide (or a guide RNA, which are used herein interchangeably) and at least one RT donor RNA. In some examples, the at least one RNA guide comprises a nuclease binding sequence and a DNA-binding sequence (spacer). The RNA guide may be capable of binding to a CRISPR nuclease (e.g., a Type V CRISPR nuclease). In some examples, the at least one RNA guide is further capable of binding to a target nucleic acid, e.g., via the spacer region. In some examples, the RT donor RNA comprises at least one primer binding site (PBS) and at least one reverse transcription template sequence. The PBS is capable of binding to one strand of a target nucleic acid, which can be either the sense strand or the anti-sense strand. The region to which a PBS binds is described herein as a PBS-targeting site. The at least one reverse transcription template sequence may comprise a sequence with at least one nucleotide variation relative to the corresponding sequence of the target nucleic acid (an encoded edit). In some instances, the at least one encoded edit is an insertion, substitution, and/or deletion.

In some embodiments, a gene editing system disclosed herein comprises at least one RNA guide, at least one RT donor RNA and at least one other sequence. In some embodiments, the at least one RNA guide comprises a nuclease binding sequence and a DNA-binding sequence. In some embodiments, the RNA guide is capable of binding to a CRISPR nuclease (e.g., a Type V CRISPR nuclease). In some embodiments, the at least one RNA guide is further capable of binding to a target nucleic acid. In some embodiments, the PBS of the at least one RT donor RNA is capable of binding to the non-PAM strand of a target nucleic acid. In some embodiments, the PBS of the at least one RT donor RNA is capable of binding to the PAM strand of a target nucleic acid.

In some embodiments, a gene editing system disclosed herein may comprises at least one of a CRISPR nuclease, reverse transcriptase, and an editing template RNA, which may comprise an RNA guide and RT donor RNA. In some examples, the at least one of a CRISPR nuclease, reverse transcriptase, and editing template RNA are provided in individual compositions. In some embodiments, the at least one of a CRISPR nuclease, reverse transcriptase, RNA guide and RT donor RNA are provided in individual compositions. In some embodiments, one or more of the at least one of a CRISPR nuclease, reverse transcriptase, and editing template RNA are provided in separate compositions. In some embodiments, a composition comprising the CRISPR nuclease and reverse transcriptase is provided separately from a composition comprising the editing template RNA. In some embodiments, one or more of the at least one of a CRISPR nuclease, reverse transcriptase, RNA guide, and RT donor RNA are provided in separate compositions. In some embodiments, a composition comprising the CRISPR nuclease and reverse transcriptase is provided separately from a composition comprising the RNA guide and RT donor RNA.

In some embodiments, a gene editing system provided herein may be capable of binding to a target nucleic acid, which can be a genomic site where gene editing is needed. In some embodiments, one or more components of the composition, such as the editing template RNA, bind a target nucleic acid. In some embodiments, one or more components of the composition, such as the RNA guide and RT donor RNA, bind a target nucleic acid. In some embodiments, the target nucleic acid is DNA. In some embodiments, a composition of the present invention modifies or is capable of modifying a target nucleic acid. In some embodiments, one or more of the components of the composition, such as the CRISPR nuclease and reverse transcriptase, modifies a target nucleic acid. In some embodiments, a composition of a present invention introduces a substitution, insertion, or deletion into a target nucleic acid. In some embodiments, a composition of a present invention is capable of introducing a substitution, insertion, or deletion into the non-PAM strand of a target nucleic acid. In some embodiments, a gene editing system as disclosed herein is capable of introducing a substitution, insertion, or deletion into the PAM strand of a target nucleic acid.

In some embodiments, a gene editing system as disclosed herein may comprise the protein components of the CRISPR nuclease, the RT polypeptide, or both. Alternatively, the gene editing system may comprise one or more nucleic acids (e.g., vectors such as viral vectors) encoding the protein components. In some examples, the gene editing system may comprise one vector encoding both the CRISPR nuclease and the RT polypeptide. Alternatively or in addition, a gene editing system as disclosed herein may comprise the RNA components of the gene editing RNA, the guide RNA, or both. Alternatively, the gene editing system may comprise one or more nucleic acids (vectors) encoding the RNA components. For example, the gene editing system may comprise one vector (e.g., a viral vector such as an AAV vector, e.g., AAV1, AAV2, AAV3, AAV4, AAV5, AAV6, AAV7, AAV8, AAV9, AAVrh10, AAV11 and AAV12) coding for both the gene editing RNA and the RNA guide.

In some examples, a gene editing system as disclosed herein may comprise the protein components of the CRISPR nuclease, the RT polypeptide, or both, and the RNA components of gene editing RNA and the RNA guide. In other examples, a gene editing system as disclosed herein may comprise the protein components of the CRISPR nuclease, the RT polypeptide, or both, and one or more nucleic acids encoding the RNA components of gene editing RNA and the RNA guide. In yet other examples, a gene editing system as disclosed herein may comprise one or more nucleic acids encoding the protein components of the CRISPR nuclease, the RT polypeptide, or both, and the RNA components of gene editing RNA and the RNA guide. Alternatively, a gene editing system as disclosed herein may comprise one or more nucleic acids encoding the protein components of the CRISPR nuclease, the RT polypeptide, or both, and one of more nucleic acids encoding the RNA components of gene editing RNA and the RNA guide. In some instances, the gene editing system may comprise one vector encoding multiple components of the gene editing system. In some instances, the nucleic acid(s) encoding the CRISPR nuclease, the RT polypeptide, and/or a fusion polypeptide thereof can be one or more mRNA molecules. In some examples, the mRNA molecule(s) may be codon optimized.

In some embodiments, the gene editing system disclosed herein comprises one or more lipid nanoparticles (LNPs) encompassing one or more of the protein and/or RNA components of the gene editing system, or their encoding nucleic acids. In other embodiments, the gene editing system may comprise one or more LNPs encompass a portion the components and one or more vectors encoding the remaining components.

### II. Preparation of Gene Editing System Components

The protein components, the RNA components, or their encoding nucleic acids (e.g., vectors or mRNAs) may be prepared by conventional methods of the methods disclosed herein.

In some embodiments, a CRISPR nuclease (e.g., a Type V nuclease such as a Cas12i polypeptide), a reverse transcriptase, or a CRISPR nuclease-reverse transcriptase fusion can be prepared by (a) culturing host cells such as bacteria cells or mammalian cells, capable of producing the proteins, isolating the proteins thus produced, and optionally, purifying the proteins. The CRISPR nuclease, the reverse transcriptase, or the fusion protein thus prepared may be complexed with the editing template RNA.

The CRISPR nuclease and the reverse transcriptase can be also prepared by (b) a known genetic engineering technique, specifically, by isolating a gene encoding the CRISPR nuclease and the reverse transcriptase of the present invention from bacteria, constructing a recombinant expression vector, and then transferring the vector into an appropriate host cell that expresses the editing template RNA for expression of a recombinant protein that complexes with the editing template RNA in the host cell. Alternatively, the CRISPR nuclease and the reverse transcriptase can be prepared by (c) an in vitro coupled transcription-translation system and then complexes with editing template RNA. Bacteria that can be used for preparation of the CRISPR nuclease and the reverse transcriptase of the present invention are not particularly limited as long as they can produce the CRISPR nuclease and the reverse transcriptase of the present invention. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

A. Vectors

The present disclosure provides one or more vectors for expressing the CRISPR nuclease, the reverse transcriptase, or their fusion polypeptide described herein or nucleic acids encoding the components described herein may be incorporated into a vector. In some embodiments, a vector disclosed herein includes a nucleotide sequence encoding CRISPR nuclease, the reverse transcriptase, or the fusion polypeptide. The present disclosure also provides one or more vectors encoding the editing template RNA or any portion thereof, e.g., the RNA guide, or the RT donor RNA. In some embodiments, the vector comprises a Pol II promoter or a Pol III promoter.

Expression of natural or synthetic polynucleotides is typically achieved by operably linking a polynucleotide encoding the gene of interest, e.g., nucleotide sequence encoding the CRISPR nuclease, the reverse transcriptase, or the fusion polypeptide, and/or the editing template RNA, to a promoter and incorporating the construct into an expression vector. The expression vector is not particularly limited as long as it includes a polynucleotide encoding the CRISPR nuclease and the reverse transcriptase and/or the editing template RNA of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method for recombinant expression vectors is not particularly limited, and examples thereof include methods using a plasmid, a phage or a cosmid.

B. Methods of Expression

The present disclosure includes a method for protein expression, comprising translating the CRISPR nuclease and the reverse transcriptase, and expressing the editing template RNA described herein.

In some embodiments, a host cell described herein is used to express the CRISPR nuclease and the reverse transcriptase and/or the editing template RNA. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the CRISPR nuclease, the reverse transcriptase and/or the editing template RNA. After expression of the CRISPR nuclease, the reverse transcriptase and/or the editing template RNA, the host cells can be collected and CRISPR nuclease, the reverse transcriptase and/or the editing template RNA purified from the cultures etc. according to conventional methods (for example, filtration, centrifugation, cell disruption, gel filtration chromatography, ion exchange chromatography, etc.).

In some embodiments, the methods for CRISPR nuclease and the reverse transcriptase expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the polypeptide(s). In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the CRISPR nuclease and the reverse transcriptase.

A variety of methods can be used to determine the level of production of a mature CRISPR nuclease, the reverse transcriptase and/or the editing template RNA in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the CRISPR nuclease and the reverse transcriptase and/or the editing template RNA in a cell, comprising providing a polyribonucleotide encoding the CRISPR nuclease, the reverse transcriptase and/or the editing template RNA to a host cell wherein the polyribonucleotide encodes the CRISPR nuclease, the reverse transcriptase and/or the editing template RNA, expressing the CRISPR nuclease, the reverse transcriptase and/or the editing template RNA in the cell, and obtaining the CRISPR nuclease, the reverse transcriptase and/or the editing template RNA from the cell.

### III. Methods for Gene Editing

Any of the gene editing systems can be used to genetically modify (edit) a target nucleic acid, which can be a genetic site of interest, e.g., a genetic site where genetic editing is needed, for example, to fix a genetic mutation, to introduce a protective mutation, to introduce modifications for modulating expression of a gene, etc.

The gene editing systems and compositions disclosed herein are applicable for editing and introducing edits into a variety of target sequences. In some embodiments, the target sequence is a DNA molecule, such as a DNA locus (referred to herein as a target sequence or an on-target sequence). In some embodiments, the target sequence is an RNA, such as an RNA locus or mRNA. In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, a single-stranded target sequence does not require a PAM sequence.

The target sequence may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target sequence may also comprise any sequence. In some embodiments, the target sequence is GC-rich, such as having at least about any one of 40%, 45%, 50%, 55%, 60%, 65%, or higher GC content. In some embodiments, the target sequence has a GC content of at least about 70%, 80%, or more. In some embodiments, the target sequence is a GC-rich fragment in a non-GC-rich target sequence. In some embodiments, the target sequence is not GC-rich. In some embodiments, the target sequence has one or more secondary structures or higher-order structures. In some embodiments, the target sequence is not in a condensed state, such as in a chromatin, to render the target sequence inaccessible by ribonucleoprotein.

In some embodiments, the target nucleic acid is a genomic site in a cell. In some instances, the target nucleic acid where the genetic edit would occur can be in a protein-coding region. Alternatively, the target nucleic acid may be in a regulatory region, such as a promoter, enhancer, a 5′ or 3′ untranslated region. In other instances, the target nucleic acid can be in In a non-coding gene, such as transposon, miRNA, tRNA, ribosomal RNA, ribozyme, or lincRNA.

A. Exemplary Genes for Genetic Editing

Any of the gene editing systems disclosed herein may be used to edit a target gene of interest, e.g., a gene involved in a disease (e.g., a genetic disease). In some embodiments, the target gene can be one that is involved in an immune response in a subject. For example, the target gene can be an immune checkpoint gene.

Exemplary target genes include, but are not limited to, BCL11A intronic erythroid enhancer, CD3, Beta-2 microglobulin (B2M), T Cell Receptor Alpha Constant (TRAC), Programmed Cell Death 1 (PDCD1), T-cell receptor alpha, T-cell receptor beta, B-cell lymphoma/leukemia 11A (BCL11A), Cytotoxic T-Lymphocyte Antigen 4 (CTLA-4), chemokine (C—C motif) receptor 5 (gene/pseudogene) (CCR5), CXCR4 gene, CD160 molecule (CD160), adenosine A2a receptor (ADORA), CD276, B7-H3, B7-H4, BTLA, nicotinamide adenine dinucleotide phosphate NADPH oxidase isoform 2 (NOX2), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), SIGLEC10, V-set domain containing T cell activation inhibitor 1 (VTCN1), B and T lymphocyte associated (BTLA), Indoleamine 2,3-dioxygenase (IDO), indoleamine 2,3-dioxygenase 1 (IDO1), Killer-cell Immunoglobulin-like Receptor (KIR), killer cell immunoglobulin-like receptor, three domains, long cytoplasmic tail, 1 (KIR3DL1), lymphocyte-activation gene 3 (LAG3), T-cell Immunoglobulin domain and Mucin domain 3 (TIM3), hepatitis A virus cellular receptor 2 (HAVCR2), natural killer cell receptor 2B4 (CD244), hypoxanthine phosphoribosyltransferase 1 (HPRT), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), adeno-associated virus integration site 1 (AAVS1), AAVS 2, AAVS3, AAVS4, AAVS5, AAVS6, AAVS7, AAVS8, transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), TGFB induced factor homeobox 1 (TGIF1), tumor necrosis factor receptor superfamily member, tumor necrosis factor receptor superfamily member 10b (TNFRSF10B), tumor necrosis factor receptor superfamily member 10a (TNFRSF10A), BY55, B7H5, caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), guanylate cyclase 1, soluble, beta 3 (GUCY1B3), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, CD27, CD28, CD40, CD122, CD137, OX40, GITR, and ICOS. In some embodiments, the modified gene is programmed death ligand 1 (PD-L1), class II major histocompatibility complex transactivator (CIITA), citramalyl-CoA lyase (CLYBL), transthyretin (TTR), lactate dehydrogenase-A (LDHA), dydroxyacid oxidase-1 (HAG1), alanine-glyoxylate and serine-pyruvate aminotransferase (AGXT), glyoxylate reductase/hydroxypyruvate reductase (GRHPR), 4-hydroxy-2-oxoglutarate aldolase (HOGA), polypyrimidine tract binding protein 1 (PTBP1), stathmin 2 (STMN2), or actin beta (ACTB).

The present disclosure provides methods for genetically editing any of the target genes as disclosed herein using the gene editing system as also disclosed herein.

B. Edits

In some aspects, provided herein are methods for introducing at least one edit into a target nucleic acid (e.g., a genomic site of interest such as in any of the target genes disclosed herein) using the gene editing system described herein. In some embodiments, the edit may include a substitution, an insertion, a deletion, or a combination thereof, into the target nucleic acid. In some examples, the edit can be a single nucleotide substitution, such as a G to T substitution, a G to A substitution, a G to C substitution, a T to G substitution, a T to A substitution, a T to C substitution, a C to G substitution, a C to T substitution, a C to A substitution, an A to T substitution, an A to G substitution, or an A to C substitution. In some examples, the edit can convert a G:C base pair to a T:A base pair, a G:C base pair to an A:T base pair, a G:C base pair to C:G base pair, a T:A base pair to a G:C base pair, a T:A base pair to an A:T base pair, a T:A base pair to a C:G base pair, a C:G base pair to a G:C base pair, a C:G base pair to a T:A base pair, a C:G base pair to an A:T base pair, an A:T base pair to a T:A base pair, an A:T base pair to a G:C base pair, or an A:T base pair to a C:G base pair.

In some embodiments, a method is described for introducing at least one edit into a target nucleic acid, where the edit is at least one substitution, at least one insertion, and/or at least one deletion. In some embodiments, the edit comprises at least one substitution, insertion, or deletion. In some embodiments, the substitution, insertion, or deletion is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In some embodiments, the substitution, insertion, or deletion is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides. In some embodiments, the substitution, insertion, or deletion is from 1 nucleotide to about 300 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides. In some embodiments, the substitution, insertion, or deletion is up to about 10,000 base pairs (10 kb) in length. For example, in some embodiments, the substitution, insertion, or deletion is 1 base pair, about 10 base pairs, about 20 base pairs, about 30 base pairs, about 40 base pairs, about 50 base pairs, about 60 base pairs, about 70 base pairs, about 80 base pairs, about 90 base pairs, about 100 base pairs, about 200 base pairs, about 300 base pairs, about 400 base pairs, about 500 base pairs, about 600 base pairs, about 700 base pairs, about 800 base pairs, about 900 base pairs, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, kb, 6 kb, 7 kb, 8 kb, 9 kb, or 10 kb in length.

In some embodiments, the insertion is or comprises a hairpin. For example, a reverse transcriptase may transcribe the hairpin, which can be incorporated into a target nucleic acid. In other embodiments, the reverse transcription template sequence includes a hairpin structure and a reverse transcriptase stops transcribing the reverse transcription template sequence at the hairpin.

In some embodiments, the edit occurs within about 500 nucleotides of a Type II PAM sequence (e.g., 5′-NGG-3′ for SpCas9) or a Type V PAM sequence (e.g., 5′-NTTN-3′ for a Cas12i polypeptide. In some embodiments, the edit occurs adjacent to a PAM sequence, e.g., within about 500 nucleotides upstream or downstream of a PAM sequence. In some embodiments, the edit occurs within about 400 nucleotides of a PAM sequence. In some embodiments, the edit occurs within about 400 nucleotides upstream or downstream of a PAM sequence. In some embodiments, the edit occurs within about 300 nucleotides of a PAM sequence. In some embodiments, the edit occurs within about 300 nucleotides upstream or downstream of a PAM sequence. In some embodiments, the edit occurs within about 200 nucleotides of a PAM sequence. In some embodiments, the edit occurs within about 200 nucleotides upstream or downstream of a PAM sequence. In some embodiments, the edit occurs within about 100 nucleotides of a PAM sequence. In some embodiments, the edit occurs within about 100 nucleotides upstream or downstream of a PAM sequence. In some embodiments, the edit occurs within about 50 nucleotides of a PAM sequence. In some embodiments, the edit occurs within about 50 nucleotides upstream or downstream of a PAM sequence. In some embodiments, the edit occurs within about 30 nucleotides of a PAM sequence. In some embodiments, the edit occurs within about 30 nucleotides upstream or downstream of a PAM sequence. In some embodiments, the edit occurs within about 20 nucleotides of a PAM sequence. In some embodiments, the edit occurs within about 20 nucleotides upstream or downstream of a PAM sequence.

In some embodiments, the edit starts within about 300 nucleotides upstream of the PAM sequence. In some embodiments, the edit starts within about 290 nucleotides upstream of the PAM sequence. In some embodiments, the edit starts within about 280 nucleotides upstream of the PAM sequence. In some embodiments, the edit starts within about 270 nucleotides upstream of the PAM sequence. In some embodiments, the edit starts within about 260 nucleotides upstream of the PAM sequence. In some embodiments, the edit starts within about 250 nucleotides upstream of the PAM sequence. In some embodiments, the edit starts within about 240 nucleotides upstream of the PAM sequence. In some embodiments, the edit starts within about 230 nucleotides upstream of the PAM sequence. In some embodiments, the edit starts within about 2020 nucleotides upstream of the PAM sequence. In some embodiments, the edit starts within about 210 nucleotides upstream of the PAM sequence. In some embodiments, the edit starts within about 200 nucleotides upstream of the PAM sequence. In some embodiments, the edit starts within about 190 nucleotides upstream of the PAM sequence. In some embodiments, the edit starts within about 180 nucleotides upstream of the PAM sequence. In some embodiments, the edit starts within about 170 nucleotides upstream of the PAM sequence. In some embodiments, the edit starts within about 160 nucleotides upstream of the PAM sequence. In some embodiments, the edit starts within about 150 nucleotides upstream of the PAM sequence. In some embodiments, the edit starts within about 140 nucleotides upstream of the PAM sequence. In some embodiments, the edit starts within about 130 nucleotides upstream of the PAM sequence. In some embodiments, the edit starts within about 120 nucleotides upstream of the PAM sequence. In some embodiments, the edit starts within about 110 nucleotides upstream of the PAM sequence. In some embodiments, the edit starts within about 100 nucleotides upstream of the PAM sequence. In some embodiments, the edit starts within about 90 nucleotides upstream of the PAM sequence. In some embodiments, the edit starts within about 80 nucleotides upstream of the PAM sequence. In some embodiments, the edit starts within about 70 nucleotides upstream of the PAM sequence. In some embodiments, the edit starts within about 60 nucleotides upstream of the PAM sequence. In some embodiments, the edit starts within about 50 nucleotides upstream of the PAM sequence. In some embodiments, the edit starts within about 40 nucleotides upstream of the PAM sequence. In some embodiments, the edit starts within about 30 nucleotides upstream of the PAM sequence. In some embodiments, the edit starts within about 20 nucleotides upstream of the PAM sequence. In some embodiments, the edit starts within about 10 nucleotides upstream of the PAM sequence. In some embodiments, the edit starts within about 9 nucleotides upstream of the PAM sequence. In some embodiments, the edit starts within about 8 nucleotides upstream of the PAM sequence. In some embodiments, the edit starts within about 7 nucleotides upstream of the PAM sequence. In some embodiments, the edit starts within about 6 nucleotides upstream of the PAM sequence. In some embodiments, the edit starts within about 5 nucleotides upstream of the PAM sequence. In some embodiments, the edit starts within about 4 nucleotides upstream of the PAM sequence. In some embodiments, the edit starts within about 3 nucleotides upstream of the PAM sequence. In some embodiments, the edit starts within about 2 nucleotides upstream of the PAM sequence. In some embodiments, the edit starts within about 1 nucleotide upstream of the PAM sequence.

In some embodiments, the edit starts at the PAM sequence. In some embodiments, the edit starts within about 1 nucleotide downstream of the PAM. In some embodiments, the edit starts within about 2 nucleotides downstream of the PAM. In some embodiments, the edit starts within about 3 nucleotides downstream of the PAM. In some embodiments, the edit starts within about 4 nucleotides downstream of the PAM. In some embodiments, the edit starts within about 5 nucleotides downstream of the PAM. In some embodiments, the edit starts within about 6 nucleotides downstream of the PAM. In some embodiments, the edit starts within about 7 nucleotides downstream of the PAM. In some embodiments, the edit starts within about 8 nucleotides downstream of the PAM. In some embodiments, the edit starts within about 9 nucleotides downstream of the PAM. In some embodiments, the edit starts within about 10 nucleotides downstream of the PAM. In some embodiments, the edit starts within about 11 nucleotides downstream of the PAM. In some embodiments, the edit starts within about 12 nucleotides downstream of the PAM. In some embodiments, the edit starts within about 13 nucleotides downstream of the PAM. In some embodiments, the edit starts within about 14 nucleotides downstream of the PAM. In some embodiments, the edit starts within about 15 nucleotides downstream of the PAM. In some embodiments, the edit starts within about 16 nucleotides downstream of the PAM. In some embodiments, the edit starts within about 17 nucleotides downstream of the PAM. In some embodiments, the edit starts within about 18 nucleotides downstream of the PAM. In some embodiments, the edit starts within about 19 nucleotides downstream of the PAM. In some embodiments, the edit starts within about 20 nucleotides downstream of the PAM. In some embodiments, the edit starts within about 21 nucleotides downstream of the PAM. In some embodiments, the edit starts within about 22 nucleotides downstream of the PAM. In some embodiments, the edit starts within about 23 nucleotides downstream of the PAM. In some embodiments, the edit starts within about 24 nucleotides downstream of the PAM. In some embodiments, the edit starts within about 25 nucleotides downstream of the PAM. In some embodiments, the edit starts within about 26 nucleotides downstream of the PAM. In some embodiments, the edit starts within about 27 nucleotides downstream of the PAM. In some embodiments, the edit starts within about 28 nucleotides downstream of the PAM. In some embodiments, the edit starts within about 29 nucleotides downstream of the PAM. In some embodiments, the edit starts within about 30 nucleotides downstream of the PAM.

In some embodiments, the edit ends within about 300 nucleotides upstream of the PAM sequence. In some embodiments, the edit ends within about 290 nucleotides upstream of the PAM sequence. In some embodiments, the edit ends within about 280 nucleotides upstream of the PAM sequence. In some embodiments, the edit ends within about 270 nucleotides upstream of the PAM sequence. In some embodiments, the edit ends within about 260 nucleotides upstream of the PAM sequence. In some embodiments, the edit ends within about 250 nucleotides upstream of the PAM sequence. In some embodiments, the edit ends within about 240 nucleotides upstream of the PAM sequence. In some embodiments, the edit ends within about 230 nucleotides upstream of the PAM sequence. In some embodiments, the edit ends within about 2020 nucleotides upstream of the PAM sequence. In some embodiments, the edit ends within about 210 nucleotides upstream of the PAM sequence. In some embodiments, the edit ends within about 200 nucleotides upstream of the PAM sequence. In some embodiments, the edit ends within about 190 nucleotides upstream of the PAM sequence. In some embodiments, the edit ends within about 180 nucleotides upstream of the PAM sequence. In some embodiments, the edit ends within about 170 nucleotides upstream of the PAM sequence. In some embodiments, the edit ends within about 160 nucleotides upstream of the PAM sequence. In some embodiments, the edit ends within about 150 nucleotides upstream of the PAM sequence. In some embodiments, the edit ends within about 140 nucleotides upstream of the PAM sequence. In some embodiments, the edit ends within about 130 nucleotides upstream of the PAM sequence. In some embodiments, the edit ends within about 120 nucleotides upstream of the PAM sequence. In some embodiments, the edit ends within about 110 nucleotides upstream of the PAM sequence. In some embodiments, the edit ends within about 100 nucleotides upstream of the PAM sequence. In some embodiments, the edit ends within about 90 nucleotides upstream of the PAM sequence. In some embodiments, the edit ends within about 80 nucleotides upstream of the PAM sequence. In some embodiments, the edit ends within about 70 nucleotides upstream of the PAM sequence. In some embodiments, the edit ends within about 60 nucleotides upstream of the PAM sequence. In some embodiments, the edit ends within about 50 nucleotides upstream of the PAM sequence. In some embodiments, the edit ends within about nucleotides upstream of the PAM sequence. In some embodiments, the edit ends within about 30 nucleotides upstream of the PAM sequence. In some embodiments, the edit ends within about 20 nucleotides upstream of the PAM sequence. In some embodiments, the edit ends within about 10 nucleotides upstream of the PAM sequence. In some embodiments, the edit ends within about 9 nucleotides upstream of the PAM sequence. In some embodiments, the edit ends within about 8 nucleotides upstream of the PAM sequence. In some embodiments, the edit ends within about 7 nucleotides upstream of the PAM sequence. In some embodiments, the edit ends within about 6 nucleotides upstream of the PAM sequence. In some embodiments, the edit ends within about 5 nucleotides upstream of the PAM sequence. In some embodiments, the edit ends within about 4 nucleotides upstream of the PAM sequence. In some embodiments, the edit ends within about 3 nucleotides upstream of the PAM sequence. In some embodiments, the edit ends within about 2 nucleotides upstream of the PAM sequence. In some embodiments, the edit ends within about 1 nucleotide upstream of the PAM sequence.

In some embodiments, the edit ends at the PAM sequence. In some embodiments, the edit ends within about 1 nucleotide downstream of the PAM. In some embodiments, the edit ends within about 2 nucleotides downstream of the PAM. In some embodiments, the edit ends within about 3 nucleotides downstream of the PAM. In some embodiments, the edit ends within about 4 nucleotides downstream of the PAM. In some embodiments, the edit ends within about 5 nucleotides downstream of the PAM. In some embodiments, the edit ends within about 6 nucleotides downstream of the PAM. In some embodiments, the edit ends within about 7 nucleotides downstream of the PAM. In some embodiments, the edit ends within about 8 nucleotides downstream of the PAM. In some embodiments, the edit ends within about 9 nucleotides downstream of the PAM. In some embodiments, the edit ends within about 10 nucleotides downstream of the PAM. In some embodiments, the edit ends within about 11 nucleotides downstream of the PAM. In some embodiments, the edit ends within about 12 nucleotides downstream of the PAM. In some embodiments, the edit ends within about 13 nucleotides downstream of the PAM. In some embodiments, the edit ends within about 14 nucleotides downstream of the PAM. In some embodiments, the edit ends within about 15 nucleotides downstream of the PAM. In some embodiments, the edit ends within about 16 nucleotides downstream of the PAM. In some embodiments, the edit ends within about 17 nucleotides downstream of the PAM. In some embodiments, the edit ends within about 18 nucleotides downstream of the PAM. In some embodiments, the edit ends within about 19 nucleotides downstream of the PAM. In some embodiments, the edit ends within about 20 nucleotides downstream of the PAM. In some embodiments, the edit ends within about 21 nucleotides downstream of the PAM. In some embodiments, the edit ends within about 22 nucleotides downstream of the PAM. In some embodiments, the edit ends within about 23 nucleotides downstream of the PAM. In some embodiments, the edit ends within about 24 nucleotides downstream of the PAM. In some embodiments, the edit ends within about 25 nucleotides downstream of the PAM. In some embodiments, the edit ends within about 26 nucleotides downstream of the PAM. In some embodiments, the edit ends within about 27 nucleotides downstream of the PAM. In some embodiments, the edit ends within about 28 nucleotides downstream of the PAM. In some embodiments, the edit ends within about 29 nucleotides downstream of the PAM. In some embodiments, the edit ends within about 30 nucleotides downstream of the PAM.

C. Non-PAM Strand Editing

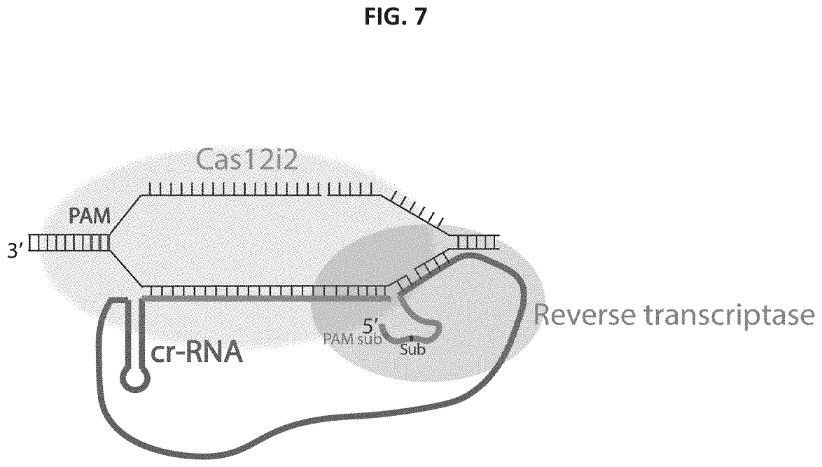

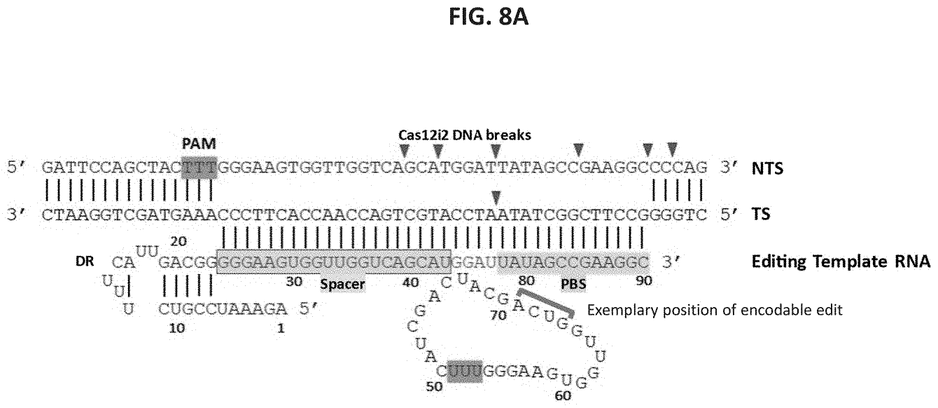

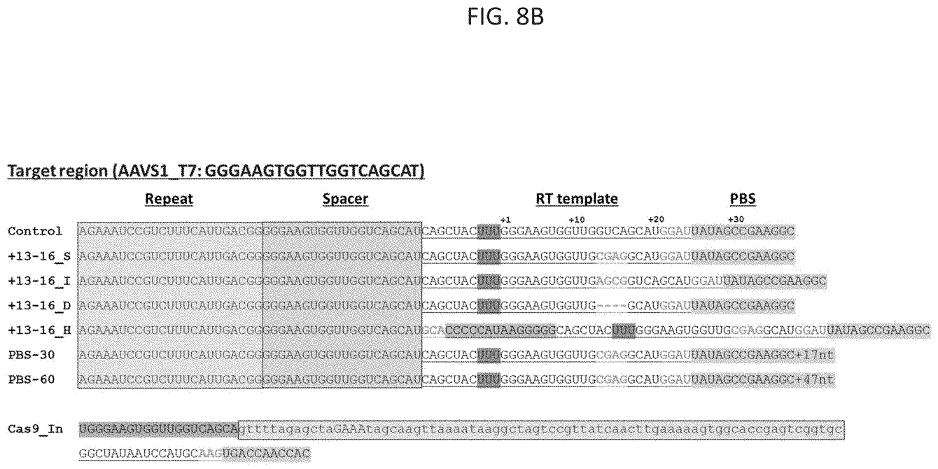

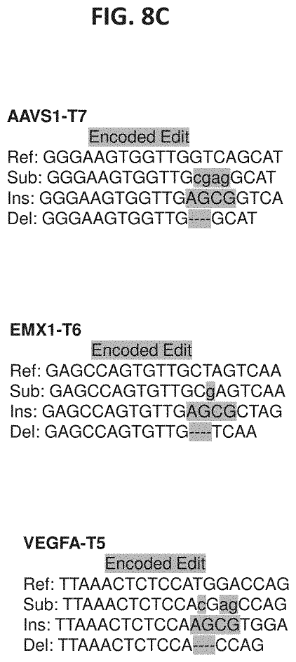

In some embodiments, provided herein is a method for introducing at least one edit into a non-PAM strand of a target nucleic acid, using suitable gene editing systems as disclosed herein, for example, those depicted in FIG. 5, FIG. 6A, FIG. 6B, FIG. 7, FIG. 8A, FIG. 12A, or FIG. 12B. The at least one edit could be introduced into the non-PAM strand initially using a reverse transcription template sequence contained in the gene editing system. Via cellular DNA repair machinery, the at least one edit would eventually be introduced into both strands of the target nucleic acid. The gene editing system may comprise an editing template RNA targeting the non-PAM strand, which comprises (a) a CRISPR nuclease binding sequence, (b) a DNA-binding sequence, and (c) and RT donor RNA. In some embodiments, the RT donor RNA comprises a PBS and a reverse transcription template sequence.

In some embodiment, a method and gene editing system or composition are described for introducing at least one edit into a non-PAM strand of a target nucleic acid through 5′ to 3′ transcription of the reverse transcription template sequence of the RT donor RNA. In some embodiment, a method and composition are described for introducing at least one edit into a non-PAM strand of a target nucleic acid through 5′ to 3′ transcription of the reverse transcription template sequence.

In some embodiments, a PBS of an RT donor RNA (e.g., an RT donor RNA of an editing template RNA) binds to a region on the non-PAM strand (the PBS-targeting site). The reverse transcription template sequence comprises an edit to be incorporated into the non-PAM strand. In some examples, the reverse transcription template comprises a sequence similarity to the PAM-strand. In some examples, the reverse transcription template comprises an edit relative to the sequence of the PAM strand. In some embodiments, the non-PAM strand binds the PBS of the RT donor RNA via base-pairing and a reverse transcriptase (e.g., a CRISPR nuclease-reverse transcriptase fusion) copies the reverse transcription template sequence. Following strand exchange back to base-pairing with the complementary genomic strand, the edit is incorporated into the target nucleic acid.

In some embodiments, the editing template RNA targeting the non-PAM strand comprises the following components from 5′ to 3′: a CRISPR nuclease binding sequence, a DNA-binding sequence, a reverse transcription template sequence, and a PBS (see, e.g., FIG. 5, FIG. 6A, FIG. 6B, FIG. 8A, and FIG. 12A). In some embodiments, the editing template RNA targeting the non-PAM strand comprises the following components from 5′ to 3′: reverse transcription template sequence, PBS, CRISPR nuclease binding sequence, and DNA-binding sequence (spacer) or the following components from 5′ to 3′: reverse transcription template sequence, PBS, linker, CRISPR nuclease binding sequence, and DNA-binding sequence (FIG. 7 and FIG. 12B).

In some embodiments, the CRISPR nuclease binding sequence is adjacent to the DNA-binding sequence. In some embodiments, the CRISPR nuclease binding sequence is a 5′ extension of the DNA-binding sequence (FIG. 5, FIG. 6A, FIG. 6B, FIG. 8A, and FIG. 12A). In some embodiments, the CRISPR nuclease binding sequence is adjacent to the DNA-binding sequence and the PBS. In some embodiments, the CRISPR nuclease binding sequence is a 3′ extension of the PBS (FIG. 7 and FIG. 12B). In some embodiments, the CRISPR nuclease binding sequence binds to a Type II CRISPR nuclease. In some embodiments, the CRISPR nuclease binding sequence binds to a Type V CRISPR nuclease (e.g., a Cas12i polypeptide such as a Cas12i1, Cas12i2, Cas12i3, or Cas12i4 polypeptide). In some embodiments, the CRISPR nuclease binding sequence binds to a CRISPR nuclease that lacks crRNA processing activity. In some embodiments, the CRISPR nuclease binding sequence is a direct repeat sequence (e.g., a Cas9 direct repeat sequence or Cas12i direct repeat sequence).

In some embodiments, the DNA-binding sequence is adjacent to the CRISPR nuclease binding sequence and the PBS. In some embodiments, the DNA-binding sequence is a 3′ extension of the CRISPR nuclease binding sequence (FIG. 5, FIG. 6A, FIG. 6B, FIG. 7, FIG. 8A, FIG. 12A, and FIG. 12B). In some embodiments, the DNA-binding sequence may comprise an RNA sequence, a DNA sequence, or an RNA/DNA hybrid sequence. In some embodiments, the DNA-binding sequence comprises about 10 nucleotides to about 50 nucleotides in length. In some embodiments, the DNA-binding sequence comprises about 15 nucleotides to about 35 nucleotides in length.

In some embodiments, the PBS is adjacent to the reverse transcription template sequence. In some embodiments, the PBS is a 3′ extension of the reverse transcription template sequence (FIG. 5, FIG. 6A, FIG. 6B, FIG. 7, FIG. 8A, FIG. 12A, and FIG. 12B). In some embodiments, the PBS is adjacent to the reverse transcription template sequence and the CRISPR nuclease binding sequence. In some embodiments, the PBS is between about 3 nucleotides and about 200 nucleotides in length. In some embodiments, the PBS is about 3, 4, 5, 6, 7, 8, 9, 10, 15, 20, 25, 30, 35, 40, 45, 50, 55, 60, 65, 70, 75, 80, 85, 90, 95, 100, 105, or 110 nucleotides in length. In some embodiments, the DNA-binding sequence and the PBS bind to a same strand of the target nucleic acid (e.g., the non-PAM strand).

In some embodiments, the reverse transcription template sequence is adjacent to the PBS and the DNA-binding sequence. In some embodiments, the reverse transcription template sequence is a 5′ extension of the PBS (FIG. 5, FIG. 6A, FIG. 6B, FIG. 8A, and FIG. 12A). In some embodiments, the reverse transcription template sequence is a 3′ extension of the DNA-targeting sequence (FIG. 5, FIG. 6A, FIG. 6B, FIG. 8A, and FIG. 12A). In some embodiments, the reverse transcription template sequence is a 5′ extension of the PBS (FIG. 7 and FIG. 12B). In some embodiments, the reverse transcription template sequence is about 10 nucleotides to about 300 nucleotides in length. In some embodiments, the reverse transcription template sequence is about 10, 15, 20, 25, 30, 35, 40, 45, 50, 55, 60, 65, 70, 75, 80, 85, 90, 95, 100, 105, 110, 115, or 120 nucleotides in length.

In some embodiments, an editing template RNA targeting the non-PAM strand comprises a loop of unpaired nucleotides when the DNA-binding sequence and PBS are bound to a target nucleic acid. See FIG. 6A, FIG. 6B, FIG. 8A, and FIG. 12A. In some embodiments, an editing template RNA targeting the non-PAM strand comprises a loop adjacent to the PBS. See FIG. 7 and FIG. 12B. In some embodiments, the loop comprises the reverse transcription template sequence and is followed by the PBS. In some embodiments, the PBS comprises complementarity to the non-PAM strand of a target nucleic acid. In some embodiments, the sequence of the loop comprises sequence similarity to the PAM strand. In some embodiments, the loop comprises an edit relative to the sequence of the PAM strand. In some embodiments, the edit is a substitution, an insertion, or a deletion. In some embodiments, the loop comprises a hairpin.

D. PAM Strand Editing

In some embodiments, provided herein a method for introducing at least one edit into a PAM strand of a target nucleic acid (e.g., a genomic site of interest), using a suitable gene editing system disclosed herein, such as those depicted in FIG. 1A, FIG. 1B, FIG. 2, FIG. 3, FIG. 4, or FIG. 10. Such a method may involve the use of an editing template RNA targeting the PAM strand, which may comprise (a) a CRISPR nuclease binding sequence, (b) a DNA-binding sequence, and (c) and RT donor RNA (FIG. 1A, FIG. 1B, FIG. 2, and FIG. 10). In some examples, a composition targeting the PAM strand comprises an RNA guide and an RT donor RNA (FIG. 3 and FIG. 4). In some examples, the RT donor RNA comprises a PBS and a reverse transcription template sequence.

In some embodiment, a method and composition are described for introducing at least one edit into a PAM strand of a target nucleic acid through 5′ to 3′ transcription of the reverse transcription template sequence. In some embodiment, a method and composition are described for introducing at least one edit into a PAM strand of a target nucleic acid through 5′ to 3′ transcription of the reverse transcription template sequence.

In some instances, a PBS of an RT donor RNA (e.g., an RT donor RNA of an editing template RNA) binds to the PAM strand. The reverse transcription template sequence of the RT donor RNA comprises an edit to be incorporated into the PAM strand. In some examples, the reverse transcription template comprises sequence similarity to the non-PAM strand. In some embodiments, the reverse transcription template comprises an edit relative to the sequence of the non-PAM strand. In some embodiments, the PAM strand can bind to the PBS of the RT donor RNA via base-paring and a reverse transcriptase (e.g., a CRISPR nuclease-reverse transcriptase fusion) copies the reverse transcription template sequence. Following strand exchange back to base-pairing with the complementary genomic strand, the edit is incorporated into the target nucleic acid.

In some embodiments, the editing template RNA targeting the PAM strand comprises the following components from 5′ to 3′: CRISPR nuclease binding sequence, DNA-binding sequence, reverse transcription template sequence, and PBS (FIG. 1A, FIG. 1B, and FIG. 10). In some embodiments, the editing template RNA targeting the PAM strand comprises the following components from 5′ to 3′: reverse transcription template sequence, PBS, CRISPR nuclease binding sequence, and DNA-binding sequence or the following components from 5′ to 3′: reverse transcription template sequence, PBS, linker, CRISPR nuclease binding sequence, and DNA-binding sequence (FIG. 2).

In some embodiments, the CRISPR nuclease binding sequence is adjacent to the DNA-binding sequence. In some embodiments, the DNA-binding sequence is a 3′ extension of the CRISPR nuclease binding sequence (FIG. 1A, FIG. 1B, FIG. 2, and FIG. 10). In some embodiments, the CRISPR nuclease binding sequence is adjacent to the DNA-binding sequence and the PBS (FIG. 2). In some embodiments the DNA-binding sequence is a 3′ extension of the PBS (FIG. 2). In some embodiments, the CRISPR nuclease binding sequence binds to a Type II CRISPR nuclease. In some embodiments, the CRISPR nuclease binding sequence binds to a Type V CRISPR nuclease (e.g., a Cas12i polypeptide such as a Cas12i1, Cas12i2, Cas12i3, or Cas12i4 polypeptide). In some embodiments, the CRISPR nuclease binding sequence binds to a CRISPR nuclease that lacks crRNA processing activity. In some embodiments, the CRISPR nuclease binding sequence is a direct repeat sequence (e.g., a Cas9 direct repeat sequence or Cas12i direct repeat sequence).

In some embodiments, the DNA-binding sequence is adjacent to the CRISPR nuclease binding sequence. In some embodiments, the DNA-binding sequence is a 3′ extension of the CRISPR nuclease binding sequence (FIG. 1A, FIG. 1B, FIG. 2, and FIG. 10). In some embodiments, the DNA-binding sequence is adjacent to the CRISPR nuclease binding sequence and the reverse transcription template sequence. In some embodiments, the reverse transcription template sequence is a 3′ extension of the DNA-binding sequence (FIG. 10). In some embodiments, the DNA-binding sequence is an RNA sequence, a DNA sequence, or an RNA/DNA hybrid sequence. In some embodiments, the DNA-binding sequence comprises about 10 nucleotides to about 50 nucleotides in length. In some embodiments, the DNA-binding sequence comprises about 15 nucleotides to about 35 nucleotides in length. In some embodiments, the DNA-binding sequence is a spacer sequence.

In some embodiments, the PBS is adjacent to the reverse transcription template sequence. In some embodiments, the PBS is a 3′ extension of the reverse transcription template sequence (FIG. 1A, FIG. 2, FIG. 1B, and FIG. 10). In some embodiments, the PBS is adjacent to the CRISPR nuclease binding sequence. In some embodiments, the CRISPR nuclease binding sequence is a 3′ extension of the PBS (FIG. 2). In some embodiments, the PBS is between about 3 nucleotides and about 200 nucleotides in length. In some embodiments, the PBS is about 3, 4, 5, 6, 7, 8, 9, 10, 15, 20, 25, 30, 35, 40, 45, 50, 55, 60, 65, 70, 75, 80, 85, 90, 95, 100, 105, or 110 nucleotides in length. In some embodiments, the DNA-binding sequence and the PBS bind to a different strand of the target nucleic acid (e.g., the DNA-binding sequence binds to the target strand, and the PBS binds to the PAM strand).

In some embodiments, the reverse transcription template sequence is adjacent to the DNA-binding sequence. In some embodiments, the reverse transcription template sequence is a 3′ extension of the DNA-binding sequence (FIG. 1A, FIG. 1B, and FIG. 10). In some embodiments, the reverse transcription template sequence is adjacent to the PBS. In some embodiments, the reverse transcription template sequence is a 5′ extension of the PBS (FIG. 1A, FIG. 1B, FIG. 2). In some embodiments, the PBS is a 3′ extension of the reverse transcription template sequence (FIG. 10). In some embodiments, the reverse transcription template sequence is about 10 nucleotides to about 300 nucleotides in length. In some embodiments, the reverse transcription template sequence is about 10, 15, 20, 25, 30, 35, 40, 45, 50, 55, 60, 65, 70, 75, 80, 85, 90, 95, 100, 105, 110, 115, or 120 nucleotides in length.

E. Gene Editing in Cells

In some aspects, provided herein are methods for editing a genomic site of interest (e.g., a target gene as disclosed herein) in cells using a suitable gene editing system as also disclosed herein. To perform this method, the gene editing system can be delivered to or introduced into a population of cells. In some instances, cells comprising the desired genetic editing may be collected and optionally cultured and expanded in vitro.

The cell described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a primate cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a mesenchymal stem cell. In some embodiments, the cell is an embryonic stem cell. In some embodiments, the cell is a hematopoietic stem cell. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a glial cell. In some embodiments, the cell is a pancreatic islet cell, including an alpha cell, beta cell, delta cell, or enterochromaffin cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or primate cell or a murine cell. In some embodiments, the murine cell is derived from a wildtype mouse, an immunosuppressed mouse, or a disease-specific mouse model. In some embodiments, the cell is a cell within a living tissue, organ, or organism.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In embodiments wherein a gene editing system disclosed herein is introduced into a plurality of cells, at least about 0.5% of the cells comprise the desired edit. In some embodiments, at least about 1% of the cells comprise the desired edit. In some embodiments, at least about 2% of the cells comprise the desired edit. In some embodiments, at least about 3% of the cells comprise the desired edit. In some embodiments, at least about 4% of the cells comprise the desired edit. In some embodiments, at least about 5% of the cells comprise the desired edit. In some embodiments, at least about 10% of the cells comprise the desired edit. In some embodiments, at least about 20% of the cells comprise the desired edit. In some embodiments, at least about 30% of the cells comprise the desired edit. In some embodiments, at least about 40% of the cells comprise the desired edit. In some embodiments, at least about 50% of the cells comprise the desired edit.

The cells carrying the desired genetic edit, e.g., produced by the method disclosed herein using any of the gene editing systems also disclosed herein, are also within the scope of the present disclosure. In some instances, the cells modified by a CRISPR nuclease, reverse transcriptase, and editing template RNA as described herein may be useful as an expression system to manufacture biomolecules. For example, the modified cells may be useful to produce biomolecules such as proteins (e.g., cytokines, antibodies, antibody-based molecules), peptides, lipids, carbohydrates, nucleic acids, amino acids, and vitamins. In other embodiments, the modified cell may be useful in the production of a viral vector such as a lentivirus, adenovirus, adeno-associated virus, and oncolytic virus vector. In some embodiments, the modified cell may be useful in cytotoxicity studies. In some embodiments, the modified cell may be useful as a disease model. In some embodiments, the modified cell may be useful in vaccine production. In some embodiments, the modified cell may be useful in therapeutics. For example, in some embodiments, the modified cell may be useful in cellular therapies such as transfusions and transplantations.

In some embodiments, the cells modified by a CRISPR nuclease, reverse transcriptase, and editing template RNA as described herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion of the modified stem cell. The disclosure further provides organisms (such as animals, plants, or fungi) comprising or produced from a modified cell of the disclosure.

F. Delivery of Gene Editing Systems to Cells

In some embodiments, any of the gene editing systems or components thereof may be formulated, for example, including a carrier, such as a carrier and/or a polymeric carrier, e.g., a liposome or lipid nanoparticle, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the CRISPR nuclease, reverse transcriptase, editing template RNA (e.g., RNA guide and RT donor RNA), etc.), one or more transcripts thereof, and/or a pre-formed ribonucleoprotein to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells. In some embodiments, a composition of the present invention is further delivered with an agent (e.g., compound, molecule, or biomolecule) that affects DNA repair or DNA repair machinery. In some embodiments, a composition of the present invention is further delivered with an agent (e.g., compound, molecule, or biomolecule) that affects the cell cycle.

In some embodiments, a first composition comprising a CRISPR nuclease or a CRISPR nuclease and a reverse transcriptase (e.g., a CRISPR nuclease-reverse transcriptase fusion) is delivered to a cell. In some embodiments, a second composition comprising an RNA guide or an RNA guide and RT donor RNA (e.g., an editing template RNA) is delivered to a cell. In some embodiments, the first composition is contacted with a cell before the second composition is contacted with the cell. In some embodiments, the first composition is contacted with a cell at the same time as the second composition is contacted with the cell. In some embodiments, the first composition is contacted with a cell after the second composition is contacted with the cell. In some embodiments, the first composition is delivered by a first delivery method and the second composition is delivered by a second delivery method. In some embodiments, the first delivery method is the same as the second delivery method. For example, in some embodiments, the first composition and the second composition are delivered via viral delivery. In some embodiments, the first delivery method is different than the second delivery method. For example, in some embodiments, the first composition is delivered by viral delivery and the second composition is delivered by lipid nanoparticle-mediated transfer and the second composition is delivered by viral delivery or the first composition is delivered by lipid nanoparticle-mediated transfer and the second composition is delivered by viral delivery.

### IV. Therapeutic Applications

Any of the gene editing systems or modified cells generated using such a gene editing system as disclosed herein may be used for treating a disease that may be benefit from the gene edit introduced by the gene editing system or carried by the modified cells. For example, the disease may be a genetic disease and the gene edit fixes the gene mutation associated with the genetic disease. Alternatively, the disease may be associated with abnormal expression of a gene and the gene edit rescues such abnormal expression.

In some embodiments, provided herein is a method for treating a disease comprising administering to a subject (e.g., a human patient) in need of the treatment any of the gene editing system disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a a cell line modified by a CRISPR nuclease, reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edits. In some examples, the cells can be suspended in a suitable media.

In some embodiments, provided herein is a composition comprising the gene editing system or components thereof or the modified cells. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits or systems that can be used, for example, to carry out a method described herein. In some embodiments, the kits or systems include a CRISPR nuclease (e.g., a Type V nuclease such as a Cas12i polypeptide) and a reverse transcriptase. In some embodiments, the kits or systems include a polynucleotide that encodes a CRISPR nuclease (e.g., a Type V nuclease such as a Cas12i polypeptide) and reverse transcriptase, and optionally the polynucleotide is comprised within a vector, e.g., as described herein. In some embodiments, the kits or systems include a Type V nuclease-reverse transcriptase fusion polypeptide (e.g., a Cas12i-reverse transcriptase fusion polypeptide such as a Cas12i2-RT fusion or a Cas12i4-RT fusion). The kits or systems also can include a reverse transcriptase, and an editing template RNA (e.g., an RNA guide and RT donor RNA) as described herein. The RNA guide and/or RT donor RNA of the kits or systems of the invention can be designed to target a sequence of interest. The CRISPR nuclease (e.g., a Type V nuclease such as a Cas12i polypeptide), reverse transcriptase, and editing template RNA (e.g., RNA guide and RT donor RNA) can be packaged within the same vial or other vessel within a kit or system or can be packaged in separate vials or other vessels, the contents of which can be mixed prior to use. The kits or systems can additionally include, optionally, a buffer and/or instructions for use of the CRISPR nuclease (e.g., a Type V nuclease such as a Cas12i polypeptide) and reverse transcriptase, along with the editing template RNA (e.g., RNA guide and RT donor RNA).

In some embodiments, the kit comprises a first composition comprising a CRISPR nuclease or a CRISPR nuclease and a reverse transcriptase (e.g., a CRISPR nuclease-reverse transcriptase fusion). In some embodiments, the kit comprises a second composition comprising an RNA guide or an RNA guide and RT donor RNA (e.g., an editing template RNA). In some embodiments, the first composition and the second composition are packaged within the same vial. In some embodiments, the first composition and the second composition are packaged within different vials.

In some embodiments, the kit may be useful for research purposes. For example, in some embodiments, the kit may be useful to study gene function.

All references and publications cited herein are hereby incorporated by reference.

### Additional Embodiments

Provided below are additional embodiments, which are also within the scope of the present disclosure.

Embodiment 1: A composition comprising:
(a) a Type V CRISPR nuclease polypeptide or a nucleic acid encoding the Type V CRISPR nuclease polypeptide, which optionally is a Cas12 polypeptide;
(b) an RNA guide or a nucleic acid encoding the RNA guide, wherein the RNA guide comprises a Type V nuclease binding sequence (e.g., a direct repeat sequence) and a DNA-binding sequence (e.g., a spacer sequence);
(c) a reverse transcriptase polypeptide or a nucleic acid encoding the reverse transcriptase polypeptide; and
(d) a reverse transcription donor RNA (RT donor RNA) comprising a primer binding

In Embodiment 1, the Type V CRISPR nuclease can be a Cas12a (Cpf1), Cas12b (C2c1), Cas12c, Cas12d, Cas12e, Cas12f, Cas12h, Cas12i, or Cas12j (CasPhi) polypeptide. In some examples, the Type V CRISPR nuclease polypeptide is a Cas12i polypeptide, which optionally comprises a Cas12i1 polypeptide or variant Cas12i1 polypeptide, a Cas12i2 polypeptide or variant Cas12i2 polypeptide, a Cas12i3 polypeptide or variant Cas12i3 polypeptide, or a Cas12i4 polypeptide or a variant Cas12i4 polypeptide.

Embodiment 2: the composition of Embodiment 1 may comprise a Cas12i polypeptide, which can be one of the following:
(a) the Cas12i1 polypeptide comprises an amino acid sequence with at least 80% identity to SEQ ID NO: 8; optionally at least 95% identity to SEQ ID NO: 8;
(b) the Cas12i2 polypeptide comprises an amino acid sequence with at least 80% identity to any one of SEQ ID NOs: 2-7; optionally at least 95% identity to any one of SEQ ID NOs: 2-7;
(c) the Cas12i3 polypeptide comprises an amino acid sequence with at least 80% identity to SEQ ID NO: 11; optionally at least 95% identity to SEQ ID NO: 11; and
(d) the Cas12i4 polypeptide comprises an amino acid sequence with at least 80% identity to SEQ ID NO: 9 or at least 80% to SEQ ID NO: 10; optionally at least 95% identity to SEQ ID NO: 9 or at least 95% to SEQ ID NO: 10.

In specific examples, the composition of Embodiment 2 comprises one of the following:
(a) the Cas12i1 polypeptide comprises the amino acid sequence set forth in SEQ ID NO: 8;
(b) the Cas12i2 polypeptide comprises the amino acid sequence set forth in any one of SEQ ID NOs: 2-7;
(c) the Cas12i3 polypeptide comprises the amino acid sequence set forth in SEQ ID NO: 11; and
(d) the Cas12i4 polypeptide comprises the amino acid sequence set forth in SEQ ID NO: 9 or SEQ ID NO: 10.

Any of the compositions of Embodiment 2 disclosed herein may comprise the Type V CRISPR nuclease polypeptide that has diminished crRNA processing activity or lacks crRNA processing activity. For example, the Type V CRISPR nuclease polypeptide is a Cas12i2 polypeptide, and wherein the Cas12i2 polypeptide comprises a substitution at position H485 or H486. In some instances, the Cas12i2 polypeptide comprises at least 80% identity to any one of SEQ ID NOs: 2-7, and wherein the Cas12i2 polypeptide comprises a substitution at position H485 or H486. In some examples, the Cas12i2 polypeptide comprises at least 95% identity to any one of SEQ ID NOs: 2-7, and wherein the Cas12i2 polypeptide comprises a substitution at position H485 or H486.

Any of the compositions of Embodiment 2 disclosed herein may comprise the Type V CRISPR nuclease polypeptide, which comprises at least one of: an epitope peptide, a nuclear localization signal, and a nuclear export signal.

In some examples, the composition of Embodiment 2 comprises one of the following:
(a) the Cas12i1 polypeptide comprises an amino acid sequence with at least 80% (e.g., at least 95%) identity to SEQ ID NO: 8, and the direct repeat sequence comprises a nucleotide sequence with at least 90% identity to any one of SEQ ID NOs: 12-14;
(b) the Cas12i2 polypeptide comprises an amino acid sequence with at least 80% e.g., at least 95%) identity to any one of SEQ ID NOs: 2-7 and the direct repeat sequence comprises a nucleotide sequence with at least 90% identity to any one of SEQ ID NOs: 15-17;
(c) the Cas12i3 polypeptide comprises an amino acid sequence with at least 80% e.g., at least 95%) identity to SEQ ID NO: 11 and the direct repeat sequence comprises a nucleotide sequence with at least 90% identity to any one of SEQ ID NOs: 18-20; and
(d) the Cas12i4 polypeptide comprises an amino acid sequence with at least 80% e.g., at least 95%) identity to SEQ ID NO: 9 or SEQ ID NO: 10 and the direct repeat sequence comprises a nucleotide sequence with at least 90% identity to any one of SEQ ID NOs: 21-24.

In some examples, the composition of Embodiment 2 comprises one of the following:
(a) the Cas12i1 polypeptide comprises an amino acid sequence with at least 95% identity to SEQ ID NO: 8 and the direct repeat sequence comprises a nucleotide sequence with at least 95% identity to any one of SEQ ID NOs: 12-14;
(b) the Cas12i2 polypeptide comprises an amino acid sequence with at least 95% identity to any one of SEQ ID NOs: 2-7 and the direct repeat sequence comprises a nucleotide sequence with at least 95% identity to any one of SEQ ID NOs: 15-17;
(c) the Cas12i3 polypeptide comprises an amino acid sequence with at least 95% identity to SEQ ID NO: 11 and the direct repeat sequence comprises a nucleotide sequence with at least 95% identity to any one of SEQ ID NOs: 18-20; and
(d) the Cas12i4 polypeptide comprises an amino acid sequence with at least 95% identity to SEQ ID NO: 9 or SEQ ID NO: 10 and the direct repeat sequence comprises a nucleotide sequence with at least 95% identity to any one of SEQ ID NOs: 21-24.

Embodiment 3: the spacer sequence of any of the compositions of Embodiment 1 or Embodiment 2 disclosed herein comprises from about 10 nucleotides to about 50 nucleotides in length. In some examples, the spacer sequence comprises from about 15 nucleotides to about 35 nucleotides in length. In some examples, the spacer sequence is substantially complementary to a target strand (e.g., the complementary sequence of a target sequence) of a target nucleic acid. In some examples, the target sequence is adjacent to a protospacer adjacent motif (PAM) sequence on the non-target strand.

Embodiment 4: any of the compositions of Embodiment 1, 2, or 3, may comprise the Type V nuclease, which is a Cas12i polypeptide, and wherein the PAM sequence comprises a sequence set forth as 5′-NTTN-3′, wherein N is any nucleotide.

Embodiment 5: in any of the compositions of any previous embodiments, the reverse transcriptase polypeptide comprises MMLV-RT, MMTV-RT, Marathon-RT, or RTX reverse transcriptase.

Embodiment 6: in any of the compositions of any of Embodiments 1-5, the reverse transcriptase polypeptide is fused to the Type V CRISPR nuclease polypeptide. In some examples, the reverse transcriptase polypeptide is fused to the N-terminus of the Type V CRISPR nuclease polypeptide. In other examples, the reverse transcriptase polypeptide is fused to the C-terminus of the Type V CRISPR nuclease polypeptide. In yet other examples, the reverse transcriptase polypeptide is inserted within a loop of the Type V CRISPR nuclease polypeptide.

Embodiment 7: in any of the compositions of any of Embodiments 1-5, the reverse transcriptase polypeptide and the Type V CRISPR nuclease polypeptide form a complex through a leucine zipper, nanobody, antibody, or coiled-coil domain.

Embodiment 8: in any of the compositions of any of Embodiments 1-7, the RT donor RNA can be fused to the RNA guide. In some examples, the RT donor RNA is fused to the 5′ end of the RNA guide. In other examples, the RT donor RNA is fused to the 3′ end of the RNA guide. In some instances, the spacer sequence of the RNA guide is adjacent to the reverse transcription template sequence in the RT donor RNA. Alternatively, the spacer sequence of the RNA guide is adjacent to the PBS in the RT donor RNA. In other instances, the direct repeat sequence of the RNA guide is adjacent to the reverse transcription template sequence in the RT RNA donor. Alternatively, the direct repeat sequence of the RNA guide is adjacent to the PBS in the RT donor RNA.

In some examples, the RT donor RNA-RNA guide fusion polynucleotide may further comprise a linker. In some instances, the linker is between the direct repeat sequence and the PBS. In other instances, the linker is between the spacer sequence in the and the reverse transcription template sequence. The linker may be between about 1 nucleotide and about 200 nucleotides in length. In some examples, the linker comprises a hairpin.

Embodiment 9: in any of the compositions of any one of Embodiments 1-8, the PBS can be between about 3 nucleotides and about 200 nucleotides in length. For example, the PBS is about 3, 4, 5, 6, 7, 8, 9, 10, 15, 20, 25, 30, 35, 40, 45, 50, 55, 60, 65, 70, 75, 80, 85, 90, 95, 100, 105, or 110 nucleotides in length. In some instances, the PBS hybridizes (binds via base-pairing) with a free 3′ end of the non-target strand (the PAM strand). In other instances, the PBS hybridizes a free 3′ end of the target strand (the non-PAM strand).

Embodiment 10: in any of the compositions of any one of Embodiments 1-9, the reverse transcription template sequence is between about 10 nucleotides and about 300 nucleotides in length. For example, the reverse transcription template sequence is about 10, 15, 20, 25, 30, 35, 40, 45, 50, 55, 60, 65, 70, 75, 80, 85, 90, 95, 100, 105, 110, 115, or 120 nucleotides in length.

Embodiment 11: in any of the compositions of any one of Embodiments 1-10, the PBS has substantial complementarity to the target strand or the non-target strand of the target nucleic acid (which is double-stranded). For example, the PBS comprises at least about 75% complementarity to the target strand or the non-target strand of the target nucleic acid. In other examples, the PBS comprises at least about 85% complementarity to the target strand or the non-target strand of the target nucleic acid. In other examples, the PBS comprises at least about 95% complementarity to the target strand or the non-target strand of the target nucleic acid.

Embodiment 12: in any of the compositions of any one of Embodiments 1-11, the reverse transcription template sequence comprises an aptamer. In some instances, the aptamer recruits the reverse transcriptase polypeptide.

Embodiment 13: in any of the compositions of any one of Embodiments 1-11, the reverse transcription template comprises a modification, e.g., at the 5′ end or at the 3′ end. In some examples, the modification is a chemical modification. In other examples, the modification is a nucleic acid sequence comprising secondary structure. In specific examples, the modification is a hairpin, a pseudoknot, a triplex structure, an exoribonuclease-resistant RNA (xrRNA), a transfer RNA (tRNA), or a truncated tRNA. In other specific examples, the modification comprises a nuclease binding sequence (e.g., one or more direct repeat sequences) or a nuclease binding sequence and a DNA-binding sequence (a spacer).

Any of the compositions of any one of Embodiments 1-13 may introduce an edit into the target strand or the non-target strand. In some examples, the edit is a substitution, insertion, or deletion. In some instances, the edit is a substitution of 1 nucleotide to about 200 nucleotides. In some instances, the edit is a substitution of 1 nucleotide to about 120 nucleotides. In some instances, the edit is a substitution of 1 nucleotide to about 20 nucleotides. In other instances, the edit is an insertion of 1 nucleotide to about 200 nucleotides, for example, an insertion of 1 nucleotide to about 120 nucleotides, an insertion of 1 nucleotide to about 20 nucleotides. In some examples, the insertion comprises a hairpin. In yet other instances, the edit is a deletion of 1 nucleotide to about 100 nucleotides. For example, the edit is a deletion of 1 nucleotide to about 120 nucleotides, or a deletion of 1 nucleotide to about 20 nucleotides.

In some examples, the edit occurs within about 200 nucleotides of the PAM sequence. In one example, the edit occurs within about 100 nucleotides of the PAM sequence. In another example, the edit occurs within about 50 nucleotides of the PAM sequence. In yet another example, the edit occurs within about 30 nucleotides of the PAM sequence. In still another example, the edit occurs within about 20 nucleotides of the PAM sequence.

In some examples, the edit starts and/or ends within about 200 nucleotides upstream of the PAM sequence, e.g., starts and/or ends within about 100 nucleotides upstream of the PAM sequence, starts and/or ends within about 50 nucleotides upstream of the PAM sequence, starts and/or ends within about 30 nucleotides upstream of the PAM sequence, starts and/or ends within about 20 nucleotides upstream of the PAM sequence, starts and/or ends within about 10 nucleotides upstream of the PAM sequence, starts and/or ends within about 5 nucleotides upstream of the PAM sequence, or starts and/or ends within about 5 nucleotides downstream of the PAM sequence.

In other examples, the edit starts and/or ends within about 10 nucleotides downstream of the PAM sequence, for example, starts and/or ends within about 25 nucleotides downstream of the PAM sequence.

In some examples, the edit removes or alters the PAM sequence. In some examples, the edit prevents retargeting by the Type V CRISPR nuclease polypeptide (e.g., prevents binding of the Type V CRISPR nuclease to the target sequence).

Embodiment 14: in any of the compositions of Embodiments 1-13, the target sequence is present in a cell.

Embodiment 15: any of the compositions of Embodiments 1-14 can be formulated for delivery to a cell. In some examples, the cell is a mammalian cell, for example, a human cell. In one example, the cell is a liver cell (e.g., a hepatocyte).

In some examples, the Type V CRISPR nuclease polypeptide or the nucleic acid encoding the Type V CRISPR nuclease polypeptide, the RNA guide or the nucleic acid encoding the RNA guide, the reverse transcriptase polypeptide or the nucleic acid encoding the reverse transcriptase polypeptide, and the RT donor RNA are formulated in a single delivery vehicle.

In other examples, the Type V CRISPR nuclease polypeptide or the nucleic acid encoding the Type V CRISPR nuclease polypeptide, the RNA guide or the nucleic acid encoding the RNA guide, the reverse transcriptase polypeptide or the nucleic acid encoding the reverse transcriptase polypeptide, and the RT donor RNA are formulated in two or more delivery vehicles.

In yet other examples, the Type V CRISPR nuclease polypeptide or the nucleic acid encoding the Type V CRISPR nuclease polypeptide and the reverse transcriptase polypeptide or the nucleic acid encoding the reverse transcriptase polypeptide are formulated in a single delivery vehicle.

In some examples, the RNA guide and the RT donor RNA are formulated in a single delivery vehicle. In some examples, the Type V CRISPR nuclease polypeptide or the nucleic acid encoding the Type V CRISPR nuclease polypeptide and the reverse transcriptase polypeptide or the nucleic acid encoding the reverse transcriptase polypeptide are formulated in a first delivery vehicle and the RNA guide and the RT donor RNA are formulated in a second delivery vehicle.

Embodiment 16: in any of the composition of any one of Embodiments 1-15 where applicable, the Type V CRISPR nuclease polypeptide, reverse transcriptase polypeptide, RNA guide, and/or RT donor RNA are encoded in a one or more vectors, e.g., one or more expression vectors.

Embodiment 17: A vector comprising a sequence encoding the Type V CRISPR nuclease polypeptide, reverse transcriptase polypeptide, RNA guide, and/or RT donor RNA of the composition of any of Embodiments 1-16.

Embodiment 18: A cell comprising the composition of any one of Embodiments 1-16 or vector of Embodiment 17. In some examples, the cell is a mammalian cell, for example, a human cell. In one example, the cell is a liver cell (e.g., a hepatocyte).

Embodiment 19: A method of expressing the vector of Embodiment 17.

Embodiment 20: A method of producing the composition of any one of Embodiments 1-17.

Embodiment 21: A method of delivering the composition of any one of Embodiments 1-16.

In some examples, the Type V CRISPR nuclease polypeptide or the nucleic acid encoding the Type V CRISPR nuclease polypeptide, the RNA guide or the nucleic acid encoding the RNA guide, the reverse transcriptase polypeptide or the nucleic acid encoding the reverse transcriptase polypeptide, and the RT donor RNA are delivered in a single delivery vehicle.

In other examples, the Type V CRISPR nuclease polypeptide or the nucleic acid encoding the Type V CRISPR nuclease polypeptide, the RNA guide or the nucleic acid encoding the RNA guide, the reverse transcriptase polypeptide or the nucleic acid encoding the reverse transcriptase polypeptide, and the RT donor RNA are delivered in two or more delivery vehicles.

In yet other examples, the Type V CRISPR nuclease polypeptide or the nucleic acid encoding the Type V CRISPR nuclease polypeptide and the reverse transcriptase polypeptide or the nucleic acid encoding the reverse transcriptase polypeptide are delivered in a single delivery vehicle.

In some instances, the RNA guide and the RT donor RNA are delivered in a single delivery vehicle.

In specific examples, the Type V CRISPR nuclease polypeptide or the nucleic acid encoding the Type V CRISPR nuclease polypeptide and the reverse transcriptase polypeptide or the nucleic acid encoding the reverse transcriptase polypeptide are delivered in a first delivery vehicle and the RNA guide and the RT donor RNA are delivered in a second delivery vehicle.

Embodiment 22: A method of binding the composition of any one of Embodiments 1-16 to a target nucleic acid. In some examples, the target nucleic acid is present in a cell, for example, a mammalian cell such as a human cell. In one example, the cell is a liver cell (e.g., a hepatocyte).

Embodiment 23: A method of introducing an edit into a target nucleic acid comprising contacting the target nucleic acid with a composition of any one of Embodiments 1-16. In some examples, the composition introduces an edit into the target strand or the non-target strand of the target nucleic acid. In some instances, the edit is a substitution, insertion, or deletion.

In specific examples, the edit is a substitution of 1 nucleotide to about 200 nucleotides, e.g., a substitution of 1 nucleotide to about 120 nucleotides, or a substitution of 1 nucleotide to about 20 nucleotides. In other specific examples, the edit is an insertion of 1 nucleotide to about 200 nucleotides, for example, an insertion of 1 nucleotide to about 120 nucleotides, or an insertion of 1 nucleotide to about 20 nucleotides. In some instances, the insertion comprises a hairpin. In yet other specific examples, the edit is a deletion of 1 nucleotide to about 100 nucleotides, for example, a deletion of 1 nucleotide to about 120 nucleotides or a deletion of 1 nucleotide to about 20 nucleotides.

In some instances, the edit occurs within about 200 nucleotides of the PAM sequence, e.g., occurs within about 100 nucleotides of the PAM sequence, occurs within about 50 nucleotides of the PAM sequence, occurs within about 30 nucleotides of the PAM sequence, or occurs within about 20 nucleotides of the PAM sequence.

In some instances, the edit starts and/or ends within about 200 nucleotides upstream of the PAM sequence, for example, starts and/or ends within about 100 nucleotides upstream of the PAM sequence, starts and/or ends within about 50 nucleotides upstream of the PAM sequence, starts and/or ends within about 30 nucleotides upstream of the PAM sequence, starts and/or ends within about 20 nucleotides upstream of the PAM sequence, starts and/or ends within about 10 nucleotides upstream of the PAM sequence, or starts and/or ends within about 5 nucleotides upstream of the PAM sequence.

Alternatively, the edit starts and/or ends within about 25 nucleotides downstream of the PAM sequence, for example, starts and/or ends within about 10 nucleotides downstream of the PAM sequence or starts and/or ends within about 5 nucleotides downstream of the PAM sequence.

In some examples, the edit removes or alters the PAM sequence.

Embodiment 24: An editing template RNA comprising:
(a) a CRISPR nuclease binding sequence;
(b) a DNA-binding sequence that is complementary to the target strand (e.g., the complementary sequence of a target sequence) of a target nucleic acid comprising a target strand and a non-target strand, wherein the target sequence is adjacent to a protospacer adjacent motif (PAM) sequence on the non-target strand; and
(c) a reverse transcription donor RNA (RT donor RNA) comprising a primer binding site (PBS) and a reverse transcription template sequence, wherein the PBS is substantially complementary to a sequence adjacent to the target sequence, and wherein the reverse transcription template sequence comprises at least one encoded edit relative to the target nucleic acid, and
   wherein the DNA-binding sequence and the PBS bind to a same strand of the target nucleic acid.

Embodiment 25: in the editing template RNA of Embodiment 24, the DNA-binding sequence and the PBS bind to a target strand (non-PAM strand) of the target nucleic acid.

Embodiment 26: in the editing template RNA of Embodiment 24, at least one encoded edit is relative to the non-target strand (PAM strand) of the target nucleic acid.

Embodiment 27: the editing template RNA of any one of Embodiments 24-26 comprises a region of unpaired nucleotides when bound to the target nucleic acid. For example, the region of unpaired nucleotides is adjacent to the DNA-binding sequence. Alternatively or in addition, the region of unpaired nucleotides is adjacent to the PBS. In some instances, the region of unpaired nucleotides comprises the reverse transcription template sequence.

Embodiment 28: in any of the editing template RNAs of any one of Embodiments 24-27, the CRISPR nuclease binding sequence, PBS, and reverse transcription template sequence are RNA sequences.

Embodiment 29: in any of the editing template RNAs of any one of Embodiments 24-28, the CRISPR nuclease binding sequence binds to a Type II CRISPR nuclease.

Embodiment 30: in any of the editing template RNAs of any one of Embodiments 24-28, the CRISPR nuclease binding sequence binds to a Type V CRISPR nuclease. In some examples, the CRISPR nuclease binding sequence binds to a Cas12i polypeptide or a variant Cas12i polypeptide. In some instances, the CRISPR nuclease binding sequence binds to a polypeptide having at least 80% identity to any one of SEQ ID NOs: 2-11. In one example, the CRISPR nuclease binding sequence binds to a polypeptide having at least 95% identity to any one of SEQ ID NOs: 2-11. In another example, the CRISPR nuclease binding sequence binds to a polypeptide comprising the amino acid sequence of any one of SEQ ID NOs: 2-11.

Embodiment 31: in any of the editing template RNAs of any one of Embodiments 24-30, the CRISPR nuclease binding sequence binds to a CRISPR nuclease with diminished crRNA processing activity or lacks crRNA processing activity.

Embodiment 32: in any of the editing template RNAs of any one of Embodiments 24-31 where applicable, the CRISPR nuclease binding sequence is a direct repeat sequence. In some examples, the CRISPR nuclease binding sequence is a Cas9 direct repeat sequence. In other examples, the CRISPR nuclease binding sequence is a Cas12i direct repeat sequence. In some instances, the CRISPR nuclease binding sequence comprises a nucleotide sequence with at least 90% identity to any one of SEQ ID NOs: 12-24. For example, the CRISPR nuclease binding sequence comprises a nucleotide sequence with at least 95% identity to any one of SEQ ID NOs: 12-24. In one example, the CRISPR nuclease binding sequence comprises the nucleotide sequence set forth in any one of SEQ ID NOs: 12-24.

Embodiment 33: in any of the editing template RNAs of any one of Embodiments 24-32, the CRISPR nuclease binding sequence is adjacent to the DNA-binding sequence. For example, the DNA-binding sequence is a 3′ extension of the CRISPR nuclease binding sequence.

Embodiment 34: in any of the editing template RNAs of any one of Embodiments 24-33, the DNA-binding sequence is an RNA sequence, a DNA sequence, or an RNA/DNA hybrid sequence.

Embodiment 35, in any of the editing template RNAs of any one of Embodiments 24-34, the DNA-binding sequence (e.g., a spacer sequence) comprises about 10 nucleotides to about 50 nucleotides in length. In some examples, the DNA-binding sequence comprises about 15 nucleotides to about 35 nucleotides in length.

Embodiment 36, in any of the editing template RNAs of any one of Embodiments 24-35, the DNA-binding sequence is adjacent to the PBS. In some examples, the PBS is a 3′ extension of the DNA-binding sequence.

Embodiment 37: in any of the editing template RNAs of any one of Embodiments 24-36, the PBS is between about 3 nucleotides and about 200 nucleotides in length. For example, the PBS is about 3, 4, 5, 6, 7, 8, 9, 10, 15, 20, 25, 30, 35, 40, 45, 50, 55, 60, 65, 70, 75, 80, 85, 90, 95, 100, 105, or 110 nucleotides in length.

Embodiment 38: in any of the editing template RNAs of any one of Embodiments 24-37, the PBS is adjacent to the reverse transcription template sequence. In some examples, the reverse transcription template sequence is a 3′ extension of the PBS.

Embodiment 39: in any of the editing template RNAs of any one of Embodiments 24-38, the reverse transcription template sequence is about 10 nucleotides to about 300 nucleotides in length. In some examples, the reverse transcription template sequence is about 10, 15, 20, 25, 30, 35, 40, 45, 50, 55, 60, 65, 70, 75, 80, 85, 90, 95, 100, 105, 110, 115, or 120 nucleotides in length.

Embodiment 40: any of the editing template RNAs of any one of Embodiments 24-39 may comprise from 5′ to 3′ the nuclease binding sequence, the DNA-binding sequence, the reverse transcription template, and the PBS.

Embodiment 41: any of the editing template RNAs of any one of Embodiments 24-39 may comprise from 5′ to 3′ the reverse transcription template, the PBS, the nuclease binding sequence, and the DNA-binding sequence.

Embodiment 42: in any of the editing template RNAs of any one of Embodiments 24-41, the 3′ end of the PBS comprises a modification.

Embodiment 43: in any of the editing template RNAs of any one of Embodiments 24-42, the 5′ end of the reverse transcription template comprises a modification.

Embodiment 44: in the editing template RNA of Embodiment 42 or 43, the modification is a chemical modification.

Embodiment 45: in the editing template RNA of Embodiment 42 or 43, the modification is a nucleic acid sequence comprising secondary structure. For example, the modification is a hairpin, a pseudoknot, a triplex structure, an xrRNA, a tRNA, or a truncated tRNA.

Embodiment 46: in the editing template RNA of Embodiment 42 or 43 the modification comprises a nuclease binding sequence or a nuclease binding sequence and a DNA-binding sequence.

Embodiment 47: any of the editing template RNA of any one of Embodiments 24-47 can cause an edit, which can be a substitution, an insertion, or a deletion. In some examples, the edit is a substitution of 1 nucleotide to about 200 nucleotides, for example, a substitution of 1 nucleotide to about 120 nucleotides, or a substitution of 1 nucleotide to about 20 nucleotides. In other examples, the edit is an insertion of 1 nucleotide to about 200 nucleotides, for example, an insertion of 1 nucleotide to about 120 nucleotides, or an insertion of 1 nucleotide to about 20 nucleotides. In some instances, the insertion comprises a hairpin. In yet other examples, the edit is a deletion of 1 nucleotide to about 100 nucleotides, for example, a deletion of 1 nucleotide to about 120 nucleotides or a deletion of 1 nucleotide to about 20 nucleotides.

In some examples, the edit is within about 200 nucleotides of the PAM sequence, for example, within about 100 nucleotides of the PAM sequence, within about 50 nucleotides of the PAM sequence, within about 30 nucleotides of the PAM sequence, or within about 20 nucleotides of the PAM sequence.

In some examples, the edit starts and/or ends within about 200 nucleotides upstream of the PAM sequence, for example, starts and/or ends within about 100 nucleotides upstream of the PAM sequence, starts and/or ends within about 50 nucleotides upstream of the PAM sequence, starts and/or ends within about 30 nucleotides upstream of the PAM sequence, starts and/or ends within about 20 nucleotides upstream of the PAM sequence, starts and/or ends within about 10 nucleotides upstream of the PAM sequence, or starts and/or ends within about 5 nucleotides upstream of the PAM sequence.

In other examples, the edit starts and/or ends within about 5 nucleotides downstream of the PAM sequence. In one example, the edit starts and/or ends within about 10 nucleotides downstream of the PAM sequence. In another example, the edit starts and/or ends within about 25 nucleotides downstream of the PAM sequence.

In some examples, the edit removes or alters the PAM sequence. Alternatively or in addition, the edit prevents retargeting by the Type V CRISPR nuclease polypeptide (e.g., prevents binding of the Type V CRISPR nuclease to the target sequence).

Embodiment 48: the editing template RNA of any one of Embodiments 24-47 is present in a cell, for example, a mammalian cell such as a human cell. In one example, the cell is a liver cell (e.g., a hepatocyte).

Embodiment 49: the editing template RNA of any one of Embodiments 24-47 is formulated for delivery to a cell for example, a mammalian cell such as a human cell. In one example, the cell is a liver cell (e.g., a hepatocyte). In some examples, the editing template RNA is formulated with a CRISPR nuclease or a nucleic acid encoding the CRISPR nuclease in a single delivery vehicle. In other examples, the editing template RNA is formulated with a CRISPR nuclease polypeptide or a nucleic acid encoding the CRISPR nuclease polypeptide and a reverse transcriptase polypeptide or a nucleic acid encoding the reverse transcriptase polypeptide in a single delivery vehicle.

Embodiment 50: the editing template RNA of any one of Embodiments 24-49 where applicable is encoded in a vector.

Embodiment 51: A vector comprising a sequence encoding the editing template RNA of any one of Embodiments 24-50.

Embodiment 52: A complex comprising the editing template RNA of any one of Embodiments 24-50. In some examples, the complex comprises a CRISPR nuclease. In other examples, the complex comprises a target sequence or a target nucleic acid. In yet other examples, the complex comprises a CRISPR nuclease and a target sequence or a target nucleic acid.

In some examples, the CRISPR nuclease is a nickase. In other examples, the CRISPR nuclease cleaves both strands of a DNA duplex. In yet other examples, the CRISPR nuclease is a blunt cutting nuclease. Alternatively, the CRISPR nuclease is a staggered cutting nuclease.

Embodiment 53: A cell comprising the editing template RNA, vector, or complex of any one of Embodiments 24-52. In some instances, the cell is a mammalian cell, such as a human cell. In one example, the cell is a liver cell (e.g., a hepatocyte).

Embodiment 54: A method of expressing the vector of the Embodiment of 31.

Embodiment 55: A method of producing the editing template RNA of any one of Embodiments 24-50.

Embodiment 56: A method of delivering the editing template RNA of any one of Embodiments 24-50. In some instances, the editing template RNA is formulated with a CRISPR nuclease or a nucleic acid encoding the CRISPR nuclease in a single delivery vehicle. In other examples, the editing template RNA is formulated with a CRISPR nuclease polypeptide or a nucleic acid encoding the CRISPR nuclease polypeptide and a reverse transcriptase polypeptide or a nucleic acid encoding the reverse transcriptase polypeptide in a single delivery vehicle.

Embodiment 57: A method of binding the editing template RNA of any one of Embodiments 24-50 with a CRISPR nuclease.

Embodiment 58: A method of binding the editing template RNA of any one of Embodiments 24-50 with a target sequence or a target nucleic acid.

Embodiment 59: A method of binding the editing template RNA of any one of Embodiments 24-50 with a CRISPR nuclease and a target sequence or a target nucleic acid.

Embodiment 60: A method of introducing an edit into a target nucleic acid comprising contacting the target nucleic acid with an editing template RNA of any one of Embodiments 24-50 and a CRISPR nuclease. In some instances, the CRISPR nuclease is a Type II CRISPR nuclease. In other instances, the CRISPR is a Type V CRISPR nuclease. For example, the CRISPR nuclease is a Cas12i polypeptide or a variant Cas12i polypeptide. In specific examples, the CRISPR nuclease is a polypeptide having at least 80% identity to any of SEQ ID NOs: 2-11, for example, at least 95% identity to any of SEQ ID NOs: 2-11. In one example, the CRISPR nuclease is a polypeptide comprising the amino acid sequence of any of SEQ ID NOs: 2-11.

In some examples, the CRISPR nuclease is a CRISPR nuclease that comprises diminished crRNA processing activity or lacks crRNA processing activity.

In some examples, the CRISPR nuclease is a nickase. Alternatively, the CRISPR nuclease cleaves both strands of a DNA duplex. In some instances, the CRISPR nuclease is a blunt cutting nuclease. Alternatively, the CRISPR nuclease is a staggered cutting nuclease.

Embodiment 61: in the method of Embodiment 60, the editing template RNA introduces an edit into the target strand of the target nucleic acid. In some examples, the edit is a substitution, insertion, or deletion. For example, the edit can be a substitution of 1 nucleotide to about 200 nucleotides, e.g., a substitution of 1 nucleotide to about 120 nucleotides, or a substitution of 1 nucleotide to about 20 nucleotides. Alternatively, the edit can be an insertion of 1 nucleotide to about 200 nucleotides, for example, an insertion of 1 nucleotide to about 120 nucleotides or an insertion of 1 nucleotide to about 20 nucleotides. In some instances, the insertion comprises a hairpin. In other examples, the edit can be a deletion of 1 nucleotide to about 100 nucleotides, for example, a deletion of 1 nucleotide to about 120 nucleotides or a deletion of 1 nucleotide to about 20 nucleotides.

In some examples, the edit is within about 200 nucleotides of the PAM sequence, for example, within about 100 nucleotides of the PAM sequence, within about 50 nucleotides of the PAM sequence, within about 30 nucleotides of the PAM sequence, or within about 20 nucleotides of the PAM sequence.

In some examples, the edit starts and/or ends within about 200 nucleotides upstream of the PAM sequence, for example, starts and/or ends within about 100 nucleotides upstream of the PAM sequence, starts and/or ends within about 50 nucleotides upstream of the PAM sequence, starts and/or ends within about 30 nucleotides upstream of the PAM sequence, starts and/or ends within about 20 nucleotides upstream of the PAM sequence, starts and/or ends within about 10 nucleotides upstream of the PAM sequence, or starts and/or ends within about 5 nucleotides upstream of the PAM sequence.

In other examples, the edit starts and/or ends within about 5 nucleotides downstream of the PAM sequence. In one example, the edit starts and/or ends within about 10 nucleotides downstream of the PAM sequence. In another example, the edit starts and/or ends within about 25 nucleotides downstream of the PAM sequence.

In some examples, the edit removes or alters the PAM sequence. Alternatively or in addition, the edit prevents retargeting by the Type V CRISPR nuclease polypeptide (e.g., prevents binding of the Type V CRISPR nuclease to the target sequence).

In some examples, the target nucleic acid is present in a cell, for example, a mammalian cell such as a human cell. In one example, the cell is a liver cell (e.g., a hepatocyte).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—RNA-Templated Editing of Target Strand in Mammalian Cells

This Example describes target strand editing of mammalian genes (e.g., using an editing template RNA that binds the non-PAM strand of selected mammalian genes).

Fusions of a variant Cas12i2 (SEQ ID NO: 4) with mutant MMLV reverse transcriptase of SEQ ID NO: 29 were cloned into the pcda3.1 backbone (Invitrogen). Configurations of the N-terminal and C-terminal RT fusion to variant Cas12i2 are shown in Table 7. A working solution of plasmid for expression of RT fusion with variant Cas12i2 was prepared in water (variant Cas12i2-RT fusion working solution).

TABLE 7
CAS-RT FUSION DESIGNS AND SEQUENCES

| Full | Shorthand | |
|---|---|---|
| Description | Description | Sequence |
| Cas12i2 | C_MMLV_Cas12i2 | MSSAIKSYKSVLRPNERKNQLLKSTIQCLEDGSA |
| variant | variant | FFFKMLQGLFGGITPEIVRFSTEQEKQQQDIALW |
| (H485A) - | | CAVNWFRPVSQDSLTHTIASDNLVEKFEEYYGG |
| NLS - | | TASDAIKQYFSASIGESYYWNDCRQQYYDLCRE |
| 2xSGGS - | | LGVEVSDLTHDLEILCREKCLAVATESNQNNSIIS |
| XTEN (16 aa) | | VLFGTGEKEDRSVKLRITKKILEAISNLKEIPKNV |
| - 2xSGGS - | | APIQEIILNVAKATKETFRQVYAGNLGAPSTLEKF |
| mutMMLVRT - | | IAKDGQKEFDLKKLQTDLKKVIRGKSKERDWCC |
| NLS | | QEELRSYVEQNTIQYDLWAWGEMFNKAHTALKI |
| | | KSTRNYNFAKQRLEQFKEIQSLNNLLVVKKLND |
| | | FFDSEFFSGEETYTICVHHLGGKDLSKLYKAWED |
| | | DPADPENAIVVLCDDLKNNFKKEPIRNILRYIFTI |
| | | RQECSAQDILAAAKYNQQLDRYKSQKANPSVLG |
| | | NQGFTWTNAVILPEKAQRNDRPNSLDLRIWLYL |
| | | KLRHPDGRWKKAHIPFYDTRFFQEIYAAGNSPV |
| | | DTCQFRTPRFGYHLPKLTDQTAIRVNKKHVKAA |
| | | KTEARIRLAIQQGTLPVSNLKITEISATINSKGQVR |
| | | IPVKFRVGRQKGTLQIGDRFCGYDQNQTASHAY |
| | | SLWEVVKEGQYHKELGCFVRFISSGDIVSITENR |
| | | GNQFDQLSYEGLAYPQYADWRKKASKFVSLWQ |
| | | ITKKNKKKEIVTVEAKEKFDAICKYQPRLYKFNK |
| | | EYAYLLRDIVRGKSLVELQQIRQEIFRFIEQDCGV |
| | | TRLGSLSLSTLETVKAVKGIIYSYFSTALNASKNN |
| | | PISDEQRKEFDPELFALLEKLELIRTRKKKQKVER |
| | | IANSLIQTCLENNIKFIRGEGDLSTTNNATKKKAN |
| | | SRSMDWLARGVFNKIRQLAPMHNITLFGCGSLY |
| | | TSHQDPLVHRNPDKAMKCRWAAIPVKDIGDWV |
| | | LRKLSQNLRAKNRGTGEYYHQGVKEFLSHYELQ |
| | | DLEEELLKWRSDRKSNIPCWVLQNRLAEKLGNK |
| | | EAVVYIPVRGGRIYFATHKVATGAVSIVFDQKQV |
| | | WVCNADHVAAANIALTGKGIGEQSSDEENPDGS |
| | | RIKLQLTSKRPAATKKAGQAKKKKSGGSSGGSS |
| | | GSETPGTSESATPESSGGSSGGSTLNIEDEYRLHE |
| | | TSKEPDVSLGSTWLSDFPQAWAETGGMGLAVR |
| | | QAPLIIPLKATSTPVSIKQYPMSQEARLGIKPHIQR |
| | | LLDQGILVPCQSPWNTPLLPVKKPGTNDYRPVQ |
| | | DLREVNKRVEDIHPTVPNPYNLLSGLPPSHQWYT |
| | | VLDLKDAFFCLRLHPTSQPLFAFEWRDPEMGISG |
| | | QLTWTRLPQGFKNSPTLFNEALHRDLADFRIQHP |
| | | DLILLQYVDDLLLAATSELDCQQGTRALLQTLG |
| | | NLGYRASAKKAQICQKQVKYLGYLLKEGQRWL |
| | | TEARKETVMGQPTPKTPRQLREFLGKAGFCRLFI |
| | | PGFAEMAAPLYPLTKPGTLFNWGPDQQKAYQEI |
| | | KQALLTAPALGLPDLTKPFELFVDEKQGYAKGV |
| | | LTQKLGPWRRPVAYLSKKLDPVAAGWPPCLRM |
| | | VAAIAVLTKDAGKLTMGQPLVILAPHAVEALVK |
| | | QPPDRWLSNARMTHYQALLLDTDRVQFGPVVA |
| | | LNPATLLPLPEEGLQHNCLDILAEAHGTRPDLTD |
| | | QPLPDADHTWYTDGSSLLQEGQRKAGAAVTTET |
| | | EVIWAKALPAGTSAQRAELIALTQALKMAEGKK |
| | | LNVYTDSRYAFATAHIHGEIYRRRGWLTSEGKEI |
| | | KNKDEILALLKALFLPKRLSIIHCPGHQKGHSAEA |
| | | RGNRMADQAARKAAITETPDTSTLLIENSSPMKR |
| | | TADGSEFESPKKKRKV (SEQ ID NO: 25) |
| NLS - | N_MMLV_Cas12i2 | MKRTADGSEFESPKKKRKVTLNIEDEYRLHETSK |
| mutMMLVRT - | variant | EPDVSLGSTWLSDFPQAWAETGGMGLAVRQAP |
| 2xSGGS - | | LIIPLKATSTPVSIKQYPMSQEARLGIKPHIQRLLD |
| XTEN (16 aa) | | QGILVPCQSPWNTPLLPVKKPGTNDYRPVQDLRE |
| - 2xSGGS - | | VNKRVEDIHPTVPNPYNLLSGLPPSHQWYTVLDL |
| Cas12i2 | | KDAFFCLRLHPTSQPLFAFEWRDPEMGISGQLTW |
| variant | | TRLPQGFKNSPTLFNEALHRDLADFRIQHPDLILL |
| (H485A) - | | QYVDDLLLAATSELDCQQGTRALLQTLGNLGYR |
| NLS | | ASAKKAQICQKQVKYLGYLLKEGQRWLTEARK |
| | | ETVMGQPTPKTPRQLREFLGKAGFCRLFIPGFAE |
| | | MAAPLYPLTKPGTLFNWGPDQQKAYQEIKQALL |
| | | TAPALGLPDLTKPFELFVDEKQGYAKGVLTQKL |
| | | GPWRRPVAYLSKKLDPVAAGWPPCLRMVAAIA |
| | | VLTKDAGKLTMGQPLVILAPHAVEALVKQPPDR |
| | | WLSNARMTHYQALLLDTDRVQFGPVVALNPAT |
| | | LLPLPEEGLQHNCLDILAEAHGTRPDLTDQPLPD |
| | | ADHTWYTDGSSLLQEGQRKAGAAVTTETEVIW |
| | | AKALPAGTSAQRAELIALTQALKMAEGKKLNVY |
| | | TDSRYAFATAHIHGEIYRRRGWLTSEGKEIKNKD |
| | | EILALLKALFLPKRLSIIHCPGHQKGHSAEARGNR |
| | | MADQAARKAAITETPDTSTLLIENSSPSGGSSGGS |
| | | SGSETPGTSESATPESSGGSSGGSMSSAIKSYKSV |
| | | LRPNERKNQLLKSTIQCLEDGSAFFFKMLQGLFG |
| | | GITPEIVRFSTEQEKQQQDIALWCAVNWFRPVSQ |
| | | DSLTHTIASDNLVEKFEEYYGGTASDAIKQYFSA |
| | | SIGESYYWNDCRQQYYDLCRELGVEVSDLTHDL |
| | | EILCREKCLAVATESNQNNSIISVLFGTGEKEDRS |
| | | VKLRITKKILEAISNLKEIPKNVAPIQEIILNVAKA |
| | | TKETFRQVYAGNLGAPSTLEKFIAKDGQKEFDLK |
| | | KLQTDLKKVIRGKSKERDWCCQEELRSYVEQNT |
| | | IQYDLWAWGEMFNKAHTALKIKSTRNYNFAKQ |
| | | RLEQFKEIQSLNNLLVVKKLNDFFDSEFFSGEETY |
| | | TICVHHLGGKDLSKLYKAWEDDPADPENAIVVL |
| | | CDDLKNNFKKEPIRNILRYIFTIRQECSAQDILAA |
| | | AKYNQQLDRYKSQKANPSVLGNQGFTWTNAVI |
| | | LPEKAQRNDRPNSLDLRIWLYLKLRHPDGRWKK |
| | | AHIPFYDTRFFQEIYAAGNSPVDTCQFRTPRFGY |
| | | HLPKLTDQTAIRVNKKHVKAAKTEARIRLAIQQG |
| | | TLPVSNLKITEISATINSKGQVRIPVKFRVGRQKG |
| | | TLQIGDRFCGYDQNQTASHAYSLWEVVKEGQY |
| | | HKELGCFVRFISSGDIVSITENRGNQFDQLSYEGL |
| | | AYPQYADWRKKASKFVSLWQITKKNKKKEIVT |
| | | VEAKEKFDAICKYQPRLYKFNKEYAYLLRDIVR |
| | | GKSLVELQQIRQEIFRFIEQDCGVTRLGSLSLSTLE |
| | | TVKAVKGIIYSYFSTALNASKNNPISDEQRKEFDP |
| | | ELFALLEKLELIRTRKKKQKVERIANSLIQTCLEN |
| | | NIKFIRGEGDLSTTNNATKKKANSRSMDWLARG |
| | | VFNKIRQLAPMHNITLFGCGSLYTSHQDPLVHRN |
| | | PDKAMKCRWAAIPVKDIGDWVLRKLSQNLRAK |
| | | NRGTGEYYHQGVKEFLSHYELQDLEEELLKWRS |
| | | DRKSNIPCWVLQNRLAEKLGNKEAVVYIPVRGG |
| | | RIYFATHKVATGAVSIVFDQKQVWVCNADHVA |
| | | AANIALTGKGIGEQSSDEENPDGSRIKLQLTSKRP |
| | | AATKKAGQAKKKK (SEQ ID NO: 26) |
| MMLV | MMLV | MTLNIEDEYRLHETSKEPDVSLGSTWLSDFPQA |
| | | WAETGGMGLAVRQAPLIIPLKATSTPVSIKQYPM |
| | | SQEARLGIKPHIQRLLDQGILVPCQSPWNTPLLPV |
| | | KKPGTNDYRPVQDLREVNKRVEDIHPTVPNPYN |
| | | LLSGLPPSHQWYTVLDLKDAFFCLRLHPTSQPLF |
| | | AFEWRDPEMGISGQLTWTRLPQGFKNSPTLFNE |
| | | ALHRDLADFRIQHPDLILLQYVDDLLLAATSELD |
| | | CQQGTRALLQTLGNLGYRASAKKAQICQKQVK |
| | | YLGYLLKEGQRWLTEARKETVMGQPTPKTPRQL |
| | | REFLGKAGFCRLFIPGFAEMAAPLYPLTKPGTLF |
| | | NWGPDQQKAYQEIKQALLTAPALGLPDLTKPFE |
| | | LFVDEKQGYAKGVLTQKLGPWRRPVAYLSKKL |
| | | DPVAAGWPPCLRMVAAIAVLTKDAGKLTMGQP |
| | | LVILAPHAVEALVKQPPDRWLSNARMTHYQALL |
| | | LDTDRVQFGPVVALNPATLLPLPEEGLQHNCLDI |
| | | LAEAHGTRPDLTDQPLPDADHTWYTDGSSLLQE |
| | | GQRKAGAAVTTETEVIWAKALPAGTSAQRAELI |
| | | ALTQALKMAEGKKLNVYTDSRYAFATAHIHGEI |
| | | YRRRGWLTSEGKEIKNKDEILALLKALFLPKRLSI |
| | | IHCPGHQKGHSAEARGNRMADQAARKAAITETP |
| | | DTSTLLIENSSP (SEQ ID NO: 29) |

Various RNA guide-RT donor RNA fusion configurations were tested, as shown in Table 8 and depicted in FIG. 8A. A reverse transcription template sequence and PBS was fused to the 3′ end of the RNA guide. The reverse transcription template sequence was designed to introduce a substitution, insertion, deletion, or hairpin into either an AAVS1_T7 target or VEGFA_T5 target. The sequences of the RNA guide-RT donor RNA fusions are shown in Table 9 and partially depicted in FIG. 8B. In Table 9 and FIG. 8B, “S” refers to substitution, “I” refers to insertion, “D” refers to deletion, and “H” refers to hairpin, and the PBS lengths are in parentheses. Sequences of RNA guides only, which were used as controls, are shown in Table 10. The RNA guide-RT donor RNA fusions or RNA guides were cloned into a plasmid backbone with a U6 promoter and maxi-prepped. A working solution of plasmid expressing each RNA guide/RT donor RNA plasmid (or RNA guide) was prepared in water (editing template RNA working solution).

TABLE 8
RNA GUIDE-RT DONOR RNA FUSION DESIGNS.

| Configuration | Nuclease | Description |
|---|---|---|
| DR - spacer - | Cas12i2 | reverse transcription template |
| reverse transcription | variant | sequence: 34 nucleotides |
| template sequence - PBS | | PBS: 13, 30, or 60 nucleotides |
| | | Hairpin: optional |
| DR - spacer | Cas12i2 | Control (No RT donor RNA) |
| | variant | |

TABLE 9
RNA GUIDE-RT DONOR FUSION SEQUENCES.

| | | RNA Guide-RT donor | RNA Guide-RT donor RNA |
|---|---|---|---|
| Target | Cas-RT Fusion | RNA Description | Sequences |
| AAVS1_T6 | N_MMLV_Variant | nuclease binding | AGAAAUCCGUCUUUCAUUG |
| GTAGCCT | Cas12i2 | sequence - DNA- | ACGGGUAGCCUCUCCCGCUC |
| CTCCCGC | or | binding sequence - | UGGUGACAUUCUUUGUAGC |
| TCTGGT | C_MMLV_Variant | reverse transcription | CUCUCCCCGAGUGGUUCAGG |
| (SEQ ID | Cas12i2 | template sequence (S) - | GCCCAGCUAGGGAUCCAGA |
| NO: 30) | | PBS (60 nt) | UCUGGGUGAUUUAGGCUCC |
| | | | CUCUGUCUGGAUCAGUCCUC |
| | | | C (SEQ ID NO: 40) |
| AAVS1_T6 | N_MMLV_Variant | nuclease binding | AGAAAUCCGUCUUUCAUUG |
| (SEQ ID | Cas12i2 | sequence - DNA- | ACGGGUAGCCUCUCCCGCUC |
| NO: 30) | or | binding sequence - | UGGUGACAUUCUUUGUAGC |
| | C_MMLV_Variant | reverse transcription | CUCUCCCCGAGUGGUUCAGG |
| | Cas12i2 | template sequence (S) - | GCCCAGCUAGGGAUCCAGA |
| | | PBS (30 nt) | UCUGGGUGAU (SEQ ID NO: |
| | | | 41) |
| AAVS1_T6 | N_MMLV_Variant | nuclease binding | AGAAAUCCGUCUUUCAUUG |
| (SEQ ID | Cas12i2 | sequence - DNA- | ACGGGUAGCCUCUCCCGCUC |
| NO: 30) | or | binding sequence - | UGGUGCACCCCCAUAAGGG |
| | C_MMLV_Variant | reverse transcription | GGGACAUUCUUUGUAGCCU |
| | Cas12i2 | template sequence (H) - | CUCCCCGAGUGGUUCAGGGC |
| | | PBS (13 nt) | CCAGCUAGGG (SEQ ID NO: |
| | | | 42) |
| AAVS1_T6 | N_MMLV_Variant | nuclease binding | AGAAAUCCGUCUUUCAUUG |
| (SEQ ID | Cas12i2 | sequence - DNA- | ACGGGUAGCCUCUCCCGCUC |
| NO: 30) | or | binding sequence - | UGGUGACAUUCUUUGUAGC |
| | C_MMLV_Variant | reverse transcription | CUCUCCCCGAGUGGUUCAGG |
| | Cas12i2 | template sequence (S) - | GCCCAGCUAGGG (SEQ ID |
| | | PBS (13 nt) | NO: 43) |
| AAVS1_T6 | N_MMLV_Variant | nuclease binding | AGAAAUCCGUCUUUCAUUG |
| (SEQ ID | Cas12i2 | sequence - DNA- | ACGGGUAGCCUCUCCCGCUC |
| NO: 30) | or | binding sequence - | UGGUGACAUUCUUUGUAGC |
| | C_MMLV_Variant | reverse transcription | CUCUCCCAGCGGCUCUGGUU |
| | Cas12i2 | template sequence (I) - | CAGGGCCCAGCUAGGG(SEQ |
| | | PBS (13 nt) | ID NO: 44) |
| AAVS1_T6 | N_MMLV_Variant | nuclease binding | AGAAAUCCGUCUUUCAUUG |
| (SEQ ID | Cas12i2 | sequence - DNA- | ACGGGUAGCCUCUCCCGCUC |
| NO: 30) | or | binding sequence - | UGGUGACAUUCUUUGUAGC |
| | C_MMLV_Variant | reverse transcription | CUCUCCCUGGUUCAGGGCCC |
| | Cas12i2 | template sequence (D) - | AGCUAGGG (SEQ ID NO: 45) |
| | | PBS (13 nt) | |
| AAVS1_T7 | N_MMLV_Variant | nuclease binding | AGAAAUCCGUCUUUCAUUG |
| GGGAAGT | Cas12i2 | sequence - DNA- | ACGGGGGAAGUGGUUGGUC |
| GGTTGGT | or | binding sequence - | AGCAUCAGCUACUUUGGGA |
| CAGCAT | C_MMLV_Variant | reverse transcription | AGUGGUUGCGAGGCAUGGA |
| (SEQ ID | Cas12i2 | template sequence (S) - | UUAUAGCCGAAGGCCCCAGC |
| NO: 32) | | PBS (60 nt) | UUUGCCUUGUUCUAGCAGU |
| | | | UCCACUCCUGGGCAGCCCGA |
| | | | GA (SEQ ID NO: 54) |
| AAVS1_T7 | N_MMLV_Variant | nuclease binding | AGAAAUCCGUCUUUCAUUG |
| (SEQ ID | Cas12i2 | sequence - DNA- | ACGGGGGAAGUGGUUGGUC |
| NO: 32) | or | binding sequence - | AGCAUCAGCUACUUUGGGA |
| | C_MMLV_Variant | reverse transcription | AGUGGUUGCGAGGCAUGGA |
| | Cas12i2 | template sequence (S) - | UUAUAGCCGAAGGCCCCAGC |
| | | PBS (30 nt) | UUUGCCUUGUU (SEQ ID NO: |
| | | | 55) |
| AAVS1_T7 | N_MMLV_Variant | nuclease binding | AGAAAUCCGUCUUUCAUUG |
| (SEQ ID | Cas12i2 | sequence - DNA- | ACGGGGGAAGUGGUUGGUC |
| NO: 32) | or | binding sequence - | AGCAUGCACCCCCAUAAGGG |
| | C_MMLV_Variant | reverse transcription | GGCAGCUACUUUGGGAAGU |
| | Cas12i2 | template sequence (H) - | GGUUGCGAGGCAUGGAUUA |
| | | PBS (13 nt) | UAGCCGAAGGC (SEQ ID NO: |
| | | | 56) |
| AAVS1_T7 | N_MMLV_Variant | nuclease binding | AGAAAUCCGUCUUUCAUUG |
| (SEQ ID | Cas12i2 | sequence - DNA- | ACGGGGGAAGUGGUUGGUC |
| NO: 32) | or | binding sequence - | AGCAUCAGCUACUUUGGGA |
| | C_MMLV_Variant | reverse transcription | AGUGGUUGCGAGGCAUGGA |
| | Cas12i2 | template sequence (S) - | UUAUAGCCGAAGGC (SEQ ID |
| | | PBS (13 nt) | NO: 57) |
| AAVS1_T7 | N_MMLV_Variant | nuclease binding | AGAAAUCCGUCUUUCAUUG |
| (SEQ ID | Cas12i2 | sequence - DNA- | ACGGGGGAAGUGGUUGGUC |
| NO: 32) | or | binding sequence - | AGCAUCAGCUACUUUGGGA |
| | C_MMLV_Variant | reverse transcription | AGUGGUUGAGCGGUCAGCA |
| | Cas12i2 | template sequence (I) - | UGGAUUAUAGCCGAAGGC |
| | | PBS (13 nt) | (SEQ ID NO: 58) |
| AAVS1_T7 | N_MMLV_Variant | nuclease binding | AGAAAUCCGUCUUUCAUUG |
| (SEQ ID | Cas12i2 | sequence - DNA- | ACGGGGGAAGUGGUUGGUC |
| NO: 32) | or | binding sequence - | AGCAUCAGCUACUUUGGGA |
| | C_MMLV_Variant | reverse transcription | AGUGGUUGGCAUGGAUUAU |
| | Cas12i2 | template sequence (D) - | AGCCGAAGGC (SEQ ID NO: |
| | | PBS (13 nt) | 59) |
| EMX1_T6 | N_MMLV_Variant | nuclease binding | AGAAAUCCGUCUUUCAUUG |
| GAGCCAG | Cas12i2 | sequence - DNA- | ACGGGAGCCAGUGUUGCUA |
| TGTTGCT | or | binding sequence - | GUCAAUUCCUUCUUUGAGC |
| AGTCAA | C_MMLV_Variant | reverse transcription | CAGUGUUGCGAGUCAAGGG |
| (SEQ ID | Cas12i2 | template sequence (S) - | CAGCAUGCUGGGCCCGUCCC |
| NO: 34) | | PBS (60 nt) | ACUACAGGCCAAUGUGACC |
| | | | GUCAGUCUCCUUCCUGAAG |
| | | | GAC (SEQ ID NO: 68) |
| EMX1_T6 | N_MMLV_Variant | nuclease binding | AGAAAUCCGUCUUUCAUUG |
| (SEQ ID | Cas12i2 | sequence - DNA- | ACGGGAGCCAGUGUUGCUA |
| NO: 34) | or | binding sequence - | GUCAAUUCCUUCUUUGAGC |
| | C_MMLV_Variant | reverse transcription | CAGUGUUGCGAGUCAAGGG |
| | Cas12i2 | template sequence (S) - | CAGCAUGCUGGGCCCGUCCC |
| | | PBS (30 nt) | ACUACAGGCCA (SEQ ID NO: |
| | | | 69) |
| EMX1_T6 | N_MMLV_Variant | nuclease binding | AGAAAUCCGUCUUUCAUUG |
| (SEQ ID | Cas12i2 | sequence - DNA- | ACGGGAGCCAGUGUUGCUA |
| NO: 34) | or | binding sequence - | GUCAAGCACCCCCAUAAGGG |
| | C_MMLV_Variant | reverse transcription | GGUUCCUUCUUUGAGCCAG |
| | Cas12i2 | template sequence (H) - | UGUUGCGAGUCAAGGGCAG |
| | | PBS (13 nt) | CAUGCUGGGCC (SEQ ID NO: |
| | | | 70) |
| EMX1_T6 | N_MMLV_Variant | nuclease binding | AGAAAUCCGUCUUUCAUUG |
| (SEQ ID | Cas12i2 | sequence - DNA- | ACGGGAGCCAGUGUUGCUA |
| NO: 34) | or | binding sequence - | GUCAAUUCCUUCUUUGAGC |
| | C_MMLV_Variant | reverse transcription | CAGUGUUGCGAGUCAAGGG |
| | Cas12i2 | template sequence (S) - | CAGCAUGCUGGGCC (SEQ ID |
| | | PBS (13 nt) | NO: 71) |
| EMX1_T6 | N_MMLV_Variant | nuclease binding | AGAAAUCCGUCUUUCAUUG |
| (SEQ ID | Cas12i2 | sequence - DNA- | ACGGGAGCCAGUGUUGCUA |
| NO: 34) | or | binding sequence - | GUCAAUUCCUUCUUUGAGC |
| | C_MMLV_Variant | reverse transcription | CAGUGUUGAGCGCUAGUCA |
| | Cas12i2 | template sequence (I) - | AGGGCAGCAUGCUGGGCC |
| | | PBS (13 nt) | (SEQ ID NO: 72) |
| EMX1_T6 | N_MMLV_Variant | nuclease binding | AGAAAUCCGUCUUUCAUUG |
| (SEQ ID | Cas12i2 | sequence - DNA- | ACGGGAGCCAGUGUUGCUA |
| NO: 34) | or | binding sequence - | GUCAAUUCCUUCUUUGAGC |
| | C_MMLV_Variant | reverse transcription | CAGUGUUGUCAAGGGCAGC |
| | Cas12i2 | template sequence (D) - | AUGCUGGGCC (SEQ ID NO: |
| | | PBS (13 nt) | 73) |
| VEGFA_T2 | N_MMLV_Variant | nuclease binding | AGAAAUCCGUCUUUCAUUG |
| AATCCTC | Cas12i2 | sequence - DNA- | ACGGAAUCCUCCACCAGUCA |
| CACCAGT | or | binding sequence - | UGGUAGAUACCUUUAAUCC |
| CATGGT | C_MMLV_Variant | reverse transcription | UCCACCACGAGUGGUGACA |
| (SEQ ID | Cas12i2 | template sequence (S) - | ACCCCAAGCAGCCCACACAU |
| NO: 36) | | PBS (60 nt) | UUUCAAGUGCCCCCAGGAU |
| | | | GCGUGGAGGGAGGGGUCUG |
| | | | UG (SEQ ID NO: 81) |
| VEGFA_T2 | N_MMLV_Variant | nuclease binding | AGAAAUCCGUCUUUCAUUG |
| (SEQ ID | Cas12i2 | sequence - DNA- | ACGGAAUCCUCCACCAGUCA |
| NO: 36) | or | binding sequence - | UGGUAGAUACCUUUAAUCC |
| | C_MMLV_Variant | reverse transcription | UCCACCACGAGUGGUGACA |
| | Cas12i2 | template sequence (S) - | ACCCCAAGCAGCCCACACAU |
| | | PBS (30 nt) | UUUCAAGUGC (SEQ ID NO: |
| | | | 82) |
| VEGFA_T2 | N_MMLV_Variant | nuclease binding | AGAAAUCCGUCUUUCAUUG |
| (SEQ ID | Cas12i2 | sequence - DNA- | ACGGAAUCCUCCACCAGUCA |
| NO: 36) | or | binding sequence - | UGGUGCACCCCCAUAAGGG |
| | C_MMLV_Variant | reverse transcription | GGAGAUACCUUUAAUCCUC |
| | Cas12i2 | template sequence (H) - | CACCACGAGUGGUGACAACC |
| | | PBS (13 nt) | CCAAGCAGCC (SEQ ID NO: |
| | | | 83) |
| VEGFA_T2 | N_MMLV_Variant | nuclease binding | AGAAAUCCGUCUUUCAUUG |
| (SEQ ID | Cas12i2 | sequence - DNA- | ACGGAAUCCUCCACCAGUCA |
| NO: 36) | or | binding sequence - | UGGUAGAUACCUUUAAUCC |
| | C_MMLV_Variant | reverse transcription | UCCACCACGAGUGGUGACA |
| | Cas12i2 | template sequence (S) - | ACCCCAAGCAGCC (SEQ ID |
| | | PBS (13 nt) | NO: 84) |
| VEGFA_T2 | N_MMLV_Variant | nuclease binding | AGAAAUCCGUCUUUCAUUG |
| (SEQ ID | Cas12i2 | sequence - DNA- | ACGGAAUCCUCCACCAGUCA |
| NO: 36) | or | binding sequence - | UGGUAGAUACCUUUAAUCC |
| | C_MMLV_Variant | reverse transcription | UCCACCAAGCGGUCAUGGU |
| | Cas12i2 | template sequence (I) - | GACAACCCCAAGCAGCC |
| | | PBS (13 nt) | (SEQ ID NO: 85) |
| VEGFA_T2 | N_MMLV_Variant | nuclease binding | AGAAAUCCGUCUUUCAUUG |
| (SEQ ID | Cas12i2 | sequence - DNA- | ACGGAAUCCUCCACCAGUCA |
| NO: 36) | or | binding sequence - | UGGUAGAUACCUUUAAUCC |
| | C_MMLV_Variant | reverse transcription | UCCACCAUGGUGACAACCCC |
| | Cas12i2 | template sequence (D) - | AAGCAGCC (SEQ ID NO: 86) |
| | | PBS (13 nt) | |
| VEGFA_T5 | N_MMLV_Variant | nuclease binding | AGAAAUCCGUCUUUCAUUG |
| TTAAACT | Cas12i2 | sequence - DNA- | ACGGUUAAACUCUCCAUGG |
| CTCCATG | or | binding sequence - | ACCAGAAAAGACUUUUUAA |
| GACCAG | C_MMLV_Variant | reverse transcription | ACUCUCCACGAGCCAGGCUC |
| (SEQ ID | Cas12i2 | template sequence (S) - | AUCCAGCUUCCCAAACAAAG |
| NO: 38) | | PBS (60 nt) | CCCCCAAGAAGGGGGGGCAC |
| | | | UCAGGACUCUCUCCAAGAG |
| | | | A (SEQ ID NO: 95) |
| VEGFA_T5 | N_MMLV_Variant | nuclease binding | AGAAAUCCGUCUUUCAUUG |
| (SEQ ID | Cas12i2 | sequence - DNA- | ACGGUUAAACUCUCCAUGG |
| NO: 38) | or | binding sequence - | ACCAGAAAAGACUUUUUAA |
| | C_MMLV_Variant | reverse transcription | ACUCUCCACGAGCCAGGCUC |
| | Cas12i2 | template sequence (S) - | AUCCAGCUUCCCAAACAAAG |
| | | PBS (30 nt) | CCCCCAAGAA (SEQ ID NO: |
| | | | 96) |
| VEGFA_T5 | N_MMLV_Variant | nuclease binding | AGAAAUCCGUCUUUCAUUG |
| (SEQ ID | Cas12i2 | sequence - DNA- | ACGGUUAAACUCUCCAUGG |
| NO: 38) | or | binding sequence - | ACCAGGCACCCCCAUAAGGG |
| | C_MMLV_Variant | reverse transcription | GGAAAAGACUUUUUAAACU |
| | Cas12i2 | template sequence (H) - | CUCCACGAGCCAGGCUCAUC |
| | | PBS (13 nt) | CAGCUUCCCA (SEQ ID NO: |
| | | | 97) |
| VEGFA_T5 | N_MMLV_Variant | nuclease binding | AGAAAUCCGUCUUUCAUUG |
| (SEQ ID | Cas12i2 | sequence - DNA- | ACGGUUAAACUCUCCAUGG |
| NO: 38) | or | binding sequence - | ACCAGAAAAGACUGUUUAA |
| | C_MMLV_Variant | reverse transcription | ACUCUCCACGAGCCAGGCUC |
| | Cas12i2 | template sequence (S) - | AUCCAGCUUCCCA (SEQ ID |
| | | PBS (13 nt) | NO: 98) |
| VEGFA_T5 | N_MMLV_Variant | nuclease binding | AGAAAUCCGUCUUUCAUUG |
| (SEQ ID | Cas12i2 | sequence - DNA- | ACGGUUAAACUCUCCAUGG |
| NO: 38) | or | binding sequence - | ACCAGAAAAGACUGUUUAA |
| | C_MMLV_Variant | reverse transcription | ACUCUCCAAGCGUGGACCAG |
| | Cas12i2 | template sequence (I) - | GCUCAUCCAGCUUCCCA |
| | | PBS (13 nt) | (SEQ ID NO: 99) |
| VEGFA_T5 | N_MMLV_Variant | nuclease binding | AGAAAUCCGUCUUUCAUUG |
| (SEQ ID | Cas12i2 | sequence - DNA- | ACGGUUAAACUCUCCAUGG |
| NO: 38) | or | binding sequence - | ACCAGAAAAGACUGUUUAA |
| | C_MMLV_Variant | reverse transcription | ACUCUCCACCAGGCUCAUCC |
| | Cas12i2 | template sequence (D) - | AGCUUCCCA (SEQ ID NO: |
| | | PBS (13 nt) | 100) |

TABLE 10
RNA GUIDE CONTROL SEQUENCES.

| | Cas or Cas-RT | |
|---|---|---|
| Target | Fusion | RNA Guide Sequence |
| AAVS1_ | VaCas12i2, | AGAAAUCCGUCUUUCAUUGACGGGUA |
| T6 | N_MMLV_Variant | GCCUCUCCCGCUCUGGU (SEQ ID |
| | Cas12i2, and | NO: 110) |
| | C_MMLV_Variant | |
| | Cas12i2 | |
| AAVS1_ | Cas12i2 variant, | AGAAAUCCGUCUUUCAUUGACGGGGG |
| T7 | N_MMLV_Cas12i2 | AAGUGGUUGGUCAGCAU (SEQ ID |
| | variant, and | NO: 112) |
| | C_MMLV_Cas12i2 | |
| | variant | |
| EMX1- | VaCas12i2, | AGAAAUCCGUCUUUCAUUGACGGGAG |
| T6 | N_MMLV_Variant | CCAGUGUUGCUAGUCAA (SEQ ID |
| | Cas12i2, and | NO: 114) |
| | C_MMLV_Variant | |
| | Cas12i2 | |
| VEGFA_ | VaCas12i2, | AGAAAUCCGUCUUUCAUUGACGGAAU |
| T2 | N_MMLV_Variant | CCUCCACCAGUCAUGGU (SEQ ID |
| | Cas12i2, and | NO: 116) |
| | C_MMLV_Variant | |
| | Cas12i2 | |
| VEGFA_ | Cas12i2 variant, | AGAAAUCCGUCUUUCAUUGACGGUUA |
| T5 | N_MMLV_Cas12i2 | AACUCUCCAUGGACCAG (SEQ ID |
| | variant, and | NO: 118) |
| | C_MMLV_Cas12i2 | |
| | variant | |

Approximately 16 hours prior to transfection, 25,000 HEK293T cells in DMEM/10% FBS+Pen/Strep were plated into each well of a 96-well plate. On the day of transfection, the cells were 70-90% confluent. For each well to be transfected, a mixture of Lipofectamine™ 2000 (Thermo Fisher) and Opti-MEM™ media (Thermo Fisher) was prepared and then incubated at room temperature for 5-20 minutes (Solution 1). After incubation, the Lipofectamine™:OptiMEM™ mixture was added to a separate mixture containing variant Cas12i2-RT fusion working solution, RNA working solution and OptiMEM™ media (Solution 2). The solution 1 and solution 2 mixtures were mixed by pipetting up and down and then incubated at room temperature for 25 minutes. Following incubation, Solution 1 and Solution 2 mixture were added dropwise to each well of a 96 well plate containing the cells. 72 hours post transfection, cells were trypsinized by adding TrypLE™ (ThermoFisher) to the center of each well and incubated for approximately 5 minutes. Growth media was then added to each well and mixed to resuspend cells. The cells were then spun down at 400 g for 10 minutes, and the supernatant was discarded. QuickExtract™ buffer (Lucigen) was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

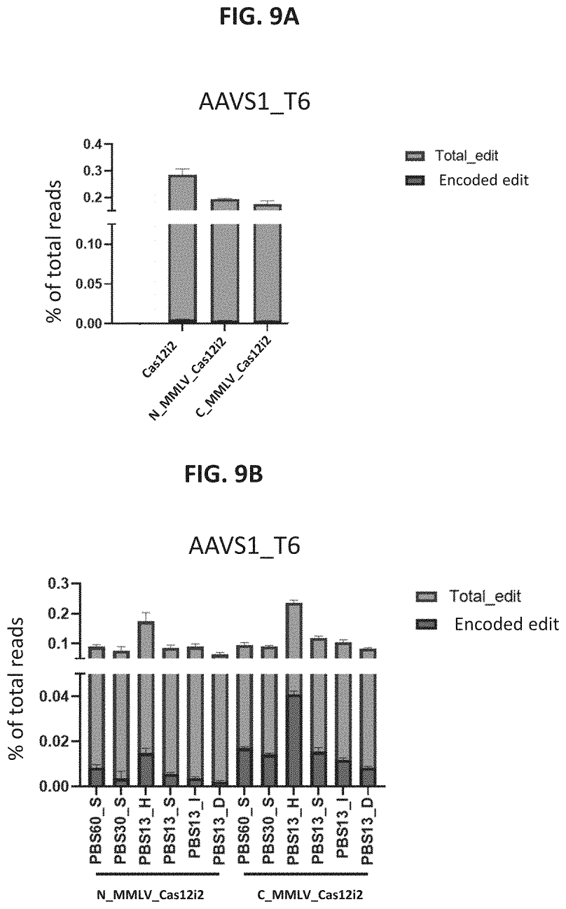

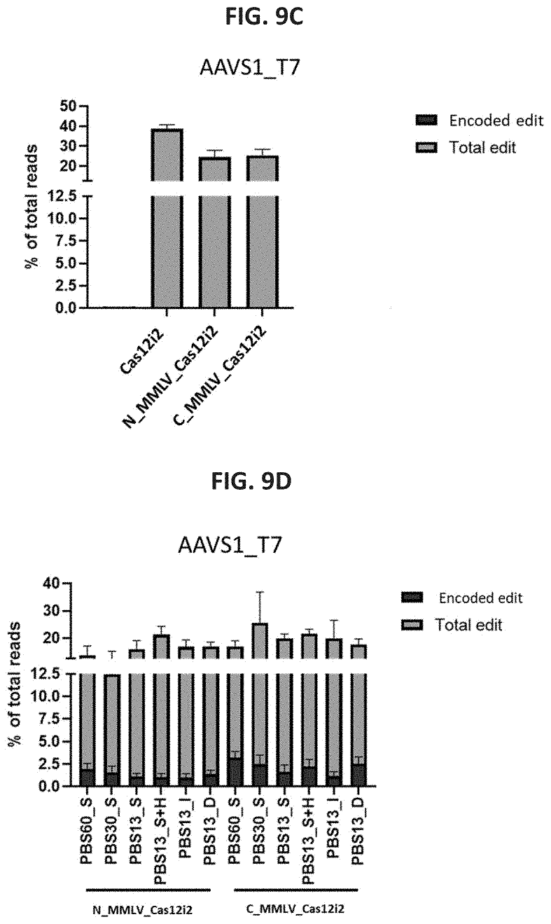

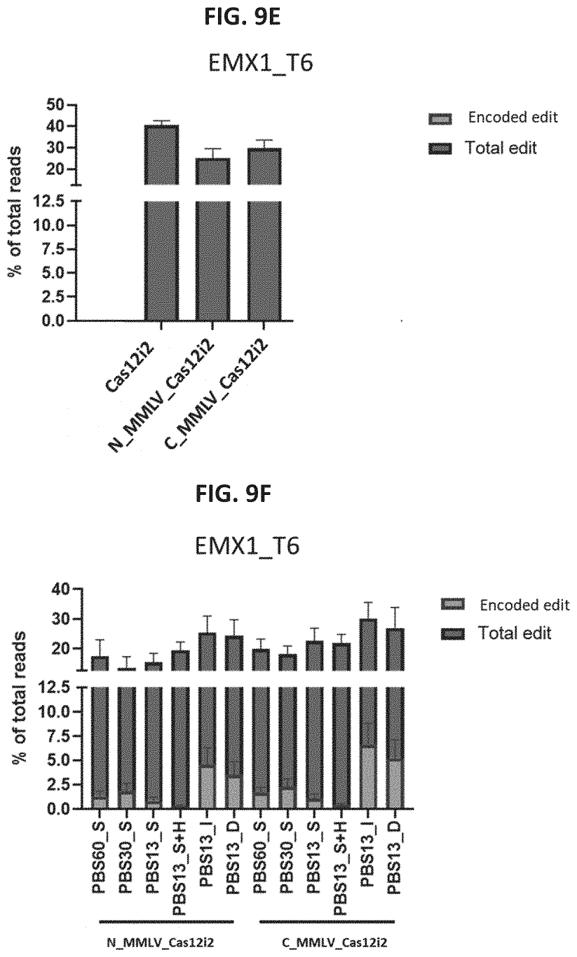

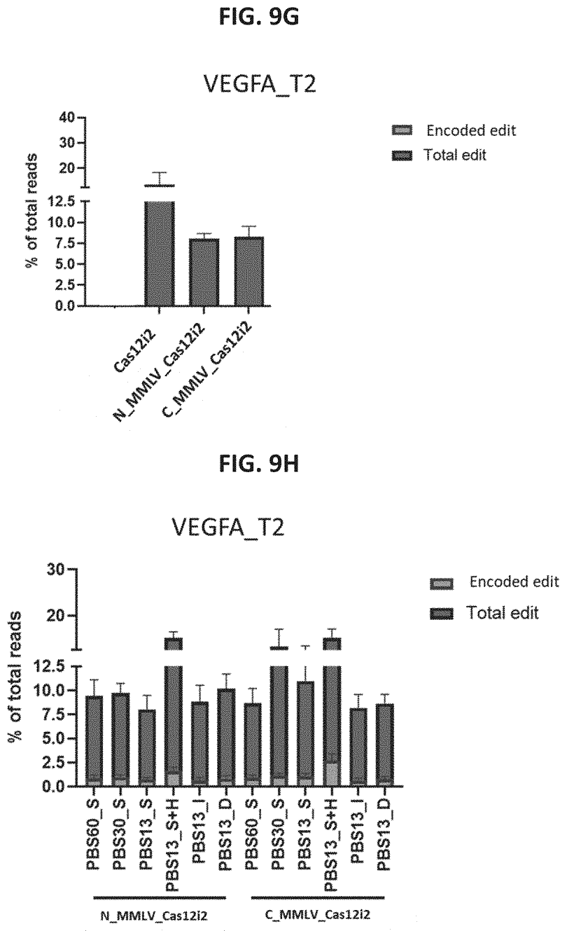

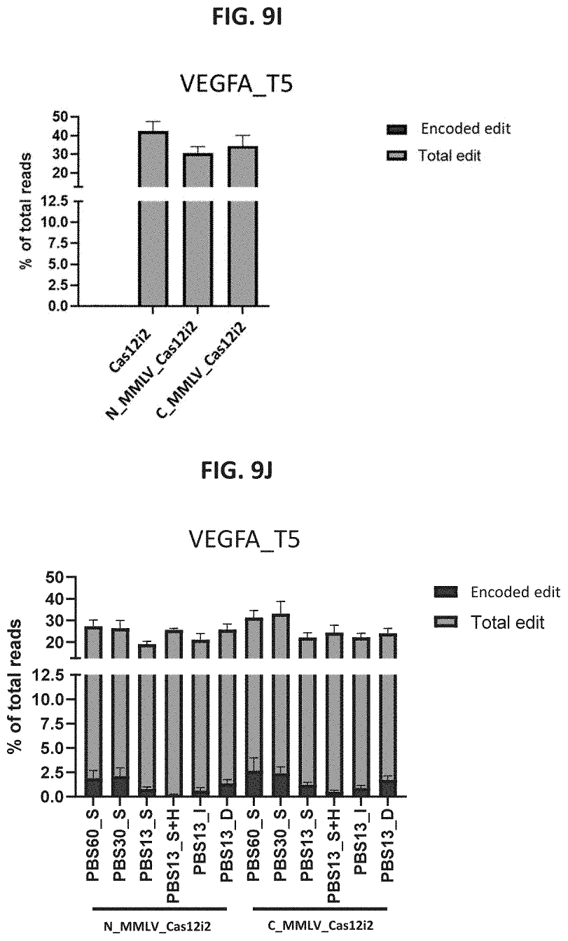

FIG. 9A and FIG. 9B show activity by variant Cas12i2 on AAVS1_T6, FIG. 9C and FIG. 9D show activity by variant Cas12i2 on AAVS1_T7, FIG. 9E and FIG. 9F show activity by variant Cas12i2 on EMX1_T6, FIG. 9G and FIG. 9H show activity by variant Cas12i2 on VEGFA_T2, and FIG. 9I and FIG. 9J show activity by variant Cas12i2 on VEGFA_T5. Percentage of NGS reads is shown on the y-axis, total edits are shown as in light grey bars, and encoded edits are shown as in dark grey bars. The data shown is an average of two bioreplicates, each of which had three technical replicates. As shown in FIG. 9A, FIG. 9C, FIG. 9E, FIG. 9G, and FIG. 9I, variant Cas12i2 and variant Cas12i2-RT fusions were active nucleases in the presence of RNA guides targeting either AAVS1_T6, AAVS1_T7, EMX1_T6, VEGFA_T2, or VEGFA_T5.

As shown in FIG. 9B, FIG. 9D, FIG. 9F, FIG. 9H, and FIG. 9J, variant Cas12i2-RT fusions in the presence of RNA guide-RT donor RNA fusion sequences were capable of introducing the encoded substitutions, insertions and deletions into AAVS1_T6, AAVS1_T7, EMX1_T6, VEGFA_T2, or VEGFA_T5. Activity was observed with PBS lengths of 13, 30, and 60 nucleotides. Editing by C-terminal MMLV RT fusions exceeded that by N-terminal MMLV RT fusions with variant Cas12i2. Editing with variant Cas12i2 ranged from about 1-5%.

This Example shows that specific edits were incorporated into the selected mammalian genomic sites using editing template RNAs and a Cas12i2-RT fusion.

### Example 2—Editing of Target Strand in Mammalian Cells Using End Protected Editing Template RNAs

This Example describes target strand editing of mammalian genes (e.g., using an editing template RNA that binds the non-PAM strand of selected mammalian genes).

Variant Cas12i2 of SEQ ID NO: 4 and the variant Cas12i2-RT fusion of SEQ ID NO: 25 were each cloned into pcda3.1 backbones (Invitrogen). A working solution of plasmids for expression of RT fusion with variant Cas12i2 were prepared in water (variant Cas12i2-RT fusion working solution).

Various RNA guide-RT donor RNA fusion configurations were tested, as shown in Table 11 and depicted in FIG. 12A and FIG. 12B. A reverse transcription template sequence and PBS were fused to either the 5′ end or the 3′ end of the RNA guide. An additional DR-spacer sequence was added on either the 5′ or 3′ end. The spacer sequence used for end protection was non-human targeting (i.e., it did not target any sequence in the human genome). The sequences of the RNA guide-RT donor RNA fusions are shown in Table 12; the desired edit encoded in the RT donor is show in lowercase letters. Sequences of RNA guides only, which were used as controls, are shown in Table 13. The RNA guide-RT donor RNA fusions or RNA guides were cloned into a plasmid backbone with a U6 promoter and maxi-prepped. A working solution of each plasmid expressing an RNA guide/RT donor RNA plasmid (or RNA guide) was prepared in water (editing template RNA working solution).

TABLE 11
RNA GUIDE-RT DONOR RNA FUSION DESIGNS

| Configuration | Description |
|---|---|
| DR - spacer - reverse | reverse transcription template sequence: |
| transcription template | 34 nucleotides |
| sequence - PBS | PBS: 13, 30, or 60 nucleotides |
| DR - spacer - reverse | reverse transcription template sequence: |
| transcription template | 34 nucleotides |
| sequence - PBS - end protection | PBS: 13, 30, or 60 nucleotides |
| | End protection: DR-Spacer(non human) |
| Reverse transcription template | reverse transcription template sequence: |
| sequence - PBS - DR - spacer | 34 nucleotides |
| | PBS: 13, 30, or 60 nucleotides |
| End protection - reverse | End protection: DR-Spacer(non human) |
| transcription template | reverse transcription template sequence: |
| sequence - PBS - DR - spacer | 34 or 40 nucleotides |
| | PBS: 13, 30, or 60 nucleotides |
| DR - spacer | Control (No RT donor RNA) |

TABLE 12
RNA GUIDE-RT DONOR FUSION SEQUENCES

| | RNA Guide-RT donor RNA | RNA Guide-RT donor RNA |
|---|---|---|
| Target | Description | Sequences |
| AAVS1_T7 | nuclease binding sequence - | AGAAAUCCGUCUUUCAU |
| GGGAAGTGGTTGGTCAG | DNA-binding sequence - | UGACGGGGGAAGUGGU |
| CAT | reverse transcription template | UGGUCAGCAUCAGCUAC |
| (SEQ ID NO: 32) | sequence (34 nt) - PBS (13 | UaUGGGAAGUGGUUGcag |
| | nt) | UGCAUGGAUUAUAGCC |
| | | GAAGGC (SEQ ID NO: |
| | | 123) |
| AAVS1_T7 | nuclease binding sequence - | AGAAAUCCGUCUUUCAU |
| (SEQ ID NO: 32) | DNA-binding sequence - | UGACGGGGGAAGUGGU |
| | reverse transcription template | UGGUCAGCAUCAGCUAC |
| | sequence (34 nt) - PBS (30 | UaUGGGAAGUGGUUGcag |
| | nt) | UGCAUGGAUUAUAGCC |
| | | GAAGGCCCCAGCUUUGC |
| | | CUUGUU (SEQ ID NO: |
| | | 124) |
| AAVS1_T7 | nuclease binding sequence - | AGAAAUCCGUCUUUCAU |
| (SEQ ID NO: 32) | DNA-binding sequence - | UGACGGGGGAAGUGGU |
| | reverse transcription template | UGGUCAGCAUCAGCUAC |
| | sequence (34 nt) - PBS (60 | UaUGGGAAGUGGUUGcag |
| | nt) | UGCAUGGAUUAUAGCC |
| | | GAAGGCCCCAGCUUUGC |
| | | CUUGUUCUAGCAGUUCC |
| | | ACUCCUGGGCAGCCCGA |
| | | GA (SEQ ID NO: 125) |
| AAVS1_T7 | nuclease binding sequence - | AGAAAUCCGUCUUUCAU |
| (SEQ ID NO: 32) | DNA-binding sequence - | UGACGGGGGAAGUGGU |
| | reverse transcription template | UGGUCAGCAUCAGCUAC |
| | sequence (34 nt) - PBS (13 | UaUGGGAAGUGGUUGcag |
| | nt) - end protection | UGCAUGGAUUAUAGCC |
| | | GAAGGCAGAAAUCCGUC |
| | | UUUCAUUGACGGGCACA |
| | | CGACGAUGUAAUCGC |
| | | (SEQ ID NO: 126) |
| AAVS1_T7 | nuclease binding sequence - | AGAAAUCCGUCUUUCAU |
| (SEQ ID NO: 32) | DNA-binding sequence - | UGACGGGGGAAGUGGU |
| | reverse transcription template | UGGUCAGCAUCAGCUAC |
| | sequence (34 nt) - PBS (30 | UaUGGGAAGUGGUUGcag |
| | nt) - end protection | UGCAUGGAUUAUAGCC |
| | | GAAGGCCCCAGCUUUGC |
| | | CUUGUUAGAAAUCCGUC |
| | | UUUCAUUGACGGGCACA |
| | | CGACGAUGUAAUCGC |
| | | (SEQ ID NO: 127) |
| AAVS1_T7 | nuclease binding sequence - | AGAAAUCCGUCUUUCAU |
| (SEQ ID NO: 32) | DNA-binding sequence - | UGACGGGGGAAGUGGU |
| | reverse transcription template | UGGUCAGCAUCAGCUAC |
| | sequence (34 nt) - PBS (60 | UaUGGGAAGUGGUUGcag |
| | nt) - end protection | UGCAUGGAUUAUAGCC |
| | | GAAGGCCCCAGCUUUGC |
| | | CUUGUUCUAGCAGUUCC |
| | | ACUCCUGGGCAGCCCGA |
| | | GAAGAAAUCCGUCUUUC |
| | | AUUGACGGGCACACGAC |
| | | GAUGUAAUCGC (SEQ ID |
| | | NO: 128) |
| AAVS1_T7 | Reverse transcription | CAGCUACUaUGGGAAGU |
| (SEQ ID NO: 32) | template sequence (34 nt) - | GGUUGcagUGCAUGGAU |
| | PBS (13 nt) - nuclease | UAUAGCCGAAGGCAGA |
| | binding sequence - DNA- | AAUCCGUCUUUCAUUGA |
| | binding sequence | CGGGGGAAGUGGUUGG |
| | | UCAGCAU (SEQ ID NO: |
| | | 129) |
| AAVS1_T7 | Reverse transcription | CAGCUACUaUGGGAAGU |
| (SEQ ID NO: 32) | template sequence (34 nt) - | GGUUGcagUGCAUGGAU |
| | PBS (30 nt) - nuclease | UAUAGCCGAAGGCCCCA |
| | binding sequence - DNA- | GCUUUGCCUUGUUAGA |
| | binding sequence | AAUCCGUCUUUCAUUGA |
| | | CGGGGGAAGUGGUUGG |
| | | UCAGCAU (SEQ ID NO: |
| | | 130) |
| AAVS1_T7 | Reverse transcription | CAGCUACUaUGGGAAGU |
| (SEQ ID NO: 32) | template sequence (34 nt) - | GGUUGcagUGCAUGGAU |
| | PBS (60 nt) - nuclease | UAUAGCCGAAGGCCCCA |
| | binding sequence - DNA- | GCUUUGCCUUGUUCUAG |
| | binding sequence | CAGUUCCACUCCUGGGC |
| | | AGCCCGAGAAGAAAUCC |
| | | GUCUUUCAUUGACGGG |
| | | GGAAGUGGUUGGUCAG |
| | | CAU (SEQ ID NO: 131) |
| AAVS1_T7 | End protection - reverse | AGAAAUCCGUCUUUCAU |
| (SEQ ID NO: 32) | transcription template | UGACGGGCACACGACGA |
| | sequence (34 nt) - PBS (13 | UGUAAUCGCCAGCUACU |
| | nt) - nuclease binding | aUGGGAAGUGGUUGcagU |
| | sequence - DNA-binding | GCAUGGAUUAUAGCCG |
| | sequence | AAGGCAGAAAUCCGUCU |
| | | UUCAUUGACGGGGGAA |
| | | GUGGUUGGUCAGCAU |
| | | (SEQ ID NO: 132) |
| AAVS1_T7 | End protection - reverse | AGAAAUCCGUCUUUCAU |
| (SEQ ID NO: 32) | transcription template | UGACGGGCACACGACGA |
| | sequence (34 nt) - PBS (30 | UGUAAUCGCCAGCUACU |
| | nt) - nuclease binding | aUGGGAAGUGGUUGcagU |
| | sequence - DNA-binding | GCAUGGAUUAUAGCCG |
| | sequence | AAGGCCCCAGCUUUGCC |
| | | UUGUUAGAAAUCCGUC |
| | | UUUCAUUGACGGGGGA |
| | | AGUGGUUGGUCAGCAU |
| | | (SEQ ID NO: 133) |
| AAVS1_T7 | End protection - reverse | AGAAAUCCGUCUUUCAU |
| (SEQ ID NO: 32) | transcription template | UGACGGGCACACGACGA |
| | sequence (34 nt) - PBS (60 | UGUAAUCGCCAGCUACU |
| | nt) - nuclease binding | aUGGGAAGUGGUUGcagU |
| | sequence - DNA-binding | GCAUGGAUUAUAGCCG |
| | sequence | AAGGCCCCAGCUUUGCC |
| | | UUGUUCUAGCAGUUCCA |
| | | CUCCUGGGCAGCCCGAG |
| | | AAGAAAUCCGUCUUUCA |
| | | UUGACGGGGGAAGUGG |
| | | UUGGUCAGCAU (SEQ ID |
| | | NO: 134) |
| AAVS1_T7 | End protection - reverse | AGAAAUCCGUCUUUCAU |
| (SEQ ID NO: 32) | transcription template | UGACGGGCACACGACGA |
| | sequence (40 nt) - PBS (13 | UGUAAUCGCUGAUUCCA |
| | nt) - nuclease binding | GCUACUaUGGGAAGUGG |
| | sequence - DNA-binding | UUGcagUGCAUGGAUUA |
| | sequence | UAGCCGAAGGCAGAAA |
| | | UCCGUCUUUCAUUGACG |
| | | GGGGAAGUGGUUGGUC |
| | | AGCAU (SEQ ID NO: 135) |
| AAVS1_T7 | End protection - reverse | AGAAAUCCGUCUUUCAU |
| (SEQ ID NO: 32) | transcription template | UGACGGGCACACGACGA |
| | sequence (40 nt) - PBS (30 | UGUAAUCGCUGAUUCCA |
| | nt) - nuclease binding | GCUACUaUGGGAAGUGG |
| | sequence - DNA-binding | UUGcagUGCAUGGAUUA |
| | sequence | UAGCCGAAGGCCCCAGC |
| | | UUUGCCUUGUUAGAAA |
| | | UCCGUCUUUCAUUGACG |
| | | GGGGAAGUGGUUGGUC |
| | | AGCAU (SEQ ID NO: 136) |
| AAVS1_T7 | End protection - reverse | AGAAAUCCGUCUUUCAU |
| (SEQ ID NO: 32) | transcription template | UGACGGGCACACGACGA |
| | sequence (40 nt) - PBS (60 | UGUAAUCGCUGAUUCCA |
| | nt) - nuclease binding | GCUACUaUGGGAAGUGG |
| | sequence - DNA-binding | UUGcagUGCAUGGAUUA |
| | sequence | UAGCCGAAGGCCCCAGC |
| | | UUUGCCUUGUUCUAGCA |
| | | GUUCCACUCCUGGGCAG |
| | | CCCGAGAAGAAAUCCGU |
| | | CUUUCAUUGACGGGGG |
| | | AAGUGGUUGGUCAGCA |
| | | U (SEQ ID NO: 137) |
| EMX1_T6 | nuclease binding sequence - | AGAAAUCCGUCUUUCAU |
| GAGCCAGTGTTGCTAGT | DNA-binding sequence - | UGACGGGAGCCAGUGU |
| CAA | reverse transcription template | UGCUAGUCAAUUCCUUC |
| (SEQ ID NO: 34) | sequence (34 nt) - PBS (13 | UaUGAGCCAGUGUUGga |
| | nt) | UcUCAAGGGCAGCAUGC |
| | | UGGGCC (SEQ ID NO: 138) |
| EMX1_T6 | nuclease binding sequence - | AGAAAUCCGUCUUUCAU |
| (SEQ ID NO: 34) | DNA-binding sequence - | UGACGGGAGCCAGUGU |
| | reverse transcription template | UGCUAGUCAAUUCCUUC |
| | sequence (34 nt) - PBS (30 | UaUGAGCCAGUGUUGga |
| | nt) | UcUCAAGGGCAGCAUGC |
| | | UGGGCCCGUCCCACUAC |
| | | AGGCCA (SEQ ID NO: 139) |
| EMX1_T6 | nuclease binding sequence - | AGAAAUCCGUCUUUCAU |
| (SEQ ID NO: 34) | DNA-binding sequence - | UGACGGGAGCCAGUGU |
| | reverse transcription template | UGCUAGUCAAUUCCUUC |
| | sequence (34 nt) - PBS (60 | UaUGAGCCAGUGUUGga |
| | nt) | UcUCAAGGGCAGCAUGC |
| | | UGGGCCCGUCCCACUAC |
| | | AGGCCAAUGUGACCGUC |
| | | AGUCUCCUUCCUGAAGG |
| | | AC (SEQ ID NO: 140) |
| EMX1_T6 | nuclease binding sequence - | AGAAAUCCGUCUUUCAU |
| (SEQ ID NO: 34) | DNA-binding sequence - | UGACGGGAGCCAGUGU |
| | reverse transcription template | UGCUAGUCAAUUCCUUC |
| | sequence (34 nt) - PBS (13 | UaUGAGCCAGUGUUGga |
| | nt) - end protection | UcUCAAGGGCAGCAUGC |
| | | UGGGCCAGAAAUCCGUC |
| | | UUUCAUUGACGGGCACA |
| | | CGACGAUGUAAUCGC |
| | | (SEQ ID NO: 141) |
| EMX1_T6 | nuclease binding sequence - | AGAAAUCCGUCUUUCAU |
| (SEQ ID NO: 34) | DNA-binding sequence - | UGACGGGAGCCAGUGU |
| | reverse transcription template | UGCUAGUCAAUUCCUUC |
| | sequence (34 nt) - PBS (30 | UaUGAGCCAGUGUUGga |
| | nt) - end protection | UcUCAAGGGCAGCAUGC |
| | | UGGGCCCGUCCCACUAC |
| | | AGGCCAAGAAAUCCGUC |
| | | UUUCAUUGACGGGCACA |
| | | CGACGAUGUAAUCGC |
| | | (SEQ ID NO: 142) |
| EMX1_T6 | nuclease binding sequence - | AGAAAUCCGUCUUUCAU |
| (SEQ ID NO: 34) | DNA-binding sequence - | UGACGGGAGCCAGUGU |
| | reverse transcription template | UGCUAGUCAAUUCCUUC |
| | sequence (34 nt) - PBS (60 | UaUGAGCCAGUGUUGga |
| | nt) - end protection | UcUCAAGGGCAGCAUGC |
| | | UGGGCCCGUCCCACUAC |
| | | AGGCCAAUGUGACCGUC |
| | | AGUCUCCUUCCUGAAGG |
| | | ACAGAAAUCCGUCUUUC |
| | | AUUGACGGGCACACGAC |
| | | GAUGUAAUCGC (SEQ ID |
| | | NO: 143) |
| EMX1_T6 | Reverse transcription | UUCCUUCUaUGAGCCAG |
| (SEQ ID NO: 34) | template sequence (34 nt) - | UGUUGgaUcUCAAGGGC |
| | PBS (13 nt) - nuclease | AGCAUGCUGGGCCAGAA |
| | binding sequence - DNA- | AUCCGUCUUUCAUUGAC |
| | binding sequence | GGGAGCCAGUGUUGCU |
| | | AGUCAA (SEQ ID NO: |
| | | 144) |
| EMX1_T6 | Reverse transcription | UUCCUUCUaUGAGCCAG |
| (SEQ ID NO: 34) | template sequence (34 nt) - | UGUUGgaUcUCAAGGGC |
| | PBS (30 nt) - nuclease | AGCAUGCUGGGCCCGUC |
| | binding sequence - DNA- | CCACUACAGGCCAAGAA |
| | binding sequence | AUCCGUCUUUCAUUGAC |
| | | GGGAGCCAGUGUUGCU |
| | | AGUCAA (SEQ ID NO: |
| | | 145) |
| EMX1_T6 | Reverse transcription | UUCCUUCUaUGAGCCAG |
| (SEQ ID NO: 34) | template sequence (34 nt) - | UGUUGgaUcUCAAGGGC |
| | PBS (60 nt) - nuclease | AGCAUGCUGGGCCCGUC |
| | binding sequence - DNA- | CCACUACAGGCCAAUGU |
| | binding sequence | GACCGUCAGUCUCCUUC |
| | | CUGAAGGACAGAAAUCC |
| | | GUCUUUCAUUGACGGG |
| | | AGCCAGUGUUGCUAGUC |
| | | AA (SEQ ID NO: 146) |
| EMX1_T6 | End protection - reverse | AGAAAUCCGUCUUUCAU |
| (SEQ ID NO: 34) | transcription template | UGACGGGCACACGACGA |
| | sequence (34 nt) - PBS (13 | UGUAAUCGCUUCCUUCU |
| | nt) - nuclease binding | aUGAGCCAGUGUUGgaUc |
| | sequence - DNA-binding | UCAAGGGCAGCAUGCUG |
| | sequence | GGCCAGAAAUCCGUCUU |
| | | UCAUUGACGGGAGCCAG |
| | | UGUUGCUAGUCAA (SEQ |
| | | ID NO: 147) |
| EMX1_T6 | End protection - reverse | AGAAAUCCGUCUUUCAU |
| (SEQ ID NO: 34) | transcription template | UGACGGGCACACGACGA |
| | sequence (34 nt) - PBS (30 | UGUAAUCGCUUCCUUCU |
| | nt) - nuclease binding | aUGAGCCAGUGUUGgaUc |
| | sequence - DNA-binding | UCAAGGGCAGCAUGCUG |
| | sequence | GGCCCGUCCCACUACAG |
| | | GCCAAGAAAUCCGUCUU |
| | | UCAUUGACGGGAGCCAG |
| | | UGUUGCUAGUCAA (SEQ |
| | | ID NO: 148) |
| EMX1_T6 | End protection - reverse | AGAAAUCCGUCUUUCAU |
| (SEQ ID NO: 34) | transcription template | UGACGGGCACACGACGA |
| | sequence (34 nt) - PBS (60 | UGUAAUCGCUUCCUUCU |
| | nt) - nuclease binding | aUGAGCCAGUGUUGgaUc |
| | sequence - DNA-binding | UCAAGGGCAGCAUGCUG |
| | sequence | GGCCCGUCCCACUACAG |
| | | GCCAAUGUGACCGUCAG |
| | | UCUCCUUCCUGAAGGAC |
| | | AGAAAUCCGUCUUUCAU |
| | | UGACGGGAGCCAGUGU |
| | | UGCUAGUCAA (SEQ ID |
| | | NO: 149) |
| EMX1_T6 | End protection - reverse | AGAAAUCCGUCUUUCAU |
| (SEQ ID NO: 34) | transcription template | UGACGGGCACACGACGA |
| | sequence (40 nt) - PBS (13 | UGUAAUCGCAAAGCUU |
| | nt) - nuclease binding | UCCUUCUaUGAGCCAGU |
| | sequence - DNA-binding | GUUGgaUcUCAAGGGCA |
| | sequence | GCAUGCUGGGCCAGAAA |
| | | UCCGUCUUUCAUUGACG |
| | | GGAGCCAGUGUUGCUA |
| | | GUCAA (SEQ ID NO: 150) |
| EMX1_T6 | End protection - reverse | AGAAAUCCGUCUUUCAU |
| (SEQ ID NO: 34) | transcription template | UGACGGGCACACGACGA |
| | sequence (40 nt) - PBS (30 | UGUAAUCGCAAAGCUU |
| | nt) - nuclease binding | UCCUUCUaUGAGCCAGU |
| | sequence - DNA-binding | GUUGgaUcUCAAGGGCA |
| | sequence | GCAUGCUGGGCCCGUCC |
| | | CACUACAGGCCAAGAAA |
| | | UCCGUCUUUCAUUGACG |
| | | GGAGCCAGUGUUGCUA |
| | | GUCAA (SEQ ID NO: 151) |
| EMX1_T6 | End protection - reverse | AGAAAUCCGUCUUUCAU |
| (SEQ ID NO: 34) | transcription template | UGACGGGCACACGACGA |
| | sequence (40 nt) - PBS (60 | UGUAAUCGCAAAGCUU |
| | nt) - nuclease binding | UCCUUCUaUGAGCCAGU |
| | sequence - DNA-binding | GUUGgaUcUCAAGGGCA |
| | sequence | GCAUGCUGGGCCCGUCC |
| | | CACUACAGGCCAAUGUG |
| | | ACCGUCAGUCUCCUUCC |
| | | UGAAGGACAGAAAUCC |
| | | GUCUUUCAUUGACGGG |
| | | AGCCAGUGUUGCUAGUC |
| | | AA (SEQ ID NO: 152) |
| VEGFA_T2 | nuclease binding sequence - | AGAAAUCCGUCUUUCAU |
| AATCCTCCACCAGTCAT | DNA-binding sequence - | UGACGGAAUCCUCCACC |
| GGT | reverse transcription template | AGUCAUGGUAGAUACC |
| (SEQ ID NO: 36) | sequence (34 nt) - PBS (13 | UaUAAUCCUCCACCAcag |
| | nt) | UUGGUGACAACCCCAAG |
| | | CAGCC (SEQ ID NO: 153) |
| VEGFA_T2 | nuclease binding sequence - | AGAAAUCCGUCUUUCAU |
| (SEQ ID NO: 36) | DNA-binding sequence - | UGACGGAAUCCUCCACC |
| | reverse transcription template | AGUCAUGGUAGAUACC |
| | sequence (34 nt) - PBS (30 | UaUAAUCCUCCACCAcag |
| | nt) | UUGGUGACAACCCCAAG |
| | | CAGCCCACACAUUcUCA |
| | | AGUGC (SEQ ID NO: 154) |
| VEGFA_T2 | nuclease binding sequence - | AGAAAUCCGUCUUUCAU |
| (SEQ ID NO: 36) | DNA-binding sequence - | UGACGGAAUCCUCCACC |
| | reverse transcription template | AGUCAUGGUAGAUACC |
| | sequence (34 nt) - PBS (60 | UaUAAUCCUCCACCAcag |
| | nt) | UUGGUGACAACCCCAAG |
| | | CAGCCCACACAUUcUCA |
| | | AGUGCCCCCAGGAUGCG |
| | | UGGAGGGAGGGGUCUG |
| | | UG (SEQ ID NO: 155) |
| VEGFA_T2 | nuclease binding sequence - | AGAAAUCCGUCUUUCAU |
| (SEQ ID NO: 36) | DNA-binding sequence - | UGACGGAAUCCUCCACC |
| | reverse transcription template | AGUCAUGGUAGAUACC |
| | sequence (34 nt) - PBS (13 | UaUAAUCCUCCACCAcag |
| | nt) - end protection | UUGGUGACAACCCCAAG |
| | | CAGCCAGAAAUCCGUCU |
| | | UUCAUUGACGGGCACAC |
| | | GACGAUGUAAUCGC |
| | | (SEQ ID NO: 156) |
| VEGFA_T2 | nuclease binding sequence - | AGAAAUCCGUCUUUCAU |
| (SEQ ID NO: 36) | DNA-binding sequence - | UGACGGAAUCCUCCACC |
| | reverse transcription template | AGUCAUGGUAGAUACC |
| | sequence (34 nt) - PBS (30 | UaUAAUCCUCCACCAcag |
| | nt) - end protection | UUGGUGACAACCCCAAG |
| | | CAGCCCACACAUUcUCA |
| | | AGUGCAGAAAUCCGUCU |
| | | UUCAUUGACGGGCACAC |
| | | GACGAUGUAAUCGC |
| | | (SEQ ID NO: 157) |
| VEGFA_T2 | nuclease binding sequence - | AGAAAUCCGUCUUUCAU |
| (SEQ ID NO: 36) | DNA-binding sequence - | UGACGGAAUCCUCCACC |
| | reverse transcription template | AGUCAUGGUAGAUACC |
| | sequence (34 nt) - PBS (60 | UaUAAUCCUCCACCAcag |
| | nt) - end protection | UUGGUGACAACCCCAAG |
| | | CAGCCCACACAUUcUCA |
| | | AGUGCCCCCAGGAUGCG |
| | | UGGAGGGAGGGGUCUG |
| | | UGAGAAAUCCGUCUUUC |
| | | AUUGACGGGCACACGAC |
| | | GAUGUAAUCGC (SEQ ID |
| | | NO: 158) |
| VEGFA_T2 | Reverse transcription | AGAUACCUaUAAUCCUC |
| (SEQ ID NO: 36) | template sequence (34 nt) - | CACCAcagUUGGUGACAA |
| | PBS (13 nt) - nuclease | CCCCAAGCAGCCAGAAA |
| | binding sequence - DNA- | UCCGUCUUUCAUUGACG |
| | binding sequence | GAAUCCUCCACCAGUCA |
| | | UGGU (SEQ ID NO: 159) |
| VEGFA_T2 | Reverse transcription | AGAUACCUaUAAUCCUC |
| (SEQ ID NO: 36) | template sequence (34 nt) - | CACCAcagUUGGUGACAA |
| | PBS (30 nt) - nuclease | CCCCAAGCAGCCCACAC |
| | binding sequence - DNA- | AUUcUCAAGUGCAGAAA |
| | binding sequence | UCCGUCUUUCAUUGACG |
| | | GAAUCCUCCACCAGUCA |
| | | UGGU (SEQ ID NO: 160) |
| VEGFA_T2 | Reverse transcription | AGAUACCUaUAAUCCUC |
| (SEQ ID NO: 36) | template sequence (34 nt) - | CACCAcagUUGGUGACAA |
| | PBS (60 nt) - nuclease | CCCCAAGCAGCCCACAC |
| | binding sequence - DNA- | AUUcUCAAGUGCCCCCA |
| | binding sequence | GGAUGCGUGGAGGGAG |
| | | GGGUCUGUGAGAAAUC |
| | | CGUCUUUCAUUGACGGA |
| | | AUCCUCCACCAGUCAUG |
| | | GU (SEQ ID NO: 161) |
| VEGFA_T2 | End protection - reverse | AGAAAUCCGUCUUUCAU |
| (SEQ ID NO: 36) | transcription template | UGACGGGCACACGACGA |
| | sequence (34 nt) - PBS (13 | UGUAAUCGCAGAUACCU |
| | nt) - nuclease binding | aUAAUCCUCCACCAcagU |
| | sequence - DNA-binding | UGGUGACAACCCCAAGC |
| | sequence | AGCCAGAAAUCCGUCUU |
| | | UCAUUGACGGAAUCCUC |
| | | CACCAGUCAUGGU(SEQ |
| | | ID NO: 162) |
| VEGFA_T2 | End protection - reverse | AGAAAUCCGUCUUUCAU |
| (SEQ ID NO: 36) | transcription template | UGACGGGCACACGACGA |
| | sequence (34 nt) - PBS (30 | UGUAAUCGCAGAUACCU |
| | nt) - nuclease binding | aUAAUCCUCCACCAcagU |
| | sequence - DNA-binding | UGGUGACAACCCCAAGC |
| | sequence | AGCCCACACAUUcUCAA |
| | | GUGCAGAAAUCCGUCUU |
| | | UCAUUGACGGAAUCCUC |
| | | CACCAGUCAUGGU(SEQ |
| | | ID NO: 163) |
| VEGFA_T2 | End protection - reverse | AGAAAUCCGUCUUUCAU |
| (SEQ ID NO: 36) | transcription template | UGACGGGCACACGACGA |
| | sequence (34 nt) - PBS (60 | UGUAAUCGCAGAUACCU |
| | nt) - nuclease binding | aUAAUCCUCCACCAcagU |
| | sequence - DNA-binding | UGGUGACAACCCCAAGC |
| | sequence | AGCCCACACAUUcUCAA |
| | | GUGCCCCCAGGAUGCGU |
| | | GGAGGGAGGGGUCUGU |
| | | GAGAAAUCCGUCUUUCA |
| | | UUGACGGAAUCCUCCAC |
| | | CAGUCAUGGU (SEQ ID |
| | | NO: 164) |
| VEGFA_T2 | End protection - reverse | AGAAAUCCGUCUUUCAU |
| (SEQ ID NO: 36) | transcription template | UGACGGGCACACGACGA |
| | sequence (40 nt) - PBS (13 | UGUAAUCGCAAUACUA |
| | nt) - nuclease binding | GAUACCUaUAAUCCUCC |
| | sequence - DNA-binding | ACCAcagUUGGUGACAAC |
| | sequence | CCCAAGCAGCCAGAAAU |
| | | CCGUCUUUCAUUGACGG |
| | | AAUCCUCCACCAGUCAU |
| | | GGU (SEQ ID NO: 165) |
| VEGFA_T2 | End protection - reverse | AGAAAUCCGUCUUUCAU |
| (SEQ ID NO: 36) | transcription template | UGACGGGCACACGACGA |
| | sequence (40 nt) - PBS (30 | UGUAAUCGCAAUACUA |
| | nt) - nuclease binding | GAUACCUaUAAUCCUCC |
| | sequence - DNA-binding | ACCAcagUUGGUGACAAC |
| | sequence | CCCAAGCAGCCCACACA |
| | | UUcUCAAGUGCAGAAAU |
| | | CCGUCUUUCAUUGACGG |
| | | AAUCCUCCACCAGUCAU |
| | | GGU (SEQ ID NO: 166) |
| VEGFA_T2 | End protection - reverse | AGAAAUCCGUCUUUCAU |
| (SEQ ID NO: 36) | transcription template | UGACGGGCACACGACGA |
| | sequence (40 nt) - PBS (60 | UGUAAUCGCAAUACUA |
| | nt) - nuclease binding | GAUACCUaUAAUCCUCC |
| | sequence - DNA-binding | ACCAcagUUGGUGACAAC |
| | sequence | CCCAAGCAGCCCACACA |
| | | UUcUCAAGUGCCCCCAG |
| | | GAUGCGUGGAGGGAGG |
| | | GGUCUGUGAGAAAUCC |
| | | GUCUUUCAUUGACGGA |
| | | AUCCUCCACCAGUCAUG |
| | | GU (SEQ ID NO: 167) |
| VEGFA_T5 | nuclease binding sequence - | AGAAAUCCGUCUUUCAU |
| TTAAACTCTCCATGGAC | DNA-binding sequence - | UGACGGUUAAACUCUCC |
| CAG | reverse transcription template | AUGGACCAGAAAAGAC |
| (SEQ ID NO: 38) | sequence (34 nt) - PBS (13 | UacUUAAACUCUCCAacc |
| | nt) | UCCAGGCUCAUCCAGCU |
| | | UCCCA (SEQ ID NO: 168) |
| VEGFA_T5 | nuclease binding sequence - | AGAAAUCCGUCUUUCAU |
| (SEQ ID NO: 38) | DNA-binding sequence - | UGACGGUUAAACUCUCC |
| | reverse transcription template | AUGGACCAGAAAAGAC |
| | sequence (34 nt) - PBS (30 | UacUUAAACUCUCCAacc |
| | nt) | UCCAGGCUCAUCCAGCU |
| | | UCCCAAACAAAGCCCCC |
| | | AAGAA (SEQ ID NO: 169) |
| VEGFA_T5 | nuclease binding sequence - | AGAAAUCCGUCUUUCAU |
| (SEQ ID NO: 38) | DNA-binding sequence - | UGACGGUUAAACUCUCC |
| | reverse transcription template | AUGGACCAGAAAAGAC |
| | sequence (34 nt) - PBS (60 | UacUUAAACUCUCCAacc |
| | nt) | UCCAGGCUCAUCCAGCU |
| | | UCCCAAACAAAGCCCCC |
| | | AAGAAGGGGGGGCACU |
| | | CAGGACUCUCUCCAAGA |
| | | GA (SEQ ID NO: 170) |
| VEGFA_T5 | nuclease binding sequence - | AGAAAUCCGUCUUUCAU |
| (SEQ ID NO: 38) | DNA-binding sequence - | UGACGGUUAAACUCUCC |
| | reverse transcription template | AUGGACCAGAAAAGAC |
| | sequence (34 nt) - PBS (13 | UacUUAAACUCUCCAacc |
| | nt) - end protection | UCCAGGCUCAUCCAGCU |
| | | UCCCAAGAAAUCCGUCU |
| | | UUCAUUGACGGGCACAC |
| | | GACGAUGUAAUCGC |
| | | (SEQ ID NO: 171) |
| VEGFA_T5 | nuclease binding sequence - | AGAAAUCCGUCUUUCAU |
| (SEQ ID NO: 38) | DNA-binding sequence - | UGACGGUUAAACUCUCC |
| | reverse transcription template | AUGGACCAGAAAAGAC |
| | sequence (34 nt) - PBS (30 | UacUUAAACUCUCCAacc |
| | nt) - end protection | UCCAGGCUCAUCCAGCU |
| | | UCCCAAACAAAGCCCCC |
| | | AAGAAAGAAAUCCGUC |
| | | UUUCAUUGACGGGCACA |
| | | CGACGAUGUAAUCGC |
| | | (SEQ ID NO: 172) |
| VEGFA_T5 | nuclease binding sequence - | AGAAAUCCGUCUUUCAU |
| (SEQ ID NO: 38) | DNA-binding sequence - | UGACGGUUAAACUCUCC |
| | reverse transcription template | AUGGACCAGAAAAGAC |
| | sequence (34 nt) - PBS (60 | UacUUAAACUCUCCAacc |
| | nt) - end protection | UCCAGGCUCAUCCAGCU |
| | | UCCCAAACAAAGCCCCC |
| | | AAGAAGGGGGGGCACU |
| | | CAGGACUCUCUCCAAGA |
| | | GAAGAAAUCCGUCUUUC |
| | | AUUGACGGGCACACGAC |
| | | GAUGUAAUCGC (SEQ ID |
| | | NO: 173) |
| VEGFA_T5 | Reverse transcription | AAAAGACUacUUAAACU |
| (SEQ ID NO: 38) | template sequence (34 nt) - | CUCCAaccUCCAGGCUCA |
| | PBS (13 nt) - nuclease | UCCAGCUUCCCAAGAAA |
| | binding sequence - DNA- | UCCGUCUUUCAUUGACG |
| | binding sequence | GUUAAACUCUCCAUGGA |
| | | CCAG (SEQ ID NO: 174) |
| VEGFA_T5 | Reverse transcription | AAAAGACUacUUAAACU |
| (SEQ ID NO: 38) | template sequence (34 nt) - | CUCCAaccUCCAGGCUCA |
| | PBS (30 nt) - nuclease | UCCAGCUUCCCAAACAA |
| | binding sequence - DNA- | AGCCCCCAAGAAAGAAA |
| | binding sequence | UCCGUCUUUCAUUGACG |
| | | GUUAAACUCUCCAUGGA |
| | | CCAG (SEQ ID NO: 175) |
| VEGFA_T5 | Reverse transcription | AAAAGACUacUUAAACU |
| (SEQ ID NO: 38) | template sequence (34 nt) - | CUCCAaccUCCAGGCUCA |
| | PBS (60 nt) - nuclease | UCCAGCUUCCCAAACAA |
| | binding sequence - DNA- | AGCCCCCAAGAAGGGGG |
| | binding sequence | GGCACUCAGGACUCUCU |
| | | CCAAGAGAAGAAAUCCG |
| | | UCUUUCAUUGACGGUU |
| | | AAACUCUCCAUGGACCA |
| | | G (SEQ ID NO: 176) |
| VEGFA_T5 | End protection - reverse | AGAAAUCCGUCUUUCAU |
| (SEQ ID NO: 38) | transcription template | UGACGGGCACACGACGA |
| | sequence (34 nt) - PBS (13 | UGUAAUCGCAAAAGAC |
| | nt) - nuclease binding | UacUUAAACUCUCCAacc |
| | sequence - DNA-binding | UCCAGGCUCAUCCAGCU |
| | sequence | UCCCAAGAAAUCCGUCU |
| | | UUCAUUGACGGUUAAA |
| | | CUCUCCAUGGACCAG |
| | | (SEQ ID NO: 177) |
| VEGFA_T5 | End protection - reverse | AGAAAUCCGUCUUUCAU |
| (SEQ ID NO: 38) | transcription template | UGACGGGCACACGACGA |
| | sequence (34 nt) - PBS (30 | UGUAAUCGCAAAAGAC |
| | nt) - nuclease binding | UacUUAAACUCUCCAacc |
| | sequence - DNA-binding | UCCAGGCUCAUCCAGCU |
| | sequence | UCCCAAACAAAGCCCCC |
| | | AAGAAAGAAAUCCGUC |
| | | UUUCAUUGACGGUUAA |
| | | ACUCUCCAUGGACCAG |
| | | (SEQ ID NO: 178) |
| VEGFA_T5 | End protection - reverse | AGAAAUCCGUCUUUCAU |
| (SEQ ID NO: 38) | transcription template | UGACGGGCACACGACGA |
| | sequence (34 nt) - PBS (60 | UGUAAUCGCAAAAGAC |
| | nt) - nuclease binding | UacUUAAACUCUCCAacc |
| | sequence - DNA-binding | UCCAGGCUCAUCCAGCU |
| | sequence | UCCCAAACAAAGCCCCC |
| | | AAGAAGGGGGGGCACU |
| | | CAGGACUCUCUCCAAGA |
| | | GAAGAAAUCCGUCUUUC |
| | | AUUGACGGUUAAACUC |
| | | UCCAUGGACCAG (SEQ |
| | | ID NO: 179) |
| VEGFA_T5 | End protection - reverse | AGAAAUCCGUCUUUCAU |
| (SEQ ID NO: 38) | transcription template | UGACGGGCACACGACGA |
| | sequence (40 nt) - PBS (13 | UGUAAUCGCAACACCAA |
| | nt) - nuclease binding | AAGACUacUUAAACUCU |
| | sequence - DNA-binding | CCAaccUCCAGGCUCAUC |
| | sequence | CAGCUUCCCAAGAAAUC |
| | | CGUCUUUCAUUGACGGU |
| | | UAAACUCUCCAUGGACC |
| | | AG (SEQ ID NO: 180) |
| VEGFA_T5 | End protection - reverse | AGAAAUCCGUCUUUCAU |
| (SEQ ID NO: 38) | transcription template | UGACGGGCACACGACGA |
| | sequence (40 nt) - PBS (30 | UGUAAUCGCAACACCAA |
| | nt) - nuclease binding | AAGACUacUUAAACUCU |
| | sequence - DNA-binding | CCAaccUCCAGGCUCAUC |
| | sequence | CAGCUUCCCAAACAAAG |
| | | CCCCCAAGAAAGAAAUC |
| | | CGUCUUUCAUUGACGGU |
| | | UAAACUCUCCAUGGACC |
| | | AG (SEQ ID NO: 181) |
| VEGFA_T5 | End protection - reverse | AGAAAUCCGUCUUUCAU |
| (SEQ ID NO: 38) | transcription template | UGACGGGCACACGACGA |
| | sequence (40 nt) - PBS (60 | UGUAAUCGCAACACCAA |
| | nt) - nuclease binding | AAGACUacUUAAACUCU |
| | sequence - DNA-binding | CCAaccUCCAGGCUCAUC |
| | sequence | CAGCUUCCCAAACAAAG |
| | | CCCCCAAGAAGGGGGGG |
| | | CACUCAGGACUCUCUCC |
| | | AAGAGAAGAAAUCCGU |
| | | CUUUCAUUGACGGUUA |
| | | AACUCUCCAUGGACCAG |
| | | (SEQ ID NO: 182) |

TABLE 13
RNA GUIDE CONTROL SEQUENCES

| Target | RNA Guide Sequence |
|---|---|
| AAVS1_T7 | AGAAAUCCGUCUUUCAUUGACGG |
| (SEQ ID | GGGAAGUGGUUGGUCAGCAU |
| NO: 32) | (SEQ ID NO: 112) |
| EMX1-T6 | AGAAAUCCGUCUUUCAUUGACGG |
| (SEQ ID | GAGCCAGUGUUGCUAGUCAA |
| NO: 34) | (SEQ ID NO: 114) |
| VEGFA_T2 | AGAAAUCCGUCUUUCAUUGACGG |
| (SEQ ID | AAUCCUCCACCAGUCAUGGU |
| NO: 36) | (SEQ ID NO: 116) |
| VEGFA_T5 | AGAAAUCCGUCUUUCAUUGACGG |
| (SEQ ID | UUAAACUCUCCAUGGACCAG |
| NO: 38) | (SEQ ID NO: 118) |

Approximately 16 hours prior to transfection, 25,000 HEK293T cells in DMEM/10% FBS+Pen/Strep were plated into each well of a 96-well plate. On the day of transfection, the cells were 70-90% confluent. For each well to be transfected, a mixture of Lipofectamine™ 2000 (Thermo Fisher) and Opti-MEM™ (Thermo Fisher) was prepared and then incubated at room temperature for 5-20 minutes (Solution 1). After incubation, the Lipofectamine™:OptiMEM™ mixture was added to a separate mixture containing variant Cas12i2-RT fusion working solution, RNA working solution and OptiMEM™ media (Solution 2). The solution 1 and solution 2 mixtures were mixed by pipetting up and down and then incubated at room temperature for 25 minutes. Following incubation, Solution 1 and Solution 2 mixture were added dropwise to each well of a 96 well plate containing the cells. 72 hours post transfection, cells were trypsinized by adding TrypLE™ (ThermoFisher) to the center of each well and incubated for approximately 5 minutes. Growth media was then added to each well and mixed to resuspend cells. The cells were then spun down at 400 g for 10 minutes, and the supernatant was discarded. QuickExtract™ buffer (Lucigen) was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 (Illumina) mid or high output kit.

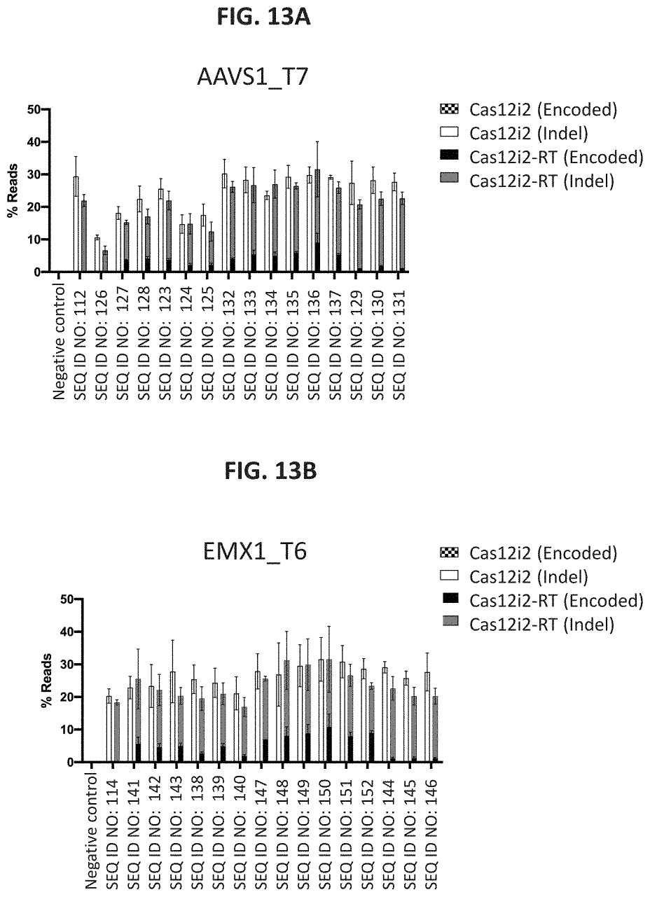

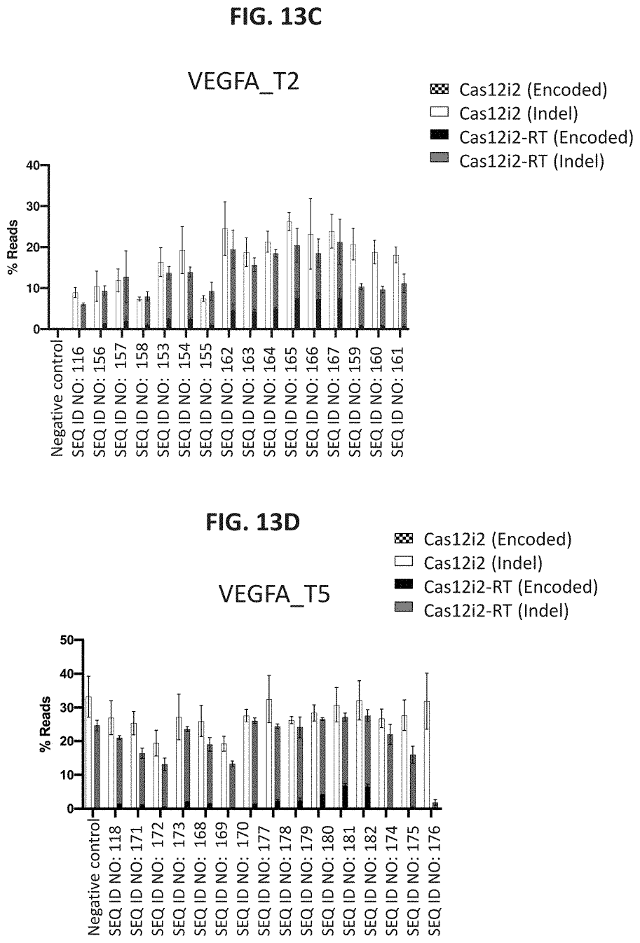

FIG. 13A shows activity for AAVS1_T7, FIG. 13B shows activity for EMX1_T6, FIG. 13C shows activity for VEGFA_T2, and FIG. 13D shows activity for VEGFA_T5. Percentage of NGS reads is shown on the y-axis. The data is an average of three technical replicates.

As shown in FIG. 13A, FIG. 13B, FIG. 13C, and FIG. 13D, variant Cas12i2 of SEQ ID NO: 4 and the variant Cas12i2-RT fusion of SEQ ID NO: 25 were active nucleases in the presence of RNA guides targeting AAVS1_T7, EMX1_T6, VEGFA_T2, or VEGFA_T5 (see gRNA samples). Desired edits were only observed in the presence of an RT (variant Cas12i2-RT fusion of SEQ ID NO: 25). Indels and encoded edits were observed for each of the tested editing template RNAs with the variant Cas12i2-RT fusion of SEQ ID NO: 25. 5′ extension editing template RNAs with end protection (End protection—reverse transcription template sequence—PBS—nuclease binding sequence—DNA-binding sequence) demonstrated higher numbers of reads with the desired edits compared to 5′ extension editing template RNAs without end protection (Reverse transcription template sequence—PBS—nuclease binding sequence—DNA-binding sequence).

This Example shows that specific edits were incorporated into the selected mammalian genomic sites using multiple configurations of editing template RNAs and Cas12i2-RT fusions.

### Example 3—Determination of In Vitro Cleavage Patterns for Cas12i2

In this Example, in vitro cleavage patterns of variant Cas12i2 with an RNA guide were determined. Determining the cleavage sites generated by Cas12i2 on double stranded DNA targets enables the design of the PBS component of editing template RNAs.

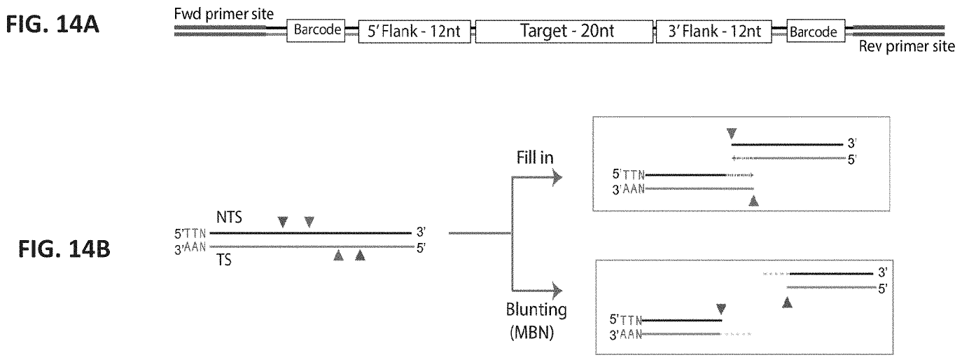

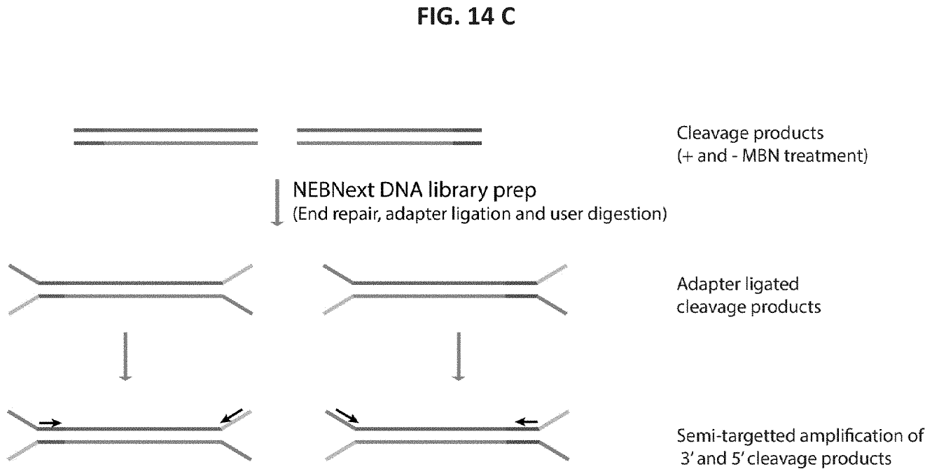

A schematic of the assay to determine the cleavage patterns is shown in FIG. 14A-C. Oligos containing target sequences for cut site analysis were first designed. The oligos comprised a target sequence with 12-nucleotide flanking sequences on both ends of the target, internal barcodes, and priming sites to allow for targeted amplification (FIG. 14A). As shown in FIG. 14B, all cleavage products were split into two halves, where one half was treated with mung bean nuclease (MBN), which blunts the 5′ and 3′ overhangs (blunting treatment), and the other half reaction was end repaired (part of NEBNext DNA library prep, New England Biolabs), where the 5′ overhangs were filled in (fill in treatment). Both halves were then subjected to NGS library preparation and semi-targeted amplification. Type V CRISPR nucleases have been shown to generate a staggered cut with 5′ overhangs as indicated by grey arrows. These cut sites were captured by the fill in treatment to fill in of any 5′ overhangs. Therefore, the 5′ and 3′ sequencing of these products indicated cleavage sites on the target strand and the non-target strand. Recent work with Cpf1 indicated additional cleavage sites, particularly on the non-target strand that were not captured by the fill in method. To capture these cleavage sites, a blunting method results in blunting of all 5′ and 3′ overhangs. As a result, 5′ cleavage products in the blunting method indicated any additional cut sites on the non-target strand and the 3′ cleavage products indicate additional cut sites on the target strand. Semi-targeted amplification after NEBNext adapter ligation allowed for specific amplification of 5′ and 3′ cleavage products; the amplified products were pooled and analyzed by NGS (FIG. 14C).

DNA substrates were generated by PCR amplification using IR800 and IR700 labelled forward and reverse primers, respectively, resulting in dsDNA targets with IR800 labelled target strand and IR700 labelled non-target strand. The PCR products were cleaned up using CleanNGS SPRI beads at a 1.8× ratio of beads-to-PCR product. Purified Cas12i2 were pre-incubated with crRNA to form RNP in NEBuffer 3 (10 mM Tris-HCl, pH 7.9, 150 mM NaCl, 10 mM MgCl₂, 1 mM DTT) at 37° C. for 10 min. In vitro cleavage reactions comprising dsDNA substrates mixed with serial diluted RNP in NEBuffer 3 were performed at 37° C. for 1 hr. The reactions were quenched with EDTA. The reactions were treated with an RNase cocktail (37° C. for 15 min), followed by Proteinase K treatment (37° C. for 15 min). The reactions were analyzed by denaturing gel electrophoresis using 15% TBE-Urea gels and imaged on an Odyssey CLx (LI-COR) imager. The DNA substrate and RNA guide sequences are shown in Table 14; the target sequence is in bold.

TABLE 14
CLEAVAGE ASSAY SEQUENCES

| Target | dsDNA substrate | crRNA |
|---|---|---|
| AAVS1_T2 | TCCATGTCTCGTTAT | AGAAATCCGTCTTTC |
| | ACGCTGTGGTTCGCC | ATTGACGGGGAGAGG |
| | AACAACACTAGTACT | TGAGGGACTTGGG |
| | ACTGATAAATTACCC | (SEQ ID NO: 216) |
| | CCCAAGTCCCTCACC | |
| | TCTCCAAAGCTGCCC | |
| | ATACTACTACACTAG | |
| | TTTGACAGCTAGCTC | |
| | AGTCCTAGGTATAAT | |
| | GCTAGC | |
| | (SEQ ID NO: 213) | |
| EMX1_T6 | TCCATGTCTCGTTAT | AGAAATCCGTCTTTC |
| | ACGCTGTGGTTCGCC | ATTGACGGGAGCCAG |
| | AACATATAGTTCACT | TGTTGCTAGTCAA |
| | ACTAGCATGCTGCCC | (SEQ ID NO: 217) |
| | TTGACTAGCAACACT | |
| | GGCTCAAAGAAGGAA | |
| | AGACTACTTATAGTT | |
| | CTTGACAGCTAGCTC | |
| | AGTCCTAGGTATAAT | |
| | GCTAGC | |
| | (SEQ ID NO: 214) | |
| VEGFA_T5 | TCCATGTCTCGTTAT | AGAAATCCGTCTTTC |
| | ACGCTGTGGTTCGCC | ATTGACGGTTAAACT |
| | AACGCAGGAACGACA | CTCCATGGACCAG |
| | CTACTAGCTGGATGA | (SEQ ID NO: 218) |
| | GCCTGGTCCATGGAG | |
| | AGTTTAAAAAGTCTT | |
| | TTGGACTACTGGAAC | |
| | GACTTGACAGCTAGC | |
| | TCAGTCCTAGGTATA | |
| | ATGCTAGC | |
| | (SEQ ID NO: 215) | |

For in vitro cleavage fragment analysis to determine the cut positions, the reaction products were purified using SPRI beads and isopropyl alcohol (IPA SPRI). The purified reaction was split into two halves. One half was treated with mung bean nuclease (New England Biolabs) at 30° C. for 30 minutes to remove all 5′ and 3′ overhangs to generate blunt ends, followed by purification with IPA SPRI. Both the mung bean nuclease-treated and untreated halves were then prepared for sequencing using NEBNext Ultra-II DNA library prep kit (New England Biolabs) using manufacturer's instructions. Semi-targeted amplification was used to amplify 5′ and 3′ cut products separately for each sample. All amplicons were pooled and gel extracted prior to sequencing. For each sample, the read lengths obtained for 5′ and 3′ cut product for mung bean nuclease and non-mung bean nuclease treated samples were plotted as histograms and mapped to the target sequence.

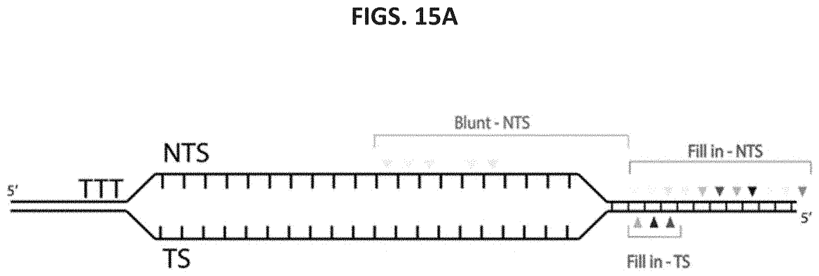

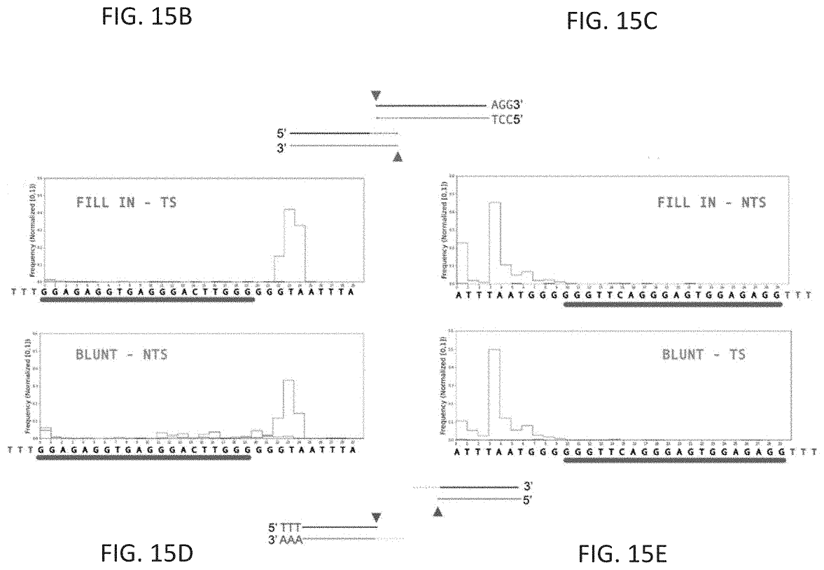

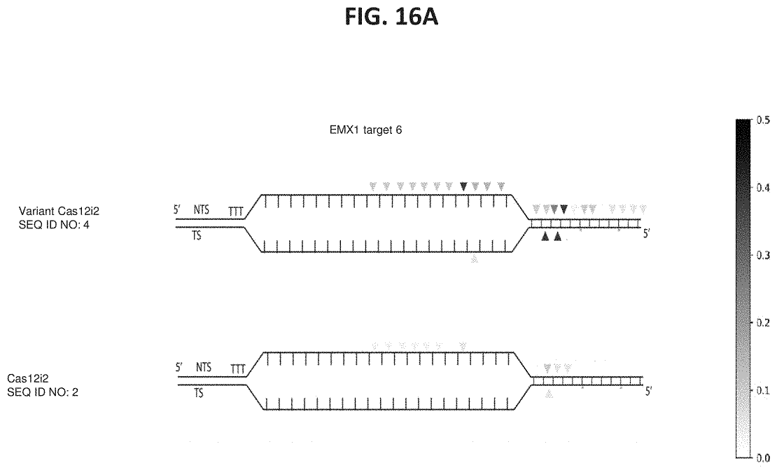

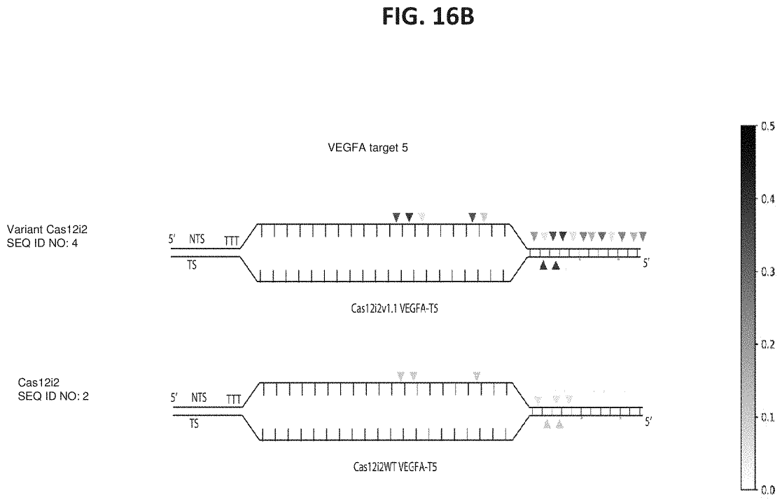

To obtain the full cleavage pattern, data from all four histograms were taken and visualized on the R-loop diagram as show in FIG. 15A-E and FIG. 16. FIG. 15B-E show histograms of read lengths obtained from semi-targeted amplification of 5′ and 3′ cleavage products for AAVS1_T2. FIG. 15B and FIG. 15D show read length histograms of 5′ cleavage products for fill-in and blunting treatment, respectively. FIG. 15C and FIG. 15E show read length histograms for 3′ cleavage products for fill-in and blunting treatment, respectively. Each read length histogram was mapped to the target sequence shown on the x-axis. The PAM sequence (5′-NTTT-3′) was also indicated. The cleavage sites obtained from the histograms are illustrated as triangles in the R-loop diagram of FIG. 15A. FIG. 16 compares the cleavage sites on AAVS1_T2 and EMX1_T6 for RNPs comprising either Cas12i2 (SEQ ID NO: 2) or variant Cas12i2 (SEQ ID NO: 4). The scale bar (right) represents the cleavage frequency as measured by the number of sequencing reads.

For all targets, multiple cleavage sites of varying frequency were observed both on the target strand and the non-target strand. On the non-target strand, a broad cleavage profile was observed with several cleavage sites detected within the spacer region (i.e., the RNA guide binding region) as well as outside of the spacer region. This indicated that upon Cas12i2 binding, double-stranded DNA was likely unwound several base pairs from the spacer region (i.e., the RNA guide binding region) and existed as single stranded DNA, making it accessible to Cas12i2 for cleavage. The cleavage sites on the target strand were consistently observed outside of the spacer region (i.e., outside of the RNA guide binding region). For AAVS1_T2 the target strand cut sites were observed at positions 22 to 24 nucleotides from the PAM sequence. For VEGFA_T5 and EMX1_T6, the target strand cut sites were observed at positions 22 to 23 nucleotides from the PAM sequence. This thus shows that editing template RNAs designed to target the target strand should comprise a PBS beginning at positions 22 to 24 nucleotides from the PAM sequence.

### Example 4—Editing of Target Strand in Mammalian Cells Using Editing Template RNA with Various PBS and Reverse Transcription Template Sequence Lengths

This Example describes target strand editing of mammalian genes using editing template RNAs with PBS lengths of 3 to 60 nucleotides and reverse transcription template sequence lengths of 14 to 54 nucleotides.

A working solution of plasmid comprising the variant Cas12i2-RT fusion of SEQ ID NO: 25 was prepared in water (variant Cas12i2-RT fusion working solution). The editing template RNA sequences are shown in Table 15. In one set of conditions, the reverse transcription template sequence was 34 nucleotides in length, and the PBS was 3, 8, 13, 30, or 60 nucleotides in length. In a second set of conditions, the PBS was 13 nucleotides in length, and the reverse transcription template sequence was 14, 24, 34, 44, or 54 nucleotides in length. Each editing template RNA was cloned into a plasmid backbone with a U6 promoter and maxi-prepped. A working solution of plasmid expressing each editing template RNA was prepared in water (editing template RNA working solution).

TABLE 15
EDITING TEMPLATE RNA SEQUENCES

| | Editing Template RNA | Editing Template RNA |
|---|---|---|
| Target | Description | Sequence |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (3 nt) | UUUGGGAAGUGGUUGC |
| | | AGUGCAUGGAUUAU |
| | | (SEQ ID NO: 183) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (8 nt) | UUUGGGAAGUGGUUGC |
| | | AGUGCAUGGAUUAUAG |
| | | CCG (SEQ ID NO: 184) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (13 | UUUGGGAAGUGGUUGC |
| | nt) | AGUGCAUGGAUUAUAG |
| | | CCGAAGGC (SEQ ID NO: |
| | | 185) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (30 | UUUGGGAAGUGGUUGC |
| | nt) | AGUGCAUGGAUUAUAG |
| | | CCGAAGGCCCCAGCUUU |
| | | GCCUUGUU (SEQ ID NO: |
| | | 186) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (60 | UUUGGGAAGUGGUUGC |
| | nt) | AGUGCAUGGAUUAUAG |
| | | CCGAAGGCCCCAGCUUU |
| | | GCCUUGUUCUAGCAGUU |
| | | CCACUCCUGGGCAGCCC |
| | | GAGA (SEQ ID NO: 187) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUUGCAGU |
| | sequence (14 nt)-PBS (13 | GCAUGGAUUAUAGCCG |
| | nt) | AAGGC (SEQ ID NO: 188) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUGGGAAG |
| | sequence (24 nt)-PBS (13 | UGGUUGCAGUGCAUGG |
| | nt) | AUUAUAGCCGAAGGC |
| | | (SEQ ID NO: 189) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUCAGCUAC |
| | sequence (34 nt)-PBS (13 | UUUGGGAAGUGGUUGC |
| | nt) | AGUGCAUGGAUUAUAG |
| | | CCGAAGGC (SEQ ID NO: |
| | | 190) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUGUGAUG |
| | sequence (44 nt)-PBS (13 | AUUCCAGCUACUUUGGG |
| | nt) | AAGUGGUUGCAGUGCA |
| | | UGGAUUAUAGCCGAAG |
| | | GC (SEQ ID NO: 191) |
| AAVS1_T7 | nuclease binding sequence- | AGAAAUCCGUCUUUCAU |
| (SEQ ID NO: 32) | DNA-binding sequence- | UGACGGGGGAAGUGGU |
| | reverse transcription template | UGGUCAGCAUGGUGGG |
| | sequence (54 nt)-PBS (13 | AAAGGUGAUGAUUCCA |
| | nt) | GCUACUUUGGGAAGUG |
| | | GUUGCAGUGCAUGGAU |
| | | UAUAGCCGAAGGC (SEQ |
| | | ID NO: 192) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (3 nt) | UUUGAGCCAGUGUUGG |
| | | AUCUCAAGGGCAGC |
| | | (SEQ ID NO: 193) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (8 nt) | UUUGAGCCAGUGUUGG |
| | | AUCUCAAGGGCAGCAUG |
| | | CU (SEQ ID NO: 194) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (13 | UUUGAGCCAGUGUUGG |
| | nt) | AUCUCAAGGGCAGCAUG |
| | | CUGGGCC (SEQ ID NO: |
| | | 195) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (30 | UUUGAGCCAGUGUUGG |
| | nt) | AUCUCAAGGGCAGCAUG |
| | | CUGGGCCCGUCCCACUA |
| | | CAGGCCA (SEQ ID NO: |
| | | 196) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (60 | UUUGAGCCAGUGUUGG |
| | nt) | AUCUCAAGGGCAGCAUG |
| | | CUGGGCCCGUCCCACUA |
| | | CAGGCCAAUGUGACCGU |
| | | CAGUCUCCUUCCUGAAG |
| | | GAC (SEQ ID NO: 197) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUGGAUC |
| | sequence (14 nt)-PBS (13 | UCAAGGGCAGCAUGCUG |
| | nt) | GGCC (SEQ ID NO: 198) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAGAGCCAG |
| | sequence (24 nt)-PBS (13 | UGUUGGAUCUCAAGGG |
| | nt) | CAGCAUGCUGGGCC |
| | | (SEQ ID NO: 199) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUUCCUUC |
| | sequence (34 nt)-PBS (13 | UUUGAGCCAGUGUUGG |
| | nt) | AUCUCAAGGGCAGCAUG |
| | | CUGGGCC (SEQ ID NO: |
| | | 200) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAAUCCAAA |
| | sequence (44 nt)-PBS (13 | GCUUUCCUUCUUUGAGC |
| | nt) | CAGUGUUGGAUCUCAA |
| | | GGGCAGCAUGCUGGGCC |
| | | (SEQ ID NO: 201) |
| EMX1_T6 | nuclease binding sequence- | AGAAAUCCGUCUUUCAU |
| (SEQ ID NO: 34) | DNA-binding sequence- | UGACGGGAGCCAGUGU |
| | reverse transcription template | UGCUAGUCAAUGAAGU |
| | sequence (54 nt)-PBS (13 | CGCCAUCCAAAGCUUUC |
| | nt) | CUUCUUUGAGCCAGUGU |
| | | UGGAUCUCAAGGGCAGC |
| | | AUGCUGGGCC (SEQ ID |
| | | NO: 202) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (3 nt) | UUCUUAAACUCUCCAAC |
| | | CUCCAGGCUCAUC(SEQ |
| | | ID NO: 203) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (8 nt) | UUCUUAAACUCUCCAAC |
| | | CUCCAGGCUCAUCCAGC |
| | | U (SEQ ID NO: 204) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (13 | UUCUUAAACUCUCCAAC |
| | nt) | CUCCAGGCUCAUCCAGC |
| | | UUCCCA (SEQ ID NO: 205) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (30 | UUCUUAAACUCUCCAAC |
| | nt) | CUCCAGGCUCAUCCAGC |
| | | UUCCCAAACAAAGCCCC |
| | | CAAGAA (SEQ ID NO: |
| | | 206) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (60 | UUCUUAAACUCUCCAAC |
| | nt) | CUCCAGGCUCAUCCAGC |
| | | UUCCCAAACAAAGCCCC |
| | | CAAGAAGGGGGGGCAC |
| | | UCAGGACUCUCUCCAAG |
| | | AGA (SEQ ID NO: 207) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGCAACCUCC |
| | sequence (14 nt)-PBS (13 | AGGCUCAUCCAGCUUCC |
| | nt) | CA (SEQ ID NO: 208) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGUUAAACUC |
| | sequence (24 nt)-PBS (13 | UCCAACCUCCAGGCUCA |
| | nt) | UCCAGCUUCCCA (SEQ |
| | | ID NO: 209) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAAAAGAC |
| | sequence (34 nt)-PBS (13 | UUCUUAAACUCUCCAAC |
| | nt) | CUCCAGGCUCAUCCAGC |
| | | UUCCCA (SEQ ID NO: 210) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGAGGUAAC |
| | sequence (44 nt)-PBS (13 | ACCAAAAGACUUCUUAA |
| | nt) | ACUCUCCAACCUCCAGG |
| | | CUCAUCCAGCUUCCCA |
| | | (SEQ ID NO: 211) |
| VEGFA_T5 | nuclease binding sequence- | AGAAAUCCGUCUUUCAU |
| (SEQ ID NO: 38) | DNA-binding sequence- | UGACGGUUAAACUCUCC |
| | reverse transcription template | AUGGACCAGCCCCAUUA |
| | sequence (54 nt)-PBS (13 | CCAGGUAACACCAAAAG |
| | nt) | ACUUCUUAAACUCUCCA |
| | | ACCUCCAGGCUCAUCCA |
| | | GCUUCCCA (SEQ ID NO: |
| | | 212) |

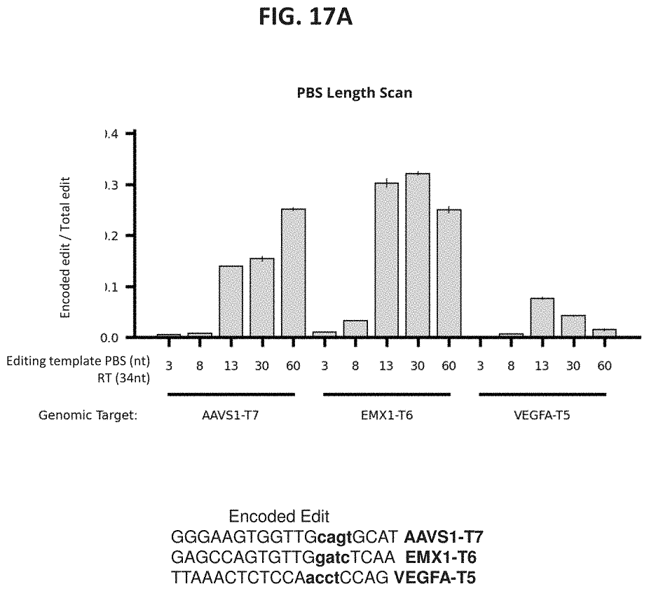

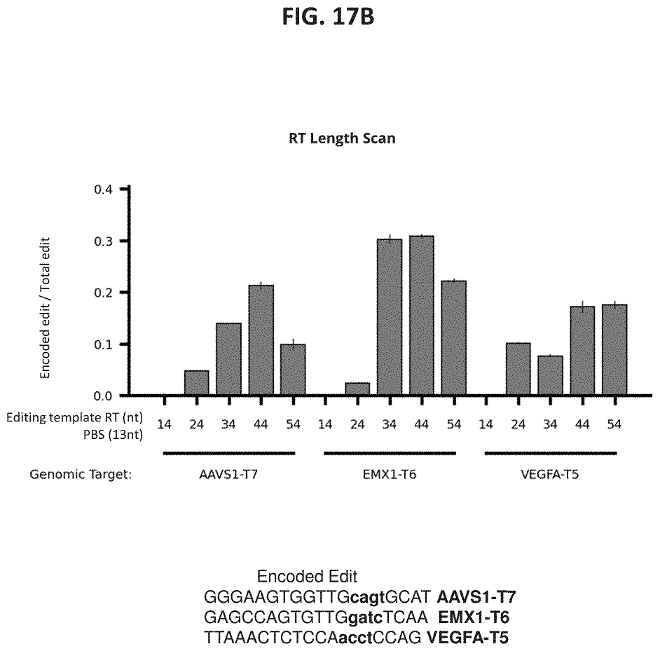

Cells were transfected and analyzed as described in Example 2. FIG. 17A shows activity of Cas12i2-RT of SEQ ID NO: 25 and the editing template RNAs of SEQ ID NOs: 183-187 for AAVS1_T7, Cas12i2-RT of SEQ ID NO: 25 and the editing template RNAs of SEQ ID NOs: 193-197 for EMX1_T6, and Cas12i2-RT of SEQ ID NO: 25 and the editing template RNAs of SEQ ID NOs: 203-207 for VEGFA_T5. FIG. 17B shows activity of Cas12i2-RT of SEQ ID NO: 25 and the editing template RNAs of SEQ ID NOs: 188-192 for AAVS1_T7, Cas12i2-RT of SEQ ID NO: 25 and the editing template RNAs of SEQ ID NOs: 198-202 for EMX1_T6, and Cas12i2-RT of SEQ ID NO: 25 and the editing template RNAs of SEQ ID NOs: 208-212 for VEGFA_T5. The ratio of encoded edits to total indels is shown on the y-axis of FIG. 17A and FIG. 17B.

As shown in FIG. 17A, each of the tested PBS lengths resulted in the incorporation of encoded edits into the selected target sites. Use of the PBS lengths of 13, 30, and 60 nucleotides resulted in the highest ratio of encoded edits to total indels. As shown in FIG. 17B, the tested reverse transcription template sequence lengths of 24, 34, 44, and 54 nucleotides resulted in presence of encoded edits. For EMX1_T6, encoded edits accounted for about 30% of the total edits using editing template RNAs having a PBS of 13 nucleotides in length and a reverse transcription template sequence of 34 or 44 nucleotides in length. This Example thus shows that editing template RNAs with various PBS and reverse transcription template sequence lengths introduced encoded edits into target sequences in mammalian cells.

### Example 5—RNA-Templated Editing of Target Strand in U2OS Cells

This Example describes target strand editing of mammalian genes in U2OS cells. A working solution of plasmid comprising the variant Cas12i2-RT fusion of SEQ ID NO: 25 was prepared in water. Each editing template RNA was cloned into a plasmid backbone with a U6 promoter and maxi-prepped. Working solutions of plasmids comprising each editing template RNA were prepared in water. The editing template RNA sequences are shown in Table 16. An additional DR-spacer sequence was added to the 3′ end, with the additional spacer sequence being non-human targeting (i.e., it did not target any sequence in the human genome). The desired edit encoded in the RT donor is shown in lowercase letters in Table 16.

TABLE 16
EDITING TEMPLATE RNA SEQUENCE

| Target | Editing Template RNA Sequences |
|---|---|
| AAVS1_T7 | AGAAAUCCGUCUUUCAUUGACGGGCACACGACGA |
| GGGAAGTGGT | UGUAAUCGCUGAUUCCAGCUACUaUGGGAAGUGG |
| TGGTCAGCAT | UUGcaguGCAUGGAUUAUAGCCGAAGGCCCCAGC |
| (SEQ ID | UUUGCCUUGUUAGAAAUCCGUCUUUCAUUGACGG |
| NO: 32) | GGGAAGUGGUUGGUCAGCAU |
| | (SEQ ID NO: 136) |
| EMX1_T6 | AGAAAUCCGUCUUUCAUUGACGGGCACACGACGA |
| GAGCCAGTGT | UGUAAUCGCAAAGCUUUCCUUCUaUGAGCCAGUG |
| TGCTAGTCAA | UUGgaucUCAAGGGCAGCAUGCUGGGCCCGUCCC |
| (SEQ ID | ACUACAGGCCAAGAAAUCCGUCUUUCAUUGACGG |
| NO: 34) | GAGCCAGUGUUGCUAGUCAA |
| | (SEQ ID NO: 151) |

U2OS cells were supplied by American Type Culture Collection and maintained below 90% confluency in McCoy's-5A media (Thermo Fisher) supplemented with 10% FBS (Corning) and 100 U/mL Penicillin-Streptomycin (HyClone™). The cells were trypsinized, resuspended, and counted using TrypLE™ Express (Thermo Fisher). A population of 400,000 cells was nucleofected using the SF Cell line nucleofector kit (Lonza) following the manufacturer's pre-set DN-100 program with a mixture of 800 ng of Cas12i2-RT fusion plasmid and 200 ng of each editing template RNA plasmid. Cells were then resuspended and replated in a 96-well plate (40,000 cells/well) with prewarmed growth media. Nucleofected cells were cultured for 72h and harvested.

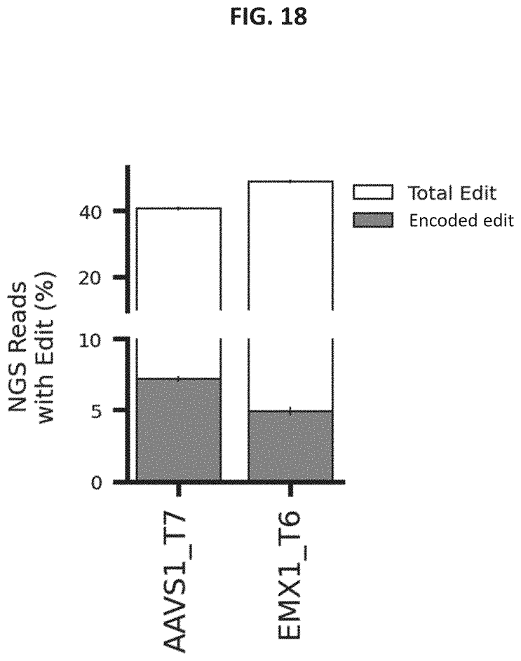

Edits were analyzed by NGS as described in Example 2. As shown in FIG. 18, the edits encoded by each reverse transcription template sequence were identified in about 5-8% of the NGS reads. Encoded edits totaled approximately 20% of the total indels for AAVS1_T7 and approximately 10% of the total indels for the EMX1_T6. This Example and the previous Examples thus show that encoded edits were capable of being introduced into genes of multiple cell lines.

### Example 6—RNA-Templated Editing of Target Strand with Cas12i2-RT Fusions

This Example describes target strand editing of mammalian genes using Cas12i2 variants fused to MMLV RT (SEQ ID NO: 29), a variant of MMLV RT of SEQ ID NO: 29 lacking an RNase H domain (SEQ ID NO: 224), or Marathon RT (SEQ ID NO: 232).

The Cas12i2-RT fusion sequences of Table 14 were cloned into a pcDNA3.1 backbone (Invitrogen). The C-terminal RT fusions comprised a His tag at the N-terminus of Cas12i2 and a bipartite nucleoplasmin NLS (npNLS) at the C-terminus of Cas12i2. Immediately following the npNLS was a GS-XTEN-GS linker. At the C-terminus of the RT was a bipartite SV40 NLS tag. The N-terminal RT fusions comprised a bipartite SV40 NLS tag at the N-terminus and a GS-XTEN-GS linker at the C-terminus of the RT followed by Cas12i2. At the C-terminus of Cas12i2 was a bipartite nucleoplasmin NLS (bpNLS). Working solutions of Cas12i2-RT plasmids were prepared in water.

TABLE 17
CAS-RT FUSION SEQUENCES

| Description | Sequence |
|---|---|
| Variant | MKIEEGKGHHHHHHMSSAIKSYKSV |
| Cas12i2 of | LRPNERKNQLLKSTIQCLEDGSAFF |
| SEQ ID NO: | FKMLQGLFGGITPEIVRFSTEQEKQ |
| 4 fused at C- | QQDIALWCAVNWFRPVSQDSLTHTI |
| terminus to | ASDNLVEKFEEYYGGTASDAIKQYF |
| MMLV RT | SASIGESYYWNDCRQQYYDLCRELG |
| | VEVSDLTHDLEILCREKCLAVATES |
| | NQNNSIISVLFGTGEKEDRSVKLRI |
| | TKKILEAISNLKEIPKNVAPIQEII |
| | LNVAKATKETFRQVYAGNLGAPSTL |
| | EKFIAKDGQKEFDLKKLQTDLKKVI |
| | RGKSKERDWCCQEELRSYVEQNTIQ |
| | YDLWAWGEMFNKAHTALKIKSTRNY |
| | NFAKQRLEQFKEIQSLNNLLVVKKL |
| | NDFFDSEFFSGEETYTICVHHLGGK |
| | DLSKLYKAWEDDPADPENAIVVLCD |
| | DLKNNFKKEPIRNILRYIFTIRQEC |
| | SAQDILAAAKYNQQLDRYKSQKANP |
| | SVLGNQGFTWTNAVILPEKAQRNDR |
| | PNSLDLRIWLYLKLRHPDGRWKKAH |
| | IPFYDTRFFQEIYAAGNSPVDTCQF |
| | RTPRFGYHLPKLTDQTAIRVNKKHV |
| | KAAKTEARIRLAIQQGTLPVSNLKI |
| | TEISATINSKGQVRIPVKFRVGRQK |
| | GTLQIGDRFCGYDQNQTASHAYSLW |
| | EVVKEGQYHKELGCFVRFISSGDIV |
| | SITENRGNQFDQLSYEGLAYPQYAD |
| | WRKKASKFVSLWQITKKNKKKEIVT |
| | VEAKEKFDAICKYQPRLYKFNKEYA |
| | YLLRDIVRGKSLVELQQIRQEIFRF |
| | IEQDCGVTRLGSLSLSTLETVKAVK |
| | GIIYSYFSTALNASKNNPISDEQRK |
| | EFDPELFALLEKLELIRTRKKKQKV |
| | ERIANSLIQTCLENNIKFIRGEGDL |
| | STTNNATKKKANSRSMDWLARGVFN |
| | KIRQLAPMHNITLFGCGSLYTSHQD |
| | PLVHRNPDKAMKCRWAAIPVKDIGD |
| | WVLRKLSQNLRAKNRGTGEYYHQGV |
| | KEFLSHYELQDLEEELLKWRSDRKS |
| | NIPCWVLQNRLAEKLGNKEAVVYIP |
| | VRGGRIYFATHKVATGAVSIVFDQK |
| | QVWVCNADHVAAANIALTGKGIGEQ |
| | SSDEENPDGSRIKLQLTSKRPAATK |
| | KAGQAKKKKSGGSSGGSSGSETPGT |
| | SESATPESSGGSSGGSTLNIEDEYR |
| | LHETSKEPDVSLGSTWLSDFPQAWA |
| | ETGGMGLAVRQAPLIIPLKATSTPV |
| | SIKQYPMSQEARLGIKPHIQRLLDQ |
| | GILVPCQSPWNTPLLPVKKPGTNDY |
| | RPVQDLREVNKRVEDIHPTVPNPYN |
| | LLSGLPPSHQWYTVLDLKDAFFCLR |
| | LHPTSQPLFAFEWRDPEMGISGQLT |
| | WTRLPQGFKNSPTLFNEALHRDLAD |
| | FRIQHPDLILLQYVDDLLLAATSEL |
| | DCQQGTRALLQTLGNLGYRASAKKA |
| | QICQKQVKYLGYLLKEGQRWLTEAR |
| | KETVMGQPTPKTPRQLREFLGKAGF |
| | CRLFIPGFAEMAAPLYPLTKPGTLF |
| | NWGPDQQKAYQEIKQALLTAPALGL |
| | PDLTKPFELFVDEKQGYAKGVLTQK |
| | LGPWRRPVAYLSKKLDPVAAGWPPC |
| | LRMVAAIAVLTKDAGKLTMGQPLVI |
| | LAPHAVEALVKQPPDRWLSNARMTH |
| | YQALLLDTDRVQFGPVVALNPATLL |
| | PLPEEGLQHNCLDILAEAHGTRPDL |
| | TDQPLPDADHTWYTDGSSLLQEGQR |
| | KAGAAVTTETEVIWAKALPAGTSAQ |
| | RAELIALTQALKMAEGKKLNVYTDS |
| | RYAFATAHIHGEIYRRRGWLTSEGK |
| | EIKNKDEILALLKALFLPKRLSIIH |
| | CPGHQKGHSAEARGNRMADQAARKA |
| | AITETPDTSTLLIENSSPMKRTADG |
| | SEFESPK |
| | KKRKV(SEQ ID NO: 219) |
| Variant | MKIEEGKGHHHHHHMSSAIKSYKSV |
| Cas12i2 of | LRPNERKNQLLKSTIQCLEDGSAFF |
| SEQ ID NO: | FKMLQGLFGGITPEIVRFSTEQEKQ |
| 7 fused at C- | QQDIALWCAVNWFRPVSQDSLTHTI |
| terminus to | ASDNLVEKFEEYYGGTASDAIKQYF |
| MMLV with | SASIGESYYWNDCRQQYYDLCRELG |
| RNase H | VEVSDLTHDLEILCREKCLAVATES |
| deletion | NQNNSIISVLFGTGEKEDRSVKLRI |
| | TKKILEAISNLKEIPKNVAPIQEII |
| | LNVAKATKETFRQVYAGNLGAPSTL |
| | EKFIAKDGQKEFDLKKLQTDLKKVI |
| | RGKSKERDWCCQEELRSYVEQNTIQ |
| | YDLWAWGEMFNKAHTALKIKSTRNY |
| | NFAKQRLEQFKEIQSLNNLLVVKKL |
| | NDFFDSEFFSGEETYTICVHHLGGK |
| | DLSKLYKAWEDDPADPENAIVVLCD |
| | DLKNNFKKEPIRNILRYIFTIRQEC |
| | SAQDILAAAKYNQQLDRYKSQKANP |
| | SVLGNQGFTWTNAVILPEKAQRNDR |
| | PNSLDLRIWLYLKLRHPDGRWKKAH |
| | IPFYDTRFFQEIYAAGNSPVDTCQF |
| | RTPRFGYHLPKLTDQTAIRVNKKHV |
| | KAAKTEARIRLAIQQGTLPVSNLKI |
| | TEISATINSKGQVRIPVKFRVGRQK |
| | GTLQIGDRFCGYDQNQTASHAYSLW |
| | EVVKEGQYHKELRCRVRFISSGDIV |
| | SITENRGNQFDQLSYEGLAYPQYAD |
| | WRKKASKFVSLWQITKKNKKKEIVT |
| | VEAKEKFDAICKYQPRLYKFNKEYA |
| | YLLRDIVRGKSLVELQQIRQEIFRF |
| | IEQDCGVTRLGSLSLSTLETVKAVK |
| | GIIYSYFSTALNASKNNPISDEQRK |
| | EFDPELFALLEKLELIRTRKKKQKV |
| | ERIANSLIQTCLENNIKFIRGEGDL |
| | STTNNATKKKANSRSMDWLARGVFN |
| | KIRQLATMHNITLFGCGSLYTSHQD |
| | PLVHRNPDKAMKCRWAAIPVKDIGD |
| | WVLRKLSQNLRAKNRGTGEYYHQGV |
| | KEFLSHYELQDLEEELLKWRSDRKS |
| | NIPCWVLQNRLAEKLGNKEAVVYIP |
| | VRGGRIYFATHKVATGAVSIVFDQK |
| | QVWVCNADHVAAANIALTGKGIGRQ |
| | SSDEENPDGGRIKLQLTSKRPAATK |
| | KAGQAKKKKSGGSSGGSSGSETPGT |
| | SESATPESSGGSSGGSTLNIEDEYR |
| | LHETSKEPDVSLGSTWLSDFPQAWA |
| | ETGGMGLAVRQAPLIIPLKATSTPV |
| | SIKQYPMSQEARLGIKPHIQRLLDQ |
| | GILVPCQSPWNTPLLPVKKPGTNDY |
| | RPVQDLREVNKRVEDIHPTVPNPYN |
| | LLSGLPPSHQWYTVLDLKDAFFCLR |
| | LHPTSQPLFAFEWRDPEMGISGQLT |
| | WTRLPQGFKNSPTLFNEALHRDLAD |
| | FRIQHPDLILLQYVDDLLLAATSEL |
| | DCQQGTRALLQTLGNLGYRASAKKA |
| | QICQKQVKYLGYLLKEGQRWLTEAR |
| | KETVMGQPTPKTPRQLREFLGKAGF |
| | CRLFIPGFAEMAAPLYPLTKPGTLF |
| | NWGPDQQKAYQEIKQALLTAPALGL |
| | PDLTKPFELFVDEKQGYAKGVLTQK |
| | LGPWRRPVAYLSKKLDPVAAGWPPC |
| | LRMVAAIAVLTKDAGKLTMGQPLVI |
| | LAPHAVEALVKQPPDRWLSNARMTH |
| | YQALLLDTDRVQFGPVVALNPATLL |
| | PMKRTADGSEFESPKKKRK |
| | V(SEQ ID NO: 220) |
| Variant | MKIEEGKGHHHHHHMSSAIKSYKSV |
| Cas12i2 of | LRPNERKNQLLKSTIQCLEDGSAFF |
| SEQ ID NO: | FKMLQGLFGGITPEIVRFSTEQEKQ |
| 7 fused at C- | QQDIALWCAVNWFRPVSQDSLTHTI |
| terminus to | ASDNLVEKFEEYYGGTASDAIKQYF |
| Marathon RT | SASIGESYYWNDCRQQYYDLCRELG |
| | VEVSDLTHDLEILCREKCLAVATES |
| | NQNNSIISVLFGTGEKEDRSVKLRI |
| | TKKILEAISNLKEIPKNVAPIQEII |
| | LNVAKATKETFRQVYAGNLGAPSTL |
| | EKFIAKDGQKEFDLKKLQTDLKKVI |
| | RGKSKERDWCCQEELRSYVEQNTIQ |
| | YDLWAWGEMFNKAHTALKIKSTRNY |
| | NFAKQRLEQFKEIQSLNNLLVVKKL |
| | NDFFDSEFFSGEETYTICVHHLGGK |
| | DLSKLYKAWEDDPADPENAIVVLCD |
| | DLKNNFKKEPIRNILRYIFTIRQEC |
| | SAQDILAAAKYNQQLDRYKSQKANP |
| | SVLGNQGFTWTNAVILPEKAQRNDR |
| | PNSLDLRIWLYLKLRHPDGRWKKAH |
| | IPFYDTRFFQEIYAAGNSPVDTCQF |
| | RTPRFGYHLPKLTDQTAIRVNKKHV |
| | KAAKTEARIRLAIQQGTLPVSNLKI |
| | TEISATINSKGQVRIPVKFRVGRQK |
| | GTLQIGDRFCGYDQNQTASHAYSLW |
| | EVVKEGQYHKELRCRVRFISSGDIV |
| | SITENRGNQFDQLSYEGLAYPQYAD |
| | WRKKASKFVSLWQITKKNKKKEIVT |
| | VEAKEKFDAICKYQPRLYKFNKEYA |
| | YLLRDIVRGKSLVELQQIRQEIFRF |
| | IEQDCGVTRLGSLSLSTLETVKAVK |
| | GIIYSYFSTALNASKNNPISDEQRK |
| | EFDPELFALLEKLELIRTRKKKQKV |
| | ERIANSLIQTCLENNIKFIRGEGDL |
| | STTNNATKKKANSRSMDWLARGVFN |
| | KIRQLATMHNITLFGCGSLYTSHQD |
| | PLVHRNPDKAMKCRWAAIPVKDIGD |
| | WVLRKLSQNLRAKNRGTGEYYHQGV |
| | KEFLSHYELQDLEEELLKWRSDRKS |
| | NIPCWVLQNRLAEKLGNKEAVVYIP |
| | VRGGRIYFATHKVATGAVSIVFDQK |
| | QVWVCNADHVAAANIALTGKGIGRQ |
| | SSDEENPDGGRIKLQLTSKRPAATK |
| | KAGQAKKKKSGGSSGGSSGSETPGT |
| | SESATPESSGGSSGGSMDTSNLMEQ |
| | ILSSDNLNRAYLQVVRNKGAEGVDG |
| | MKYTELKEHLAKNGETIKGQLRTRK |
| | YKPQPARRVEIPKPDGGVRNLGVPT |
| | VTDRFIQQAIAQVLTPIYEEQFHDH |
| | SYGFRPNRCAQQAILTALNIMNDGN |
| | DWIVDIDLEKFFDTVNHDKLMTLIG |
| | RTIKDGDVISIVRKYLVSGIMIDDE |
| | YEDSIVGTPQGGNLSPLLANIMLNE |
| | LDKEMEKRGLNFVRYADDCIIMVGS |
| | EMSANRVMRNISRFIEEKLGLKVNM |
| | TKSKVDRPSGLKYLGFGFYFDPRAH |
| | QFKAKPHAKSVAKFKKRMKELTCRS |
| | WGVSNSYKVEKLNQLIRGWINYFKI |
| | GSMKTLCKELDSRIRYRLRMCIWKQ |
| | WKTPQNQEKNLVKLGIDRNTARRVA |
| | YTGKRIAYVCNKGAVNVAISNKRLA |
| | SFGLISMLDYYIEKCVTCMKRTADG |
| | SEFESPKKKRKV |
| | (SEQ ID NO: 221) |
| Variant | MKRTADGSEFESPKKKRKVMDTSNL |
| Cas12i2 of | MEQILSSDNLNRAYLQVVRNKGAEG |
| SEQ ID NO: | VDGMKYTELKEHLAKNGETIKGQLR |
| 4 fused at N- | TRKYKPQPARRVEIPKPDGGVRNLG |
| terminus to | VPTVTDRFIQQAIAQVLTPIYEEQF |
| Marathon RT | HDHSYGFRPNRCAQQAILTALNIMN |
| | DGNDWIVDIDLEKFFDTVNHDKLMT |
| | LIGRTIKDGDVISIVRKYLVSGIMI |
| | DDEYEDSIVGTPQGGNLSPLLANIM |
| | LNELDKEMEKRGLNFVRYADDCIIM |
| | VGSEMSANRVMRNISRFIEEKLGLK |
| | VNMTKSKVDRPSGLKYLGFGFYFDP |
| | RAHQFKAKPHAKSVAKFKKRMKELT |
| | CRSWGVSNSYKVEKLNQLIRGWINY |
| | FKIGSMKTLCKELDSRIRYRLRMCI |
| | WKQWKTPQNQEKNLVKLGIDRNTAR |
| | RVAYTGKRIAYVCNKGAVNVAISNK |
| | RLASFGLISMLDYYIEKCVTCSGGS |
| | SGGSSGSETPGTSESATPESSGGSS |
| | GGSMSSAIKSYKSVLRPNERKNQLL |
| | KSTIQCLEDGSAFFFKMLQGLFGGI |
| | TPEIVRFSTEQEKQQQDIALWCAVN |
| | WFRPVSQDSLTHTIASDNLVEKFEE |
| | YYGGTASDAIKQYFSASIGESYYWN |
| | DCRQQYYDLCRELGVEVSDLTHDLE |
| | ILCREKCLAVATESNQNNSIISVLF |
| | GTGEKEDRSVKLRITKKILEAISNL |
| | KEIPKNVAPIQEIILNVAKATKETF |
| | RQVYAGNLGAPSTLEKFIAKDGQKE |
| | FDLKKLQTDLKKVIRGKSKERDWCC |
| | QEELRSYVEQNTIQYDLWAWGEMFN |
| | KAHTALKIKSTRNYNFAKQRLEQFK |
| | EIQSLNNLLVVKKLNDFFDSEFFSG |
| | EETYTICVHHLGGKDLSKLYKAWED |
| | DPADPENAIVVLCDDLKNNFKKEPI |
| | RNILRYIFTIRQECSAQDILAAAKY |
| | NQQLDRYKSQKANPSVLGNQGFTWT |
| | NAVILPEKAQRNDRPNSLDLRIWLY |
| | LKLRHPDGRWKKAHIPFYDTRFFQE |
| | IYAAGNSPVDTCQFRTPRFGYHLPK |
| | LTDQTAIRVNKKHVKAAKTEARIRL |
| | AIQQGTLPVSNLKITEISATINSKG |
| | QVRIPVKFRVGRQKGTLQIGDRFCG |
| | YDQNQTASHAYSLWEVVKEGQYHKE |
| | LGCFVRFISSGDIVSITENRGNQFD |
| | QLSYEGLAYPQYADWRKKASKFVSL |
| | WQITKKNKKKEIVTVEAKEKFDAIC |
| | KYQPRLYKFNKEYAYLLRDIVRGKS |
| | LVELQQIRQEIFRFIEQDCGVTRLG |
| | SLSLSTLETVKAVKGIIYSYFSTAL |
| | NASKNNPISDEQRKEFDPELFALLE |
| | KLELIRTRKKKQKVERIANSLIQTC |
| | LENNIKFIRGEGDLSTTNNATKKKA |
| | NSRSMDWLARGVFNKIRQLAPMHNI |
| | TLFGCGSLYTSHQDPLVHRNPDKAM |
| | KCRWAAIPVKDIGDWVLRKLSQNLR |
| | AKNRGTGEYYHQGVKEFLSHYELQD |
| | LEEELLKWRSDRKSNIPCWVLQNRL |
| | AEKLGNKEAVVYIPVRGGRIYFATH |
| | KVATGAVSIVFDQKQVWVCNADHVA |
| | AANIALTGKGIGEQSSDEENPDGSR |
| | IKLQLTSKRPAATKKAGQAKKKK |
| | (SEQ ID NO: 222) |
| Variant | MKRTADGSEFESPKKKRKVMDTSNL |
| Cas12i2 of | MEQILSSDNLNRAYLQVVRNKGAEG |
| SEQ ID NO: | VDGMKYTELKEHLAKNGETIKGQLR |
| 7 fused at N- | TRKYKPQPARRVEIPKPDGGVRNLG |
| terminus to | VPTVTDRFIQQAIAQVLTPIYEEQF |
| Marathon RT | HDHSYGFRPNRCAQQAILTALNIMN |
| | DGNDWIVDIDLEKFFDTVNHDKLMT |
| | LIGRTIKDGDVISIVRKYLVSGIMI |
| | DDEYEDSIVGTPQGGNLSPLLANIM |
| | LNELDKEMEKRGLNFVRYADDCIIM |
| | VGSEMSANRVMRNISRFIEEKLGLK |
| | VNMTKSKVDRPSGLKYLGFGFYFDP |
| | RAHQFKAKPHAKSVAKFKKRMKELT |
| | CRSWGVSNSYKVEKLNQLIRGWINY |
| | FKIGSMKTLCKELDSRIRYRLRMCI |
| | WKQWKTPQNQEKNLVKLGIDRNTAR |
| | RVAYTGKRIAYVCNKGAVNVAISNK |
| | RLASFGLISMLDYYIEKCVTCSGGS |
| | SGGSSGSETPGTSESATPESSGGSS |
| | GGSMSSAIKSYKSVLRPNERKNQLL |
| | KSTIQCLEDGSAFFFKMLQGLFGGI |
| | TPEIVRFSTEQEKQQQDIALWCAVN |
| | WFRPVSQDSLTHTIASDNLVEKFEE |
| | YYGGTASDAIKQYFSASIGESYYWN |
| | DCRQQYYDLCRELGVEVSDLTHDLE |
| | ILCREKCLAVATESNQNNSIISVLF |
| | GTGEKEDRSVKLRITKKILEAISNL |
| | KEIPKNVAPIQEIILNVAKATKETF |
| | RQVYAGNLGAPSTLEKFIAKDGQKE |
| | FDLKKLQTDLKKVIRGKSKERDWCC |
| | QEELRSYVEQNTIQYDLWAWGEMFN |
| | KAHTALKIKSTRNYNFAKQRLEQFK |
| | EIQSLNNLLVVKKLNDFFDSEFFSG |
| | EETYTICVHHLGGKDLSKLYKAWED |
| | DPADPENAIVVLCDDLKNNFKKEPI |
| | RNILRYIFTIRQECSAQDILAAAKY |
| | NQQLDRYKSQKANPSVLGNQGFTWT |
| | NAVILPEKAQRNDRPNSLDLRIWLY |
| | LKLRHPDGRWKKAHIPFYDTRFFQE |
| | IYAAGNSPVDTCQFRTPRFGYHLPK |
| | LTDQTAIRVNKKHVKAAKTEARIRL |
| | AIQQGTLPVSNLKITEISATINSKG |
| | QVRIPVKFRVGRQKGTLQIGDRFCG |
| | YDQNQTASHAYSLWEVVKEGQYHKE |
| | LRCRVRFISSGDIVSITENRGNQFD |
| | QLSYEGLAYPQYADWRKKASKFVSL |
| | WQITKKNKKKEIVTVEAKEKFDAIC |
| | KYQPRLYKFNKEYAYLLRDIVRGKS |
| | LVELQQIRQEIFRFIEQDCGVTRLG |
| | SLSLSTLETVKAVKGIIYSYFSTAL |
| | NASKNNPISDEQRKEFDPELFALLE |
| | KLELIRTRKKKQKVERIANSLIQTC |
| | LENNIKFIRGEGDLSTTNNATKKKA |
| | NSRSMDWLARGVFNKIRQLATMHNI |
| | TLFGCGSLYTSHQDPLVHRNPDKAM |
| | KCRWAAIPVKDIGDWVLRKLSQNLR |
| | AKNRGTGEYYHQGVKEFLSHYELQD |
| | LEEELLKWRSDRKSNIPCWVLQNRL |
| | AEKLGNKEAVVYIPVRGGRIYFATH |
| | KVATGAVSIVFDQKQVWVCNADHVA |
| | AANIALTGKGIGRQSSDEENPDGGR |
| | IKLQLTSKRPAATKKAGQAKKKK |
| | (SEQ ID NO: 223) |

The target and corresponding editing template RNA sequences are shown in Table 18. The RT template was 40 nucleotides in length, and the PBS was 13 nucleotides in length. The encoded edit was a 4-nucleotide substitution as well as a single base substitution to remove the PAM sequences. The editing template RNA was further end protected with an additional direct repeat sequence and a non-targeting spacer sequence. The editing template RNAs were cloned into a plasmid backbone with a U6 promoter and maxi-prepped, and a working solution of each editing template RNA plasmid was prepared in water.

TABLE 18
EDITING TEMPLATE RNA SEQUENCES

| Target | Editing template RNA sequences |
|---|---|
| AAVS1_T7 | AGAAAUCCGUCUUUCAUUGACGGGCACAC- |
| GGGAAGTGGT | GACGAUGUAAUCGCUGAUUCCAGCUAC- |
| TGGTCAGCAT | UAUGGGAAGUGGUUGCAGUGCAUGGAUUAU |
| (SEQ ID | AGCCGAAGGCAGAAAUCCGUCUUUCAU- |
| NO: 32) | UGACGGGGGAAGUGGUUGGUCAGCAU |
| | (SEQ ID NO: 135) |
| EMX1_T6 | AGAAAUCCGUCUUUCAUUGACGGGCACAC- |
| GAGCCAGTGT | GACGAUGUAAUCGCAAA- |
| TGCTAGTCAA | GCUUUCCUUCUAUGAGCCAGUGUUGGAUCU |
| (SEQ ID | CAAGGGCAG- |
| NO: 34) | CAUGCUGGGCCAGAAAUCCGUCUUUCAU- |
| | UGACGGGAGCCAGUGUUGCUAGUCAA |
| | (SEQ ID NO: 150) |
| VEGFA_T5 | AGAAAUCCGUCUUUCAUUGACGGGCACAC- |
| TTAAACTCTC | GACGAUGUAAUCGCAACACCAAAAGACUAC- |
| CATGGACCAG | U- |
| (SEQ ID | UAAACUCUCCAACCUCCAGGCUCAUCCAGC |
| NO: 38) | UUCCCAAGAAAUCCGUCUUUCAUUGAC- |
| | GGUUAAACUCUCCAUGGACCAG |
| | (SEQ ID NO: 180) |

HEK293T cells were supplied by American Type Culture Collection and maintained below 90% confluency in D10 media: DMEM (Thermo Fisher) plus GlutaMAX™ (Thermo Fisher) and pyruvate (Thermo Fisher) supplemented with 10% FBS (Corning) and 100 U/mL Penicillin-Streptomycin (HyClone™). Prior to transduction, HEK293T cells were plated in tissue culture treated 96-well plates at 25,000 cells per well. After 15-18h, cells were transfected. Each Cas12i2-RT fusion plasmid and editing template RNA plasmid was diluted in Opti-MEM™ media (Thermo Fisher) and then mixed with Lipofectamine™ 2000 (Thermo Fisher) diluted in Opti-MEM™. The Lipofectamine™ 2000 solution was added dropwise to the wells, and the transfected cells were cultured for 72h before harvesting.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

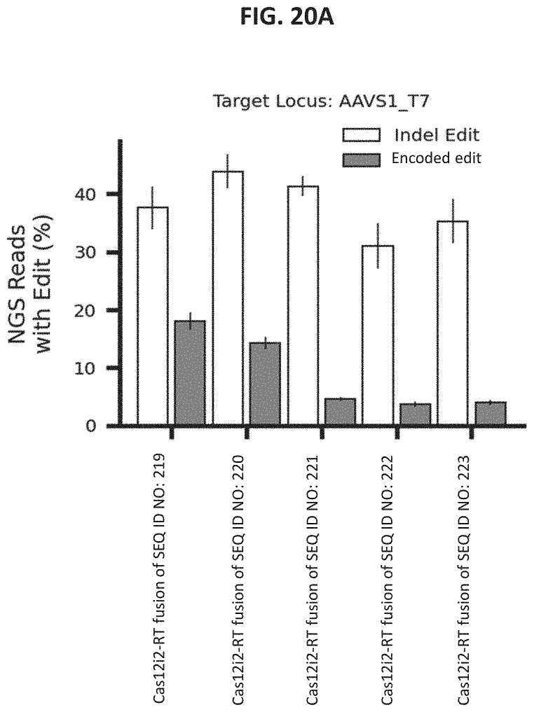

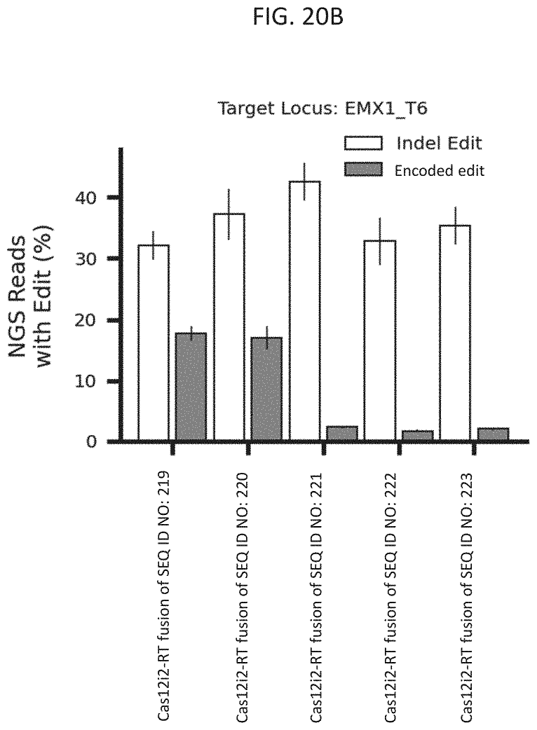

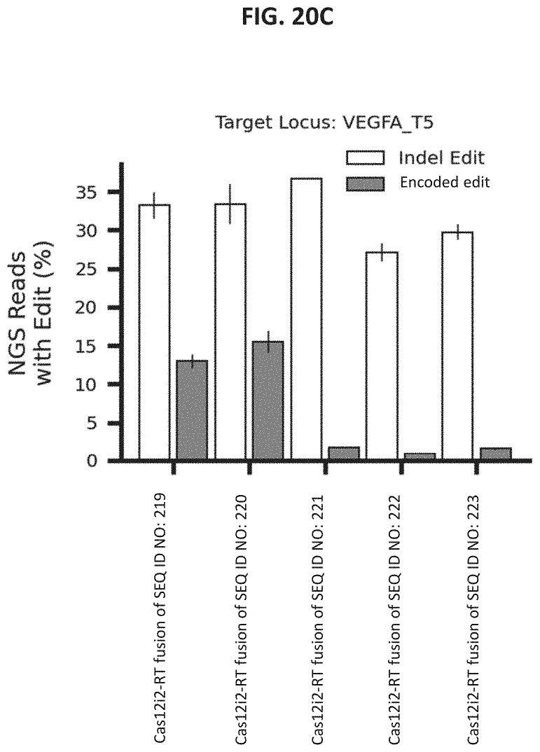

FIG. 20A, FIG. 20B, and FIG. 20C show activity by the Cas12i2-RT fusions of Table 19 on AAVS1_T7, EMX1_T6, and VEGFA_T5, respectively. Indel edit (percentage of total NGS reads comprising an insertion or deletion within or adjacent to the target sequence) and precise edit (percentage of total NGS reads comprising the edit encoded by the editing template RNA) is shown on the y-axis. Indel edits are shown as white bars, and encoded edits are shown as grey bars. The data shown is an average of two bioreplicates. As shown in FIG. 20A, FIG. 20B, and FIG. 20C, the Cas12i2-RT fusions were active nucleases in the presence of the editing template RNAs. Furthermore, each of the Cas12i2-RT fusions introduced edits encoded by the editing template RNAs into the target sequence. For each of the three targets edited with the Cas12i2-RT fusion of SEQ ID NO: 220, approximately 15% of NGS reads comprised the edit encoded by the editing template RNAs. Therefore, deletion of the RNase H domain of MMLV did not appear to have a significant effect on the ability of the Cas12i2-RT fusion to introduce indels and precise edits into the mammalian genome. Furthermore, Cas12i2-RT fusions comprising Marathon RT were capable of introducing encoded edits into the target sequences (FIG. 20A, FIG. 20B, and FIG. 20C).

This Example thus shows that encoded edits are capable of being incorporated into the target strand of mammalian genes using multiple RT sequences and Cas12i2-RT fusions.

### Example 7—RNA-Templated Editing Using Chemically Modified Editing Template RNAs

This Example describes target strand editing of a mammalian gene, VEGFA, using the plasmid-encoded Cas12i2-RT fusion of SEQ ID NO: 219 and editing template RNAs comprising terminal phosphorothioate backbone linkages and/or 2′O-methyl nucleotides. The target sequence was TTAAACTCTCCATGGACCAG (SEQ ID NO: 38).

TABLE 19
RNA GUIDE AND EDITING TEMPLATE RNA SEQUENCES.

| RNA Construct | RNA Sequence |
|---|---|
| Unmodified RNA guide | rArGrArArArUrCrCrGrUrCrUrUrUrCrArUrUrGrArCrGrGrU |
| | rUrArArArCrUrCrUrCrCrArUrGrGrArCrCrArG (SEQ ID |
| | NO: 27) |
| PS modified RNA guide | rA*rG*rA*rArArUrCrCrGrUrCrUrUrUrCrArUrUrGrArCrGr |
| | GrUrUrArArArCrUrCrUrCrCrArUrGrGrArC*rC*rA*rG |
| | (SEQ ID NO: 28) |
| 2′-O-Me modified RNA | mAmGmArArArUrCrCrGrUrCrUrUrUrCrArUrUrGrArCrGrG |
| guide | rUrUrArArArCrUrCrUrCrCrArUrGrGrAmCmCmArG (SEQ |
| | ID NO: (SEQ ID NO: 31) |
| PS-2′-O-Me modified RNA | mA*mG*mA*rArArUrCrCrGrUrCrUrUrUrCrArUrUrGrArCr |
| guide | GrGrUrUrArArArCrUrCrUrCrCrArUrGrGrAmC*mC*mA*r |
| | G (SEQ ID NO: 33) |
| Unmodified editing template | rArGrArArArUrCrCrGrUrCrUrUrUrCrArUrUrGrArCrGrGrU |
| RNA (PBS 13 nt) | rUrArArArCrUrCrUrCrCrArUrGrGrArCrCrArGrArArArArGr |
| | ArCrUrUrUrUrUrArArArCrUrCrUrCrCrArArCrCrUrCrCrAr |
| | GrGrCrUrCrArUrCrCrArGrCrUrUrCrCrCrA (SEQ ID NO: |
| | 35) |
| PS modified editing template | rA*rG*rA*rArArUrCrCrGrUrCrUrUrUrCrArUrUrGrArCrGr |
| RNA (PBS 13 nt) | GrUrUrArArArCrUrCrUrCrCrArUrGrGrArCrCrArGrArArAr |
| | ArGrArCrUrUrUrUrUrArArArCrUrCrUrCrCrArArCrCrUrCr |
| | CrArGrGrCrUrCrArUrCrCrArGrCrUrUrC*rC*rC*rA (SEQ |
| | ID NO: 37) |
| 2′-O-Me modified editing | mAmGmArArArUrCrCrGrUrCrUrUrUrCrArUrUrGrArCrGrG |
| template RNA (PBS 13 nt) | rUrUrArArArCrUrCrUrCrCrArUrGrGrArCrCrArGrArArArAr |
| | GrArCrUrUrUrUrUrArArArCrUrCrUrCrCrArArCrCrUrCrCr |
| | ArGrGrCrUrCrArUrCrCrArGrCrUrUmCmCmCrA (SEQ ID |
| | NO: 39) |
| PS-2′-O-Me editing template | mA*mG*mA*rArArUrCrCrGrUrCrUrUrUrCrArUrUrGrArCr |
| RNA (PBS 13 nt) | GrGrUrUrArArArCrUrCrUrCrCrArUrGrGrArCrCrArGrArAr |
| | ArArGrArCrUrUrUrUrUrArArArCrUrCrUrCrCrArArCrCrUr |
| | CrCrArGrGrCrUrCrArUrCrCrArGrCrUrUmC*mC*mC*rA |
| | (SEQ ID NO: 46) |
| Unmodified editing template | rArGrArArArUrCrCrGrUrCrUrUrUrCrArUrUrGrArCrGrGrU |
| RNA (PBS 30 nt) | rUrArArArCrUrCrUrCrCrArUrGrGrArCrCrArGrArArArArGr |
| | ArCrUrUrUrUrUrArArArCrUrCrUrCrCrArArCrCrUrCrCrAr |
| | GrGrCrUrCrArUrCrCrArGrCrUrUrCrCrCrArArArCrArArAr |
| | GrCrCrCrCrCrArArGrArA (SEQ ID NO: 47) |
| PS modified editing template | rA*rG*rA*rArArUrCrCrGrUrCrUrUrUrCrArUrUrGrArCrGr |
| RNA (PBS 30 nt) | GrUrUrArArArCrUrCrUrCrCrArUrGrGrArCrCrArGrArArAr |
| | ArGrArCrUrUrUrUrUrArArArCrUrCrUrCrCrArArCrCrUrCr |
| | CrArGrGrCrUrCrArUrCrCrArGrCrUrUrCrCrCrArArArCrAr |
| | ArArGrCrCrCrCrCrArA*rG*rA*rA (SEQ ID NO: 48) |
| 2′-O-Me modified editing | mAmGmArArArUrCrCrGrUrCrUrUrUrCrArUrUrGrArCrGrG |
| template RNA (PBS 30 nt) | rUrUrArArArCrUrCrUrCrCrArUrGrGrArCrCrArGrArArArAr |
| | GrArCrUrUrUrUrUrArArArCrUrCrUrCrCrArArCrCrUrCrCr |
| | ArGrGrCrUrCrArUrCrCrArGrCrUrUrCrCrCrArArArCrArAr |
| | ArGrCrCrCrCrCrAmAmGmArA (SEQ ID NO: 49) |
| PS-2′-O-Me editing template | mA*mG*mA*rArArUrCrCrGrUrCrUrUrUrCrArUrUrGrArCr |
| RNA (PBS 30 nt) | GrGrUrUrArArArCrUrCrUrCrCrArUrGrGrArCrCrArGrArAr |
| | ArArGrArCrUrUrUrUrUrArArArCrUrCrUrCrCrArArCrCrUr |
| | CrCrArGrGrCrUrCrArUrCrCrArGrCrUrUrCrCrCrArArArCr |
| | ArArArGrCrCrCrCrCrAmA*mG*mA*rA (SEQ ID NO: 50) |

Variant Cas12i2 of SEQ ID NO: 4 and the Cas12i2-RT fusion of SEQ ID NO: 219 were individually cloned into a pcDNA3.1 backbone (Invitrogen). The RNA guide and editing template RNA sequences were synthesized by IDT. HEK293T cells were supplied by American Type Culture Collection and maintained below 90% confluency D10 media: DMEM (Thermo Fisher) plus GlutaMAX™ (Thermo Fisher) and pyruvate (Thermo Fisher) supplemented with 10% FBS (Corning) and 100 U/mL Penicillin-Streptomycin (HyClone™) Prior to transduction, HEK293T cells were plated in tissue culture treated 96-well plates at 25,000 cells per well in D10. After 15-18h, cells were transfected by TransIT-X2® (Mirus Bio). The DNA plus transfection reagent solution was then added dropwise to a well of cells. A mixture of 100 ng of Cas12i2 or Cas12i2-RT plasmid DNA and 9 pmol of synthesized RNA guide (IDT) was diluted in Opti-MEM™ media (Thermo Fisher) and then mixed with Lipofectamine™ 2000 diluted in Opti-MEM™ following the manufacturer's instructions. Transfected cells were cultured for 72h before harvesting.

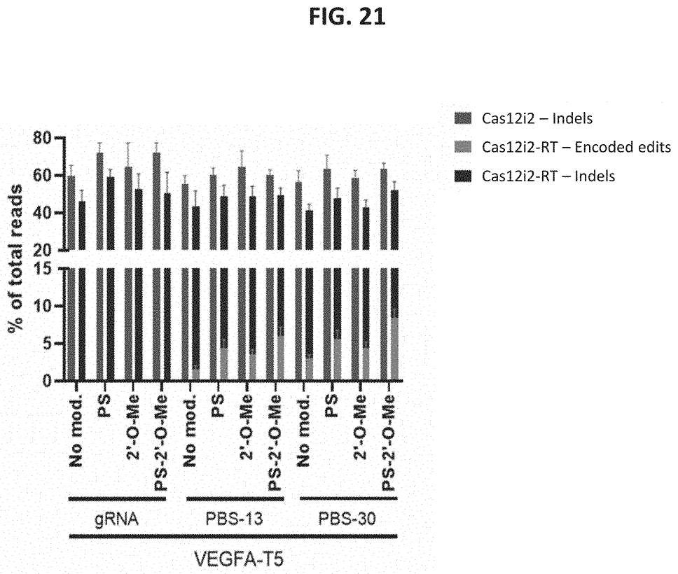

Edits were analyzed by NGS, as described in previous Examples. As shown in FIG. 21, encoded edits at the VEGFA-T5 target site were detected with each of the editing template RNAs and Cas12i2-RT fusion of SEQ ID NO: 219. Encoded edits were not detected in the control (gRNA and editing template RNA+Cas12i2) samples. Encoded edits were detected in a higher percentage of NGS reads using modified editing template RNAs compared to unmodified editing template RNAs. Use of PS-2′-O-Me modifications resulted in the highest percentage of NGS reads comprising the encoded edit.

Therefore, this Example shows that genomic sites of interest are capable of being edited by chemically modified editing template RNAs and Cas12i2-RT fusions.

### Example 8—RNA-Templated Editing Using Cas12i4-RT Fusions

This Example describes target strand editing of AAVS1 using a Cas12i4 variant fused to MMLV RT (SEQ ID NO: 29).

The Cas12i4-RT fusion sequences of Table 20 were cloned into a pcDNA3.1 backbone (Invitrogen). The C-terminal RT fusion comprised a His tag at the N-terminus of Cas12i4 and a nucleoplasmin NLS at the C-terminus of Cas12i4. Immediately following the NLS was a Flex XTEN linker. At the C-terminus of the RT was a bipartite SV40 NLS tag. The N-terminal RT fusion comprised a bipartite SV40 NLS tag at the N-terminus and a Flex XTEN linker at the C-terminus of the RT followed by Cas12i4. At the C-terminus of Cas12i4 was a nucleoplasmin NLS. Working solutions of Cas12i4-RT plasmids were prepared in water.

TABLE 20
VARIANT CAS12I4 AND VARIANT
CAS12I4-RT FUSION SEQUENCES

| Description | Sequence |
|---|---|
| Variant | MASISRPYGTKLRPDARKKEMLDKF |
| Cas12i4 | FNTLTKGQRVFADLALCIYGSLTLE |
| | MAKSLEPESDSELVCAIGWFRLVDK |
| | TIWSKDGIKQENLVKQYEAYSGKEA |
| | SEVVKTYLNSPSSDKYVWIDCRQKF |
| | LRFQRELGTRNLSEDFECMLFEQYI |
| | RLTKGEIEGYAAISNMFGNGEKEDR |
| | SKKRMYATRMKDWLEANENITWEQY |
| | REALKNQLNAKNLEQVVANYKGNAG |
| | GADPFFKYSFSKEGMVSKKEHAQQL |
| | DKFKTVLKNKARDLNFPNKEKLKQY |
| | LEAEIGIPVDANVYSQMFSNGVSEV |
| | QPKTTRNMSFSNEKLDLLTELKDLN |
| | KGDGFEYAREVLNGFFDSELHTTED |
| | KFNITSRYLGGDKSNRLSKLYKIWK |
| | KEGVDCEEGIQQFCEAVKDKMGQIP |
| | IRNVLKYLWQFRETVSAEDFEAAAK |
| | ANHLEEKISRVKAHPIVISNRYWAF |
| | GTSALVGNIMPADKRHQGEYAGQNF |
| | KMWLRAELHYDGKKAKAHLPFYNAR |
| | FFEEVYCYHPSVAEITPFKTKQFGC |
| | EIGKDIPDYVSVALKDNPYKKATKR |
| | ILRAIYNPVANTTRVDKTTNCSFMI |
| | KRENDEYKLVINRKISRDRPKRIEV |
| | GRTIMGYDRNQTASDTYWIGRLVPP |
| | GTRGAYRIGEWSVQYIKSGPVLSST |
| | QGVNNSTTDQLVYNGMPSSSERFKA |
| | WKKARMAFIRKLIRQLNDEGLESKG |
| | QDYIPENPSSFDVRGETLYVFNSNY |
| | LKALVSKHRKAKKPVEGILDEIEAW |
| | TSKDKDSCSLMRLSSLSDASMQGIA |
| | SLKSLINSYFNKNGCKTIEDKEKFN |
| | PVLYAKLVEVEQRRTNKRSEKVGRI |
| | AGSLEQLALLNGVEVVIGEADLGEV |
| | EKGKSKKQNSRNMDWCAKQVAQRLE |
| | YKLAFHGIGYFGVNPMYTSHQDPFE |
| | HRRVADHIVMRARFEEVNVENIAEW |
| | HVRNFSNYLRADSGTGLYYKQATMD |
| | FLKHYGLEEHAEGLENKKIKFYDFR |
| | KILEDKNLTSVIIPKRGGRIYMATN |
| | PVTSDSTPITYAGKTYNRCNADEVA |
| | AANIVISVLAPRSKKNREQDDIPLI |
| | TKKAESKSPPKDRKRSKTSQLPQK |
| | (SEQ ID NO: 51) |
| Variant | MKIEEGKGHHHHHHMASISRPYGTK |
| | LRPDARKKEMLDKFFNTLTKGQRVF |
| | ADLALCIYGSLTLEMAKSLEPESDS |
| | ELVCAIGWFRLVDKTIWSKDGIKQE |
| | NLVKQYEAYSGKEASEVVKTYLNSP |
| | SSDKYVWIDCRQKFLRFQRELGTRN |
| | LSEDFECMLFEQYIRLTKGEIEGYA |
| | AISNMFGNGEKEDRSKKRMYATRMK |
| | DWLEANENITWEQYREALKNQLNAK |
| | NLEQVVANYKGNAGGADPFFKYSFS |
| | KEGMVSKKEHAQQLDKFKTVLKNKA |
| | RDLNFPNKEKLKQYLEAEIGIPVDA |
| | NVYSQMFSNGVSEVQPKTTRNMSFS |
| | NEKLDLLTELKDLNKGDGFEYAREV |
| | LNGFFDSELHTTEDKFNITSRYLGG |
| | DKSNRLSKLYKIWKKEGVDCEEGIQ |
| | QFCEAVKDKMGQIPIRNVLKYLWQF |
| | RETVSAEDFEAAAKANHLEEKISRV |
| | KAHPIVISNRYWAFGTSALVGNIMP |
| | ADKRHQGEYAGQNFKMWLRAELHYD |
| | GKKAKAHLPFYNARFFEEVYCYHPS |
| | VAEITPFKTKQFGCEIGKDIPDYVS |
| | VALKDNPYKKATKRILRAIYNPVAN |
| | TTRVDKTTNCSFMIKRENDEYKLVI |
| | NRKISRDRPKRIEVGRTIMGYDRNQ |
| | TASDTYWIGRLVPPGTRGAYRIGEW |
| | SVQYIKSGPVLSSTQGVNNSTTDQL |
| | VYNGMPSSSERFKAWKKARMAFIRK |
| | LIRQLNDEGLESKGQDYIPENPSSF |
| | DVRGETLYVFNSNYLKALVSKHRKA |
| | KKPVEGILDEIEAWTSKDKDSCSLM |
| | RLSSLSDASMQGIASLKSLINSYFN |
| | KNGCKTIEDKEKFNPVLYAKLVEVE |
| | QRRTNKRSEKVGRIAGSLEQLALLN |
| | GVEVVIGEADLGEVEKGKSKKQNSR |
| | NMDWCAKQVAQRLEYKLAFHGIGYF |
| | GVNPMYTSHQDPFEHRRVADHIVMR |
| | ARFEEVNVENIAEWHVRNFSNYLRA |
| | DSGTGLYYKQATMDFLKHYGLEEHA |
| Cas12i4 fused | EGLENKKIKFYDFRKILEDKNLTSV |
| at C-terminus | IIPKRGGRIYMATNPVTSDSTPITY |
| to MMLV RT | AGKTYNRCNADEVAAANIVISVLAP |
| | RSKKNREQDDIPLITKKAESKSPPK |
| | DRKRSKTSQLPQKKRPAATKKAGQA |
| | KKKKSGGSSGGSSGSETPGTSESAT |
| | PESSGGSSGGSTLNIEDEYRLHETS |
| | KEPDVSLGSTWLSDFPQAWAETGGM |
| | GLAVRQAPLIIPLKATSTPVSIKQY |
| | PMSQEARLGIKPHIQRLLDQGILVP |
| | CQSPWNTPLLPVKKPGTNDYRPVQD |
| | LREVNKRVEDIHPTVPNPYNLLSGL |
| | PPSHQWYTVLDLKDAFFCLRLHPTS |
| | QPLFAFEWRDPEMGISGQLTWTRLP |
| | QGFKNSPTLFNEALHRDLADFRIQH |
| | PDLILLQYVDDLLLAATSELDCQQG |
| | TRALLQTLGNLGYRASAKKAQICQK |
| | QVKYLGYLLKEGQRWLTEARKETVM |
| | GQPTPKTPRQLREFLGKAGFCRLFI |
| | PGFAEMAAPLYPLTKPGTLFNWGPD |
| | QQKAYQEIKQALLTAPALGLPDLTK |
| | PFELFVDEKQGYAKGVLTQKLGPWR |
| | RPVAYLSKKLDPVAAGWPPCLRMVA |
| | AIAVLTKDAGKLTMGQPLVILAPHA |
| | VEALVKQPPDRWLSNARMTHYQALL |
| | LDTDRVQFGPVVALNPATLLPLPEE |
| | GLQHNCLDILAEAHGTRPDLTDQPL |
| | PDADHTWYTDGSSLLQEGQRKAGAA |
| | VTTETEVIWAKALPAGTSAQRAELI |
| | ALTQALKMAEGKKLNVYTDSRYAFA |
| | TAHIHGEIYRRRGWLTSEGKEIKNK |
| | DEILALLKALFLPKRLSIIHCPGHQ |
| | KGHSAEARGNRMADQAARKAAITET |
| | PDTSTLLIENSSPMKRTADGSEFES |
| | PKKKRKV |
| | (SEQ ID NO: 52) |
| Variant | MKRTADGSEFESPKKKRKVTLNIED |
| | EYRLHETSKEPDVSLGSTWLSDFPQ |
| | AWAETGGMGLAVRQAPLIIPLKATS |
| | TPVSIKQYPMSQEARLGIKPHIQRL |
| | LDQGILVPCQSPWNTPLLPVKKPGT |
| | NDYRPVQDLREVNKRVEDIHPTVPN |
| | PYNLLSGLPPSHQWYTVLDLKDAFF |
| | CLRLHPTSQPLFAFEWRDPEMGISG |
| | QLTWTRLPQGFKNSPTLFNEALHRD |
| | LADFRIQHPDLILLQYVDDLLLAAT |
| | SELDCQQGTRALLQTLGNLGYRASA |
| | KKAQICQKQVKYLGYLLKEGQRWLT |
| | EARKETVMGQPTPKTPRQLREFLGK |
| | AGFCRLFIPGFAEMAAPLYPLTKPG |
| | TLFNWGPDQQKAYQEIKQALLTAPA |
| | LGLPDLTKPFELFVDEKQGYAKGVL |
| | TQKLGPWRRPVAYLSKKLDPVAAGW |
| | PPCLRMVAAIAVLTKDAGKLTMGQP |
| | LVILAPHAVEALVKQPPDRWLSNAR |
| | MTHYQALLLDTDRVQFGPVVALNPA |
| | TLLPLPEEGLQHNCLDILAEAHGTR |
| | PDLTDQPLPDADHTWYTDGSSLLQE |
| | GQRKAGAAVTTETEVIWAKALPAGT |
| | SAQRAELIALTQALKMAEGKKLNVY |
| | TDSRYAFATAHIHGEIYRRRGWLTS |
| | EGKEIKNKDEILALLKALFLPKRLS |
| | IIHCPGHQKGHSAEARGNRMADQAA |
| | RKAAITETPDTSTLLIENSSPSGGS |
| | SGGSSGSETPGTSESATPESSGGSS |
| | GGSMASISRPYGTKLRPDARKKEML |
| | DKFFNTLTKGQRVFADLALCIYGSL |
| | TLEMAKSLEPESDSELVCAIGWFRL |
| | VDKTIWSKDGIKQENLVKQYEAYSG |
| | KEASEVVKTYLNSPSSDKYVWIDCR |
| | QKFLRFQRELGTRNLSEDFECMLFE |
| | QYIRLTKGEIEGYAAISNMFGNGEK |
| | EDRSKKRMYATRMKDWLEANENITW |
| | EQYREALKNQLNAKNLEQVVANYKG |
| | NAGGADPFFKYSFSKEGMVSKKEHA |
| | QQLDKFKTVLKNKARDLNFPNKEKL |
| | KQYLEAEIGIPVDANVYSQMFSNGV |
| | SEVQPKTTRNMSFSNEKLDLLTELK |
| Cas12i4 fused | DLNKGDGFEYAREVLNGFFDSELHT |
| at N-terminus | TEDKFNITSRYLGGDKSNRLSKLYK |
| to MMLV RT | IWKKEGVDCEEGIQQFCEAVKDKMG |
| | QIPIRNVLKYLWQFRETVSAEDFEA |
| | AAKANHLEEKISRVKAHPIVISNRY |
| | WAFGTSALVGNIMPADKRHQGEYAG |
| | QNFKMWLRAELHYDGKKAKAHLPFY |
| | NARFFEEVYCYHPSVAEITPFKTKQ |
| | FGCEIGKDIPDYVSVALKDNPYKKA |
| | TKRILRAIYNPVANTTRVDKTTNCS |
| | FMIKRENDEYKLVINRKISRDRPKR |
| | IEVGRTIMGYDRNQTASDTYWIGRL |
| | VPPGTRGAYRIGEWSVQYIKSGPVL |
| | SSTQGVNNSTTDQLVYNGMPSSSER |
| | FKAWKKARMAFIRKLIRQLNDEGLE |
| | SKGQDYIPENPSSFDVRGETLYVFN |
| | SNYLKALVSKHRKAKKPVEGILDEI |
| | EAWTSKDKDSCSLMRLSSLSDASMQ |
| | GIASLKSLINSYFNKNGCKTIEDKE |
| | KFNPVLYAKLVEVEQRRTNKRSEKV |
| | GRIAGSLEQLALLNGVEVVIGEADL |
| | GEVEKGKSKKQNSRNMDWCAKQVAQ |
| | RLEYKLAFHGIGYFGVNPMYTSHQD |
| | PFEHRRVADHIVMRARFEEVNVENI |
| | AEWHVRNFSNYLRADSGTGLYYKQA |
| | TMDFLKHYGLEEHAEGLENKKIKFY |
| | DFRKILEDKNLTSVIIPKRGGRIYM |
| | ATNPVTSDSTPITYAGKTYNRCNAD |
| | EVAAANIVISVLAPRSKKNREQDDI |
| | PLITKKAESKSPPKDRKRSKTSQLP |
| | QKKRPAATKKAGQAKKKK |
| | (SEQ ID NO: 53) |

The target, RNA guide, and editing template RNA sequences are shown in Table 21. The RT template was 46 nucleotides in length, and the PBS was 13 nucleotides in length. The encoded edit was a 4-nucleotide substitution as well as a single base substitution to remove the PAM sequences. The editing template RNA and RNA guide were individually cloned into a plasmid backbone with a U6 promoter and maxi-prepped, and a working solution of each RNA guide or editing template RNA plasmid was prepared in water.

TABLE 21
TARGET AND RNA SEQUENCES.

| Description | Sequence |
|---|---|
| AAVSl_T7 Target | GGGAAGTGGTTG |
| | GTCAGCAT |
| | (SEQ ID NO: 32) |
| Cas12i4 RNA | AGACAUGUGUCCU |
| guide | CAGUGACACGGGA |
| | AGUGGUUGGU |
| | CAGCAU |
| | (SEQ ID NO: 60) |
| Cas12i4 Editing | AGACAUGUGUCCU |
| Template RNA | CAGUGACACGGGA |
| | AGUGGUUGGU |
| | CAGCAUUGAUUCC |
| | AGCUACUcUGGGA |
| | AGUGGUUGcag |
| | UGCAUGGAUUAUA |
| | GCCGAAGGC |
| | (SEQ ID NO: 61) |

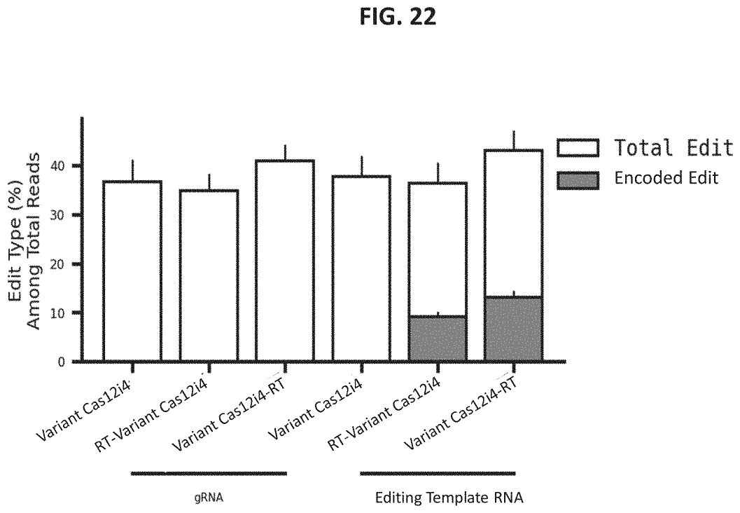

HEK293T cells were transfected and harvested as described in Example 6. NGS was further performed as described in previous examples. As shown in FIG. 22, encoded edits at the AAVS1_T7 target site were detected with the editing template RNAs and either of the Cas12i4-RT fusions. Encoded edits were not detected in the control (gRNA and editing template RNA+Cas12i4) samples. Encoded edits were detected in a higher percentage of NGS reads using the C-terminal fusion of MMLV to variant Cas12i4 compared to the N-terminal fusion of MMLV to variant Cas12i4.

Therefore, this Example shows that genomic sites of interest are capable of being edited by editing template RNAs and Cas12i4-RT fusions.

### Example 9—RNA-Templated Editing Using a Cas12i2-RT Fusion, an RNA Guide, and an RT Donor RNA

This Example describes target strand editing of mammalian genes using a Cas12i2-RT fusion, an RNA guide, and an RT donor RNA.

The Cas12i2-RT fusion of SEQ ID NO: 219 was cloned into a pcDNA3.1 backbone (Invitrogen). A working solution of Cas12i2-RT plasmid was prepared in water. The RNA guides and RT donor RNAs of Table 22 were individually cloned into a plasmid backbone with a U6 promoter and maxi-prepped, and a working solution of each RNA guide or RT donor RNA plasmid was prepared in water. The RT donor RNAs comprised the following components in order from 5′ to 3′: direct repeat—nontargeting spacer—RT template—PBS—direct repeat—nontargeting spacer. The direct repeat and spacer sequences flanking the RT template and PBS served as end protection.

TABLE 22
Target, RNA guide, and RT donor
RNA sequences

| | RNA guide | RT Donor RNA |
|---|---|---|
| Target | sequence | sequence |
| AAVS1_T7 | AGAAAUCCGU | AGAAAUCCGUCUUUCAUUGA |
| (SEQ ID | CUUUCAUUGA | CGGGCACACGACGAUGUAAU |
| NO: 32) | CGGGGGAAGU | CGCCAGCUACUaUGGGAAGU |
| | GGUUGGUCAG | GGUUGcagUGCAUGGAUUAU |
| | CAU | AGCCGAAG |
| | (SEQ ID | GCCCCAGCUUUGCCUUGUUA |
| | NO: 112) | GAAAUCCGUCUUUCAUUGAC |
| | | GGGACGCUACUAUAGCUGCA |
| | | C |
| | | (SEQ ID NO: 62) |
| EMX1_T6 | AGAAAUCCGU | AGAAAUCCGUCUUUCAUUGA |
| (SEQ ID | CUUUCAUUGA | CGGGCACACGACGAUGUAAU |
| NO: 34) | CGGGAGCCAG | CGCUUCCUUCUaUGAGCCAG |
| | UGUUGCUAGU | UGUUGgaUcUCAAGGGCAGC |
| | CAA | AUGCUGGGC |
| | (SEQ ID | CCGUCCCACUACAGGCCAAG |
| | NO: 114) | AAAUCCGUCUUUCAUUGACG |
| | | GGACGCUACUAUAGCUGCAC |
| | | (SEQ ID NO: 63) |
| VEGFA_T2 | AGAAAUCCGU | AGAAAUCCGUCUUUCAUUGA |
| (SEQ ID | CUUUCAUUGA | CGGGCACACGACGAUGUAAU |
| NO: 36) | CGGAAUCCUC | CGCAGAUACCUaUAAUCCUC |
| | CACCAGUCAU | CACCAcagUUGGUGACAACC |
| | GGU | CCAAGCAGCCCACACAUUcU |
| | (SEQ ID | CAAGUGCAGAAAUCCGUCU |
| | NO: 116) | UUCAUUGACGGGACGCUACU |
| | | AUAGCUGCAC |
| | | (SEQ ID NO: 64) |

HEK293T cells were supplied by American Type Culture Collection and maintained below 90% confluency in D10 media: DMEM (Thermo Fisher) plus GlutaMAX™ (Thermo Fisher) and pyruvate (Thermo Fisher) supplemented with 10% FBS (Corning) and 100 U/mL Penicillin-Streptomycin (HyClone™). Prior to transduction, HEK293T cells were plated in tissue culture treated 96-well plates at 25,000 cells per well. After 15-18h, cells were transfected. Each Cas12i2-RT fusion plasmid, RNA guide plasmid, and RT donor RNA plasmid was diluted in Opti-MEM™ media (Thermo Fisher) and then mixed with Lipofectamine™ 2000 (Thermo Fisher) diluted in Opti-MEM™. The Lipofectamine™ 2000 solution was added dropwise to the wells, and the transfected cells were cultured for 72h before harvesting.

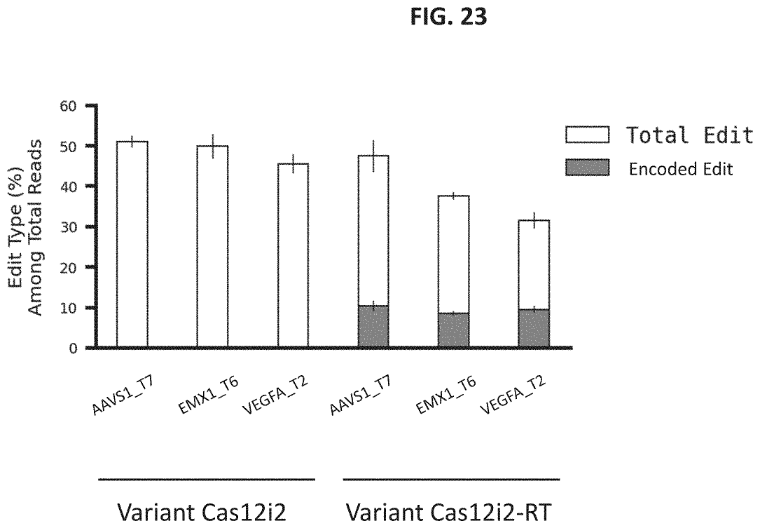

NGS was further performed as described in previous examples. As shown in FIG. 23, encoded edits at each of the target sites were detected following transfection with the Cas12i2-RT fusion, respective RNA guide, and respective RT donor RNA. Encoded edits were not detected in the control (Cas12i2) samples. This Example thus shows that selected genomic sites are capable of being edited by a Cas12i2-RT fusion and two RNA components, an RNA guide and an RT donor RNA. An RNA guide and RT donor RNA need not be fused for incorporation of encoded edits into a genomic site of interest.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system comprising:
(a) a Type V CRISPR nuclease polypeptide or a first nucleic acid encoding the Type V CRISPR nuclease polypeptide;
(b) a reverse transcriptase (RT) polypeptide or a second nucleic acid encoding the RT polypeptide;
(c) a guide RNA (gRNA) or a third nucleic acid encoding the gRNA, wherein the gRNA comprises one or more binding sites recognizable by the Type V CRISPR nuclease (CRISPR nuclease binding sites) and a spacer sequence specific to a target sequence within a genomic site of interest, the target sequence being adjacent to a protospacer adjacent motif (PAM); and
(d) a reverse transcription donor RNA (RT donor RNA) or a fourth nucleic acid encoding the RT donor RNA, wherein the RT donor RNA comprises a primer binding site (PBS) and a template sequence.

2. The gene editing system of claim 1, wherein the Type V CRISPR nuclease polypeptide is a Cas12i polypeptide.

3. The gene editing system of claim 2, wherein the Cas12i polypeptide is a Cas12i2 polypeptide or a Cas12i4 polypeptide.

4. The gene editing system of claim 3, wherein the Cas12i polypeptide is a Cas12i2 polypeptide, which comprises an amino acid sequence at least 95% identical to SEQ ID NO: 2; and/or one or more mutations at positions H485, H486, D581, G624, F626, P868, I926, V1030, E1035, and/or S1046 of SEQ ID NO: 2; or
wherein the Cas12i polypeptide is a Cas12i4 polypeptide, which comprises an amino acid sequence at least 95% identical to SEQ ID NO: 9 or SEQ ID NO: 10.

5-6. (canceled)

7. The gene editing system of claim 4, wherein the Cas12i2 polypeptide comprises:
(i) mutations at positions D581, D911, I926, and V1030, which optionally are amino acid substitutions of D581R, D911R, 1926R, and V1030G;
(ii) mutations at positions D581, I926, and V1030, which optionally are amino acid substitutions of D581R, 1926R, and V1030G;
(iii) mutations at positions D581, I926, V1030, and S1046, which optionally are amino acid substitutions of D581R, I926R, V1030G, and S1046G;
(iv) mutations at positions D581, G624, F626, I926, V1030, E1035, and S1046, which optionally are amino acid substitutions of D581R, G624R, F626R, I926R, V1030G, E1035R, and S1046G; or
(v) mutations at positions D581, G624, F626, P868, I926, V1030, E1035, and S1046, which optionally are amino acid substitutions of D581R, G624R, F626R, P868T, I926R, V1030G, E1035R, and S1046G;
and/or wherein the Cas12i2 polypeptide comprises a mutation at position H485 and/or H486, optionally at position H485.

8-12. (canceled)

13. The gene editing system of claim 1, wherein the gene editing system comprises the first nucleic acid, which is a first messenger RNA (mRNA).

14. The gene editing system of claim 1, wherein the RT polypeptide is Moloney Murine Leukemia Virus (MMLV)-RT, mouse mammary tumor virus (MMTV)-RT, Marathon-RT, or RTx-RT.

15-20. (canceled)

21. The gene editing system of claim 1, wherein the gene editing system comprises a fusion polypeptide, which comprises the Type V CRISPR nuclease polypeptide and the RT polypeptide.

22-24. (canceled)

25. The gene editing system of claim 3, wherein the Cas12i polypeptide is the Cas12i2 polypeptide and the one or more CRISPR nuclease binding sites are direct repeat sequence(s), which is at least 90% identical to any one of SEQ ID NOs: 15-17 and 241-247, or a fragment thereof that is at least 23-nucleotide in length; or
wherein the Cas12i polypeptide is the Cas12i4 polypeptide and the one or more CRISPR nuclease binding sites are direct repeat sequence(s), which is at least 90% identical to any one of SEQ ID NOs: 21-24.

26-27. (canceled)

28. The gene editing system of claim 25, wherein the direct repeat sequence is any one of SEQ ID NOs: 15-17 and 241-247, or a fragment thereof that is at least 23-nucleotide in length; optionally wherein the direct repeat sequence is SEQ ID NO: 17.

29-32. (canceled)

33. The gene editing system of claim 1, wherein the PBS is 5-100-nucleotide in length, optionally 10-60-nucleotide in length, preferably 10-30-nucleotide in length; and/or wherein the template sequence is 5-100-nucleotide in length, optionally 30-50-nucleotide in length.

34. The gene editing system of claim 1, wherein the PBS binds a PBS-targeting site that:
(i) is adjacent to the complementary region of the target sequence, wherein the PBS-targeting site is upstream to the complementary region of the target sequence,
(ii) overlaps with the complementary region of the target sequence, or
(iii) is adjacent to or overlaps with the target sequence.

35. The gene editing system of claim 34, wherein the PBS-targeting site is 3-10-nucleotide upstream to the complementary region of the target sequence.

36-45. (canceled)

46. The gene editing system of claim 1, wherein the gRNA and the RT donor RNA are located on a single RNA molecule, which comprises the CRISPR nuclease binding site, the spacer sequence, the PBS, and the template sequence.

47-48. (canceled)

49. The gene editing system of claim 46, wherein the single RNA molecule comprises, from 5′ to 3′:
(i) the CRISPR nuclease binding site, the spacer sequence, the template sequence, and the PBS;
(ii) the CRISPR nuclease binding site, the spacer sequence, the linker, the template sequence, and the PBS;
(iii) the template sequence, the PBS, the CRISPR nuclease binding site, and the spacer sequence; or
(iv) the template sequence, the PBS, the linker, the CRISPR nuclease binding site, and the spacer sequence.

50. The gene editing system of claim 46, wherein the single RNA molecule further comprises a 5′ end protection fragment, a 3′ end protection fragment, or both, each of the 5′ end protection fragment and the 3′ end protection fragment forming a secondary structure, which optionally is a hairpin, a pseudoknot, or a triplex structure; or
wherein the 5′ end protection fragment and/or the 3′ end protection fragment comprises one or more of the CRISPR nuclease binding site, and optionally one or more segments that are not homologous to any human sequence.

51-52. (canceled)

53. The gene editing system of claim 1, wherein the gRNA and the RT donor RNA are two separate RNA molecules.

54-56. (canceled)

57. The gene editing system of claim 1, wherein the system comprises one or more lipid nanoparticles (LNPs), which encompass element (a), (b), (c), (d), or any combination thereof.

58-61. (canceled)

62. A pharmaceutical composition comprising the system of claim 1.

63. A kit comprising the elements of (a)-(d) of the system set forth in claim 1.

64. A method for genetically editing a cell, the method comprising contacting a host cell with the gene editing system of claim 1 or a pharmaceutical composition comprising the gene editing system to genetically edit the host cell.

65-66. (canceled)

67. A population of genetically modified cells, which is produced by the gene editing system of claim 1.

68-69. (canceled)

70. A gene editing RNA molecule, or a set of gene editing RNA molecules, comprising or collectively comprising:
(i) one or more binding sites recognizable by a Type V CRISPR nuclease (CRISPR nuclease binding sites);
(ii) a spacer sequence specific to a target sequence within a genetic site, the target sequence being adjacent to a protospacer adjacent motif (PAM);
(iii) a primer binding site (PBS); and
(iv) a template sequence.

71-88. (canceled)
